SEQUENCE LISTING (169 sequences)

SEQ ID NO: 1 - DNA, 9 bp, Homo sapiens
caagtaagt

SEQ ID NO: 2 - DNA, 12 bp, Homo sapiens
gattaagtgggt

SEQ ID NO: 3 - DNA, 12 bp, Homo sapiens
cactaggtgaga

SEQ ID NO: 4 - DNA, 10 bp, Homo sapiens
ccagtgagga

SEQ ID NO: 5 - RNA, 9 nt, Homo sapiens
caaguaagu

SEQ ID NO: 6 - RNA, 12 nt, Homo sapiens
gauuaagugggu

SEQ ID NO: 7 - RNA, 12 nt, Homo sapiens
cacuaggugaga

SEQ ID NO: 8 - RNA, 10 nt, Homo sapiens
ccagugagga

SEQ ID NO: 9 - DNA, 9 bp, Homo sapiens
caagtaagc

SEQ ID NO: 10 - DNA, 12 bp, Homo sapiens
gattaagtaggt

SEQ ID NO: 11 - DNA, 12 bp, Homo sapiens
cactaggtgagc

SEQ ID NO: 12 - DNA, 10 bp, Homo sapiens
ccagttagga

SEQ ID NO: 13 - DNA, 12 bp, Homo sapiens
agccaagtatgt

SEQ ID NO: 14 - DNA, 12 bp, Homo sapiens
atccaagtatgt

SEQ ID NO: 15 - DNA, 11 bp, Homo sapiens
ctgaagtcagt

SEQ ID NO: 16 - DNA, 10 bp, Homo sapiens
agtgtaagta

SEQ ID NO: 17 - RNA, 9 nt, Homo sapiens
caaguaagc

SEQ ID NO: 18 - RNA, 12 nt, Homo sapiens
gauuaaguaggu

SEQ ID NO: 19 - RNA, 12 nt, Homo sapiens
cacuaggugagc

SEQ ID NO: 20 - RNA, 10 nt, Homo sapiens
ccaguuagga

SEQ ID NO: 21 - RNA, 12 nt, Homo sapiens
agccaaguaugu

SEQ ID NO: 22 - RNA, 12 nt, Homo sapiens
auccaaguaugu

SEQ ID NO: 23 - RNA, 11 nt, Homo sapiens
cugaagucagu

SEQ ID NO: 24 - RNA, 10 nt, Homo sapiens
aguguaagua

SEQ ID NO: 25 - DNA, 5 bp, Artificial Sequence
  Synthetic oligonucleotide
cttag

SEQ ID NO: 26 - DNA, 5 bp, Artificial Sequence
  Synthetic oligonucleotide
  misc_feature (1)..(2): n is a, c, g, or t
nnagc

SEQ ID NO: 27 - DNA, 5 bp, Artificial Sequence
  Synthetic oligonucleotide
  misc_feature (1)..(1): n is a, c, g, or t
ncaga

SEQ ID NO: 28 - DNA, 5 bp, Artificial Sequence
  Synthetic oligonucleotide
ccctt

SEQ ID NO: 29 - DNA, 5 bp, Artificial Sequence
  Synthetic oligonucleotide
  misc_feature (2)..(3): n is a, c, g, or t
tnnct

SEQ ID NO: 30 - DNA, 5 bp, Artificial Sequence
  Synthetic oligonucleotide
gcaga

SEQ ID NO: 31 - DNA, 5 bp, Artificial Sequence
  Synthetic oligonucleotide
  misc_feature (2)..(2): n is a, c, g, or t
tntga

SEQ ID NO: 32 - DNA, 5 bp, Artificial Sequence
  Synthetic oligonucleotide
tgagc

SEQ ID NO: 33 - DNA, 5 bp, Artificial Sequence
  Synthetic oligonucleotide
ctctc

SEQ ID NO: 34 - DNA, 5 bp, Artificial Sequence
  Synthetic oligonucleotide
  misc_feature (2)..(4): n is a, c, g, or t
tnnnt

SEQ ID NO: 35 - DNA, 5 bp, Artificial Sequence
  Synthetic oligonucleotide
ttttt

SEQ ID NO: 36 - DNA, 5 bp, Artificial Sequence
  Synthetic oligonucleotide
  misc_feature (5)..(5): n is a, c, g, or t
atggn

SEQ ID NO: 37 - DNA, 5 bp, Artificial Sequence
  Synthetic oligonucleotide
tctca

SEQ ID NO: 38 - DNA, 5 bp, Artificial Sequence
  Synthetic oligonucleotide
aagct

SEQ ID NO: 39 - DNA, 5 bp, Artificial Sequence
  Synthetic oligonucleotide
gtcat

SEQ ID NO: 40 - DNA, 5 bp, Artificial Sequence
  Synthetic oligonucleotide
gagag

SEQ ID NO: 41 - DNA, 5 bp, Artificial Sequence
  Synthetic oligonucleotide
  misc_feature (5)..(5): n is a, c, g, or t
aggan

SEQ ID NO: 42 - DNA, 5 bp, Artificial Sequence
  Synthetic oligonucleotide
agacc

SEQ ID NO: 43 - DNA, 5 bp, Artificial Sequence
  Synthetic oligonucleotide
aaggt

SEQ ID NO: 44 - DNA, 5 bp, Artificial Sequence
  Synthetic oligonucleotide
gatta

SEQ ID NO: 45 - DNA, 5 bp, Artificial Sequence
  Synthetic oligonucleotide
tcttt

SEQ ID NO: 46 - DNA, 5 bp, Artificial Sequence
  Synthetic oligonucleotide
  misc_feature (1)..(2): n is a, c, g, or t
  misc_feature (5)..(5): n is a, c, g, or t
nnagn

SEQ ID NO: 47 - DNA, 5 bp, Artificial Sequence
  Synthetic oligonucleotide
tcgtg

SEQ ID NO: 48 - DNA, 5 bp, Artificial Sequence
  Synthetic oligonucleotide
  misc_feature (2)..(2): n is a, c, g, or t
angaa

SEQ ID NO: 49 - DNA, 5 bp, Artificial Sequence
  Synthetic oligonucleotide
ttaaa

SEQ ID NO: 50 - DNA, 5 bp, Artificial Sequence
  Synthetic oligonucleotide
agact

SEQ ID NO: 51 - DNA, 5 bp, Artificial Sequence
  Synthetic oligonucleotide
gggtg

SEQ ID NO: 52 - DNA, 5 bp, Artificial Sequence
  Synthetic oligonucleotide
ttccc

SEQ ID NO: 53 - DNA, 5 bp, Artificial Sequence
  Synthetic oligonucleotide
  misc_feature (4)..(4): n is a, c, g, or t
agtna

SEQ ID NO: 54 - DNA, 5 bp, Artificial Sequence
  Synthetic oligonucleotide
tatgt

SEQ ID NO: 55 - DNA, 5 bp, Artificial Sequence
  Synthetic oligonucleotide
gggaa

SEQ ID NO: 56 - DNA, 5 bp, Artificial Sequence
  Synthetic oligonucleotide
  misc_feature (1)..(1): n is a, c, g, or t
  misc_feature (4)..(5): n is a, c, g, or t
ntgnn

SEQ ID NO: 57 - DNA, 5 bp, Artificial Sequence
  Synthetic oligonucleotide
  misc_feature (1)..(1): n is a, c, g, or t
ntccc

SEQ ID NO: 58 - DNA, 5 bp, Artificial Sequence
  Synthetic oligonucleotide
caggc

SEQ ID NO: 59 - DNA, 5 bp, Artificial Sequence
  Synthetic oligonucleotide
  misc_feature (1)..(1): n is a, c, g, or t
  misc_feature (5)..(5): n is a, c, g, or t
ntgtn

SEQ ID NO: 60 - DNA, 5 bp, Artificial Sequence
  Synthetic oligonucleotide
  misc_feature (1)..(1): n is a, c, g, or t
ntcct

SEQ ID NO: 61 - DNA, 5 bp, Artificial Sequence
  Synthetic oligonucleotide
aacct

SEQ ID NO: 62 - DNA, 5 bp, Artificial Sequence
  Synthetic oligonucleotide
  misc_feature (5)..(5): n is a, c, g, or t
actcn

SEQ ID NO: 63 - DNA, 5 bp, Artificial Sequence
  Synthetic oligonucleotide
ctgta

SEQ ID NO: 64 - RNA, 5 nt, Artificial Sequence
  Synthetic oligonucleotide
cuuag

SEQ ID NO: 65 - RNA, 5 nt, Artificial Sequence
  Synthetic oligonucleotide
  misc_feature (1)..(2): n is a, c, g, or u
nnagc

SEQ ID NO: 66 - RNA, 5 nt, Artificial Sequence
  Synthetic oligonucleotide
  misc_feature (1)..(1): n is a, c, g, or u
ncaga

SEQ ID NO: 67 - RNA, 5 nt, Artificial Sequence
  Synthetic oligonucleotide
cccuu

SEQ ID NO: 68 - RNA, 5 nt, Artificial Sequence
  Synthetic oligonucleotide
  misc_feature (2)..(3): n is a, c, g, or u
unncu

SEQ ID NO: 69 - RNA, 5 nt, Artificial Sequence
  Synthetic oligonucleotide
gcaga

SEQ ID NO: 70 - RNA, 5 nt, Artificial Sequence
  Synthetic oligonucleotide
  misc_feature (2)..(2): n is a, c, g, or u
unuga

SEQ ID NO: 71 - RNA, 5 nt, Artificial Sequence
  Synthetic oligonucleotide
ugagc

SEQ ID NO: 72 - RNA, 5 nt, Artificial Sequence
  Synthetic oligonucleotide
cucuc

SEQ ID NO: 73 - RNA, 5 nt, Artificial Sequence
  Synthetic oligonucleotide
  misc_feature (2)..(4): n is a, c, g, or u
unnnu

SEQ ID NO: 74 - RNA, 5 nt, Artificial Sequence
  Synthetic oligonucleotide
uuuuu

SEQ ID NO: 75 - RNA, 5 nt, Artificial Sequence
  Synthetic oligonucleotide
  misc_feature (5)..(5): n is a, c, g, or u
auggn

SEQ ID NO: 76 - RNA, 5 nt, Artificial Sequence
  Synthetic oligonucleotide
ucuca

SEQ ID NO: 77 - RNA, 5 nt, Artificial Sequence
  Synthetic oligonucleotide
aagcu

SEQ ID NO: 78 - RNA, 5 nt, Artificial Sequence
  Synthetic oligonucleotide
gucau

SEQ ID NO: 79 - RNA, 5 nt, Artificial Sequence
  Synthetic oligonucleotide
gagag

SEQ ID NO: 80 - RNA, 5 nt, Artificial Sequence
  Synthetic oligonucleotide
  misc_feature (5)..(5): n is a, c, g, or u
aggan

SEQ ID NO: 81 - RNA, 5 nt, Artificial Sequence
  Synthetic oligonucleotide
agacc

SEQ ID NO: 82 - RNA, 5 nt, Artificial Sequence
  Synthetic oligonucleotide
aaggu

SEQ ID NO: 83 - RNA, 5 nt, Artificial Sequence
  Synthetic oligonucleotide
gauua

SEQ ID NO: 84 - RNA, 5 nt, Artificial Sequence
  Synthetic oligonucleotide
ucuuu

SEQ ID NO: 85 - RNA, 5 nt, Artificial Sequence
  Synthetic oligonucleotide
  misc_feature (1)..(2): n is a, c, g, or u
  misc_feature (5)..(5): n is a, c, g, or u
nnagn

SEQ ID NO: 86 - RNA, 5 nt, Artificial Sequence
  Synthetic oligonucleotide
ucgug

SEQ ID NO: 87 - RNA, 5 nt, Artificial Sequence
  Synthetic oligonucleotide
  misc_feature (2)..(2): n is a, c, g, or u
angaa

SEQ ID NO: 88 - RNA, 5 nt, Artificial Sequence
  Synthetic oligonucleotide
uuaaa

SEQ ID NO: 89 - RNA, 5 nt, Artificial Sequence
  Synthetic oligonucleotide
agacu

SEQ ID NO: 90 - RNA, 5 nt, Artificial Sequence
  Synthetic oligonucleotide
gggug

SEQ ID NO: 91 - RNA, 5 nt, Artificial Sequence
  Synthetic oligonucleotide
uuccc

SEQ ID NO: 92 - RNA, 5 nt, Artificial Sequence
  Synthetic oligonucleotide
  misc_feature (4)..(4): n is a, c, g, or u
aguna

SEQ ID NO: 93 - RNA, 5 nt, Artificial Sequence
  Synthetic oligonucleotide
uaugu

SEQ ID NO: 94 - RNA, 5 nt, Artificial Sequence
  Synthetic oligonucleotide
gggaa

SEQ ID NO: 95 - RNA, 5 nt, Artificial Sequence
  Synthetic oligonucleotide
  misc_feature (1)..(1): n is a, c, g, or u
  misc_feature (4)..(5): n is a, c, g, or u
nugnn

SEQ ID NO: 96 - RNA, 5 nt, Artificial Sequence
  Synthetic oligonucleotide
  misc_feature (1)..(1): n is a, c, g, or u
nuccc

SEQ ID NO: 97 - RNA, 5 nt, Artificial Sequence
  Synthetic oligonucleotide
caggc

SEQ ID NO: 98 - RNA, 5 nt, Artificial Sequence
  Synthetic oligonucleotide
  misc_feature (1)..(1): n is a, c, g, or u
  misc_feature (5)..(5): n is a, c, g, or u
nugun

SEQ ID NO: 99 - RNA, 5 nt, Artificial Sequence
  Synthetic oligonucleotide
  misc_feature (1)..(1): n is a, c, g, or u
nuccu

SEQ ID NO: 100 - RNA, 5 nt, Artificial Sequence
  Synthetic oligonucleotide
aaccu

SEQ ID NO: 101 - RNA, 5 nt, Artificial Sequence
  Synthetic oligonucleotide
  misc_feature (5)..(5): n is a, c, g, or u
acucn

SEQ ID NO: 102 - RNA, 5 nt, Artificial Sequence
  Synthetic oligonucleotide
cugua

SEQ ID NO: 103 - DNA, 15 bp, Artificial Sequence
  Synthetic oligonucleotide
ttttttttttttttt

SEQ ID NO: 104 - DNA, 6 bp, Artificial Sequence
  Synthetic oligonucleotide; mutated 5′ splice site
gtaagc

SEQ ID NO: 105 - DNA, 5 bp, Artificial Sequence
  Synthetic oligonucleotide; enrichment motif
ttaca

SEQ ID NO: 106 - DNA, 5 bp, Artificial Sequence
  Synthetic oligonucleotide; enrichment motif
aggta

SEQ ID NO: 107 - DNA, 12 bp, Artificial Sequence
  Synthetic oligonucleotide; mutated 5′ splice site
agccaagtaggc

SEQ ID NO: 108 - DNA, 18 bp, Artificial Sequence
  Synthetic oligonucleotide; mutated 5′ splice site
agtgtgagtccttcacat

SEQ ID NO: 109 - DNA, 18 bp, Artificial Sequence
  Synthetic oligonucleotide; enrichment motif
agtgtgagtccttcacat

SEQ ID NO: 110 - DNA, 5 bp, Artificial Sequence
  Synthetic oligonucleotide; enrichment motif
aaggt

SEQ ID NO: 111 - DNA, 19 bp, Artificial Sequence
  Synthetic oligonucleotide; primer
cctgagcagcaatcatgtg

SEQ ID NO: 112 - DNA, 18 bp, Artificial Sequence
  Synthetic oligonucleotide; primer
cgcagtggttcctttagc

SEQ ID NO: 113 - DNA, 23 bp, Artificial Sequence
  Synthetic oligonucleotide; primer
gctgtgatttaccctcagtcagc

SEQ ID NO: 114 - DNA, 18 bp, Artificial Sequence
  Synthetic oligonucleotide; primer
gcagaaagaggagcgtta

SEQ ID NO: 115 - DNA, 18 bp, Artificial Sequence
  Synthetic oligonucleotide; primer
gctacagaggactggacg

SEQ ID NO: 116 - DNA, 20 bp, Artificial Sequence
  Synthetic oligonucleotide; primer
gatatagtgctgcagcgtgc

SEQ ID NO: 117 - DNA, 23 bp, Artificial Sequence
  Synthetic oligonucleotide; primer
gcctttcttcctgttctttcacg

SEQ ID NO: 118 - DNA, 21 bp, Artificial Sequence
  Synthetic oligonucleotide; primer
cgacccagcacctcttaattc

SEQ ID NO: 119 - DNA, 21 bp, Artificial Sequence
  Synthetic oligonucleotide; primer
gcactgctggtactgaggaag

SEQ ID NO: 120 - DNA, 20 bp, Artificial Sequence
  Synthetic oligonucleotide; primer
aacaagccaaggagccaaga

SEQ ID NO: 121 - DNA, 20 bp, Artificial Sequence
  Synthetic oligonucleotide; primer
tgccctgaagctgatgaagg

SEQ ID NO: 122 - DNA, 21 bp, Artificial Sequence
  Synthetic oligonucleotide; primer
ggagaagtacctgatgccgac

SEQ ID NO: 123 - DNA, 20 bp, Artificial Sequence
  Synthetic oligonucleotide; primer
gatggttctacacagccacc

SEQ ID NO: 124 - DNA, 20 bp, Artificial Sequence
  Synthetic oligonucleotide; primer
ctggtttggagaggcctgtg

SEQ ID NO: 125 - DNA, 20 bp, Artificial Sequence
  Synthetic oligonucleotide; primer
gtaccgtggtgcgatgacta

SEQ ID NO: 126 - DNA, 20 bp, Artificial Sequence
  Synthetic oligonucleotide; primer
gcctcgagttgtcctgactc

SEQ ID NO: 127 - DNA, 20 bp, Artificial Sequence
  Synthetic oligonucleotide; primer
cgatcgtcagggttttgtgc

SEQ ID NO: 128 - DNA, 20 bp, Artificial Sequence
  Synthetic oligonucleotide; primer
ctgagggacagtctggggtt

SEQ ID NO: 129 - DNA, 20 bp, Artificial Sequence
  Synthetic oligonucleotide; primer
gtcagtgagcggagtctgag

SEQ ID NO: 130 - DNA, 20 bp, Artificial Sequence
  Synthetic oligonucleotide; primer
aggcagagatggaacgatct

SEQ ID NO: 131 - DNA, 20 bp, Artificial Sequence
  Synthetic oligonucleotide; primer
tcatggttgaactcgcagct

SEQ ID NO: 132 - DNA, 19 bp, Artificial Sequence
  Synthetic oligonucleotide; primer
cccagagtgcgtttttgaa

SEQ ID NO: 133 - DNA, 18 bp, Artificial Sequence
  Synthetic oligonucleotide; primer
gcagtgattatcaccagc

SEQ ID NO: 134 - DNA, 25 bp, Artificial Sequence
  Synthetic oligonucleotide; primer
ttgcacaatatccttttgaagacca

SEQ ID NO: 135 - DNA, 20 bp, Artificial Sequence
  Synthetic oligonucleotide; primer
attgcccttgacccagagtt

SEQ ID NO: 136 - DNA, 20 bp, Artificial Sequence
  Synthetic oligonucleotide; primer
tgtggtggaattgcccttac

SEQ ID NO: 137 - DNA, 18 bp, Artificial Sequence
  Synthetic oligonucleotide; primer
gtggaattgcccttgagc

SEQ ID NO: 138 - DNA, 20 bp, Artificial Sequence
  Synthetic oligonucleotide; primer
ggaattgcccttgatcatct

SEQ ID NO: 139 - DNA, 22 bp, Artificial Sequence
  Synthetic oligonucleotide; primer
acccaagtcgccgtcttccgcc

SEQ ID NO: 140 - DNA, 20 bp, Artificial Sequence
  Synthetic oligonucleotide; primer
tacatggtcttcgtgacatc

SEQ ID NO: 141 - DNA, 17 bp, Artificial Sequence
  Synthetic oligonucleotide; primer
cactgctgctgctgtcg

SEQ ID NO: 142 - DNA, 19 bp, Artificial Sequence
  Synthetic oligonucleotide; primer
cttagcagcctgcggcagc

SEQ ID NO: 143 - DNA, 18 bp, Artificial Sequence
  Synthetic oligonucleotide; primer
gttggccactgcttttcc

SEQ ID NO: 144 - DNA, 21 bp, Artificial Sequence
  Synthetic oligonucleotide; primer
ctagaactggacctgtgctcc

SEQ ID NO: 145 - DNA, 23 bp, Artificial Sequence
  Synthetic oligonucleotide; primer
caactatgttatttgagtgcagg

SEQ ID NO: 146 - DNA, 20 bp, Artificial Sequence
  Synthetic oligonucleotide; primer
ggtgagcacgttgtccttct

SEQ ID NO: 147 - DNA, 20 bp, Artificial Sequence
  Synthetic oligonucleotide; primer
cggtggaatcaggctgcttg

SEQ ID NO: 148 - DNA, 21 bp, Artificial Sequence
  Synthetic oligonucleotide; primer
ctttgccatggtgtctcgtgg

SEQ ID NO: 149 - DNA, 20 bp, Artificial Sequence
  Synthetic oligonucleotide; primer
attcaggccacacttccctg

SEQ ID NO: 150 - DNA, 18 bp, Artificial Sequence
  Synthetic oligonucleotide; primer
ggtgccagcgtctgcttc

SEQ ID NO: 151 - DNA, 19 bp, Artificial Sequence
  Synthetic oligonucleotide; primer
ctcactctcactgctgctg

SEQ ID NO: 152 - DNA, 22 bp, Artificial Sequence
  Synthetic oligonucleotide; primer
cttcccagtagggtgaagtgac

SEQ ID NO: 153 - DNA, 19 bp, Artificial Sequence
  Synthetic oligonucleotide; primer
gggacggactgcaacctca

SEQ ID NO: 154 - DNA, 22 bp, Artificial Sequence
  Synthetic oligonucleotide; primer
tctgacaaatatctggttgcct

SEQ ID NO: 155 - DNA, 20 bp, Artificial Sequence
  Synthetic oligonucleotide; primer
ttcaggcaggaggaagagga

SEQ ID NO: 156 - DNA, 20 bp, Artificial Sequence
  Synthetic oligonucleotide; primer
ttctggagcacctcaacctg

SEQ ID NO: 157 - DNA, 25 bp, Artificial Sequence
  Synthetic oligonucleotide; primer
atggagcaaatataaagctagaagc

SEQ ID NO: 158 - DNA, 20 bp, Artificial Sequence
  Synthetic oligonucleotide; primer
tgaggaggaatccatgctgg

SEQ ID NO: 159 - DNA, 22 bp, Artificial Sequence
  Synthetic oligonucleotide; primer
tgcaaatcaccagctaccttct

SEQ ID NO: 160 - DNA, 22 bp, Artificial Sequence
  Synthetic oligonucleotide; primer
tgaggcacacactcattactga

SEQ ID NO: 161 - DNA, 20 bp, Artificial Sequence
  Synthetic oligonucleotide; primer
cccagtcaaaggcttgaaac

SEQ ID NO: 162 - DNA, 20 bp, Artificial Sequence
  Synthetic oligonucleotide; primer
ggaggaaatatgctctcaac

SEQ ID NO: 163 - DNA, 24 bp, Artificial Sequence
  Synthetic oligonucleotide; primer
ttagcatcttgttctgtttgtgga

SEQ ID NO: 164 - DNA, 21 bp, Artificial Sequence
  Synthetic oligonucleotide; primer
gagcgaataacctggatcagc

SEQ ID NO: 165 - DNA, 20 bp, Artificial Sequence
  Synthetic oligonucleotide; primer
gcagaattgcccttctgata

SEQ ID NO: 166 - DNA, 21 bp, Artificial Sequence
  Synthetic oligonucleotide; primer
cactgtgctggatatctgctg

SEQ ID NO: 167 - DNA, 20 bp, Artificial Sequence
  Synthetic oligonucleotide; primer
aattgcccttctgcagtttg

SEQ ID NO: 168 - DNA, 22 bp, Artificial Sequence
  Synthetic oligonucleotide; primer
caccttgctcaggtcaactggt

SEQ ID NO: 169 - DNA, 4 bp, Artificial Sequence
  Synthetic oligonucleotide; enrichment motif
gtaa

(19) United States
(12) Patent Application Publication
(10) Pub. No.: US 2023/0047208 A1
(43) Pub. Date: Feb. 16, 2023

(54) RNA Splicing Modulation

(71) Applicants: The General Hospital Corporation, Boston, MA (US); PTC Therapeutics, Inc., South Plainfield, NJ (US)

(72) Inventors: Dadi Gao, Cambridge, MA (US); Elisabetta Morini, Cambridge, MA (US); Michael E. Talkowski, Lexington, MA (US); Susan A. Slaugenhaupt, Hull, MA (US); Wencheng Li, Martinsville, NJ (US); Kerstin Annemarie Effenberger, Metuchen, NJ (US); Christopher Robert Trotta, Somerset, NJ (US); Yong Yu, Westfield, NJ (US); Vijayalakshmi Gabbeta, Bridgewater, NJ (US); Amal Dakka, Whitehouse Station, NJ (US); Nikolai A. Naryshkin, East Brunswick, NJ (US)

(21) Appl. No.: 17/790,328

(22) PCT Filed: Jan. 4, 2021
(86) PCT No.: PCT/US2021/012103

§ 371 Date: Jun. 30, 2022

Related U.S. Application Data
(60) Provisional application No. 62/956,616, filed on Jan. 2, 2020.

(51) Int. Cl. A61K 31/519

(52) U.S. Cl. CPC A61K 31/519; C12N 2320/33

(57) ABSTRACT

One aspect described herein is use of a compound to modulate the production of one or more mature RNA isoforms from a gene transcript in a cell. Another aspect described herein is use of a compound in a method to modulate exon inclusion or exon exclusion in one or more mature mRNA isoforms from a gene transcript in a cell.

### PRIORITY CLAIM

This application claims priority to U.S. Provisional Application Ser. No. 62/956,616, filed Jan. 2, 2020, the entire contents of which is hereby incorporated by reference in its entirety.

### JOINT RESEARCH AGREEMENT STATEMENT

This application is subject to a Joint Research Agreement between Applicant, The General Hospital Corporation, and Co-Applicant, PTC Therapeutics, Inc., which was in effect on or before the Jan. 2, 2020 filing date of U.S. Provisional Application Ser. No. 62/956,616.

### SEQUENCE LISTING

This application contains a Sequence Listing submitted electronically in ASCII format, the entire contents of which is hereby incorporated by reference in its entirety.

### INTRODUCTION

One aspect described herein is use of a compound to modulate the production of one or more mature RNA isoforms from a gene transcript in a cell. Another aspect described herein is use of a compound in a method to modulate exon inclusion or exon exclusion in one or more mature RNA isoforms from a gene transcript in a cell.

### BACKGROUND

Splicing is a key control point in human gene expression. Disturbances in splicing due to mutation or aberrant splicing regulatory networks lead to dysregulated protein expression and to a substantial fraction of human diseases. These disturbances represent a promising opportunity for therapeutic intervention. Several classes of active and selective small molecule splicing modulator compounds (SMCs) have recently been identified validating pre-mRNA splicing as a viable intervention point. However, to date there have been few examples of SMCs that effectively target splicing defects and ameliorate pathogenesis.

RNA splicing is a complex and tightly regulated process that removes introns from pre-mRNA transcripts to generate mature mRNA. Differential processing of pre-mRNA is one of the principal mechanisms generating diversity in different cell and tissue types. This process can give rise to functionally different proteins or, can also generate mRNAs with different localization, stability and efficiency of translation through alternative splicing of UTRs. RNA splicing requires the widely conserved spliceosome machinery along with multiple splicing factors¹⁻². The splicing reaction is directed by specific sequences, including the 5′ and 3′ splice sites, the intron branch site, and splice site enhancers and silencers found in both exons and introns¹⁻². Changes in the sequences of these elements, through inherited or sporadic mutations, can result in deficient or aberrant splice site recognition by the spliceosome and lead to mis-splicing of the pre-mRNA transcript. Disruption of splicing regulatory elements can generate aberrant transcripts through complete or partial exon skipping, intron inclusion or mis-regulation of alternative splicing, the outcome of which often generates premature termination codons (PTCs) that lead to nonsense-mediated mRNA decay (NMD) of the transcript or the production of a truncated protein. Conversely, mutations in the UTRs may affect transcript localization, stability or efficiency of translation. Mutations that alter mRNA splicing are known to lead to many human monogenic diseases including spinal muscular atrophy (SMA), neurofibromatosis type 1 (NF1), cystic fibrosis (CF), familial dysautonomia (FD), Duchenne muscular dystrophy (DMD) and myotonic dystrophy (DM), as well as steer to complex diseases such as cancer and diabetes³⁻¹⁸.

The development of drugs that can increase the amount of normal transcript in patients is a new, precisely targeted treatment approach aimed directly at the primary molecular disease mechanism without altering the genome. The development of splicing modulation therapies for DMD (EXONDYS 51™) and SMA (Nusinersen, Risdiplam, Branaplam)⁴²⁻⁴³ has validated the utility of splicing modification as a valuable therapeutic strategy for human disorders.

New therapeutic approaches aimed at identifying and correcting pre-mRNA splicing defects have shown significant promise in many diseases¹⁹⁻⁵⁵. Small molecule SMCs are attractive therapeutic options because they can be orally administered and are typically systemic and therefore distributed in all tissues. Advances in precision medicine and the capability to discover patient-specific mutations have provided a strong impetus to develop new methods to predict drug selectivity to a target disease gene of interest while improving the drug development process and enhancing patient safety in a personalized medicine paradigm.

### SUMMARY

One aspect described herein is a compound for use in a method to modulate the production of one or more mature RNA isoforms from a gene transcript in a cell, wherein the method comprises, contacting the cell with the compound, and wherein the compound is 2-chloro-N-(pyridin-4-ylmethyl)-7H-pyrrolo[2,3-d]pyrimidin-4-amine, having the formula of Compound (I):

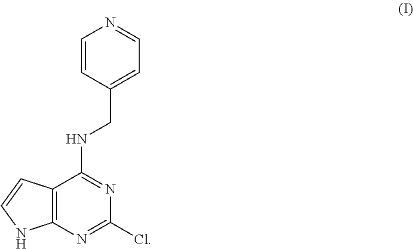 [SMILES: Clc1nc(NCc2ccncc2)c2cc[nH]c2n1]

Another aspect described herein is Compound (I) for use in the method, wherein the gene transcript is a wildtype gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is Compound (I) for use in the method, wherein the 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is Compound (I) for use in the method, wherein the gene transcript is a mutant gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is Compound (I) for use in the method, wherein the 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is Compound (I) for use in the method, wherein Compound (I) modulates exon inclusion or exon exclusion to produce one or more mature RNA isoforms from a gene transcript in a cell, and wherein the method comprises, contacting the cell with Compound (I).

Another aspect described herein is Compound (I) for use in the method, wherein Compound (I) increases exon inclusion to produce one or more mature RNA isoforms from a gene transcript in a cell, and wherein the method comprises, contacting the cell with Compound (I).

Another aspect described herein is Compound (I) for use in the method, wherein Compound (I) increases exon exclusion to produce one or more mature RNA isoforms from a gene transcript in a cell, and wherein the method comprises, contacting the cell with Compound (I).

Another aspect described herein is Compound (I) for use in the method, wherein Compound (I) modulates exon inclusion or exon exclusion to produce one or more mature RNA isoforms from a gene transcript in a cell, and wherein the method comprises, contacting the cell in vivo or in vitro with Compound (I).

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

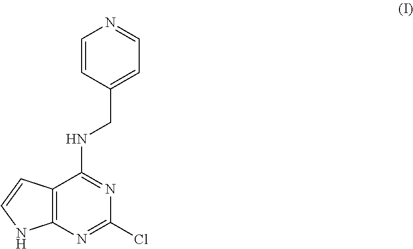 [SMILES: Clc1nc(NCc2ccncc2)c2cc[nH]c2n1]

wherein exon inclusion or exon exclusion is modulated in one or more mature RNA isoforms produced from a gene transcript in a cell.

Another aspect described herein is Compound (I) for use in the method, wherein a gene having the mutated transcript is selected from the group consisting of Table 14 and 15.

Another aspect described herein is Compound (I) for use in the method, wherein a gene having the mutated transcript is selected from the group consisting of Table 14.

Another aspect described herein is Compound (I) for use in the method, wherein a gene having the mutated transcript is other than the gene selected from the group consisting of Table 15.

One aspect described herein is use of a compound to modulate the production of one or more mature RNA isoforms from a gene transcript in a cell comprising, contacting the cell with the compound 2-chloro-N-(pyridin-4-ylmethyl)-7H-pyrrolo[2,3-d]pyrimidin-4-amine, having the formula of Compound (I):

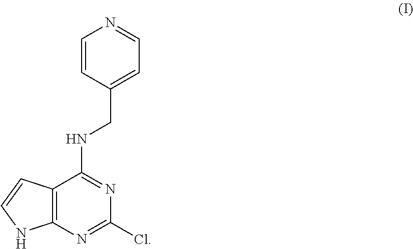 [SMILES: Clc1nc(NCc2ccncc2)c2cc[nH]c2n1]

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is use of Compound (I) to modulate exon inclusion or exon exclusion in one or more mature RNA isoforms from a gene transcript in a cell comprising, contacting the cell with Compound (I).

Another aspect described herein is use of Compound (I) to increase exon inclusion in one or more mature RNA isoforms from a gene transcript in a cell comprising, contacting the cell with Compound (I).

Another aspect described herein is use of Compound (I) to increase exon exclusion in one or more mature RNA isoforms from a gene transcript in a cell comprising, contacting the cell with Compound (I).

Another aspect described herein is use of Compound (I) to modulate exon inclusion or exon exclusion in one or more mature RNA isoforms from a gene transcript in a cell comprising, contacting the cell in vivo or in vitro with Compound (I).

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is use of Compound (I) to treat a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript.

Another aspect described herein is use of Compound (I), wherein a gene having the mutated transcript is selected from the group consisting of Table 14 and 15.

Another aspect described herein is use of Compound (I), wherein a gene having the mutated transcript is selected from the group consisting of Table 14.

Another aspect described herein is use of Compound (I), wherein a gene having the mutated transcript is other than the gene selected from the group consisting of Table 15.

### BRIEF DESCRIPTION OF THE DRAWINGS

FIGS. 1A-1C: Differences Between Compound (I) and Kinetin

FIG. 1A shows the structure of kinetin compared with Compound (I).

FIG. 1B provides a representation of the dual-reporter minigene used to test splicing modulation activity of kinetin and Compound (I). Rluc and Fluc indicate Renilla and Firefly luciferase, respectively. A/C indicates the start codon mutation in Fluc and gtaagC (SEQ ID NO: 104) indicates the location of the mutation that results in exclusion of Exon 20. Dose response curves for kinetin and Compound (I) are shown for the dual-luciferase assay in Rluc-FD-Fluc transfected HEK293T cells treated for 24 hours with kinetin or Compound (I). Exon 20 inclusion, measured by normalized relative luciferase units (RLU), is plotted as a function of compound concentration. Normalized RLU refers to the ratio between firefly and Renilla luciferase and provides a measure of exon 20 inclusion. Assays were run in triplicate and curves were created by nonlinear regression using Prism4 (GraphPad Software Inc.).

FIG. 1C shows Compound (I) splicing modulation activity in human FD fibroblasts. Cells were treated for 24 hours at the concentrations indicated. The experiment was performed in triplicate.

FIGS. 2A-2G: Transcriptome Changes in Response to Compound (I).

FIG. 2A provides a representation of an exon triplet. Exon U is upstream of Exon X, which is upstream of Exon D. R₁, R₂ and R₃ represent RNA sequencing (RNASeq) reads spanning the regions between two adjacent exons. UI₁, I₁X, XI₂ and I₂D are four regions flanking the four splice sites of the exon-triplet. Each flanking region consists of 25 exonic base pairs and 75 intronic base pairs.

FIG. 2B provides a volcano plot showing the ψ (PSI: Percent Spliced-In) changes in splicing after treatment with Compound (I). Each dot represents one of the 161,097 expressed exon triplets in human fibroblasts. The x axis represents the ψ changes after treatment and the y axis represents the False Discovery Rate (FDR) (log 10 transformed). The two vertical dashed lines indicate ψ changes of 0.1 and −0.1 as thresholds for exclusion and inclusion, respectively. The horizontal dashed line indicates an FDR of 0.1. The dots on the right side of the right vertical dashed line and above the horizontal dashed line represent the exon-triplets with an increase for Exon X inclusion (Δψ≥0.1 and FDR<0.1) while the dots on left side of the left vertical dashed line and above the horizontal dashed line represent an increase for Exon X exclusion (Δψ≤−0.1 and FDR<0.1). Black dots in-between the two vertical dashed lines represent exon-triplets unchanged from the treatment.

FIG. 2C shows independent RT-PCR validation of splicing changes of twenty randomly selected candidates after three independent experiments run in duplicate. For each validated exon-triplet, ψ change measured by RNASeq (x axis) is plotted against the splicing changes measured by RT-PCR (y axis). The R² value indicates the coefficient of Pearson correlation. The solid line shows the estimated linear regression. The grey zone indicates the 95% confidence interval for predictions from the estimated linear regression.

FIGS. 2D-2G shows RT-PCR results comparing Compound (I) (Cpd(I)) and kinetin for the splicing response of the four highlighted genes in FIG. 2C: LPINJ (FIG. 2D), HSD17B4 (FIG. 2E), SLC4A7 (FIG. 2F) and CRYZ (FIG. 2G) in human fibroblasts. The upper bands indicate the isoform in which Exon X is included while the lower bands indicate the isoform in which Exon X is skipped.

FIGS. 3A-3G: Convolutional Neural Network (CNN) Prediction of Compound (I) Response

FIGS. 3A1-3A2 show a heatmap of 12 Motifs, with FIG. 3A1 showing CNN Motifs ranked according to predicted contribution in the CNN Model toward the inclusion Class (Motifs 25, 49 and 18), and Motifs ranked according to predicted contribution in the Model toward the exclusion Class (Motifs 47, 21, 29, 9 and 10), whereas FIG. 3A2 shows the Motifs predicted to contribute toward an unchanged response Class (Motifs 01, 22, 27 and 37). The gradience bar indicates the directional contribution of each Motif used to build the dendrogram rows and columns representing the predicted response of each Motif to treatment. The right domain (progressively darker shaded range from 0 to +1) indicates a Motifs predicted positive contribution while the left domain indicates a Motifs predicted negative contribution in a progressively darker shaded range from 0 to −1. The LOGO plot for each Motif is shown on the left side of the heatmap with the corresponding Motif number shown on the right.

FIG. 3A3 shows a heatmap of positional importance for each of the 12 Motifs from FIGS. 3A1-3A2 within the XI₂ region of the 5′ splice site of Exon X. The thick vertical line shows the exon-intron boundary. The gradience bar indicates positional importance, as measured by positional activation in the first layer of the CNN Model, where the right positive domain (shadowed-no slash) suggests the necessity of the Motif and the left domain (shadowed-slash) suggests the absence of the Motif.

FIGS. 3B1-3B2 show a heatmap of 13 Motifs, with FIG. 3B1 showing the Motifs ranked according to predicted contribution in the CNN Model toward the inclusion Class (Motifs 25 and 49), and Motifs ranked according to predicted contribution in the model toward the exclusion Class (Motifs 10, 21, 47, 9 and 40), whereas FIG. 3B2 shows the Motifs ranked according to predicted contribution in the model toward an unchanged response Class (Motifs 16, 33, 27, 35, 22 and 37). The gradience bar indicates the directional contribution of each Motif used to build the dendrogram rows and columns representing the predicted response of each Motif to treatment. The right domain (progressively darker shaded range from 0 to +1) indicates a Motifs predicted positive contribution while the left domain (in a progressively darker shaded range from 0 to −1) indicates a Motifs predicted negative contribution. The LOGO plot for each Motif is shown on the left side of the heatmap with the corresponding Motif number shown on the right.

FIG. 3B3 shows a heatmap of positional importance for each of the 13 Motifs from FIGS. 3B1-3B2 within the XI₂ region of the 5′ splice site of Exon X. The thick vertical line shows the exon-intron boundary. The gradience bar indicates positional importance, as measured by positional activation in the first layer of the CNN model, where the right positive domain (shadowed) suggests the necessity of the Motif and the left domain (progressively less shadowed) suggests the absence of the Motif.

FIG. 3C shows box plots indicating splicing strength for each splice junction along the exon triplets for inclusion (light grey), exclusion (dark grey) and unchanged (black) group, as defined by the RNASeq data based on positional importance as shown in FIGS. 3A3 and 3B3. The middle lines inside the boxes indicate the median and the lower and upper hinges correspond to the first and third quartiles. Each box extends to 1.5 times inter-quartile range (IQR) from upper and lower hinges respectively. Outliers are not shown. Only comparisons with significant difference are marked by stars (two-tailed, unpaired Welch's t test with Bonferroni correction).

FIG. 3D shows the LOGO plot results of Enrichment Motifs from a 5-mer enrichment analysis in adjacent nucleotides from the −3 to +7 position of the 5′ splice sites of the middle exon, and shows their most similar CNN Motifs from the CNN Model having the same Class Effect (e.g. inclusion, exclusion and unchanged) beneath them. Enrichment Motifs AAGGT (SEQ ID NO: 110) and AGTAA (SEQ ID NO: 109) were enriched in inclusion Class sequences detected in the 5-mer enrichment analysis. These Enrichment Motifs looked similar to inclusion Class sequences for CNN Motif 25 (AAGGT) (SEQ ID NO: 43) and CNN Motif 49 (CTGTA) (SEQ ID NO: 63) identified by the CNN model, respectively. Similarly, Enrichment Motif TTACA (SEQ ID NO: 105) was enriched in exclusion Class sequences detected in the 5-mer enrichment analysis. This Enrichment Motif looked similar to the exclusion Class sequence for CNN Motif 29 (TCGTG) (SEQ ID NO: 47) identified by the CNN model. Enrichment Motif AGGTA (SEQ ID NO: 106) was enriched in unchanged Class sequences detected in the 5-mer enrichment analysis. This Enrichment Motif looked similar to the unchanged Class sequence for CNN Motif 22 (AGGAN)(SEQ ID NO: 41) identified by the CNN model.

FIGS. 3E-3G Upper row: The length of the exon triplets cloned into minigenes constructs are shown. The flanking sequences of the 5′ splice sites of Exon X are shown in LOGO plots. The height of each nucleotide was estimated using in silico saturated mutagenesis, with the corresponding closely matched CNN Motifs indicated below the plots. The CPSF7 Minigene in FIG. 3E shows the wildtype 5′ splice site −6 to +6 sequence GATTAAgtgggt (SEQ ID NO: 2), correlated with Motif 25: AAGGT (SEQ ID NO: 43) and compared with the mutated 5′ splice site −6 to +6 sequence GATTAAGTAGGT (SEQ ID NO: 10), correlated with Motif 25: AAGGT (SEQ ID NO: 43) and Motif 49: CTGTA (SEQ ID NO: 63). The SETD5 Minigene in FIG. 3F shows the wildtype 5′ splice site −6 to +6 sequence CACTAGgtgaga (SEQ ID NO: 3), correlated with Motif 10: TGAGC (SEQ ID NO: 32) and Motif 21: GAGAG (SEQ ID NO: 40) and compared with the mutated 5′ splice site −6 to +6 sequence CACTAGgtgagc (SEQ ID NO: 11), correlated with Motif 10: TGAGC (SEQ ID NO: 32). The PARP6 Minigene in FIG. 3G shows the wildtype 5′ splice site −3 to +7 sequence CCAgtgagga (SEQ ID NO: 4), correlated with Motif 26: GATTA (SEQ ID NO: 44) and compared with the mutated 5′ splice site −3 to +7 sequence CCAgttagga (SEQ ID NO: 12), correlated with Motif 32: TTAAA (SEQ ID NO: 49). Middle row: Splicing changes for Exon X in both wildtype and mutated exon triplets, predicted by the CNN model (left) and measured by RT-PCR of the minigene (right). The RT-PCR experiments were performed in duplicate and independently repeated three times for each minigene to make the bar plots (two-tailed, unpaired Student's t test). Bottom row: Example of splicing changes induced by treatment with Compound (I) in the minigene splicing assays. The percentage for Exon X inclusion is indicated beneath each lane, from treated and untreated conditions in both wildtype and mutated minigenes. The upper bands indicate the isoforms in which Exon X is included while the lower bands indicate the isoforms in which Exon X is skipped. The statistical significance, as shown in FIGS. 3E-3G, was determined via two-tailed, unpaired Student's test: *p<0.05; **p<0.01; ***p<0.001.

FIGS. 4A-4F Identification of Compound (I) Targets

FIG. 4A shows the workflow for identification of potential therapeutic targets for Compound (I) as derived from ClinVar pathogenic mutations (CV-pMUTs). SpliceAI was applied to identify all ClinVar pathogenic mutations (CV-pMUTs) and the CNN model was used to determine whether counts per million (CPMs) disrupting annotated splice sites would be rescued by Compound (I) treatment (left). The bar plot shows the percentage of each filtered result out of the total number of disrupting annotated splice sites (right).

FIG. 4B shows the violin plot of the distance from either all CV-pMUTs or the CV-pMUTs disrupting annotated splicing to the closest splice junction. The y axis is in a log₁₀-transformed scale. Each violin shape shows the distribution of distance. The horizontal dashed line indicates 75 nucleotides from the closest splice junction. The significance of difference is determined using the Kolmogorov-Smirnov (K-S) test.

FIGS. 4C-4F Upper row: The sequences at the 5′ splice site of Exon X in patient cells and minigene constructs are shown (FIGS. 4C-4F). The sequences around the 5′ splice site of Exon X are shown in LOGO plots, with the closely matched CNN motifs indicated beneath the plots. The LIPA patient cell minigene in FIG. 4C shows the cell line mutated 5′ splice site −6 to +6 sequence AGCCAAgtaggc (SEQ ID NO: 107), correlated with Motif 25: AAGGT (SEQ ID NO: 43) and Motif 49: CTGTA (SEQ ID NO: 63). The CFTR patient cell minigene in FIG. 4D shows the cell line mutated 5′ splice site −6 to +6 sequence ATCCAAgtatgt (SEQ ID NO: 14), correlated with Motif 25: AAGGT (SEQ ID NO: 43) and Motif 49: CTGTA (SEQ ID NO: 63). The MLH1 patient cell minigene in FIG. 4E shows the cell line mutated 5′ splice site −5 to +6 sequence CTGAAGtcagt (SEQ ID NO: 15), correlated with Motif 18: AAGCT (SEQ ID NO: 38). The MAPT patient cell minigene in FIG. 4F shows the cell line mutated 5′ splice site −3 to +16 sequence AGTgtgagtccttcacat (SEQ ID NO: 108), correlated with Motif 44: NTGNN (SEQ ID NO: 56) and Motif 38: TATGT (SEQ ID NO: 54). The bar plots demonstrate the CNN model prediction of Compound (I) response for the mutated sequences. Middle row: RT-PCR experiments validated treatment responses in patient cell lines carrying specific splice site mutations. The upper bands indicate the isoform in which the middle Exon X is included while the lower bands indicate the isoform in which the middle Exon X is skipped. Each set of gels is one of the triplicates used to generate the bar plots beneath. Bottom row: The bar plots demonstrate the splicing change promoted by Compound (I) treatment. The statistical significance, as shown in FIGS. 4C-4F, was determined via two-tailed, unpaired Student's test: *p<0.05; **p<0.01; ***p<0.001.

FIGS. 5A-5C CNN Model Training Process

FIGS. 5A1-5A2 show details of the CNN model workflow. For each exon-triplet, as shown in FIG. 5A1, the sequences from UI₁, I₁X, XI₂ and I₂D are concatenated and then one-hot coded. In the model, two rounds of convolution were applied before the hidden layer. Each round of convolution consists of a convolution layer of fifty filters, a ReLU activation layer and a max pooling layer of size 2. After two rounds of convolution, the output is converted connected to a hidden layer with 90% dropout rate. The output from the hidden layer shown in FIG. 5A2 is ReLU transformed again and is then linearly transformed into a vector of three, representing three different treatment responses. The final sigmoid nonlinearity maps each element in the vector to a value between 0 and 1, considered as the probability of Compound (I) responsiveness.

FIG. 5B shows the training progress of the CNN model. The x axis represents the number of Epochs iterated during training. The leftmost y axis shows loss score measured by binary entropy while the rightmost y axis shows the average AUC of prediction from three classes. The vertical dashed midline at approximately 12 Epochs on the x axis indicates the stop of training to avoid overfitting. The light grey negative slope plot line shows training loss and the darker grey negative slope plot line shows validation loss respectively along the growth of Epochs shown on the x-axis. The positive slope black plot line shows the improvement of AUC along the growth of Epochs shown on the x-axis.

FIG. 5C shows the AUC curves of prediction for each class using the test set. The x axis represents specificity while the y axis represents sensitivity. The grey negative slope diagonal line indicates the boundary beneath which the prediction is no better than a random guess. The AUC for inclusion, exclusion and unchanged response are shown as the rightmost grey plot line, the middle darker grey plot line and the leftmost black plot line adjacent to the grey diagonal line, respectively.

FIGS. 6A-6B: Motifs Identified by the CNN Model

FIGS. 6A1-6A7 show a heatmap of all CNN Motifs identified by the CNN Model. The color of the Drug Response Contribution in the gradience bar indicates the directional contribution of each motif. The right side domain (progressively shaded in the darker range from 0 to +1) indicates positive contribution while the left side domain (progressively shaded in the lighter range from 0 to −1) indicates negative contribution. The LOGO plot of each motif is shown on the left side of the heatmap, with the motif number shown on the right. The motif response is built in three columns.

FIGS. 6A1-6A2 indicate a Class Effect, having contribution toward the inclusion Class (darker positive range in left column).

FIGS. 6A3-6A5 indicate a Class Effect, having contribution toward the exclusion Class (darker positive range in middle column).

FIGS. 6A6-6A7 indicate a Class Effect, having contribution toward an unchanged Class (darker positive range in right column).

FIGS. 6B1-6B4 show a heatmap of motif importance at each 100 nucleotides in the UI₁, I₁X, XI₂ and I₂D regions, each of which consists of 25 nucleotides in the exon and 75 nucleotides in the intron. Each thick vertical line shows the exon-intron boundary. The gradience bar indicates positional importance, as measured by positional activation in the first layer of the CNN model, where the right positive domain (shadowed) suggests the necessity of the motif and the left negative domain (shadowed) suggests the absence of the motif. Only the top twelve motifs are shown according to FIGS. 3A1-3A2. The motifs are grouped based on their response classification, representing inclusion (Motifs 18, 25 and 49), exclusion (Motifs 09, 10, 21, 29 and 47) and unchanged (Motifs 01, 22, 27 and 37).

FIGS. 7A-7G show the relative expression of full-length (FL) and Δ20 ELP1 mRNA (left panel), and ELP1 protein quantification (right panel) in brain (FIG. 7A) and liver (FIG. 7B) after oral doses of Compound (I) ranging from 10 to 100 mg/kg in adult transgenic TgFD9 mouse (n=4-6 mice in each treatment group). Comparisons were done within the same group, against the vehicle-treated mice. In the figure, *p<0.05; **p<0.01; ***p<0.001.

FIGS. 7C-E show the relative expression of full-length (FL) and Δ20 ELP1 mRNA (left graphs), and ELP1 protein quantification (right graphs) in kidney (FIG. 7C), heart (FIG. 7D) and skin (FIG. 7E) after oral doses of Compound (I) ranging from 10 to 100 mg/kg in adult transgenic TgFD9 mouse (n=4-6 mice in each treatment group). Comparisons were done within the same group, against the vehicle-treated mice. FIG. 7F shows weight assessment of TgFD9 mice in different treatment groups. FIG. 7G shows Compound (I) distribution in the brain, liver, kidney and plasma. The levels of compound were measured using mass spectrometry. In the figure, *p<0.05; **p<0.01; ***p<0.001.

FIG. 8A shows a western blot analysis of LIPA protein in patient fibroblasts carrying the c.894G>A mutation. The top and bottom panels show the blot probed with anti-LIPA and anti-β-Actin antibody, respectively.

FIG. 8B shows a bar chart providing a densitometric analysis of the western blot expressed as percentage of wild-type (WT). LIPA was normalized to β-Actin. To generate the bar plot, the experiment was performed in duplicate and independently repeated three times. The statistical significance is determined via two-tailed, unpaired Student's test: where * p<0.05; ** p<0.01; *** p<0.001.

FIG. 9A shows a western blot analysis of CFTR protein in 293-Flpin cells stably expressing WT-EMG-i14-il8 or c.2988G>A-EMG-i14-il8. 293Flpin cells with no endogenous expression of CFTR protein served as negative control. The top and bottom panels show the blot probed with anti-CFTR and anti-Na+K+ATPase antibody, respectively.

FIG. 9B shows a bar plot providing a densitometric analysis of the western blot expressed as percentage of mature CFTR protein, Band C. The amount of mature CFTR protein was normalized to Na+K+ATPase. To generate the bar plot, the experiment was performed in duplicate and independently repeated three times. the statistical significance is determined via two-tailed, unpaired Student's test: where * p<0.05; ** p<0.01; *** p<0.001.

FIG. 9C shows CFTR chloride channel analysis in CFBE-Flpin cells stably expressing c.2988G>A-EMG-i14-i18 with a representative tracing of short-circuit current (Isc) measurements recorded in Ussing chambers after treatment of cells with either DMSO (vehicle) or variable doses of Compound (I) for 72 h, as indicated by the labeled dose levels for the treatment response. Cells were mounted on Ussing chambers to measure CFTR mediated chloride channel. After stabilization of the basal current, forskolin (10 μM) was added to the basolateral chambers followed by CFTR potentiator, Ivacaftor (10 μM), and CFTR Inhibitor 172 (10 μM) added to the apical chambers.

FIG. 9D shows a stacked bar graph indicating recovery of CFTR function upon treatment of cells with Compound (I). Change in Isc (ΔIsc), a measure of CFTR function, was defined as the current inhibited by Inh-172 after sustained Isc responses were achieved upon stimulation with forskolin alone, indicated by a dark stack within each bar or sequentially with ivacaftor, indicated by a white stack within each bar (n=2 Isc measurements per treatment). The statistical significance is determined via one-way ANOVA when compared with forskolin stimulated CFTR function in DMSO (vehicle) treated cells: where * p<0.05; ** p<0.01; *** p<0.001.

FIGS. 10A-10F show the results of an in silico saturation mutagenesis analysis on selected 100-mer portions of interrogated 400-mer sequences of interest. The indices of the selected sequences in the XI₂ boundary are provided. Each plot includes three parts: 1. The wild-type sequence where letter heights indicate the importance of each nucleotide: The taller the letter, the more important the wild-type nucleotide; 2. A heatmap showing the changes of prediction score (probability of drug response) from the CNN Model when a nucleotide is mutated into the other three nucleotide alternatives: the darker the color, the stronger the change of the prediction score; and, 3. A curve plot demonstrating the highest gain (black line) and loss (grey line) of prediction score (probability of drug response) at each position. FIGS. 10A and 10B represent the XI₂ regions of two Motif sequences known to have an inclusion Class drug response, respectively. FIGS. 10C and 10D represent the XI₂ regions of two Motif sequences known to have an exclusion Class drug response, respectively. FIGS. 10E and 10F represent the XI₂ regions of two Motif sequences known to have an unchanged Class drug response, respectively. The results of this analysis on sequences known to have a particular Class Effect confirm the usefulness of the CNN Model to accurately predict and identify sequences capable of splicing modulation by a small molecule SMC.

FIG. 11 shows a heatmap of Pearson correlation between the Enrichment Motif (each row) in adjacent nucleotides from the −3 to +7 position of the 5′ splice sites of the middle exon and 39 CNN motifs (each column). The range for positive and negative correlation is from 0 to 0.5 and from 0 to −0.5, respectively, where the lightest color indicates a neutral correlation and the progressively darker color toward a positive or negative correlation indicates a progressively stronger correlation. For example, Enrichment Motif AAGGT (SEQ ID NO: 110), known to have an exon inclusion Effect, has the highest average Positive correlation to CNN Motifs (18, 25, 26, 28, 32 and 49) that have a predictive contribution toward an exon inclusion Effect. Enrichment Motif TTACA (SEQ ID NO: 105), known to have an exon exclusion Effect, has the highest average Positive correlation to CNN Motifs (01, 02, 03, 04, 05, 09, 10, 12, 17, 20, 21, 29, 38, 40, 41, 42, 43, 44 and 47) that have a predictive contribution toward an exon exclusion Effect. Enrichment Motif AGGTA (SEQ ID NO: 106), enriched in sequences known to have an unchanged exon Effect against drug treatment, has the highest average correlation with CNN motifs (06, 14, 15, 16, 22, 23, 27, 31, 33, 34, 35, 37, 46 and 48) for no treatment response. Comparatively, Enrichment Motif AGTAA (SEQ ID NO: 109), known to have an exon inclusion Effect, has the highest average correlation with CNN motifs that are responsible for no drug response. This result suggests a potentially false-positive Enrichment Motif detected during the enrichment analysis, where evenly distributed nucleotides A, C, T and G were used as background. Notably, the human genome is biased towards the presence of GTAA (SEQ ID NO: 169), in the +1 to +4 position of the 5′ splice site, thus suggesting Enrichment Motif AGTAA (SEQ ID NO: 109) is close to random. These observations suggest the CNN Model is more capable of efficiently identifying motifs amenable to splicing modulation in the presence of a small molecule SMC, with additional prediction power, than slower classical methods.

FIG. 12A shows the AUCs for the different treatment responses obtained from 1,000 random-initiated CNN models. The diamond indicates the performance of the CNN Model described herein.

FIG. 12B shows the distribution of Pearson correlation between the Motifs shown in FIGS. 3B1-3B2 in the CNN Model described herein and predictive motifs having similar rates of probability in each of the 1,000 random-initiated CNN models.

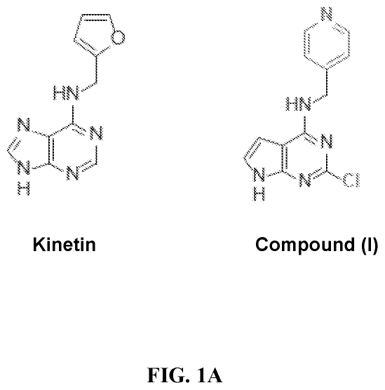

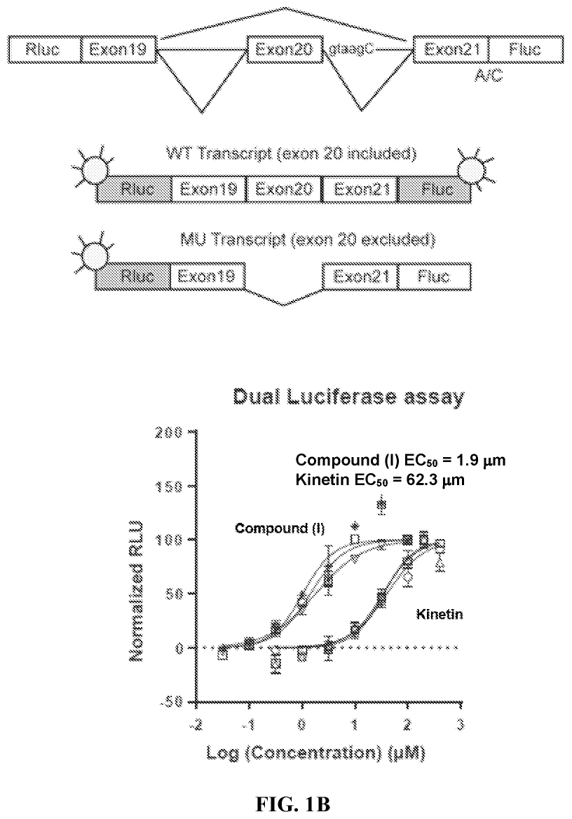

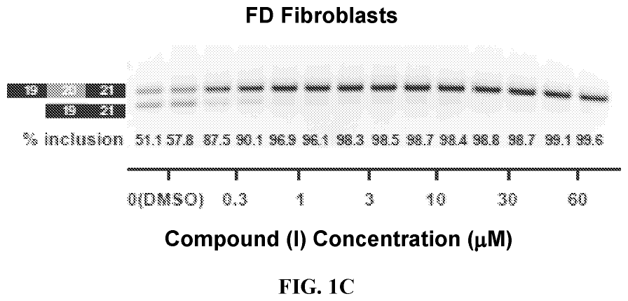

### DETAILED DESCRIPTION

One aspect described herein is a compound for use in a method to modulate the production of one or more mature RNA isoforms from a gene transcript in a cell, wherein the method comprises, contacting the cell with the compound, and wherein the compound is 2-chloro-N-(pyridin-4-ylmethyl)-7H-pyrrolo[2,3-d]pyrimidin-4-amine, having the formula of Compound (I):

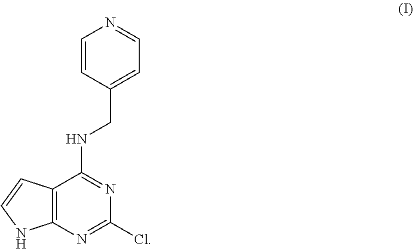 [SMILES: Clc1nc(NCc2ccncc2)c2cc[nH]c2n1]

Another aspect described herein is Compound (I) for use in the method, wherein the gene transcript is a wildtype gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of:

(SEQ ID NO: 1)
CAAgtaagt,
(SEQ ID NO: 2)
GATTAAgtgggt,
(SEQ ID NO: 3)
CACTAGgtgaga,
and
(SEQ ID NO: 4)
CCAgtgagga.

Another aspect described herein is Compound (I) for use in the method, wherein the 5′ splice site of Exon 2 comprises, a sequence selected from the group consisting of:

(SEQ ID NO: 5)
CAAguaagu,
(SEQ ID NO: 6)
GAUUAAgugggu,
(SEQ ID NO: 7)
CACUAGgugaga,
and
(SEQ ID NO: 8)
CCAgugagga.

Another aspect described herein is Compound (I) for use in the method, wherein the gene transcript is a mutant gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of:

(SEQ ID NO: 9)
CAAgtaagc,
(SEQ ID NO: 10)
GATTAAgtaggt,
(SEQ ID NO: 11)
CACTAGgtgagc,
(SEQ ID NO: 12)
CCAgttagga,
(SEQ ID NO: 13)
AGCCAAgtatgt,
(SEQ ID NO: 14)
ATCCAAgtatgt,
(SEQ ID NO: 15)
CTGAAgtcagt,
and
(SEQ ID NO: 16)
AGTgtaagta.

Another aspect described herein is Compound (I) for use in the method, wherein the 5′ splice site of Exon 2 comprises, a sequence selected from the group consisting of:

(SEQ ID NO: 17)
CAAguaagc,
(SEQ ID NO: 18)
GAUUAAguaggu,
(SEQ ID NO: 19)
CACUAGgugagc,
(SEQ ID NO: 20)
CCAguuagga,
(SEQ ID NO: 21)
AGCCAAguaugu,
(SEQ ID NO: 22)
AUCCAAguaugu,
(SEQ ID NO: 23)
CUGAAgucagu,
and
(SEQ ID NO: 24)
AGUguaagua.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is Compound (I) for use in the method, wherein Compound (I) modulates exon inclusion or exon exclusion to produce one or more mature RNA isoforms from a gene transcript in a cell, and wherein the method comprises, contacting the cell with Compound (I).

Another aspect described herein is Compound (I) for use in the method, wherein Compound (I) increases exon inclusion to produce one or more mature RNA isoforms from a gene transcript in a cell, and wherein the method comprises, contacting the cell with Compound (I).

Another aspect described herein is Compound (I) for use in the method, wherein Compound (I) increases exon exclusion to produce one or more mature RNA isoforms from a gene transcript in a cell, and wherein the method comprises, contacting the cell with Compound (I).

Another aspect described herein is Compound (I) for use in the method, wherein Compound (I) modulates exon inclusion or exon exclusion to produce one or more mature RNA isoforms from a gene transcript in a cell, and wherein the method comprises, contacting the cell in vivo or in vitro with Compound (I).

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human,

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

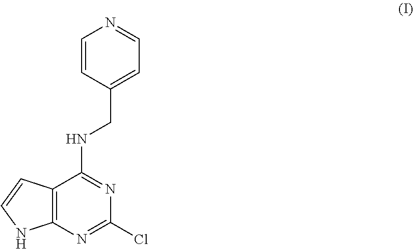 [SMILES: Clc1nc(NCc2ccncc2)c2cc[nH]c2n1]

wherein exon inclusion or exon exclusion is modulated in one or more mature RNA isoforms produced from a gene transcript in a cell.

Another aspect described herein is Compound (I) for use in the method, wherein a gene having the mutated transcript is selected from the group consisting of Table 14 and 15.

Another aspect described herein is Compound (I) for use in the method, wherein a gene having the mutated transcript is selected from the group consisting of Table 14.

Another aspect described herein is Compound (I) for use in the method, wherein a gene having the mutated transcript is other than the gene selected from the group consisting of Table 15.

One aspect described herein is use of a compound to modulate the production of one or more mature RNA isoforms from a gene transcript in a cell comprising, contacting the cell with the compound 2-chloro-N-(pyridin-4-ylmethyl)-7H-pyrrolo[2,3-d]pyrimidin-4-amine, having the formula of Compound (I):

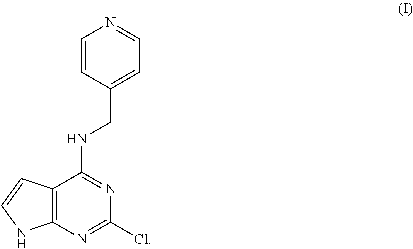 [SMILES: Clc1nc(NCc2ccncc2)c2cc[nH]c2n1]

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from comprises, a sequence the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence s nucleotide sequences of the predicted gene transcript are the 5′ splice site of Exon 2 is selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is use of Compound (I) to modulate exon inclusion or exon exclusion in one or more mature RNA isoforms from a gene transcript in a cell comprising, contacting the cell with Compound (I).

Another aspect described herein is use of Compound (I) to increase exon inclusion in one or more mature RNA isoforms from a gene transcript in a cell comprising, contacting the cell with Compound (I).

Another aspect described herein is use of Compound (I) to increase exon exclusion in one or more mature RNA isoforms from a gene transcript in a cell comprising, contacting the cell with Compound (I).

Another aspect described herein is use of Compound (I) to modulate exon inclusion or exon exclusion in one or more mature RNA isoforms from a gene transcript in a cell comprising, contacting the cell in vivo or in vitro with Compound (I).

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is use of Compound (I) to treat a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript.

Another aspect described herein is use of Compound (I), wherein a gene having a mutation in a gene transcript thereof is selected from the group consisting of Table 14 and 15.

Another aspect described herein is use of Compound (I), wherein a gene having a mutation in a gene transcript thereof is selected from the group consisting of Table 14.

Another aspect described herein is use of Compound (I), wherein a gene having a mutation in a gene transcript thereof is other than the gene selected from the group consisting of Table 15.

### Methods of Modulating Wildtype Gene Isoforms

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 1.

Table 1 lists wildtype genes identified by RNA sequencing and mutated genes predicted by SpliceAI scores and the CNN model, as described herein, which may be modulated by Compound (I) toward either or both exon inclusion and exon exclusion.

TABLE 1
A1BG-AS1, AAAS, ABCA1, ABCA4, ABCA6,
ABCA9, ABCB6, ABCC3, ABCC9,
ABHD14A-ACY1, ABI1, ABI2, ABI3BP, AC024560.3,
AC027612.6, AC037459.4, ACAA1,
ACACA, ACAD10, ACADSB, ACBD4, ACBD5,
ACIN1, ACLY, ACOX1, ACSL3, ACSL4,
ACTN1, ACY1, AD000671.6, ADA, ADAM10,
ADAM33, ADAMTSL4, ADARB1, ADK,
AFMID, AGK, AHI1, AKAP11, AKAP13, AKIP1,
AKR1A1, AL163636.6, ALDH3A2,
ALDH3B1, ALDH4A1, ALKBH1, ALKBH3,
ALMS1, AMMECR1L, AMPD2, ANK2,
ANKRD10, ANKRD11, ANKRD36, ANO6,
ANTXR2, ANXA2, AP1G1, AP2M1, APBB2,
APC, APOL1, APP, ARHGAP12, ARHGAP23,
ARHGEF10, ARHGEF10L, ARHGEF12,
ARHGEF25, ARID4B, ARIH2, ARMC9,
ARSK, ASAH1, ASAP2, ASCC2, ASL, ASPH,
ASPM, ASUN, ASXL1, ATG10, ATG16L1,
ATG7, ATG9A, ATM, ATP2C1, ATP5SL,
ATPAF1, ATRX, ATXN1, ATXN3, AUP1,
AUTS2, AVL9, B3GALNT1, BAG6, BANP,
BAX, BAZ1A, BBS4, BBS5, BBX, BCAR1, BCOR,
BFSP1, BIN1, BIRC6, BLOC1S6,
BMP2K, BMPR2, BOK, BPTF, BRCA1,
BRCA2, BRD8, BRIP1, BROX, BTBD19,
C11orf30, C11orf70, C12orf29, C16orf13,
C1orf85, C1RL, C2CD5, C3orf18, C5orf42,
C5orf45, C8orf59, C9orf156, C9orf85, CA5A,
CA5BP1, CALU, CAMK2D, CAMTA1,
CAPN3, CAPN7, CAPRIN2, CARD8,
CARKD, CASP3, CAST, CCBL2, CCDC25,
CCDC90B, CCNDBP1, CCNL1, CD3D, CD44,
CD46, CD55, CD99P1, CDAN1, CDC14B,
CDC16, CDH1, CDH23, CDK16, CEP290, CERKL,
CFTR, CHD3, CHD7, CHEK2, CHKB,
CKLF, CLASP1, CLCN1, CLCN6, CLK1,
CLMP, CLN3, CMC1, CNGB3, CNOT10, COG6,
COL11A1, COL12A1, COL16A1, COL3A1,
COL4A3, COL4A5, COL5A2, COL6A1,
COL6A3, COL7A1, COPB2, COPS7A, COPS8,
COQ6, COX20, CPEB2, CPSF7, CPT1C,
CRAT, CREBBP, CRLS1, CRNDE, CRYZ,
CSAD, CSTB, CTDSPL, CTNS, CTSK, CTU2,
CUBN, CUTC, CWC25, CWC27, CYB561D2,
CYBB, CYLD, CYP20A1, DAB2, DBT,
DCAF10, DCAF11, DCAF17, DCAF8, DCP1A,
DCUN1D5, DCX, DCX, DDB2, DGKA,
DGKE, DGUOK, DHRSX, DIMT1, DIS3,
DLG1, DMD, DMTF1, DNAH5, DNAH9,
DNAJC19, DNAJC2, DNM1L, DNMT1,
DOCK5, DOCK7, DPH3, DPY19L4, DRAM2,
DSCR3, DTNBP1, DUSP11, DUSP22, DYNC2H1,
DYNC2LI1, EBF3, EBP, EBPL, EDC3,
EDEM2, EDRF1, EFEMP1, EHBP1, EHMT2,
EIF4A2, EIF4E2, EIF4H, ELK1, ELN,
ELOVL1, ENC1, ENOSF1, EP300, EPB41L1,
EPB41L2, ERBB2IP, ERCC6, ERLEC1,
ERMAP, ETHE1, EVC, EVI5L, F10, F13A1, F8,
FAM104A, FAM111A, FAM134C,
FAM149B1, FAM172A, FAM204A, FAM211A-
AS1, FAM21C, FAP, FBN1, FBXL12,
FBXL3, FBXO25, FBXW11, FGF5, FGFR1OP,
FHL2, FIG4, FIP1L1, FLAD1, FLNA, FLT3,
FN1, FNBP4, FNIP2, FOXN3, FRYL, FUT8,
FXR2, FYN, G6PC, GAA, GAB1, GABPB2,
GABRE, GABRG2, GALNS, GCK, GFPT2,
GGCT, GIT2, GK, GLA, GLS, GNB5,
GNPTAB, GOLGA4, GOLGB1, GOLT1B,
GPR133, GPR143, GPR180, GPX4, GRHPR,
GRIPAP1, GRN, GTF2I, GTF3A, GUCY2C, GUF1,
GUSB, GYPA, HACL1, HAUS7, HBB,
HCFC1R1, HDAC10, HDAC7, HECTD3,
HERC3, HFE, HIPK3, HMGXB4, HNRNPA2B1,
HNRNPD, HNRNPDL, HPS1, HSD17B4, HSF2,
HUWE1, IDH1, IFT57, IFT88, IGF2BP2,
IL15RA, IL36RN, INF2, ING4, INO80E, INPP1,
IP6K2, IPO8, IQCB1, IRAK4, ISOC2,
IST1, ITGB1BP1, KATNBL1, KDM4C,
KDM5A, KDSR, KIAA0100, KIAA0586,
KIAA1109, KIF14, KIF3A, KIT, KITLG, KLC1,
KMT2D, KRIT1, KTN1, L3HYPDH,
LACC1, LAMB3, LAMTOR3, LAS1L, LDLR,
LENG8, LETMD1, LGALS8, LHCGR,
LINC00963, LIPA, LMAN2L, LMNA, LMO7,
LONRF1, LOXL3, LPHN2, LPIN1, LRCH3,
LRRC28, LRRC32, LSM1, LTA4H, LTBP1,
LTBP3, LTBP4, LUC7L, LYPLAL1, LYRM7,
LZTFL1, MACF1, MADD, MAGOHB,
MAN2C1, MAP3K3, MAP4K4, MAPK11,
MAPK12, MAPKAP1, MAPT, MARK3,
MBD5, MBNL1, MBOAT2, MCFD2, ME3,
MED13, MED15, MEG3, MEGF6, METAP1,
METTL14, MFF, MFSD12, MGEA5, MGST2,
MINK1, MIR22HG, MKS1, MLF1, MLH1,
MLLT6, MLST8, MMAB, MMP19, MORF4L2,
MOSPD1, MPC1, MPDZ, MPI, MPV17,
MRPL33, MSH2, MSRB3, MTCH2, MTM1,
MTMR2, MTMR3, MTMR6, MTRR, MTSS1L,
MYBL1, MYBPC3, MYCBP2, MYEF2,
MYLK, MYNN, MYO18A, MYO19, MYO1B,
MYO5A, MYO7A, NABP1, NADK2, NAE1,
NBN, NEK1, NEO1, NEXN, NF1, NF2,
NFE2L1, NIPA2, NIPBL, NLRC5, NLRX1,
NPEPPS, NPHP1, NPR2, NPRL3, NR3C2,
NRG1, NSD1, NSFL1C, NSMCE2, NSUN4,
NT5C2, NUB1, NUP43, NUP62, NUPL2,
NUTM2A-AS1, OARD1, OCA2, OCRL, ODF2,
ODF2L, OFD1, OGT, OPTN, ORC6, ORMDL1,
OS9, OSBPL8, OTC, OTOGL, PACRGL,
PAFAH1B1, PAH, PAM, PARD3, PARL, PARN,
PARP11, PARP6, PATL1, PCBP2, PCID2,
PCM1, PCNXL2, PCNXL4, PCYT2, PDCD10,
PDE4DIP, PDLIM2, PDZD11, PEAK1,
PEX1, PEX11A, PEX5, PFDN1, PFDN6, PFKM,
PHC3, PHKG2, PHLDB1, PI4KB, PIGG,
PIGN, PIGT, PIK3C2A, PIKFYVE, PILRB,
PIN1, PKIG, PLA2G12A, PLA2R1, PLAGL1,
PLBD2, PMS2, PMS2P3, PNPLA8, PODNL1,
POLG, POLR3GL, POMGNT1, PPAP2A,
PPFIBP1, PPIL3, PPIP5K2, PPM1M, PPP1R12A,
PPP1R21, PPP2R3C, PPP3CB, PPP3CC,
PPP4C, PPP6R2, PPRC1, PPT1, PREB, PREPL,
PRKAG1, PRMT2, PRMT7, PRPF39,
PRPF40B, PRR16, PRR4, PRRX1, PRUNE,
PSME4, PTEN, PTPN13, PTPN21, PTPN23,
PTPN4, PTPRA, PVR, PXDN, QTRTD1, R3HCC1L,
R3HDM4, RAB11FIP2, RABEPK,
RAD1, RAD51B, RAD51C, RALGPS2, RALY,
RB1, RBBP9, RBCK1, RBM41, RCOR3,
REPS1, REV3L, RFWD2, RGL2, RGN, RHAG,
RHBDD2, RHOBTB1, RHOT1, RIF1,
RIPK2, RNASE4, RNF146, RNF170, RNF214,
RNF216, RNF34, RNFT1, ROBO1, RP11-
1055B8.7, RP11-14N7.2, RP11-274B21.1,
RP11-33B1.1, RP11-383H13.1, RP11-773D16.1,
RP13-279N23.2, RPAIN, RPL7L1, RPP14,
RPS6KB2, RRBP1, RSU1, RTEL1, RTEL1-
TNFRSF6B, RTTN, RUFY2, RWDD1, RWDD2B,
RWDD4, SBF1, SCMH1, SCN1A,
SCN5A, SDCCAG8, SDHD, SEC31B, SECISBP2L,
SEMA4F, SENP1, SENP6, SERAC1,
SETD5, SETX, SFXN4, SGSM3, SH3D19,
SHMT2, SIPA1L1, SLC10A7, SLC12A1,
SLC15A4, SLC22A17, SLC25A17, SLC25A32,
SLC25A36, SLC35A1, SLC35A3,
SLC35D2, SLC37A3, SLC38A2, SLC38A6,
SLC4A11, SLC4A7, SLC5A6, SLC7A6,
SLC9B2, SLIT2, SLMAP, SLMO1, SLTM,
SMAD5, SMARCA1, SMARCD1, SMC5,
SMEK2, SMPDL3A, SMS, SMURF2, SNAPC5,
SNED1, SNHG14, SNHG15, SNRNP70,
SNRPA1, SNRPG, SNX10, SNX14, SNX21,
SPAST, SPATA20, SPATA7, SPDL1, SPG11,
SPPL2A, SPTA1, SPTAN1, SPTB, SRD5A2,
ST6GALNAC6, ST7, STARD3NL, STARD5,
STAT6, STK11, STK16, STK19, STK40,
STRA13, STRADA, STRN3, STX16, STX16-
NPEPL1, STX3, STXBP1, STXBP3, STYXL1,
SUCO, SUGP2, SUGT1, SULF1, SUN2,
SUPT20H, SYNGAP1, SYNRG, TAF1, TAF2,
TANGO2, TARBP2, TAS2R14, TAZ,
TBC1D14, TBC1D17, TBC1D25, TBC1D5,
TBCK, TBPL1, TBX15, TCERG1, TCIRG1,
TEAD2, TECTA, TEP1, TFDP1, TFDP2,
TFPI, TFR2, TGFBR2, THAP6, THAP9-AS1,
THBS3, THOC2, TIA1, TIAL1, TIMM17B,
TIMM21, TJP2, TM7SF3, TMCO4, TMEM11,
TMEM120A, TMEM126B, TMEM138,
TMEM18, TMEM194A, TMEM234, TMEM260,
TMEM39B, TMEM62, TMOD2, TMPRSS6,
TMUB2, TMX3, TOM1, TP53, TP53INP1,
TP53TG1, TPD52L1, TPD52L2, TPO, TPRA1,
TPT1-AS1, TRAPPC12, TRAPPC2, TREX2,
TRIM16, TRIM37, TRIM65, TRIO, TRIP10,
TRIP12, TROVE2, TRPC1, TRPM4, TRPT1,
TSEN15, TSR1, TSTD3, TUBG2, TULP3,
TXNDC11, TXNL4A, TYR, U2AF1L4, UACA,
UBA7, UBE2A, UBE2D1, UBN2, UBP1,
UBQLN1, UFD1L, UNC5B, URGCP, UROD,
USMG5, USP24, USP25, USP33, USP53,
USP8, VAMP7, VCAN, VEZT, VMA21, VPS13B,
VPS29, WBP1, WDFY2, WDR35, WDR73, XDTP53,
XPC, YEATS2, YIPF1, YME1L1,
YY1AP1, ZBTB8OS, ZC2HC1A, ZC3H11A,
ZCCHC6, ZCCHC8, ZDHHC16, ZDHHC20,
ZDHHC7, ZFAND1, ZFAND5, ZFAS1, ZMIZ1,
ZMYM4, ZMYM5, ZNF160, ZNF207,
ZNF248, ZNF280D, ZNF384, ZNF512,
ZNF516, ZNF532, ZNF720, and ZZZ3.

Another aspect described herein is Compound (I) for use in the method, wherein the gene transcript is a wildtype gene transcript comprising, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 1.

Another aspect described herein is Compound (I) for use in the method, wherein the 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 1.

Another aspect described herein is Compound (I) for use in the method, wherein the gene transcript is a mutant gene transcript comprising, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 1.

Another aspect described herein is Compound (I) for use in the method, wherein the 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 1.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 1.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 1.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 1.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 1.

Another aspect described herein is Compound (I) for use in the method, wherein the gene transcript is a wildtype gene transcript comprising, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 1.

Another aspect described herein is Compound (I) for use in the method, wherein the gene transcript is a wildtype gene transcript comprising, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 1.

Another aspect described herein is Compound (I) for use in the method, wherein the gene transcript is a mutant gene transcript comprising, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 1.

Another aspect described herein is Compound (I) for use in the method, wherein the gene transcript is a mutant gene transcript comprising, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 1.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 1.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 1.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 1.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 1.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 1.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 1.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

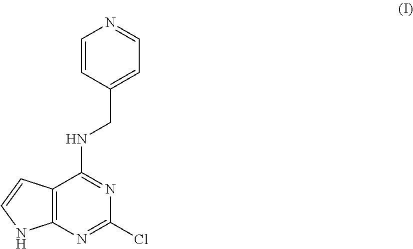 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 1.

Another aspect described herein is Compound (I) for use in the method, wherein a gene having a mutation in a gene transcript thereof is selected from the group consisting of Table 1.

One aspect described herein is use of Compound (I) to modulate the production of one or more mature RNA isoforms from a gene transcript in a cell comprising, contacting the cell with Compound (I), wherein the gene transcript is transcribed from a gene selected from Table 1.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 1.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 1.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 1.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 1.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 1.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 1.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 1.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 1.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 1.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 1.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 1.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 1.

Another aspect described herein is use of Compound (I) to modulate exon inclusion or exon exclusion in one or more mature RNA isoforms from a gene transcript in a cell comprising, contacting the cell with Compound (I), and wherein the gene transcript is transcribed from a gene selected from Table 1.

Another aspect described herein is use of Compound (I) to increase exon inclusion in one or more mature RNA isoforms from a gene transcript in a cell comprising, contacting the cell with Compound (I), and wherein the gene transcript is transcribed from a gene selected from Table 1.

Another aspect described herein is use of Compound (I) to increase exon exclusion in one or more mature RNA isoforms from a gene transcript in a cell comprising, contacting the cell with Compound (I), and wherein the gene transcript is transcribed from a gene selected from Table 1.

Another aspect described herein is use of Compound (I) to modulate exon inclusion or exon exclusion in one or more mature RNA isoforms from a gene transcript in a cell comprising, contacting the cell in vivo or in vitro with Compound (I), and wherein the gene transcript is transcribed from a gene selected from Table 1.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 1.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 1.

Another aspect described herein is use of Compound (I) to treat a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 1.

Another aspect described herein is use of Compound (I), wherein a gene having a mutation in a gene transcript thereof is selected from the group consisting of Table 1.

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 2.

Table 2 lists wildtype genes identified by RNA sequencing and mutated genes predicted by SpliceAI scores and the CNN model, as described herein, which may be modulated by Compound (I) toward both exon inclusion and exon exclusion.

TABLE 2
ASPH, BBS4, BPTF, BRCA1, BRIP1, CD46,
CDC14B, COL6A3, DIS3, DMD, DNM1L,
DRAM2, DYNC2H1, FAM172A, FBN1, FIP1L1,
GIT2, GLS, KLC1, LMAN2L, LPHN2,
MACF1, MAPT, MEG3, MFF, MLH1, MOSPD1,
MSRB3, MTMR2, MYLK, MYNN, MYO5A, OPTN, OTC,
RPAIN, SDCCAG8, SIPA1L1, SLC38A6, SUPT20H, TBCK,
TSEN15, and ZNF207.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 2.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 2.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 2.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 2.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 2.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 2.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 2.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 2.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CTGAAgtcagt (SEQ ID NO: 15), and AGTgtaagta (SEQ ID NO: 16), and wherein the mutant gene transcript is transcribed from a gene selected from Table 2.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CUGAAgucagu (SEQ ID NO: 23), and AGUguaagua (SEQ ID NO: 24), and wherein the mutant gene transcript is transcribed from a gene selected from Table 2.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 2.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 2.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 2.

Another aspect described herein is Compound (I) for use in a method to modulate exon inclusion or exon exclusion in one or more mature RNA isoforms from a gene transcript in a cell, wherein the method comprises, contacting the cell in vivo or in vitro with Compound (I), and wherein the gene transcript is transcribed from a gene selected from Table 2.

Another aspect described herein is Compound (I) for use in a method to treat a disease associated with or mediated by a mutation in a gene transcript in a subject in need thereof, wherein the method comprises, administering Compound (I) to the subject, wherein exon inclusion or exon exclusion is modulated in one or more mature RNA isoforms produced from the gene transcript, wherein the subject is human, and wherein the gene is selected from Table 2.

Another aspect described herein is Compound (I) for use in a method to treat a disease associated with or mediated by a mutation in a gene transcript in a subject in need thereof, wherein the method comprises, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 2.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

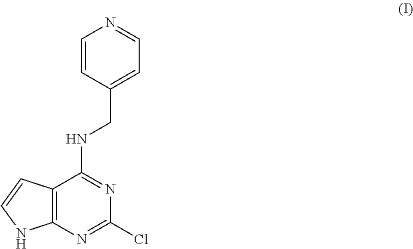 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 2.

Another aspect described herein is Compound (I) for use in the method, wherein a gene having a mutation in a gene transcript thereof is selected from the group consisting of Table 2.

One aspect described herein is use of Compound (I) to modulate the production of one or more mature RNA isoforms from a gene transcript in a cell comprising, contacting the cell with Compound (I), wherein the gene transcript is transcribed from a gene selected from Table 2.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 2.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 2.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 2.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 2.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 2.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 2.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 2.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 2.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CTGAAgtcagt (SEQ ID NO: 15), and AGTgtaagta (SEQ ID NO: 16), and wherein the mutant gene transcript is transcribed from a gene selected from Table 2.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CUGAAgucagu (SEQ ID NO: 23), and AGUguaagua (SEQ ID NO: 24), and wherein the mutant gene transcript is transcribed from a gene selected from Table 2.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 2.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 2.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 2.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 2.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 2.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 2.

Another aspect described herein is use of Compound (I) to treat a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 2.

Another aspect described herein is use of Compound (I), wherein a gene having a mutation in a gene transcript thereof is selected from the group consisting of Table 2.

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 3.

Table 3 lists wildtype genes identified by RNA sequencing, as described herein, that may be modulated by Compound (I) toward either or both exon inclusion and exon exclusion.

TABLE 3
A1BG-AS1, AAAS, ABCA1, ABCA6, ABCA9,
ABCB6, ABCC3, ABHD14A-ACY1, ABI1,
ABI2, ABI3BP, AC024560.3, AC027612.6,
AC037459.4, ACAA1, ACACA, ACAD10,
ACBD4, ACBD5, ACIN1, ACLY, ACOX1, ACSL3,
ACSL4, ACTN1, ACY1, AD000671.6,
ADA, ADAM33, ADAMTSL4, ADARB1, ADK,
AFMID, AHI1, AKAP11, AKAP13,
AKIP1, AKR1A1, AL163636.6, ALDH3B1, ALDH4A1,
ALKBH1, ALKBH3, AMMECR1L,
AMPD2, ANK2, ANKRD10, ANKRD11,
ANKRD36, ANO6, ANXA2, AP1G1, AP2M1,
APBB2, APOL1, APP, ARHGAP12, ARHGAP23,
ARHGEF10, ARHGEF10L, ARHGEF12,
ARHGEF25, ARID4B, ARIH2, ARSK,
ASAP2, ASCC2, ASL, ASPH, ASUN, ASXL1,
ATG10, ATG16L1, ATG7, ATG9A, ATP2C1,
ATP5SL, ATPAF1, ATXN1, ATXN3, AUP1,
AUTS2, AVL9, B3GALNT1, BAG6, BANP,
BAX, BAZ1A, BBS4, BBS5, BBX, BCAR1,
BCOR, BIN1, BIRC6, BLOC1S6, BMP2K,
BOK, BPTF, BRD8, BROX, BTBD19, C11orf30,
C11orf70, C12orf29, C16orf13, C1orf85, C1RL,
C2CD5, C3orf18, C5orf42, C5orf45,
C8orf59, C9orf156, C9orf85, CA5BP1,
CALU, CAMK2D, CAMTA1, CAPN7, CAPRIN2,
CARD8, CARKD, CASP3, CAST, CCBL2,
CCDC25, CCDC90B, CCNDBP1, CCNL1,
CD44, CD46, CD55, CD99P1, CDC14B,
CDC16, CDK16, CEP290, CHD3, CHEK2, CHKB,
CKLF, CLASP1, CLCN6, CLK1, CMC1,
CNOT10, COL12A1, COL16A1, COL6A3,
COPB2, COPS7A, COPS8, COQ6, COX20,
CPEB2, CPSF7, CPT1C, CRAT, CREBBP,
CRLS1, CRNDE, CRYZ, CSAD, CTDSPL,
CUTC, CWC25, CYB561D2, CYLD, CYP20A1,
DAB2, DBT, DCAF10, DCAF11, DCAF17,
DCAF8, DCP1A, DCUN1D5, DDB2, DGKA,
DHRSX, DIMT1, DIS3, DLG1, DMD, DMTF1,
DNAJC19, DNAJC2, DNM1L, DNMT1,
DOCK5, DOCK7, DPH3, DPY19L4, DRAM2,
DSCR3, DTNBP1, DUSP11, DUSP22,
DYNC2H1, EBPL, EDC3, EDEM2, EDRF1,
EFEMP1, EHBP1, EHMT2, EIF4A2, EIF4E2,
EIF4H, ELK1, ELN, ELOVL1, ENC1,
ENOSF1, EPB41L1, EPB41L2, ERBB2IP, ERLEC1,
ERMAP, ETHE1, EVC, EVI5L, FAM104A,
FAM111A, FAM134C, FAM149B1, FAM172A,
FAM204A, FAM211A-AS1, FAM21C, FAP,
FBXL12, FBXL3, FBXO25, FBXW11, FGF5,
FGFR1OP, FHL2, FIP1L1, FLAD1, FLNA, FN1,
FNBP4, FNIP2, FOXN3, FRYL, FUT8,
FXR2, FYN, GAB1, GABPB2, GABRE, GALNS,
GFPT2, GGCT, GIT2, GK, GLS,
GOLGA4, GOLGB1, GOLT1B, GPR133,
GPR180, GRIPAP1, GTF2I, GTF3A, GUF1,
GUSB, HACL1, HAUS7, HCFC1R1, HDAC10,
HDAC7, HECTD3, HERC3, HIPK3,
HMGXB4, HNRNPA2B1, HNRNPD, HNRNPDL,
HPS1, HSD17B4, HSF2, HUWE1, IFT88,
IGF2BP2, IL15RA, INF2, ING4, INO80E, INPP1,
IP6K2, IPO8, IQCB1, IRAK4, ISOC2,
IST1, ITGB1BP1, KATNBL1, KDM4C,
KDM5A, KIAA0100, KIF3A, KITLG, KLC1,
KTN1, L3HYPDH, LACC1, LAMTOR3, LAS1L,
LENG8, LETMD1, LGALS8, LINC00963,
LMAN2L, LMO7, LONRF1, LOXL3,
LPHN2, LPIN1, LRCH3, LRRC28, LRRC32, LSM1,
LTA4H, LTBP1, LTBP3, LTBP4, LUC7L,
LYPLAL1, LYRM7, LZTFL1, MACF1, MADD,
MAGOHB, MAN2C1, MAP3K3, MAP4K4,
MAPK11, MAPK12, MAPKAP1, MARK3,
MBD5, MBNL1, MBOAT2, ME3, MED13,
MED15, MEG3, MEG3, MEGF6, METAP1,
METTL14, MFF, MFSD12, MGEA5,
MGST2, MINK1, MIR22HG, MKS1, MLF1, MLH1,
MLLT6, MLST8, MMP19, MORF4L2, MOSPD1,
MPDZ, MPI, MPV17, MRPL33, MSRB3,
MSRB3, MTCH2, MTMR2, MTMR3,
MTMR6, MTRR, MTSS1L, MYBL1, MYCBP2,
MYEF2, MYLK, MYNN, MYO18A, MYO19,
MYO1B, MYO5A, NABP1, NADK2, NAE1,
NBN, NEK1, NEO1, NEXN, NF2, NFE2L1,
NIPA2, NLRC5, NLRX1, NPEPPS, NPR2,
NPRL3, NRG1, NSFL1C, NSMCE2, NSUN4,
NT5C2, NUB1, NUP43, NUP62, NUPL2,
NUTM2A-AS1, OARD1, OCRL, ODF2,
ODF2L, OFD1, OPTN, ORMDL1, OS9, OSBPL8,
PACRGL, PAM, PARD3, PARL, PARP11,
PARP6, PATL1, PCBP2, PCID2, PCM1,
PCNXL2, PCNXL4, PCYT2, PDE4DIP,
PDLIM2, PDZD11, PEAK1, PEX1, PEX11A,
PEX5, PFDN1, PFDN6, PFKM, PHC3, PHKG2,
PHLDB1, PI4KB, PIGG, PIGN, PIGT,
PIK3C2A, PIKFYVE, PILRB, PIN1, PKIG,
PLA2G12A, PLA2R1, PLAGL1, PLBD2,
PMS2P3, PNPLA8, PODNL1, POLR3GL, PPAP2A,
PPFIBP1, PPIL3, PPIP5K2, PPM1M,
PPP1R12A, PPP1R21, PPP2R3C, PPP3CB, PPP3CC,
PPP4C, PPP6R2, PPRC1, PREB,
PREPL, PRKAG1, PRMT2, PRPF39, PRPF40B,
PRR16, PRR4, PRRX1, PRUNE, PSME4,
PTPN13, PTPN21, PTPN23, PTPN4, PTPRA, PVR,
PXDN, QTRTD1, R3HCC1L, R3HDM4,
RAB11FIP2, RABEPK, RAD1, RALGPS2,
RALY, RBBP9, RBCK1, RBM41, RCOR3,
REPS1, REV3L, RFWD2, RGL2, RGN, RHBDD2,
RHOBTB1, RHOT1, RIF1, RIPK2,
RNASE4, RNF146, RNF170, RNF214, RNF34,
RNFT1, ROBO1, RP11-1055B8.7, RP11-
14N7.2, RP11-274B21.1, RP11-33B1.1, RP11-
383H13.1, RP11-773D16.1, RP13-279N23.2,
RPAIN, RPL7L1, RPP14, RPS6KB2, RRBP1,
RSU1, RTEL1, RTEL1-TNFRSF6B, RUFY2,
RWDD1, RWDD2B, RWDD4, SBF1, SCMH1,
SDCCAG8, SEC31B, SECISBP2L,
SEMA4F, SENP1, SENP6, SERAC1, SETD5,
SETX, SFXN4, SGSM3, SH3D19, SHMT2,
SIPA1L1, SLC15A4, SLC22A17, SLC25A17,
SLC25A32, SLC25A36, SLC35A1, SLC35A3,
SLC35D2, SLC37A3, SLC38A2, SLC38A6, SLC4A7,
SLC5A6, SLC7A6, SLC9B2, SLIT2,
SLMAP, SLMO1, SLTM, SMAD5, SMARCA1,
SMARCD1, SMC5, SMEK2, SMPDL3A,
SMURF2, SNAPC5, SNED1, SNHG14, SNHG15,
SNRNP70, SNRPA1, SNRPG, SNX14,
SNX21, SPATA20, SPATA7, SPDL1,
SPPL2A, SPTAN1, ST6GALNAC6, ST7,
STARD3NL, STARD5, STAT6, STK16, STK19,
STK40, STRA13, STRADA, STRN3,
STX16, STX16-NPEPL1, STX3, STXBP3,
STYXL1, SUCO, SUGP2, SUGT1, SULF1,
SUN2, SUPT20H, SYNRG, TAF1, TAF2,
TANGO2, TARBP2, TAS2R14, TAZ, TBC1D14,
TBC1D17, TBC1D25, TBC1D5, TBCK, TBPL1,
TBX15, TCERG1, TEAD2, TEP1, TFDP1,
TFDP2, TFPI, TGFBR2, THAP6, THAP9-AS1,
THBS3, THOC2, TIA1, TIAL1, TIMM17B,
TIMM21, TM7SF3, TMCO4, TMEM11,
TMEM120A, TMEM126B, TMEM18,
TMEM194A, TMEM234, TMEM260, TMEM39B,
TMEM62, TMOD2, TMUB2, TMX3,
TOM1, TP53INP1, TP53TG1, TPD52L1,
TPD52L2, TPRA1, TPT1-AS1, TRAPPC12,
TREX2, TRIM16, TRIM65, TRIO, TRIP10,
TRIP12, TROVE2, TRPC1, TRPM4, TRPT1,
TSEN15, TSR1, TSTD3, TUBG2, TULP3,
TXNDC11, TXNL4A, U2AF1L4, UACA, UBA7,
UBE2A, UBE2D1, UBN2, UBP1, UBQLN1,
UFD1L, UNC5B, URGCP, USMG5, USP24,
USP25, USP33, USP53, USP8, VAMP7, VCAN,
VEZT, VPS29, WBP1, WDFY2, YEATS2,
YIPF1, YME1L1, YY1AP1, ZBTB8OS,
ZC2HC1A, ZC3H11A, ZCCHC6, ZCCHC8,
ZDHHC16, ZDHHC20, ZDHHC7, ZFAND1,
ZFAND5, ZFAS1, ZMIZ1, ZMYM4, ZMYM5,
ZNF160, ZNF207, ZNF248, ZNF280D, ZNF384,
ZNF512, ZNF516, ZNF532, ZNF720, and ZZZ3.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 3.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 3.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutated wildtype gene transcript is transcribed from a gene selected from Table 3.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutated wildtype gene transcript is transcribed from a gene selected from Table 3.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype gene transcript is transcribed from a gene selected from Table 3.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype gene transcript is transcribed from a gene selected from Table 3.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype gene transcript is transcribed from a gene selected from Table 3.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype gene transcript is transcribed from a gene selected from Table 3.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtgggt (SEQ ID NO: 2), and CACTAGgtgaga (SEQ ID NO: 3), and CCAgtgagga (SEQ ID NO: 4), and wherein the wildtype gene transcript is transcribed from a gene selected from Table 3.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 3.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CTGAAgtcagt (SEQ ID NO: 15), and wherein the mutated wildtype gene transcript is transcribed from a gene selected from Table 3.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from CUGAAgucagu (SEQ ID NO: 23), and wherein the mutated wildtype gene transcript is transcribed from a gene selected from Table 3.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 3.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 3.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 3.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 3.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 3.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 3.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

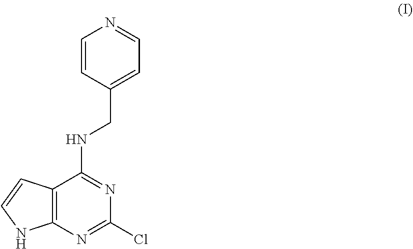 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 3.

Another aspect described herein is Compound (I) for use in the method, wherein a gene having a mutation in a gene transcript thereof is selected from Table 3.

One aspect described herein is use of Compound (I) to modulate the production of one or more mature RNA isoforms from a gene transcript in a cell comprising, contacting the cell with Compound (I), wherein the gene transcript is transcribed from a gene selected from Table 3.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 3.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 3.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutated wildtype gene transcript is transcribed from a gene selected from Table 3.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutated wildtype gene transcript is transcribed from a gene selected from Table 3.

Another aspect described herein is use of Compound (I) to modulate the production of one or more mature RNA isoforms from a predicted wildtype gene transcript in a cell comprising, contacting the cell with Compound (I), wherein the predicted wildtype gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype gene transcript is transcribed from a gene selected from Table 3.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 3.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 3.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 3.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtgggt (SEQ ID NO: 2), and CACTAGgtgaga (SEQ ID NO: 3), and CCAgtgagga (SEQ ID NO: 4), and wherein the wildtype gene transcript is transcribed from a gene selected from Table 3.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 3.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CTGAAgtcagt (SEQ ID NO: 15), and wherein the mutated wildtype gene transcript is transcribed from a gene selected from Table 3.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from CUGAAgucagu (SEQ ID NO: 23), and wherein the mutated wildtype gene transcript is transcribed from a gene selected from Table 3.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 3.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 3.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 3.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 3.

Another aspect described herein is use of Compound (I) to treat a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 3.

Another aspect described herein is use of Compound (I) to treat a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 3.

Another aspect described herein is use of Compound (I) to treat a disease associated with or mediated by a mutation in a wildtype gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 3.

Another aspect described herein is use of Compound (I), wherein a gene having a mutation in a gene transcript thereof is selected from the group consisting of Table 3.

### Methods of Modulating Mutated Gene Isoforms

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 4.

Table 4 lists mutated genes predicted by SpliceAI scores and the CNN Model, as described herein, that may be modulated by Compound (I) toward either or both exon inclusion and exon exclusion.

TABLE 4
ABCA4, ABCC9, ACADSB, ADAM10, AGK,
ALDH3A2, ALMS1, ANTXR2, APC,
ARMC9, ASAH1, ASPM, ATM, ATRX, BBS4,
BFSP1, BMPR2, BRCA1, BRCA2, BRIP1,
CA5A, CAPN3, CD3D, CDAN1, CDH1, CDH23,
CERKL, CFTR, CHD7, CLCN1, CLMP,
CLN3, CNGB3, COG6, COL11A1, COL3A1,
COL4A3, COL4A5, COL5A2, COL6A1,
COL7A1, CSTB, CTNS, CTSK, CTU2, CUBN,
CWC27, CYBB, DCX, DGKE, DGUOK,
DMD, DNAH5, DNAH9, DYNC2H1, DYNC2LI1,
EBF3, EBP, EP300, ERCC6, F10, F13A1,
F8, FBN1, FIG4, FLT3, FUT8, G6PC, GAA,
GABRG2, GCK, GLA, GNB5, GNPTAB,
GPR143, GPX4, GRHPR, GRN, GUCY2C,
GYPA, HBB, HFE, IDH1, IFT57, IL36RN,
KDSR, KIAA0586, KIAA1109, KIF14, KIT,
KMT2D, KRIT1, LAMB3, LDLR, LHCGR,
LIPA, LMNA, MAPT, MCFD2, MLH1, MMAB,
MPC1, MSH2, MTM1, MYBPC3,
MYO7A, NF1, NIPBL, NPHP1, NR3C2, NSD1,
OCA2, OFD1, OGT, ORC6, OTC, OTOGL,
PAFAH1B1, PAH, PARN, PDCD10, PIGN,
PMS2, POLG, POMGNT1, PPT1, PRMT7,
PTEN, RAD51B, RAD51C, RB1, RHAG, RNF216,
RTTN, SCN1A, SCN5A, SDCCAG8,
SDHD, SLC10A7, SLC12A1, SLC4A11, SMS,
SNX10, SPAST, SPG11, SPTA1, SPTB,
SRD5A2, STK11, STXBP1, SYNGAP1,
TCIRG1, TECTA, TFR2, TGFBR2, TJP2,
TMEM138, TMPRSS6, TP53, TPO, TRAPPC2,
TRIM37, TYR, UROD, VMA21, VPS13B,
WDR35, WDR73, and XPC.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the mutated wildtype gene transcript is transcribed from a gene selected from Table 4.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the mutated wildtype gene transcript is transcribed from a gene selected from Table 4.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 4.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 4.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted mutant gene transcript is transcribed from a gene selected from Table 4.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted mutant gene transcript is transcribed from a gene selected from Table 4.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted mutant gene transcript is transcribed from a gene selected from Table 4.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted mutant gene transcript is transcribed from a gene selected from Table 4.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 4.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 4.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 4.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 4.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 4.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 4.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 4.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 4.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

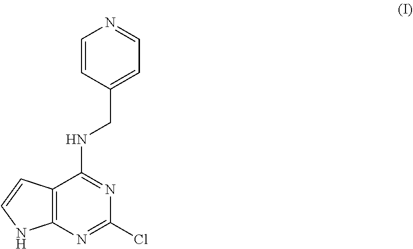 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 4.

Another aspect described herein is Compound (I) for use in the method, wherein a gene having a mutation in a gene transcript thereof is selected from Table 4.

One aspect described herein is use of Compound (I) to modulate the production of one or more mature RNA isoforms from a gene transcript in a cell comprising, contacting the cell with Compound (I), wherein the gene transcript is transcribed from a gene selected from Table 4.

Another aspect described herein is use of Compound (I) to modulate the production of one or more mature RNA isoforms from a mutated wildtype gene transcript in a cell comprising, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the mutated wildtype gene transcript is transcribed from a gene selected from Table 4.

Another aspect described herein is use of Compound (I) to modulate the production of one or more mature RNA isoforms from a mutated wildtype gene transcript in a cell comprising, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the mutated wildtype gene transcript is transcribed from a gene selected from Table 4.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 4.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 4.

Another aspect described herein is use of Compound (I) to modulate the production of one or more mature RNA isoforms from a predicted mutant gene transcript in a cell comprising, contacting the cell with Compound (I), wherein the predicted mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted mutant gene transcript is transcribed from a gene selected from Table 4.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 4.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 4.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 4.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 4.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 4.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 4.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 4.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 4.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 4.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 4.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 4.

Another aspect described herein is use of Compound (I) to treat a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 4.

Another aspect described herein is use of Compound (I), wherein a gene having a mutation in a gene transcript thereof is selected from the group consisting of Table 4.

### Methods of Modulating Wildtype Gene Isoforms

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 5.

Table 5 lists wildtype genes identified by RNA sequencing, as described herein, that may be modulated by Compound (I) toward exon inclusion.

TABLE 5
ABI1, ABI2, AC027612.6, ACBD5, ACIN1,
ACLY, ACOX1, ACSL3, AFMID, AHI1,
AKAP13, AL163636.6, ANKRD11, ANXA2,
AP1G1, ARHGAP12, ARHGEF10, ARSK,
ASAP2, ASPH, ATG10, ATP2C1, ATP5SL,
B3GALNT1, BANP, BBX, BPTF, BROX,
C11orf30, C12orf29, C2CD5, C3orf18, C5orf45,
C8orf59, C9orf156, CA5BP1, CALU,
CAMTA1, CASP3, CCBL2, CD44, CD46,
CDC14B, CDK16, CEP290, CLASP1, CLK1,
CMC1, COL12A1, COL6A3, COPB2, COPS8,
CPSF7, CREBBP, CYLD, DAB2, DCAF8,
DCP1A, DCUN1D5, DIS3, DNAJC19, DNM1L,
DOCK5, DPH3, DRAM2, EDEM2,
EFEMP1, EIF4H, ENC1, EPB41L2, FAM172A,
FAM21C, FBXL3, FGFR1OP, FIP1L1,
FNBP4, FRYL, FUT8, FXR2, GGCT, GIT2,
GLS, GTF2I, HNRNPA2B1, HNRNPDL,
HSD17B4, HSF2, IL15RA, INF2, INO80E,
INPP1, IP6K2, IQCB1, ITGB1BP1, KATNBL1,
KDM5A, KLC1, KTN1, L3HYPDH, LGALS8,
LMAN2L, LONRF1, LPHN2, LPIN1,
LRCH3, LSM1, MACF1, MAGOHB, MAPKAP1,
MARK3, MBNL1, ME3, MEG3,
METAP1, MFF, MOSPD1, MSRB3, MTMR2,
MYEF2, MYLK, MYNN, MYO18A,
MYO1B, MYO5A, NABP1, NEO1, NF2, NPEPPS,
NRG1, NSMCE2, NUP62, OCRL,
OFD1, OPTN, OSBPL8, PARP6, PDE4DIP,
PDZD11, PHC3, PHLDB1, PIGN, PIN1,
PNPLA8, PPP3CB, PPP3CC, PREPL, PRPF39,
PRRX1, PRUNE, QTRTD1, R3HCC1L,
RALY, RBBP9, RBM41, REV3L, RFWD2,
RNASE4, RNF34, RP11-14N7.2, RP11-
274B21.1, RPAIN, RRBP1, RWDD1, SDCCAG8,
SETX, SH3D19, SIPA1L1, SLC15A4,
SLC35A3, SLC38A6, SLTM, SMARCA1,
SNRNP70, SPATA7, SPDL1, SPPL2A,
ST6GALNAC6, STARD3NL, STK40, STRN3,
SUCO, SUGP2, SUPT20H, SYNRG, TAF2,
TBCK, TBPL1, TGFBR2, THAP9-AS1, TIA1,
TIAL1, TIMM17B, TM7SF3, TMEM126B,
TMEM18, TMEM234, TPD52L1, TPT1-AS1,
TRIM16, TRIM65, TRIP12, TROVE2,
TSEN15, TXNL4A, UBP1, USP33, USP8,
VEZT, VPS29, ZC2HC1A, ZC3H11A,
ZDHHC20, ZNF207, ZNF516, and ZNF532.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 5.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 5.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutated wildtype gene transcript is transcribed from a gene selected from Table 5.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutated wildtype gene transcript is transcribed from a gene selected from Table 5.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype gene transcript is transcribed from a gene selected from Table 5.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype gene transcript is transcribed from a gene selected from Table 5.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype gene transcript is transcribed from a gene selected from Table 5.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype gene transcript is transcribed from a gene selected from Table 5.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtgggt (SEQ ID NO: 2), and CCAgtgagga (SEQ ID NO: 4), and wherein the wildtype gene transcript is transcribed from a gene selected from Table 5.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAgugggu (SEQ ID NO: 6), and CCAgugagga (SEQ ID NO: 8), and wherein the wildtype gene transcript is transcribed from a gene selected from Table 5.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 5.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 5.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 5.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 5.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 5.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 5.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

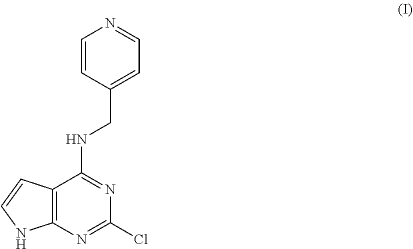 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 5.

Another aspect described herein is Compound (I) for use in the method, wherein a gene having a mutation in a gene transcript thereof is selected from Table 5.

One aspect described herein is use of Compound (I) to modulate the production of one or more mature RNA isoforms from a gene transcript in a cell comprising, contacting the cell with Compound (I), wherein the gene transcript is transcribed from a gene selected from Table 5.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 5.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 5.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutated wildtype gene transcript is transcribed from a gene selected from Table 5.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutated wildtype gene transcript is transcribed from a gene selected from Table 5.

Another aspect described herein is use of Compound (I) to modulate the production of one or more mature RNA isoforms from a predicted wildtype gene transcript in a cell comprising, contacting the cell with Compound (I), wherein the predicted wildtype gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype gene transcript is transcribed from a gene selected from Table 5.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 5.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 5.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 5.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GATTAAgtgggt (SEQ ID NO: 2), and CCAgtgagga (SEQ ID NO: 4), and wherein the wildtype gene transcript is transcribed from a gene selected from Table 5.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: GAUUAAgugggu (SEQ ID NO: 6), and CCAgugagga (SEQ ID NO: 8), and wherein the wildtype gene transcript is transcribed from a gene selected from Table 5.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 5.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 5.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 5.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 5.

Another aspect described herein is use of Compound (I) to treat a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 5.

Another aspect described herein is use of Compound (I) to treat a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 5.

Another aspect described herein is use of Compound (I) to treat a disease associated with or mediated by a mutation in a wildtype gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 5.

Another aspect described herein is use of Compound (I), wherein a gene having a mutation in a gene transcript thereof is selected from the group consisting of Table 5.

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 6.

Table 6 lists wildtype genes identified by RNA sequencing, as described herein, that may be modulated by Compound (I) toward exon exclusion.

TABLE 6
A1BG-AS1, AAAS, ABCA1, ABCA6, ABCA9,
ABCB6, ABCC3, ABHD14A-ACY1,
ABI3BP, AC024560.3, AC037459.4, ACAA1,
ACACA, ACAD10, ACBD4, ACSL4,
ACTN1, ACY1, AD000671.6, ADA, ADAM33,
ADAMTSL4, ADARB1, ADK, AKAP11,
AKIP1, AKR1A1, ALDH3B1, ALDH4A1,
ALKBH1, ALKBH3, AMMECR1L, AMPD2,
ANK2, ANKRD10, ANKRD36, ANO6,
AP2M1, APBB2, APOL1, APP, ARHGAP23,
ARHGEF10L, ARHGEF12, ARHGEF25,
ARID4B, ARIH2, ASCC2, ASL, ASPH, ASUN,
ASXL1, ATG16L1, ATG7, ATG9A, ATPAF1,
ATXN1, ATXN3, AUP1, AUTS2, AVL9,
BAG6, BAX, BAZ1A, BBS4, BBS5, BCAR1,
BCOR, BIN1, BIRC6, BLOC1S6, BMP2K,
BOK, BPTF, BRD8, BTBD19, C11orf70, C16orf13,
C1orf85, C1RL, C5orf42, C9orf85,
CAMK2D, CAPN7, CAPRIN2, CARD8, CARKD,
CAST, CCDC25, CCDC90B, CCNDBP1,
CCNL1, CD46, CD55, CD99P1, CDC14B, CDC16,
CHD3, CHEK2, CHKB, CKLF, CLCN6,
CNOT10, COL16A1, COL6A3, COPS7A,
COQ6, COX20, CPEB2, CPT1C, CRAT, CRLS1,
CRNDE, CRYZ, CSAD, CTDSPL, CUTC, CWC25,
CYB561D2, CYP20A1, DBT, DCAF10,
DCAF11, DCAF17, DDB2, DGKA, DHRSX,
DIMT1, DIS3, DLG1, DMD, DMTF1,
DNAJC2, DNM1L, DNMT1, DOCK7, DPY19L4,
DRAM2, DSCR3, DTNBP1, DUSP11,
DUSP22, DYNC2H1, EBPL, EDC3, EDRF1,
EHBP1, EHMT2, EIF4A2, EIF4E2, ELK1,
ELN, ELOVL1, ENOSF1, EPB41L1, ERBB2IP,
ERLEC1, ERMAP, ETHE1, EVC, EVI5L,
FAM104A, FAM111A, FAM134C, FAM149B1,
FAM172A, FAM204A, FAM211A-AS1,
FAP, FBXL12, FBXO25, FBXW11, FGF5,
FHL2, FIP1L1, FLAD1, FLNA, FN1, FNIP2,
FOXN3, FYN, GAB1, GABPB2, GABRE,
GALNS, GFPT2, GIT2, GK, GLS, GOLGA4,
GOLGB1, GOLTIB, GPR133, GPR180, GRIPAP1,
GTF3A, GUF1, GUSB, HACL1,
HAUS7, HCFC1R1, HDAC10, HDAC7,
HECTD3, HERC3, HIPK3, HMGXB4, HNRNPD,
HPS1, HUWE1, IFT88, IGF2BP2, ING4, IPO8,
IRAK4, ISOC2, IST1, KDM4C, KIAA0100,
KIF3A, KITLG, KLC1, LACC1, LAMTOR3,
LAS1L, LENG8, LETMD1, LINC00963,
LMAN2L, LMO7, LOXL3, LPHN2, LRRC28,
LRRC32, LTA4H, LTBP1, LTBP3, LTBP4,
LUC7L, LYPLAL1, LYRM7, LZTFL1, MACF1,
MADD, MAN2C1, MAP3K3, MAP4K4,
MAPK11, MAPK12, MBD5, MBOAT2,
MED13, MED15, MEG3, MEGF6, METTL14,
MFF, MFSD12, MGEA5, MGST2, MINK1,
MIR22HG, MKS1, MLF1, MLH1, MLLT6,
MLST8, MMP19, MORF4L2, MOSPD1,
MPDZ, MPI, MPV17, MRPL33, MSRB3, MTCH2,
MTMR2, MTMR3, MTMR6, MTRR, MTSS1L,
MYBL1, MYCBP2, MYLK, MYNN,
MYO19, MYO5A, NADK2, NAE1, NBN,
NEK1, NEXN, NFE2L1, NIPA2, NLRC5,
NLRX1, NPR2, NPRL3, NSFL1C, NSUN4,
NT5C2, NUB1, NUP43, NUPL2, NUTM2A-
AS1, OARD1, ODF2, ODF2L, OPTN, ORMDL1,
OS9, PACRGL, PAM, PARD3, PARL,
PARP11, PATL1, PCBP2, PCID2, PCM1,
PCNXL2, PCNXL4, PCYT2, PDLIM2, PEAK1,
PEX1, PEX11A, PEX5, PFDN1, PFDN6, PFKM,
PHKG2, PI4KB, PIGG, PIGT, PIK3C2A,
PIKFYVE, PILRB, PKIG, PLA2G12A,
PLA2R1, PLAGL1, PLBD2, PMS2P3, PODNL1,
POLR3GL, PPAP2A, PPFIBP1, PPIL3, PPIP5K2,
PPM1M, PPP1R12A, PPP1R21, PPP2R3C,
PPP4C, PPP6R2, PPRC1, PREB, PRKAG1,
PRMT2, PRPF40B, PRR16, PRR4, PSME4,
PTPN13, PTPN21, PTPN23, PTPN4,
PTPRA, PVR, PXDN, R3HDM4, RAB1 1FIP2,
RABEPK, RAD1, RALGPS2, RBCK1, RCOR3,
REPS1, RGL2, RGN, RHBDD2,
RHOBTB1, RHOT1, RIF1, RIPK2, RNF146,
RNF170, RNF214, RNFT1, ROBO1, RP11-
1055B8.7, RP11-33B1.1, RP11-383H13.1,
RP11-773D16.1, RP13-279N23.2, RPAIN,
RPL7L1, RPP14, RPS6KB2, RSU1,
RTEL1, RTEL1-TNFRSF6B, RUFY2, RWDD2B,
RWDD4, SBF1, SCMH1, SEC31B, SECISBP2L,
SEMA4F, SENP1, SENP6, SERAC1,
SETD5, SFXN4, SGSM3, SHMT2, SIPA1L1,
SLC22A17, SLC25A17, SLC25A32,
SLC25A36, SLC35A1, SLC35D2,
SLC37A3, SLC38A2, SLC38A6, SLC4A7, SLC5A6,
SLC7A6, SLC9B2, SLIT2, SLMAP, SLMO1,
SMAD5, SMARCD1, SMC5, SMEK2,
SMPDL3A, SMURF2, SNAPC5, SNED1,
SNHG14, SNHG15, SNRPA1, SNRPG, SNX14,
SNX21, SPATA20, SPTAN1, ST7, STARD5,
STAT6, STK16, STK19, STRA13, STRADA,
STX16, STX16-NPEPL1, STX3, STXBP3,
STYXL1, SUGT1, SULF1, SUN2, SUPT20H,
TAF1, TANGO2, TARBP2, TAS2R14, TAZ,
TBC1D14, TBC1D17, TBC1D25, TBC1D5,
TBCK, TBX15, TCERG1, TEAD2, TEP1,
TFDP1, TFDP2, TFP1, THAP6, THBS3, THOC2,
TIMM21, TMCO4, TMEM11, TMEM120A,
TMEM194A, TMEM260, TMEM39B,
TMEM62, TMOD2, TMUB2, TMX3,
TOM1, TP53INP1, TP53TG1, TPD52L2, TPRA1,
TRAPPC12, TREX2, TRIO, TRIP10,
TRPC1, TRPM4, TRPT1, TSEN15, TSR1, TSTD3,
TUBG2, TULP3, TXNDC11, U2AF1L4,
UACA, UBA7, UBE2A, UBE2D1, UBN2,
UBQLN1, UFD1L, UNC5B, URGCP, USMG5,
USP24, USP25, USP53, VAMP7, VCAN,
WBP1, WDFY2, YEATS2, YIPF1, YME1L1,
YY1AP1, ZBTB80S, ZCCHC6, ZCCHC8,
ZDHHC16, ZDHHC7, ZFAND1, ZFAND5,
ZFAS1, ZMIZ1, ZMYM4, ZMYM5, ZNF160,
ZNF207, ZNF248, ZNF280D, ZNF384,
ZNF512, ZNF720, and ZZZ3.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 6.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 6.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutated wildtype gene transcript is transcribed from a gene selected from Table 6.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutated wildtype gene transcript is transcribed from a gene selected from Table 6.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype gene transcript is transcribed from a gene selected from Table 6.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype gene transcript is transcribed from a gene selected from Table 6.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype gene transcript is transcribed from a gene selected from Table 6.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype gene transcript is transcribed from a gene selected from Table 6.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CACTAGgtgaga (SEQ ID NO: 3), and wherein the wildtype gene transcript is transcribed from a gene selected from Table 6.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CACUAGgugaga (SEQ ID NO: 7), and wherein the wildtype gene transcript is transcribed from a gene selected from Table 6.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CACTAGgtgagc (SEQ ID NO: 11), and CTGAAgtcagt (SEQ ID NO: 15), and wherein the mutated wildtype gene transcript is transcribed from a gene selected from Table 6.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CACUAGgugagc (SEQ ID NO: 19), and CUGAAgucagu (SEQ ID NO: 23), and wherein the mutated wildtype gene transcript is transcribed from a gene selected from Table 6.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 6.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 6.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 6.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 6.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 6.

Another aspect described herein is Compound (I) for use in the method, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 6.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

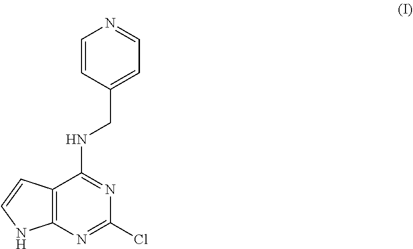 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 6.

Another aspect described herein is Compound (I) for use in the method, wherein a gene having a mutation in a gene transcript thereof is selected from Table 6.

One aspect described herein is use of Compound (I) to modulate the production of one or more mature RNA isoforms from a gene transcript in a cell comprising, contacting the cell with Compound (I), wherein the gene transcript is transcribed from a gene selected from Table 6.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 6.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 6.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutated wildtype gene transcript is transcribed from a gene selected from Table 6.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutated wildtype gene transcript is transcribed from a gene selected from Table 6.

Another aspect described herein is use of Compound (I) to modulate the production of one or more mature RNA isoforms from a predicted gene transcript in a cell comprising, contacting the cell with Compound (I), wherein the predicted wildtype gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype gene transcript is transcribed from a gene selected from Table 6.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 6.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 6.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 6.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CACTAGgtgaga (SEQ ID NO: 3), and wherein the wildtype gene transcript is transcribed from a gene selected from Table 6.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CACUAGgugaga (SEQ ID NO: 7), and wherein the wildtype gene transcript is transcribed from a gene selected from Table 6.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CACTAGgtgagc (SEQ ID NO: 11), and CTGAAgtcagt (SEQ ID NO: 15), and wherein the mutated wildtype gene transcript is transcribed from a gene selected from Table 6.

Another aspect described herein is use of Compound (I) to modulate the production of one or more mature RNA isoforms from a mutated wildtype gene transcript in a cell comprising, contacting the cell with Compound (I), wherein the mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CACUAGgugagc (SEQ ID NO: 19), and CUGAAgucagu (SEQ ID NO: 23), and wherein the mutated wildtype gene transcript is transcribed from a gene selected from Table 6.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 6.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 6.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 6.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 6.

Another aspect described herein is use of Compound (I) to treat a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 6.

Another aspect described herein is use of Compound (I) to treat a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 6.

Another aspect described herein is use of Compound (I) to treat a disease associated with or mediated by a mutation in a wildtype gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 6.

Another aspect described herein is use of Compound (I), wherein a gene having a mutation in a gene transcript thereof is selected from the group consisting of Table 6.

### Methods of Modulating Mutated Gene Isoforms

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 7.

Table 7 lists mutated genes predicted by SpliceAI scores and the CNN Model, as described herein, that may be modulated by Compound (I) toward exon inclusion.

TABLE 7
ABCA4, ABCC9, ACADSB, ADAM10, AGK,
ALDH3A2, ALMS1, ANTXR2, APC,
ARMC9, ASAH1, ASPM, ATM, ATRX, BBS4,
BFSP1, BMPR2, BRCA1, BRCA2, CA5A,
CAPN3, CD3D, CDH1, CDH23, CERKL, CFTR,
CHD7, CLCN1, CLMP, CLN3, CNGB3,
COG6, COL11A1, COL3A1, COL4A3, COL4A5,
COL5A2, CSTB, CTNS, CTU2, CUBN,
CWC27, CYBB, DCX, DGKE, DGUOK, DMD,
DNAH5, DNAH9, DYNC2H1, DYNC2LI1,
EBF3, EP300, F10, F13A1, FBN1, FUT8, G6PC,
GAA, GABRG2, GCK, GLA, GNB5,
GNPTAB, GPR143, GPX4, GRHPR, GRN, GUCY2C,
GYPA, HBB, IDH1, IFT57, IL36RN,
KDSR, KIAA0586, KIAA1109, KIF14, KIT,
KMT2D, KRIT1, LAMB3, LDLR, LIPA,
LMNA, MCFD2, MLH1, MMAB, MPC1, MSH2,
MTM1, MYBPC3, MYO7A, NF1, NIPBL,
NPHP1, NR3C2, NSD1, OCA2, OFD1, OGT,
ORC6, OTC, OTOGL, PAFAH1B1, PAH,
PARN, PDCD10, PIGN, PMS2, POLG, POMGNT1,
PPT1, PRMT7, PTEN, RAD51B,
RAD51C, RB1, RHAG, RNF216, RTTN, SCN1A,
SLC10A7, SLC12A1, SLC4A11, SMS,
SPAST, SPG11, SPTA1, SPTB, SRD5A2, STXBP1,
SYNGAP1, TGFBR2, TJP2, TMEM138,
TMPRSS6, TP53, TPO, TRAPPC2, TRIM37,
TYR, UROD, VMA21, VPS13B, WDR35,
WDR73, and XPC.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the mutated wildtype gene transcript is transcribed from a gene selected from Table 7.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the mutated wildtype gene transcript is transcribed from a gene selected from Table 7.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 7.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 7.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted mutant gene transcript is transcribed from a gene selected from Table 7.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted mutant gene transcript is transcribed from a gene selected from Table 7.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted mutant gene transcript is transcribed from a gene selected from Table 7.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted mutant gene transcript is transcribed from a gene selected from Table 7.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 7.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 7.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 7.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 7.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 7.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 7.

Another aspect described herein a the use of Compound (I) to treat a disease associated with or mediated by a mutation in a gene transcript in a subject in need thereof, wherein the method comprises, administering Compound (I) to the subject, wherein exon inclusion or exon exclusion is modulated in one or more mature RNA isoforms produced from the gene transcript, wherein the subject is human, and wherein the gene is selected from Table 7.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 7.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

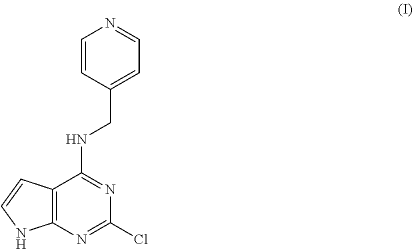 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 7.

Another aspect described herein is Compound (I) for use in the method, wherein a gene having a mutation in a gene transcript thereof is selected from Table 7.

One aspect described herein is use of Compound (I) to modulate the production of one or more mature RNA isoforms from a gene transcript in a cell comprising, contacting the cell with Compound (I), wherein the gene transcript is transcribed from a gene selected from Table 7.

Another aspect described herein is use of Compound (I) to modulate the production of one or more mature RNA isoforms from a mutated wildtype gene transcript in a cell comprising, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the mutated wildtype gene transcript is transcribed from a gene selected from Table 7.

Another aspect described herein is use of Compound (I) to modulate the production of one or more mature RNA isoforms from a mutated wildtype gene transcript in a cell comprising, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the mutated wildtype gene transcript is transcribed from a gene selected from Table 7.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 7.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 7.

Another aspect described herein is use of Compound (I) to modulate the production of one or more mature RNA isoforms from a predicted mutant gene transcript in a cell comprising, contacting the cell with Compound (I), wherein the predicted mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted mutant gene transcript is transcribed from a gene selected from Table 7.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 7.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 7.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 7.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 7.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 7.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 7.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 7.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 7.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 7.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 7.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 7.

Another aspect described herein is use of Compound (I) to treat a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 7.

Another aspect described herein is use of Compound (I), wherein a gene having a mutation in a gene transcript thereof is selected from the group consisting of Table 7.

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 8.

Table 8 lists mutated genes predicted by SpliceAI scores and the CNN Model, as described herein, that may be modulated by Compound (I) toward exon exclusion.

TABLE 8
BRCA1, BRIP1, CDAN1, CLN3, COL6A1,
COL7A1, CTSK, EBP, ERCC6, F8, FBN1,
FIG4, FLT3, GLA, HFE, LHCGR, MAPT,
OTC, SCN5A, SDCCAG8, SDHD, SNX10,
STK11, TCIRG1, TECTA, TFR2, and TP53.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the mutated wildtype gene transcript is transcribed from a gene selected from Table 8.

Another aspect described herein is Compound (I) for use in the method, wherein Compound (I) modulates the production of one or more mature RNA isoforms from a mutated wildtype gene transcript in a cell, wherein the method comprises, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the mutated wildtype gene transcript is transcribed from a gene selected from Table 8.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 8.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 8.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted mutant gene transcript is transcribed from a gene selected from Table 8.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted mutant gene transcript is transcribed from a gene selected from Table 8.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted mutant gene transcript is transcribed from a gene selected from Table 8.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted mutant gene transcript is transcribed from a gene selected from Table 8.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from: AGTgtaagta (SEQ ID NO: 16), and wherein the mutant gene transcript is transcribed from a gene selected from Table 8.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from: AGUguaagua (SEQ ID NO: 24), and wherein the mutant gene transcript is transcribed from a gene selected from Table 8.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 8.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 8.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 8.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 8.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 8.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 8.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

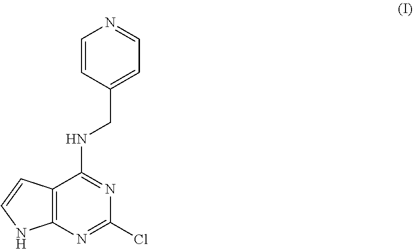 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 8.

Another aspect described herein is Compound (I) for use in the method, wherein a gene having a mutation in a gene transcript thereof is selected from Table 8.

One aspect described herein is use of Compound (I) to modulate the production of one or more mature RNA isoforms from a gene transcript in a cell comprising, contacting the cell with Compound (I), wherein the gene transcript is transcribed from a gene selected from Table 8.

Another aspect described herein is use of Compound (I) to modulate the production of one or more mature RNA isoforms from a mutated wildtype gene transcript in a cell comprising, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the mutated wildtype gene transcript is transcribed from a gene selected from Table 8.

Another aspect described herein is use of Compound (I) to modulate the production of one or more mature RNA isoforms from a mutated wildtype gene transcript in a cell comprising, contacting the cell with Compound (I), wherein the non-mutated wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the mutated wildtype gene transcript is transcribed from a gene selected from Table 8.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 8.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 8.

Another aspect described herein is use of Compound (I) to modulate the production of one or more mature RNA isoforms from a predicted mutant gene transcript in a cell comprising, contacting the cell with Compound (I), wherein the predicted mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted mutant gene transcript is transcribed from a gene selected from Table 8.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 8.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 8.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 8.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from: AGTgtaagta (SEQ ID NO: 16), and wherein the mutant gene transcript is transcribed from a gene selected from Table 8.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from: AGUguaagua (SEQ ID NO: 24), and wherein the mutant gene transcript is transcribed from a gene selected from Table 8.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 8.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 8.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 8.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 8.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 8.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 8.

Another aspect described herein is use of Compound (I) to treat a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 8.

Another aspect described herein is use of Compound (I), wherein a gene having a mutation in a gene transcript thereof is selected from the group consisting of Table 8.

### Methods of Modulating Wildtype and Mutated Gene Isoforms

One aspect described herein is Compound (I) for use in a method for modulating the production of one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7 or 8.

Another aspect described herein is Compound (I) for use in a method for modulating the production of one or more mature RNA isoforms from a gene transcript in a cell, wherein the method comprises, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the gene transcript is transcribed from a gene selected from Tables 1, 2, 3, 4, 5, 6, 7 or 8.

Another aspect described herein is Compound (I) for use in a method for modulating the production of one or more mature RNA isoforms from a gene transcript in a cell, wherein the method comprises, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the gene transcript is transcribed from a gene selected from Tables 1, 2, 3, 4, 5, 6, 7 or 8.

Another aspect described herein is Compound (I) for use in a method for modulating the production of one or more mature RNA isoforms from a gene transcript in a cell, wherein the method comprises, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the gene transcript is transcribed from a gene selected from Tables 1, 2, 3, 4, 5, 6, 7 or 8.

Another aspect described herein is Compound (I) for use in a method for modulating the production of one or more mature RNA isoforms from a gene transcript in a cell, wherein the method comprises, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the gene transcript is transcribed from a gene selected from Tables 1, 2, 3, 4, 5, 6, 7 or 8.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted gene transcript is transcribed from a gene selected from Tables 1, 2, 3, 4, 5, 6, 7 or 8.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7 or 8.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7 or 8.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7 or 8.

Another aspect described herein is Compound (I) for use in a method for modulating exon inclusion or exon exclusion in one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7 or 8.

Another aspect described herein is Compound (I) for use in a method for increasing exon inclusion in one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7 or 8.

Another aspect described herein is Compound (I) for use in a method for increasing exon exclusion in one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7 or 8.

Another aspect described herein is Compound (I) for use in a method for modulating exon inclusion or exon exclusion in one or more mature RNA isoforms from a gene transcript in a cell, wherein the method comprises, contacting the cell in vivo or in vitro with Compound (I), and wherein the gene transcript is transcribed from a gene selected from Tables 1, 2, 3, 4, 5, 6, 7 or 8.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene transcript is transcribed from a gene selected from Tables 1, 2, 3, 4, 5, 6, 7 or 8.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Tables 1, 2, 3, 4, 5, 6, 7 or 8.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

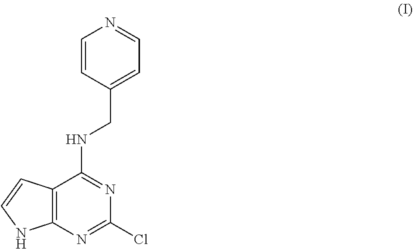 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Tables 1, 2, 3, 4, 5, 6, 7 or 8.

Another aspect described herein is Compound (I) for use in the method, wherein a gene having the mutated transcript is selected from Tables 1, 2, 3, 4, 5, 6, 7 or 8.

One aspect described herein is a method for modulating the production of one or more mature RNA isoforms from a gene transcript in a cell comprising, contacting the cell with Compound (I), wherein the gene transcript is transcribed from a gene selected from Tables 1, 2, 3, 4, 5, 6, 7 or 8.

Another aspect described herein is a method for modulating the production of one or more mature RNA isoforms from a gene transcript in a cell comprising, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the gene transcript is transcribed from a gene selected from Tables 1, 2, 3, 4, 5, 6, 7 or 8.

Another aspect described herein is a method for modulating the production of one or more mature RNA isoforms from a gene transcript in a cell comprising, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the gene transcript is transcribed from a gene selected from Tables 1, 2, 3, 4, 5, 6, 7 or 8.

Another aspect described herein is a method for modulating the production of one or more mature RNA isoforms from a gene transcript in a cell comprising, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the gene transcript is transcribed from a gene selected from Tables 1, 2, 3, 4, 5, 6, 7 or 8.

Another aspect described herein is a method for modulating the production of one or more mature RNA isoforms from a gene transcript in a cell comprising, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the gene transcript is transcribed from a gene selected from Tables 1, 2, 3, 4, 5, 6, 7 or 8.

Another aspect described herein is a method for modulating the production of one or more mature RNA isoforms from a predicted gene transcript in a cell comprising, contacting the cell with Compound (I), wherein the predicted gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted gene transcript is transcribed from a gene selected from Tables 1, 2, 3, 4, 5, 6, 7 or 8.

Another aspect described herein is a method for modulating the production of one or more mature RNA isoforms from a predicted gene transcript in a cell comprising, contacting the cell with Compound (I), wherein the predicted gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7 or 8.

Another aspect described herein is a method for modulating the production of one or more mature RNA isoforms from a predicted gene transcript in a cell comprising, contacting the cell with Compound (I), wherein the predicted gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7 or 8.

Another aspect described herein is a method for modulating the production of one or more mature RNA isoforms from a predicted gene transcript in a cell comprising, contacting the cell with Compound (I), wherein the predicted gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7 or 8.

Another aspect described herein is a method for modulating exon inclusion or exon exclusion in one or more mature RNA isoforms from a gene transcript in a cell comprising, contacting the cell with Compound (I), wherein the gene transcript is transcribed from a gene selected from Tables 1, 2, 3, 4, 5, 6, 7 or 8.

Another aspect described herein is a method for increasing exon inclusion in one or more mature RNA isoforms from a gene transcript in a cell comprising, contacting the cell with Compound (I), wherein the gene transcript is transcribed from a gene selected from Tables 1, 2, 3, 4, 5, 6, 7 or 8.

Another aspect described herein is a method for increasing exon exclusion in one or more mature RNA isoforms from a gene transcript in a cell comprising, contacting the cell with Compound (I), wherein the gene transcript is transcribed from a gene selected from Tables 1, 2, 3, 4, 5, 6, 7 or 8.

Another aspect described herein is a method for modulating exon inclusion or exon exclusion in one or more mature RNA isoforms from a gene transcript in a cell comprising, contacting the cell in vivo or in vitro with Compound (I), wherein the gene transcript is transcribed from a gene selected from Tables 1, 2, 3, 4, 5, 6, 7 or 8.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene transcript is transcribed from a gene selected from Tables 1, 2, 3, 4, 5, 6, 7 or 8.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Tables 1, 2, 3, 4, 5, 6, 7 or 8.

Another aspect described herein is a method for treating a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Tables 1, 2, 3, 4, 5, 6, 7 or 8.

Another aspect described herein is use of Compound (I), wherein a gene having a mutation in a gene transcript thereof is selected from the group consisting of Tables 1, 2, 3, 4, 5, 6, 7 or 8.

### Methods of Modulating Wildtype Gene Isoforms

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 9.

Table 9 lists wildtype genes identified by RNA sequencing, as described herein, that may be unchanged by Compound (I) toward either or both exon inclusion and exon exclusion.

TABLE 9
AAMDC, ABCA2, ABCC3, ABCD4, ABHD16A,
ABI3BP, ACAD10, ACAP3, ACCS,
ACOT9, ACTN1, ADAM15, AFTPH, AK4,
AMBRA1, AMDHD2, AMIGO2, AMOTL1,
AMZ2P1, ANAPC11, ANKRD17, ANO6,
AOX1, ARHGEF10, ARID5A, ARIH2, ARMC10,
ARSJ, ASCC2, ATG12, ATG13, ATG4B, ATP5C1,
ATP6C, B3GALNT1, BAZ2A, BCL2L2,
BEND6, BIRC6, BLOC1S6, BRD8, BTBD3,
BTF3L4, C10orf118, C11orf30, C11orf57,
C14orf159, C2orf76, C9orf156, CACNA1C,
CAMK2G, CAMSAP1, CAPN10, CARF,
CCDC126, CCDC136, CCDC25, CCNL2,
CCNT1, CCNYL1, CD27-AS1, CDC42BPA,
CDCA7L, CDK5RAP2, CEP164, CIRBP,
CLASP2, CPNE1, CSDE1, CSNK1A1, CTIF,
CTSB, DCAF8, DCTD, DGUOK, DLGAP1-AS1,
DMKN, DMWD, DNAAF2, DNAJC14,
DNAJC24, DPH7, DPM1, DUS2, ECHDC2,
EHBP1L1, EIF4G2, ELMOD3, EMC4, ENC1,
EP400, EPS15L1, ERMARD, EVA1A, FAM114A1,
FAM13B, FAM160B2, FAM173A,
FAM175A, FAM208B, FAM35A, FAM45A,
FDPS, FGFR1, FHOD3, FLNB, FNBP1,
FOSL1, FOXRED2, FRS2, GAS7, GBP3,
GEMIN8, GLIS3, GLRB, GLT8D1, GNAS,
GPATCH8, GPBP1, GRB10, HDAC9, HMCN1,
HNRNPK, HSCB, IL17RA, IL17RC, IL6,
INTS9, IP6K2, KANSL3, KAT6A, KCTD20,
KIAA0232, KIAA0368, KIAA0753,
KIAA1551, KIAA1586, KIAA1731, KIFC3,
KLHL12, KTN1, LBR, LDB2, LRP12, MADD,
MAP2K5, MAP7D1, MAPK9, MAZ, MBD1,
MCCC2, MECR, MED23, MEGF6, MEIS1,
METTL10, METTL21A, MGLL, MICU3,
MKNK2, MLLT10, MPPE1, MRI1, MRPL55,
MTIF2, MXI1, MXRA7, MYCBP2, MYO18A,
MYO5A, N6AMT2, NCK2, NCOR2,
NEDD1, NIN, NPRL3, NTMT1, NTPCR,
NUBP2, NUMB, NUP98, NUTF2, OPN3, OSER1-
AS1, P4HA2, PARD3, PCNXL2, PDGFC,
PDP1, PDPR, PDXDC2P, PEAK1, PFDN5,
PHLPP2, PIGF, PIGP, PIGQ, PINX1, PLD3,
PLSCR4, PPP1R18, PPP6R3, PRDM5,
PRRC2B, PRUNE, PTAR1, PTPRG, PTPRS,
PUF60, PUM2, R3HDM1, RAD51D, RAF1,
RAPH1, RBM10, RBM27, RBM4, RBM5,
RCC1, RFC2, RGL1, RIF1, RILP, RILPL2,
RNF14, RNF8, RNGTT, RP11-705C15.2,
RP1-178F15.4, RPAIN, RPP38, RPS24, RPUSD1,
RRNAD1, RTN2, RUNX1, SCRN3, SEC13,
SEC31A, SETD2, SEZ6L2, SGSM2, SIKE1,
SLAIN2, SLC30A6, SLC38A9, SLC39A9,
SLFN11, SLMAP, SLTM, SMEK2, SMG7,
SMURF2, SNHG14, SPEN, SPIDR, SRSF2,
STAG1, STAG2, STARD3, STARD4, STK38L,
STOML1, STXBP5, SUOX, SYNE1, SYNJ2BP,
TAMM41, TBC1D32, TBCE, TBCEL,
TCF20, TCF7L2, TENM2, TET2, TFB2M,
TFDP2, TGIF1, THADA, THTPA, THTPA,
TJAP1, TJP1, TLK2, TMEM119, TMEM161B,
TMEM175, TMEM230, TMUB2,
TOR1AIP2, TOR3A, TP53BP2, TRA2A,
TRIM37, TRMU, TROVE2, TSPAN5, TULP3,
U2AF1, UCHL5, UPF3A, UPP1, USP3, VDR,
VEGFA, VIPAS39, VPS13D, WARS,
WDR27, WHSC1, WNK1, XIAP, XPNPEP3,
YTHDF2, YWHAB, ZBTB38, ZCCHC6,
ZEB1, ZFAT, ZFX, ZMYM1, ZNF140,
ZNF217, ZNF260, ZNF266, ZNF3, ZNF346,
ZNF37A, ZNF419, ZNF426, ZNF529, ZNF585B, ZNF638,
ZNF655, ZNF75A, ZNF767, and ZSCAN32.

Another aspect described herein is Compound (I) for use in a method to modulate the production of one or more mature RNA isoforms from a wildtype gene transcript in a cell, wherein the method comprises, contacting the cell with Compound (I), wherein the wildtype gene transcript is transcribed from a gene other than a gene selected from Table 9.

One aspect described herein is use of Compound (I) to modulate the production of one or more mature RNA isoforms from a gene transcript in a cell comprising, contacting the cell with Compound (I), wherein the gene transcript is transcribed from a gene selected from Table 9.

Another aspect described herein is use of Compound (I) to modulate the production of one or more mature RNA isoforms from a wildtype gene transcript in a cell comprising, contacting the cell with Compound (I), wherein the wildtype gene transcript is transcribed from a gene other than a gene selected from Table 9.

One aspect described herein is Compound (I) for use in a method, wherein Compound (I) modulates the production of one or more mature RNA isoforms from a gene transcript in a cell, wherein the method comprises, contacting the cell with Compound (I), and wherein the gene transcript is transcribed from a gene selected from Table 9a.

Table 9a lists wildtype genes identified by RNA sequencing and mutated genes predicted by SpliceAI scores and the CNN model, as described herein, which may be modulated by Compound (I) toward either or both exon inclusion or exon exclusion and toward remaining unchanged.

TABLE 9a
ABCC3, ABI3BP, ACAD10, ACTN1, ANO6,
ARHGEF10, ARIH2, ASCC2, B3GALNT1,
BIRC6, BLOC1S6, BRD8, C11orf30, C9orf156,
CCDC25, DCAF8, DGUOK, ENC1, IP6K2,
KTN1, MADD, MEGF6, MYO18A, MYO5A,
NPRL3, PARD3, PCNXL2, PEAK1, PRUNE,
RIF1, RPAIN, SLMAP, SLTM, SMEK2,
SMURF2, SNHG14, TFDP2, TMUB2, TRIM37,
TROVE2, TULP3, and ZCCHC6.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 9a.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 9a.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 9a.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 9a.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 9a.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 9a.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 9a.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 9a.

Another aspect described herein is Compound (I) for use in the method, wherein Compound (I) modulates the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Table 9a.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Table 9a.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Table 9a.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Table 9a.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 9a.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 9a.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

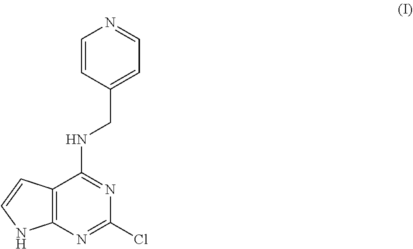 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 9a.

Another aspect described herein is Compound (I) for use in the method, wherein a gene having a mutation in a gene transcript thereof is selected from Table 9a.

One aspect described herein is use of Compound (I) to modulate the production of one or more mature RNA isoforms from a gene transcript in a cell comprising, contacting the cell with Compound (I), wherein the gene transcript is transcribed from a gene selected from Table 9a.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 9a.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 9a.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 9a.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 9a.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 9a.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 9a.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 9a.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 9a.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Table 9a.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Table 9a.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Table 9a.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Table 9a.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene is selected from Table 9a.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Table 9a.

Another aspect described herein is use of Compound (I) to treat a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Table 9a.

Another aspect described herein is use of Compound (I), wherein a gene having a mutation in a gene transcript thereof is selected from the group consisting of Table 9a.

One aspect described herein is Compound (I) for use in a method to modulate the production of one or more mature RNA isoforms from a wildtype gene transcript in a cell, wherein the method comprises, contacting the cell with Compound (I), wherein the wildtype gene transcript is transcribed from a gene selected from Table 10.

Table 10 lists wildtype genes identified by RNA sequencing, modulated by kinetin toward either or both exon inclusion and exon exclusion⁹³.

TABLE 10
ANKRD10, BRD8, C2CD5, CAPRIN2, CHD3,
CRYZ, CYLD, DUSP11, EDEM2,
HSD17B4, IKBKAP, KIF3A, KLC1, L3HYPDH,
LETMD1, MORF4L2, NABP1, NEK1,
NEXN, PLA2G12A, PPFIBP1, PPIP5K2,
RHOT1, RPL7L1, RUFY2, SBF1, SDCCAG8,
SECISBP2L, SLC4A7, SNX14, STARD4, STX16,
SUCO, SUPT20H, TEAD2, TEP1, TIA1,
ZFAND1, and ZNF207.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 10.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 10.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 10.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 10.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 10.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 10.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 10.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 10.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAgtaagt (SEQ ID NO: 1), and wherein the wildtype gene transcript is transcribed from a gene selected from Table 10.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAguaagu (SEQ ID NO: 5), and wherein the wildtype gene transcript is transcribed from a gene selected from Table 10.

Another aspect described herein is Compound (I) for use in the method, wherein Compound (I) modulates the production of one or more mature RNA isoforms from a gene transcript in a cell, wherein the method comprises, contacting the cell with Compound (I), and wherein the gene transcript is transcribed from a gene other than a gene selected from Table 10.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

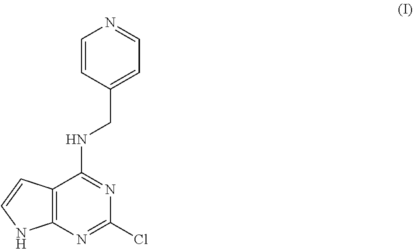 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 10.

Another aspect described herein is Compound (I) for use in the method, wherein a gene having a mutation in a gene transcript thereof is selected from the group consisting of Table 10.

Another aspect described herein is Compound (I) for use in the method, wherein a gene having a mutation in a gene transcript thereof is a gene other than the gene selected from the group consisting of Table 10.

One aspect described herein is use of Compound (I) to modulate the production of one or more mature RNA isoforms from a wildtype gene transcript in a cell comprising, contacting the cell with Compound (I), wherein the wildtype gene transcript is transcribed from a gene selected from Table 10.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 10.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 10.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 10.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 10.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 10.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 10.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 10.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 10.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAgtaagt (SEQ ID NO: 1), and wherein the wildtype gene transcript is transcribed from a gene selected from Table 10.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAguaagu (SEQ ID NO: 5), and wherein the wildtype gene transcript is transcribed from a gene selected from Table 10.

Another aspect described herein is use of Compound (I) to modulate the production of one or more mature RNA isoforms from a gene transcript in a cell comprising, contacting the cell with Compound (I), wherein the gene transcript is transcribed from a gene selected from Table 10.

Another aspect described herein is use of Compound (I) to modulate the production of one or more mature RNA isoforms from a gene transcript in a cell comprising, contacting the cell with Compound (I), wherein the gene transcript is transcribed from a gene other than a gene selected from Table 10.

One aspect described herein is Compound (I) for use in a method to modulate the production of one or more mature RNA isoforms from a gene transcript in a cell, wherein the method comprises, contacting the cell with Compound (I), wherein the gene transcript is transcribed from a gene selected from Table 11.

Table 11 lists wildtype genes identified by RNA sequencing, modulated by kinetin toward exon inclusion⁹³.

TABLE 11
CYLD, EDEM2, HSD17B4, IKBKAP, KLC1,
L3HYPDH, NABP1, SDCCAG8, SUCO,
SUPT20H, TIA1, and ZNF207.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 11.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 11.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 11.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 11.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 11.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 11.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 11.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 11.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAgtaagt (SEQ ID NO: 1), and wherein the wildtype gene transcript is transcribed from a gene selected from Table 11.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAguaagu (SEQ ID NO: 5), and wherein the wildtype gene transcript is transcribed from a gene selected from Table 11.

Another aspect described herein is Compound (I) for use in the method, wherein Compound (I) modulates the production of one or more mature RNA isoforms from a gene transcript in a cell, wherein the method comprises, contacting the cell with Compound (I), and wherein the gene transcript is transcribed from a gene selected from Table 11.

Another aspect described herein is Compound (I) for use in the method, wherein Compound (I) modulates the production of one or more mature RNA isoforms from a gene transcript in a cell, wherein the method comprises, contacting the cell with Compound (I), and wherein the gene transcript is transcribed from a gene other than a gene selected from Table 11.

One aspect described herein is use of Compound (I) to modulate the production of one or more mature RNA isoforms from a gene transcript in a cell comprising, contacting the cell with Compound (I), wherein the gene transcript is transcribed from a gene selected from Table 11.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 11.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 11.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 11.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 11.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 11.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 11.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 11.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 11.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAgtaagt (SEQ ID NO: 1), and wherein the wildtype gene transcript is transcribed from a gene selected from Table 11.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAguaagu (SEQ ID NO: 5), and wherein the wildtype gene transcript is transcribed from a gene selected from Table 11.

Another aspect described herein is use of Compound (I) to modulate the production of one or more mature RNA isoforms from a gene transcript in a cell comprising, contacting the cell with Compound (I), wherein the gene transcript is transcribed from a gene other than a gene selected from Table 11.

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 12.

Table 12 lists wildtype genes identified by RNA sequencing, modulated by kinetin toward exon exclusion⁹³.

TABLE 12
ANKRD10, BRD8, C2CD5, CAPRIN2, CHD3,
CRYZ, DUSP11, KIF3A, LETMD1,
MORF4L2, NEK1, NEXN, PLA2G12A, PPFIBP1,
PPIP5K2, RHOT1, RPL7L1, RUFY2,
SBF1, SECISBP2L, SLC4A7, SNX14, STARD4,
STX16, SUPT20H, TEAD2, TEP1,
ZFAND1, and ZNF207.

Another aspect described herein is Compound (I) for use in the method, wherein the gene transcript is a predicted wildtype or mutant gene transcript in a cell comprising, a nucleotide sequence selected from the group consisting of: aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 12.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 12.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 12.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 12.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 12.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 12.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 12.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 12.

Another aspect described herein is Compound (I) for use in the method, wherein Compound (I) modulates the production of one or more mature RNA isoforms from a gene transcript in a cell, wherein the method comprises, contacting the cell with Compound (I), and wherein the gene transcript is transcribed from a gene other than a gene selected from Table 12.

One aspect described herein is use of Compound (I) to modulate the production of one or more mature RNA isoforms from a gene transcript in a cell comprising, contacting the cell with Compound (I), wherein the gene transcript is transcribed from a gene selected from Table 12.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 12.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 12.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 12.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 12.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 12.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 12.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 12.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 12.

Another aspect described herein is use of Compound (I) to modulate the production of one or more mature RNA isoforms from a gene transcript in a cell comprising, contacting the cell with Compound (I), wherein the gene transcript is transcribed from a gene other than a gene selected from Table 12.

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Table 13.

Table 13 lists wildtype genes identified by RNA sequencing, as described in International Application No. PCT/US2016/013553, filed on Jan. 15, 2016, and published as International Publication No. WO2016/115434 on Jul. 21, 2016, the entire contents which are incorporated herein by reference, that may be modulated by Compound (I) toward exon inclusion.

TABLE 13
ABI2, ARFGEF2, ARHGEF6, BMP2K, C19orf12,
CACNA1S, CC2D2A, CDKL5, CHD2,
CHD7, CHD8, CHRNA4, COL6A3, CUL4B,
DEPDC5, DES, DMD, DNAJC6, DYNC2H1,
FBN1, FIG4, FKTN, FMR1, GOSR2, GRIN2A,
HDAC8, IGHMBP2, IKBKAP, KDM5C,
LAMA2, LRRK2, LRSAM1, MBD5, MECP2,
MICU1, MTM1, NEB, OPHN1, PGAP1,
PLEC, RB1, RYR1, SBF2, SCN1A, SCN9A,
SLC35A3, SLC6A8, SLC9A9, SMCHD1,
STXBP1, SYNGAP1, SZT2, TSC1, TSC2, and WDR45.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 13.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 13.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 13.

Another aspect described herein is Compound (I) for use in the method, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 13.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 13.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 13.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAgtaagt (SEQ ID NO: 1), and wherein the wildtype gene transcript is transcribed from a gene selected from Table 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAguaagu (SEQ ID NO: 5), and wherein the wildtype gene transcript is transcribed from a gene selected from Table 13.

Another aspect described herein is Compound (I) for use in the method, wherein Compound (I) modulates the production of one or more mature RNA isoforms from a gene transcript in a cell, wherein the method comprises, contacting the cell with Compound (I), and wherein the gene transcript is transcribed from a gene other than a gene selected from Table 13.

One aspect described herein is use of Compound (I) to modulate the production of one or more mature RNA isoforms from a gene transcript in a cell comprising, contacting the cell with Compound (I), wherein the gene transcript is transcribed from a gene selected from Table 13.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 13.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 13.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 13.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 8.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAgtaagt (SEQ ID NO: 1), and wherein the wildtype gene transcript is transcribed from a gene selected from Table 13.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from: CAAguaagu (SEQ ID NO: 5), and wherein the wildtype gene transcript is transcribed from a gene selected from Table 13.

Another aspect described herein is use of Compound (I) to modulate the production of one or more mature RNA isoforms from a gene transcript in a cell comprising, contacting the cell with Compound (I), wherein the gene transcript is transcribed from a gene other than a gene selected from Table 13.

### Preparation of Compound (I)

The small molecule splicing modulator compound described herein as Compound (I) has been disclosed in International Publication No. WO2016/115434 as Compound 100. Compound (I) can be prepared using the methods provided in International Publication WO2016/155434 and as described herein.

### Preparation of Compound (I) (2-chloro-N-(4-pyridylmethyl)-7H-pyrrolo[2,3-d]pyrimidin-4-amine)

In brief, 4-(aminomethyl)pyridine (2, 3420 mg, 3.20 mL, 31.6 mmol, 1.19 eq.) was added to a stirred suspension of 2,4-dichloro-7H-pyrrolo[2,3-d]pyrimidine (5001 mg, 26.60 mmol, 1.000 eq.) (obtained from AstaTech Inc., Bristol, Pa.) in 1,4-dioxane (50.0 mL) followed by addition of N,N-diisopropylethylamine (4450 mg, 6.00 mL, 34.1 mmol, 1.28 eq.) at room temperature. The reaction mixture was then heated to 90° C. and stirred at 90° C. overnight.

The reaction progress was monitored by LC-MS analysis of an aliquot of the reaction mixture. After about 12 h, approximately 6% of starting material was detected by LC-MS. The reaction was quenched by water resulting in an emulsion. The mixture was filtered through Celite and washed with EtOAc (3×80 mL). The organic phase was separated and the aqueous phase was extracted with EtOAc (3×40 mL). The combined organic phases were washed with brine (50 mL) and then dried over sodium sulfate

The volatiles were removed under reduced pressure to produce a crude product as a dark brown solid. EtOAc (100 mL) was added to the crude solid and the mixture was heated at reflux for 15 min before slowly cooled to room temperature. The resulting precipitate was collected by filtration, washed with cold EtOAc (30 mL) and diethyl ether (100 mL). The solid was dried under high vacuum overnight to produce 2-chloro-N-(4-pyridylmethyl)-7H-pyrrolo[2,3-d]pyrimidin-4-amine (Compound (I) as a light brown solid (3450 mg, 13.3 mmol, 50% yield.)

LC-MS: 0.63 min (254 nm), m/z 260.3, 262.3 [M+H]⁺, 258.2, 260.2 [M−H]⁻; ¹H NMR (DMSO-d₆) δ: 11.65-11.85 (m, 1H), 8.51 (d, J=6.0 Hz, 2H), 8.45-8.50 (m, 1H), 7.28-7.40 (m, 2H), 7.09-7.21 (m, 1H), 6.53-6.74 (m, 1H), 4.61-4.81 (m, 2H).

As used herein, Compound (I) may have a form selected from the group consisting of a free acid, free base, prodrug, salt, hydrate, solvate, clathrate, isotopologue, racemate, enantiomer, diastereomer, stereoisomer, polymorph and tautomer form thereof.

In certain aspects described herein, the form of Compound (I) is a free acid, free base or salt form thereof.

In certain aspects described herein, Compound (I) is a salt form.

In certain aspects described herein, the salt form of Compound (I) is a pharmaceutically acceptable salt.

In certain aspects described herein, Compound (I) is isolated for use.

The term “pharmaceutically acceptable salt(s)”, as used herein, means a salt of Compound (I) that is safe and effective (i.e., non-toxic, physiologically acceptable) for use in mammals and possesses biological activity, although other salts may be found useful. A salt of Compound (I) may be formed, for example, by reacting Compound (I) with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. In certain aspects, acid addition salts may include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, hydrochloride, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate and the like. Certain aspects of acid addition salts may further include acetate, bromide, chloride, dichloride, trichloride, hydrochloride, dihydrochloride, formate or trifluoroacetate salts.

All such acid salts and base salts are intended to be included within the scope of pharmaceutically acceptable salts as described herein. In addition, all such acid and base salts are considered equivalent to the free forms of Compound (I).

The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or isotopologues of the instant compounds.

Another aspect, described herein includes Compound (I) selected from a polymorphic crystalline and amorphous form of Compound (I) and a salt, solvate, hydrate or ester of Compound (I).

Nomenclature for Compound (I) may differ slightly from other chemical names known to those skilled in the art; however, such differences will be recognized by one skilled in the art as equivalents for the structure of Compound (I) provided herein.

### Terminology

As used herein, the singular forms “a,” “an,” and “the,” are intended to include the plural forms as well, unless the context clearly indicates otherwise.

The phrase “and/or,” as used herein and in the claims, is understood to mean “either or both” of the elements so conjoined, i.e., elements that are conjunctively present in some cases and disjunctively present in other cases. Thus, as a non-limiting example, a reference to “A and/or B,” when used in conjunction with open-ended language such as “comprising” can refer, in one aspect, to A only (optionally including elements other than B); in another aspect, to B only (optionally including elements other than A); in yet another aspect, to both A and B (optionally including other elements); etc

As used herein and in the claims, the phrase “at least one,” in reference to a list of one or more elements, should be understood to mean at least one element selected from any one or more of the elements in the list of elements, but not necessarily including at least one of each and every element specifically listed within the list of elements, and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain aspects, the term “about” is used to modify a numerical value above and below the stated value by a variance of 10%. In certain aspects, the term “about” is used to modify a numerical value above and below the stated value by a variance of 5%. In certain aspects, the term “about” is used to modify a numerical value above and below the stated value by a variance of 1%.

As used herein, the term “substantial change” in the context of the amount of one or more RNA transcripts, an alternative splice variant thereof or an isoform thereof, or one or more proteins thereof, each expressed as the product of one or more of genes, means that the amount of such products changes by a statistically significant amount such as, in a nonlimiting example, a p value less than a value selected from 0.1, 0.01, 0.001, or 0.0001.

As used herein, the terms “subject” and “patient” are used interchangeably to refer to an animal or any living organism having sensation and the power of voluntary movement, and which requires for its existence oxygen and organic food. Non-limiting examples include members of the human, equine, porcine, bovine, rattus, murine, canine and feline species. In some aspects, the subject is a mammal or a warm-blooded vertebrate animal. In certain aspects, the subject is a non-human animal. In specific aspects, the subject is a human.

When a range of values is listed herein, it is intended to encompass each value and sub-range within that range. For example, “1-5 ng” or a range of “1 ng to 5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, the terms “treat,” “treatment,” “treating” refer to therapeutic treatments, wherein the object is to reverse, alleviate, ameliorate, inhibit, slow down or stop the progression or severity of a disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the terms “subject” and “patient” are used interchangeably to refer to an animal or any living organism having sensation and the power of voluntary movement, and which requires for its existence oxygen and organic food. Non-limiting examples include members of the human, equine, porcine, bovine, rattus, murine, canine and feline species. In one aspect, the subject is a mammal or a warm-blooded vertebrate animal. In another aspect, the subject is a non-human animal. In another aspect, the subject is a human.

As used herein, the term “RNA” means a molecule comprising at least one ribonucleotide residue. By “ribonucleotide” is meant a nucleotide with a hydroxyl group at the 2′ position of a beta-D-ribo-furanose moiety. The terms include double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. RNAs can be synthesized in a cell by RNA polymerase I, II or III.

The term “mRNA” refers to any RNA that is produced in a cell by RNA polymerase II transcription of a gene. In one aspect, the mRNA of the disclosure is capped and polyadenylated. In one aspect, an mRNA of the disclosure encodes one or more proteins. In one aspect, the mRNA does not encode a protein. In another aspect, mRNA can refer to processed or unprocessed pre-mRNA. In another aspect, the mRNA of this disclosure includes, but is not limited to, pre-mRNA, spliced mRNA, partially spliced mRNA and alternatively spliced mRNA. In one aspect, the mRNA of the disclosure is a transcript that undergoes nonsense-mediated decay (NMD) in the presence of a compound as described herein.

Splicing is a natural biological mechanism that may occur within human cells. Splicing processes primary messenger ribonucleic acid (mRNA) that has been transcribed from deoxyribonucleic acid (DNA) before the mRNA is translated into a protein. Splicing involves removing one or more contiguous segments of mRNA and is directed, in part, by a spliceosome. The segments that are removed are often referred to as introns, but the spliceosome may remove segments that contain both introns and exons.

As used herein, the term “functional protein” refers to a form of a protein that retains a certain biological function or the functions of a full length protein or protein isoform encoded by a gene. As used herein, in the context of the use of Compound (I) to modulate the production of one or more mature RNA isoforms from a gene transcript in a cell comprising, contacting the cell with Compound (I) to produce a functional protein, wherein the amount of functional protein produced in the absence of Compound (I) is at least 10%, 15%, 20%, 25%, 30%, 35%, 40%, 45%, 50%, 55%, 60%, 70%, 75%, 80%, 85%, 90%, 95%, 98%, or 100% less than the amount of functional protein produced in the presence of Compound (I).

As used herein, in the context of Compound (I) for use in the method, wherein Compound (I) modulates the production of one or more mature RNA isoforms from a gene transcript in a cell, wherein the method comprises, contacting the cell with Compound (I) to produce a functional protein, and wherein the amount of functional protein produced in the absence of Compound (I) is at least 10%, 15%, 20%, 25%, 30%, 35%, 40%, 45%, 50%, 55%, 60%, 70%, 75%, 80%, 85%, 90%, 95%, 98%, or 100% less than the amount of functional protein produced in the presence of Compound (I).

As used herein, the term “exon” refers to any part of a gene that is a part of the final mature RNA produced by that gene after introns have been removed by RNA splicing. The term “exon” refers to both the DNA sequence within a gene and to the corresponding sequence in RNA transcripts.

As used herein, the term “intron” refers to both the DNA sequence within a gene and the corresponding sequence in the unprocessed RNA transcript. As part of the RNA processing pathway, introns can be removed by RNA splicing either shortly after or concurrent with transcription.

As used herein, the term “isolated” means the physical state of Compound (I) after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “exon triplet” refers to three consecutive exons in a gene transcript, each separated by adjacent introns. One aspect described herein includes a gene transcript of interest comprising an exon triplet for interrogation by the CNN model as described herein.

As used herein, the term “5-mer enrichment analysis” refers to 5-mer sets of nucleotides identified as adjacent nucleotides from the −3 to +7 position in a splice site junction known by those skilled in the art to have an observed frequency of Class Effect greater than mere chance toward modulating the splicing reaction. Such 5-mer sets of nucleotides having a frequency p value<0.05 toward a particular Class Effect were deemed to be enriched.

As used herein, the term “LOGO plot” refers to the presentation of a nucleotide sequence known by those skilled in the art to show an alignment of nucleotides having a particular Class Effect. In one aspect described herein, the representation of nucleotides in a LOGO plot sequence may show only one nucleotide in a LOGO plot position. In another aspect described herein, the representation of nucleotides in a LOGO plot sequence may show one or more nucleotides having differing heights in a LOGO plot position. In another aspect described herein, the representation of nucleotides in a LOGO plot sequence may show no visible nucleotide in a LOGO plot position. Accordingly, nucleotides shown in a LOGO plot position represent potential variability for the nucleotide to be in that position. It is assumed by those skilled in the art that the probability for each one of the four possible nucleotides to be present in any one position is assumed to be equally random, at least 25% of the time. As used herein, the term “constant,” in reference to a nucleotide in a particular LOGO plot position having only one nucleotide, indicates that the probability of the nucleotide to be present in that position is predicted to be 100%. In a LOGO plot presented herein, the constant nucleotide is shown by a capital letter representing the corresponding nucleotide. As used herein, the term “most dominant,” in reference to a nucleotide in a particular LOGO plot position having no clear constant nucleotide, where more than one nucleotide is present as shown in a vertical nucleotide stack, indicates that the probability of one or more nucleotides to be present in that position is predicted to be less than 100%. In a LOGO plot presented herein, the most dominant nucleotide is taken to be the topmost nucleotide in the LOGO plot position and is shown by a capital letter representing the corresponding nucleotide. In other instances, where no visible nucleotide appears in a LOGO plot position, the position is shown by the capital letter “N”.

As used herein, the term “Class” refers to exclusion or inclusion of an exon in an mRNA splicing reaction in the presence of a small molecule splicing compound to produce one or more mature RNA isoforms that are unlike an isoform produced by the wildtype or mutant gene transcript. In another aspect described herein, the effect may be the production of one or more mature RNA isoforms unchanged from the wildtype or mutant spliced isoform.

As used herein, the term “Class Effect” refers to the production of one or more mature RNA isoforms from a wildtype or mutant gene transcript in the presence of a 5-mer nucleotide sequence identified or predicted to have an effect toward exclusion or inclusion of an exon in an mRNA splicing reaction while in the presence of a small molecule splicing compound. In another aspect described herein, the predicted effect may be the production of one or more mature RNA isoforms unchanged from the wildtype or mutant spliced isoform.

As used herein, the terms “Active Class” and “Active Class Effect” refer to a set of 5-mer nucleotide sequences having a Class Effect on mRNA splicing in the presence of a small molecule splicing compound toward exclusion or inclusion of an exon in an mRNA splicing reaction in the presence of a small molecule splicing compound to produce one or more mature RNA isoforms from the wildtype or mutant gene transcript.

As used herein, the terms “Unchanged Class” and “Unchanged Class Effect” refer to a set of 5-mer nucleotide sequences having a Class Effect toward exclusion or inclusion of an exon in an mRNA splicing reaction in the presence of a small molecule splicing compound to produce one or more mature RNA isoforms that are unchanged from those produced by the wildtype or mutant gene transcript in the absence of the small molecule splicing compound.

As used herein, the term “Enrichment Motif” refers to a 5-mer nucleotide sequence identified in an enrichment analysis as described herein are known to have a certain Class Effect on mRNA splicing in the presence of a small molecule splicing compound. In one aspect described herein, an Enrichment Motif relates to a set of 5-mer nucleotide sequences identified as having an Active Class Effect. In another aspect described herein, an Enrichment Motif relates to a set of 5-mer nucleotide sequences identified as having an Unchanged Class Effect. In another aspect described herein, all nucleotides in an Enrichment Motif are assumed to contribute to the frequency of the Enrichment Motif as a unit toward a particular Class Effect. In another aspect described herein, LOGO plots for Enrichment Motifs may show nucleotides having varying heights, where the nucleotide in the position may have either a greater or lesser frequency toward having a certain Class Effect. In another aspect described herein, Constant nucleotides or More Dominant nucleotides shown in Enrichment Motif LOGO plots indicate those single nucleotides have a greater frequency toward having an Active Class Effect. In another aspect described herein, positions absent a nucleotide shown in the Enrichment Motif LOGO plot indicate nucleotides in those positions have an Unchanged Class Effect, having no frequency contribution toward an Active Class Effect.

As used herein, the term “CNN Motif” refers to a 5-mer nucleotide sequence identified and predicted by the CNN model described herein to have a Class Effect on mRNA splicing in the presence of a small molecule splicing compound. In one aspect described herein, the CNN Model was taught to convolute 400-mer sequences containing Enrichment Motifs into separate elements, where the position of each element could be analyzed as to whether the element in that position could contribute to a correct prediction toward a particular Class Effect. In another aspect described herein, once built, the CNN Model was used to weight the predictive contribution of every five nucleotides (5-mer) in the 400-mer through a convolution process. Each position within a 5-mer was individually weighted according to potential contribution to a correct prediction toward a particular Class Effect. As a result, certain 5-mer nucleotide positions having a more predictive effect were identified, enabling these 5-mer positions to be more preferentially weighted. In another aspect described herein, without being limited in any way by theory in any aspect of the CNN Model, the positional analysis convoluted by the CNN Model suggested that nucleotides in the −3 to +2 region of the splice junction were more heavily weighted. In another aspect described herein, the convoluted, highly weighted 5-mer nucleotide positions were extracted from the CNN model and converted into CNN motifs according to the weight at each position. In another aspect described herein, multiple CNN Motifs may be convoluted using the CNN Model described herein to weight nucleotides in a gene sequence of interest to predict whether a Class Effect will occur. For each CNN Motif, a single overall Class Effect for the CNN Motif was calculated, ranking the statistical probability of the CNN Motif to correctly predict a single overall Class Effect based on the statistical probability resulting from the particular analytical method used. In another aspect described herein, the frequency of an Enrichment Motif to have a particular Class Effect may be compared with the probability of a similar or dissimilar CNN motif to correctly predict a particular Class Effect using a Pearson Correlation. In one aspect described herein, a CNN Motif relates to a set of 5-mer nucleotide sequences identified as having an Active Class Effect. In one aspect described herein, a CNN Motif relates to a set of 5-mer nucleotide sequences identified as having an Unchanged Class Effect. In another aspect described herein, LOGO plots for CNN Motifs may show nucleotides having varying heights, where the nucleotide in the position may have either a greater or lesser positional importance toward a predicting a certain Class Effect. In another aspect described herein, Constant nucleotides and More Dominant nucleotides in a CNN Motif identified by the CNN model may be expected to have a positional importance toward a Class Effect. In another aspect described herein, one or more of either or both Constant nucleotides and More Dominant nucleotides in a deconvoluted CNN Motif LOGO plot may influence prediction toward a variety of Active Class Effects, the convoluted CNN Motif may have a greater positional importance toward predicting a single overall Active Class Effect. In another aspect described herein, positions absent a nucleotide shown in the CNN Motif LOGO plot indicate single nucleotides in those positions have an Unchanged Class Effect, where the open positions have no positional importance toward predicting an overall Active Class Effect.

As used herein, the term “Pearson Correlation” refers to a statistical correlation comparing the Class Effect of an Enrichment Motif with a CNN Motif. The Pearson Correlation compares the overall Class Effect predicted by an individual CNN Motif with the frequency of Class Effect for an individual Enrichment Motif. In another aspect described herein, the Pearson Correlation for an Enrichment Motif may show a Positive or Negative Correlation to a Class Effect for a similar or dissimilar CNN Motif.

As used herein, the phrase “predicted wildtype or mutant gene transcript” refers to a gene transcript containing a 5-mer nucleotide sequence identified as having a predicted Class Effect toward exclusion or inclusion of an exon in an mRNA splicing reaction in the presence of a small molecule splicing compound to produce one or more mature RNA isoforms from the wildtype or mutant gene transcript. In one aspect described herein, the predicted effect of the 5-mer nucleotide sequence may be identified using a CNN model or equivalents thereof which are within the scope of one skilled in the art to design. In another aspect described herein, the predicted effect of the 5-mer nucleotide sequence may be identified in an enrichment analysis using methods known to those skilled in the art.

### Pharmaceutical Compositions and Modes of Administration

When administered to a patient, Compound (I) is preferably administered as a component of a composition that optionally comprises a pharmaceutically acceptable carrier, excipient or diluent. The composition can be administered orally, or by any other convenient route, for example, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., encapsulation in liposomes, microparticles, microcapsules, capsules, and can be used to administer the compound.

Methods of administration include, but are not limited to, parenteral, intradermal, intramuscular, intraperitoneal, intravenous, subcutaneous, intranasal, epidural, oral, sublingual, intranasal, intraocular, intratumoral, intracerebral, intravaginal, transdermal, ocularly, rectally, by inhalation, or topically, particularly to the ears, nose, eyes, or skin. The mode of administration is left to the discretion of the practitioner. In most instances, administration will result in the release of a compound into the bloodstream, tissue or cell(s). In a specific aspect, a compound is administered orally.

The amount of Compound (I) that will be effective in the treatment of a disease resulting from an aberrant amount of mRNA transcripts depends, e.g., on the route of administration, the disease being treated, the general health of the subject, ethnicity, age, weight, and gender of the subject, diet, time, and the severity of disease progress, and should be decided according to the judgment of the practitioner and each patient's or subject's circumstances.

In specific aspects, an “effective amount” in the context of the administration of Compound (I), or composition or medicament thereof refers to an amount of Compound (I) to a patient which has a therapeutic effect and/or beneficial effect. In certain specific aspects, an “effective amount” in the context of the administration of Compound (I), or composition or medicament thereof to a patient results in one, two or more of the following effects: (i) reduces or ameliorates the severity of a disease; (ii) delays onset of a disease; (iii) inhibits the progression of a disease; (iv) reduces hospitalization of a subject; (v) reduces hospitalization length for a subject; (vi) increases the survival of a subject; (vii) improves the quality of life of a subject; (viii) reduces the number of symptoms associated with a disease; (ix) reduces or ameliorates the severity of a symptom(s) associated with a disease; (x) reduces the duration of a symptom associated with a disease associated; (xi) prevents the recurrence of a symptom associated with a disease; (xii) inhibits the development or onset of a symptom of a disease; and/or (xiii) inhibits of the progression of a symptom associated with a disease. In certain aspects, an effective amount of Compound (I) is an amount effective to restore the amount of a RNA transcript of a gene to the amount of the RNA transcript detectable in healthy patients or cells from healthy patients. In other aspects, an effective amount of Compound (I) is an amount effective to restore the amount an RNA isoform and/or protein isoform of gene to the amount of the RNA isoform and/or protein isoform detectable in healthy patients or cells from healthy patients.

In certain aspects, an effective amount of Compound (I) is an amount effective to decrease the aberrant amount of an RNA transcript of a gene which associated with a disease. In certain aspects, an effective amount of Compound (I) is an amount effective to decrease the amount of the aberrant expression of an isoform of a gene. In some aspects, an effective amount of Compound (I) is an amount effective to result in a substantial change in the amount of an RNA transcript (e.g., mRNA transcript), alternative splice variant or isoform.

In certain aspects, an effective amount of Compound (I) is an amount effective to increase or decrease the amount of an RNA transcript (e.g., an mRNA transcript) of gene which is beneficial for the prevention and/or treatment of a disease. In certain aspects, an effective amount of Compound (I) is an amount effective to increase or decrease the amount of an alternative splice variant of an RNA transcript of gene which is beneficial for the prevention and/or treatment of a disease. In certain aspects, an effective amount of Compound (I) is an amount effective to increase or decrease the amount of an isoform of gene which is beneficial for the prevention and/or treatment of a disease. Non-limiting examples of effective amounts of Compound (I) are described herein.

For example, the effective amount may be the amount required to prevent and/or treat a disease associated with the aberrant amount of an mRNA transcript of gene in a human subject.

In general, the effective amount will be in a range of from about 0.001 mg/kg/day to about 500 mg/kg/day for a patient having a weight in a range of between about 1 kg to about 200 kg. The typical adult subject is expected to have a median weight in a range of between about 70 and about 100 kg.

Within the scope of the present description, the “effective amount” of Compound (I) for use in the manufacture of a medicament, the preparation of a pharmaceutical kit or in a method for preventing and/or treating a disease in a human subject in need thereof, is intended to include an amount in a range of from about 0.001 mg to about 35,000 mg.

The compositions described herein are formulated for administration to the subject via any drug delivery route known in the art. Non-limiting examples include oral, ocular, rectal, buccal, topical, nasal, ophthalmic, subcutaneous, intramuscular, intravenous (bolus and infusion), intracerebral, transdermal, and pulmonary routes of administration.

Aspects described herein include the use of Compound (I) in a pharmaceutical composition. In a specific aspect, described herein is the use of Compound (I) in a pharmaceutical composition for preventing and/or treating a disease in a human subject in need thereof comprising administering an effective amount of Compound (I) in admixture with a pharmaceutically acceptable carrier, excipient or diluent. In a specific aspect, the human subject is a patient with a disease associated with the aberrant amount of an mRNA transcript(s).

Compound (I) may optionally be in the form of a composition comprising the compound or a form thereof and an optional carrier, excipient, or diluent. Other aspects provided herein include pharmaceutical compositions comprising an effective amount of Compound (I) and a pharmaceutically acceptable carrier, excipient, or diluent. In a specific aspect, the pharmaceutical compositions are suitable for veterinary and/or human administration. The pharmaceutical compositions provided herein can be in any form that allows for the composition to be administered to a subject.

In a specific aspect and in this context, the term “pharmaceutically acceptable carrier, excipient or diluent” means a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which a therapeutic agent is administered. Such pharmaceutical carriers can be sterile liquids, such as water and oils, including those of petroleum, animal, vegetable, or synthetic origin, such as peanut oil, soybean oil, mineral oil, sesame oil and the like. Water is a specific carrier for intravenously administered pharmaceutical compositions. Saline solutions and aqueous dextrose and glycerol solutions can also be employed as liquid carriers, particularly for injectable solutions.

Typical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. Further provided herein are anhydrous pharmaceutical compositions and dosage forms comprising Compound (I) as described herein. The compositions and single unit dosage forms can take the form of solutions or syrups (optionally with a flavoring agent), suspensions (optionally with a flavoring agent), emulsions, tablets (e.g., chewable tablets), pills, capsules, granules, powder (optionally for reconstitution), taste-masked or sustained-release formulations and the like.

Pharmaceutical compositions provided herein that are suitable for oral administration can be presented as discrete dosage forms, such as, but are not limited to, tablets, caplets, capsules, granules, powder, and liquids. Such dosage forms contain predetermined amounts of active ingredients, and may be prepared by methods of pharmacy well known to those skilled in the art.

Examples of excipients that can be used in oral dosage forms provided herein include, but are not limited to, binders, fillers, disintegrants, and lubricants.

In another aspect, the method for modulating the amount of one, two, three or more RNA transcripts of a gene described herein, comprising contacting a cell with Compound (I) includes a cell in a cell culture. In other aspects, the cell is contacted with Compound (I) in a subject (e.g., a non-human animal subject or a human subject).

In certain aspects described herein, the cell(s) is contacted or cultured with Compound (I) with Compound (I) for a period of 15 minutes, 30 minutes, 45 minutes, 1 hour, 2 hours, 3 hours, 4 hours, 5 hours, 6 hours, 8 hours, 12 hours, 18 hours, 24 hours, 48 hours, 72 hours or more. In other aspects described herein, the cell(s) is contacted or cultured with Compound (I) with Compound (I) for a period of 15 minutes to 1 hour, 1 to 2 hours, 2 to 4 hours, 6 to 12 hours, 12 to 18 hours, 12 to 24 hours, 28 to 24 hours, 24 to 48 hours, 48 to 72 hours.

In certain aspects described herein, the cell(s) is contacted or cultured with a certain concentration of Compound (I), wherein the certain concentration is 0.01 μM, 0.05 μM, 1 μM, 2 μM, 5 μM, 10 μM, 15 μM, 20 μM, 25 μM, 50 μM, 75 μM, 100 μM, or 150 μM. In other aspects described herein, the cell(s) is contacted or cultured with a certain concentration of Compound (I), wherein the certain concentration is 175 μM, 200 μM, 250 μM, 275 μM, 300 μM, 350 μM, 400 μM, 450 μM, 500 μM, 550 μM 600 μM, 650 μM, 700 μM, 750 μM, 800 μM, 850 μM, 900 μM, 950 μM or 1 mM. In some aspects described herein, the cell(s) is contacted or cultured with a certain concentration of Compound (I), wherein the certain concentration is 5 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, or 950 nM. In certain aspects described herein, the cell(s) is contacted or cultured with a certain concentration of Compound (I), wherein the certain concentration is between 0.01 μM to 0.1 μM, 0.1 μM to 1 μM, 1 μM to 50 μM, 50 μM to 100 μM, 100 μM to 500 μM, 500 μM to 1 nM, 1 nM to 10 nM, 10 nM to 50 nM, 50 nM to 100 nM, 100 nM to 500 nM, 500 nM to 1000 nM. In certain aspects described herein, the cell(s) is contacted or cultured with a certain concentration of Compound (I) that results in a substantial change in the amount of an RNA transcript (e.g., an mRNA transcript), an alternatively spliced variant, or an isoform of a gene (e.g., a gene described herein, infra).

In another aspect, provided herein are methods for modulating the amount of one, two, three or more RNA transcripts of a gene, comprising administering to a human or non-human subject Compound (I), or a pharmaceutical composition comprising Compound (I) and a pharmaceutically acceptable carrier, excipient or diluent.

In one aspect, provided herein are methods for modulating the amount of one, two, three or more RNA transcripts of a gene described herein, the methods comprising administering to a human or non-human subject Compound (I), or a pharmaceutical composition comprising Compound (I) and a pharmaceutically acceptable carrier, excipient or diluent.

In certain aspects, Compound (I) contacted or cultured with a cell(s) or administered to a subject is a compound as described herein.

### Compound (I) Use in a Method

One aspect described herein is Compound (I) for use in a method to modulate the production of one or more mature RNA isoforms from a gene transcript in a cell, wherein the method comprises, contacting the cell with Compound (I).

Another aspect described herein is Compound (I) for use in the method, wherein the gene transcript is a wildtype gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

(SEQ ID NO: 1)
CAAgtaagt,
(SEQ ID NO: 2)
GATTAAgtgggt,
(SEQ ID NO: 3)
CACTAGgtgaga,
and
(SEQ ID NO: 4)
CCAgtgagga.

Another aspect described herein is Compound (I) for use in the method, wherein the 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

(SEQ ID NO: 5)
CAAguaagu,
(SEQ ID NO: 6)
GAUUAAgugggu,
(SEQ ID NO: 7)
CACUAGgugaga,
and
(SEQ ID NO: 8)
CCAgugagga.

Another aspect described herein is Compound (I) for use in the method, wherein the gene transcript is a mutant gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of:

(SEQ ID NO: 9)
CAAgtaagc,
(SEQ ID NO: 10)
GATTAAgtaggt,
(SEQ ID NO: 11)
CACTAGgtgagc,
(SEQ ID NO: 12)
CCAgttagga,
(SEQ ID NO: 13)
AGCCAAgtatgt,
(SEQ ID NO: 14)
ATCCAAgtatgt,
(SEQ ID NO: 15)
CTGAAgtcagt,
and
(SEQ ID NO: 16)
AGTgtaagta.

Another aspect described herein is Compound (I) for use in the method, wherein the 5′ splice site of Exon 2 comprises, a sequence selected from the group consisting of:

(SEQ ID NO: 17)
CAAguaagc,
(SEQ ID NO: 18)
GAUUAAguaggu,
(SEQ ID NO: 19)
CACUAGgugagc,
(SEQ ID NO: 20)
CCAguuagga,
(SEQ ID NO: 21)
AGCCAAguaugu,
(SEQ ID NO: 22)
AUCCAAguaugu,
(SEQ ID NO: 23)
CUGAAgucagu,
and
(SEQ ID NO: 24)
AGUguaagua.

Another aspect described herein is Compound (I) for use in the method, wherein the gene transcript is a predicted wildtype or mutant gene transcript in a cell comprising, a sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is Compound (I) for use in the method, wherein the gene transcript is a predicted wildtype or mutant gene transcript in a cell comprising, a sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 5)
CAAguaagu,
(SEQ ID NO: 6)
GAUUAAgugggu,
(SEQ ID NO: 7)
CACUAGgugaga,
and
(SEQ ID NO: 8)
CCAgugagga.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 9)
CAAgtaagc,
(SEQ ID NO: 10)
GATTAAgtaggt,
(SEQ ID NO: 11)
CACTAGgtgagc,
(SEQ ID NO: 12)
CCAgttagga,
(SEQ ID NO: 13)
AGCCAAgtatgt,
(SEQ ID NO: 14)
ATCCAAgtatgt,
(SEQ ID NO: 15)
CTGAAgtcagt,
and
(SEQ ID NO: 16)
AGTgtaagta.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 17)
CAAguaagc,
(SEQ ID NO: 18)
GAUUAAguaggu,
(SEQ ID NO: 19)
CACUAGgugagc,
(SEQ ID NO: 20)
CCAguuagga,
(SEQ ID NO: 21)
AGCCAAguaugu,
(SEQ ID NO: 22)
AUCCAAguaugu,
(SEQ ID NO: 23)
CUGAAgucagu,
and
(SEQ ID NO: 24)
AGUguaagua.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I).

Another aspect described herein is Compound (I) for use in the method, wherein Compound (I) increases exon inclusion to produce one or more mature RNA isoforms from a gene transcript in a cell, wherein the method comprises, contacting the cell with Compound (I).

Another aspect described herein is Compound (I) for use in the method, wherein Compound (I) increases exon exclusion to produce one or more mature RNA isoforms from a gene transcript in a cell, wherein the method comprises, contacting the cell with Compound (I).

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I).

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

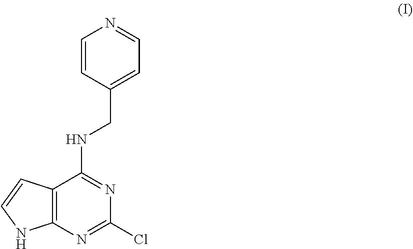 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 14.

Another aspect described herein is Compound (I) for use in the method, wherein a gene having the mutated transcript is selected from the group consisting of Table 14 and 15.

Another aspect described herein is Compound (I) for use in the method, wherein a gene having the mutated transcript is selected from the group consisting of Table 14.

Another aspect described herein is Compound (I) for use in the method, wherein a gene having the mutated transcript is other than the gene selected from the group consisting of Table 15.

### Use of Compound (I)

One aspect described herein is use of Compound (I) to modulate the production of one or more mature RNA isoforms from a gene transcript in a cell comprising, contacting the cell with Compound (I).

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is use of Compound (I) to modulate exon inclusion or exon exclusion in one or more mature RNA isoforms from a gene transcript in a cell comprising, contacting the cell with Compound (I).

Another aspect described herein is use of Compound (I) to increase exon inclusion in one or more mature RNA isoforms from a gene transcript in a cell comprising, contacting the cell with Compound (I).

Another aspect described herein is use of Compound (I) to increase exon exclusion in one or more mature RNA isoforms from a gene transcript in a cell comprising, contacting the cell with Compound (I).

Another aspect described herein is use of Compound (I) to modulate exon inclusion or exon exclusion in one or more mature RNA isoforms from a gene transcript in a cell comprising, contacting the cell in vivo or in vitro with Compound (I).

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is use of Compound (I) to treat a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript.

Another aspect described herein is use of Compound (I), wherein a gene having the mutated transcript is selected from the group consisting of Table 14 and 15.

Another aspect described herein is use of Compound (I), wherein a gene having the mutated transcript is selected from the group consisting of Table 14.

Another aspect described herein is use of Compound (I), wherein a gene having the mutated transcript is other than the gene selected from the group consisting of Table 15.

### Compound (I) Use in a Method

One aspect described herein is Compound (I) for use in a method for modulating the production of one or more mature RNA isoforms from a gene transcript in a cell, wherein the method comprises, contacting the cell with Compound (I).

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is Compound (I) for use in a method for modulating the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is Compound (I) for use in a method for modulating the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is Compound (I) for use in a method for modulating the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is Compound (I) for use in a method for modulating the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is Compound (I) for use in a method for modulating the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), GCAGA (SEQ ID NO: 69), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), GAGAG (SEQ ID NO: 79), AGGAN (SEQ ID NO: 80), AGACC (SEQ ID NO: 81), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), GGGAA (SEQ ID NO: 94), NUGNN (SEQ ID NO: 95), NUCCC (SEQ ID NO: 96), CAGGC (SEQ ID NO: 97), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 1)
CAAgtaagt,
(SEQ ID NO: 2)
GATTAAgtgggt,
(SEQ ID NO: 3)
CACTAGgtgaga,
and
(SEQ ID NO: 4)
CCAgtgagga.

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 5)
CAAguaagu,
(SEQ ID NO: 6)
GAUUAAgugggu,
(SEQ ID NO: 7)
CACUAGgugaga,
and
(SEQ ID NO: 8)
CCAgugagga.

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 9)
CAAgtaagc,
(SEQ ID NO: 10)
GATTAAgtaggt,
(SEQ ID NO: 11)
CACTAGgtgagc,
(SEQ ID NO: 12)
CCAgttagga,
(SEQ ID NO: 13)
AGCCAAgtatgt,
(SEQ ID NO: 14)
ATCCAAgtatgt,
(SEQ ID NO: 15)
CTGAAgtcagt,
and
(SEQ ID NO: 16)
AGTgtaagta.

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of:

(SEQ ID NO: 17)
CAAguaagc,
(SEQ ID NO: 18)
GAUUAAguaggu,
(SEQ ID NO: 19)
CACUAGgugagc,
(SEQ ID NO: 20)
CCAguuagga,
(SEQ ID NO: 21)
AGCCAAguaugu,
(SEQ ID NO: 22)
AUCCAAguaugu,
(SEQ ID NO: 23)
CUGAAgucagu,
and
(SEQ ID NO: 24)
AGUguaagua.

Another aspect described herein is Compound (I) for use in a method for modulating exon inclusion or exon exclusion in one or more mature RNA isoforms from a gene transcript in a cell, wherein the method comprises, contacting the cell with Compound (I).

Another aspect described herein is Compound (I) for use in a method for increasing exon inclusion in one or more mature RNA isoforms from a gene transcript in a cell, wherein the method comprises, contacting the cell with Compound (I).

Another aspect described herein is Compound (I) for use in a method for increasing exon exclusion in one or more mature RNA isoforms from a gene transcript in a cell, wherein the method comprises, contacting the cell with Compound (I).

Another aspect described herein is Compound (I) for use in a method for modulating exon inclusion or exon exclusion in one or more mature RNA isoforms from a gene transcript in a cell, wherein the method comprises, contacting the cell in vivo or in vitro with Compound (I).

Another aspect described herein is Compound (I) for use in a method for treating a disease associated with or mediated by a mutation in a gene transcript in a subject in need thereof, wherein the method comprises, administering Compound (I) to the subject, wherein exon inclusion or exon exclusion is modulated in one or more mature RNA isoforms produced from the gene transcript, and wherein the subject is human.

Another aspect described herein is Compound (I) for use in a method for treating a disease associated with or mediated by a mutation in a gene transcript in a subject in need thereof, wherein the method comprises, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is Compound (I) for use in a method for treating a disease associated with or mediated by a mutation in a gene transcript in a subject in need thereof, wherein the method comprises, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript.

### Methods for Modulating Isoform Production

One aspect described herein is a method for modulating the production of one or more mature RNA isoforms from a gene transcript in a cell comprising, contacting the cell with Compound (I).

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, and wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

Another aspect described herein is a method for modulating exon inclusion or exon exclusion in one or more mature RNA isoforms from a gene transcript in a cell comprising, contacting the cell with Compound (I).

Another aspect described herein is a method for increasing exon inclusion in one or more mature RNA isoforms from a gene transcript in a cell comprising, contacting the cell with Compound (I).

Another aspect described herein is a method for increasing exon exclusion in one or more mature RNA isoforms from a gene transcript in a cell comprising, contacting the cell with Compound (I).

Another aspect described herein is a method for modulating exon inclusion or exon exclusion in one or more mature RNA isoforms from a gene transcript in a cell comprising, contacting the cell in vivo or in vitro with Compound (I).

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

Another aspect described herein is a method for treating a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript.

### Compound (I) Use in a Method

One aspect described herein is Compound (I) for use in a method to modulate the production of one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) increases exon inclusion to produce one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) increases exon exclusion to produce one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein Compound (I) modulates exon inclusion or exon exclusion to produce one or more mature RNA isoforms from a gene transcript in a cell, wherein the method comprises, contacting the cell in vivo or in vitro with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene transcript is transcribed from a gene selected from Tables 1, 2, 3, 4, 5, 6, 7, 8, 9a, 10, 11, 12 or 13.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

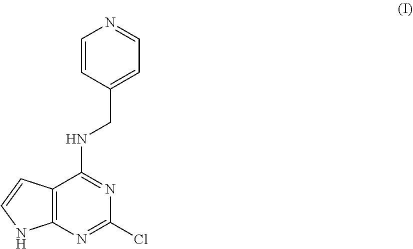 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein a gene having the mutated transcript is selected from the group consisting of Tables 1, 2, 3, 4, 5, 6, 7, 8, 9a, 10, 11, 12 or 13.

### Methods for Modulating Isoform Production

One aspect described herein is use of Compound (I) to modulate the production of one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is a use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is use of Compound (I) to modulate exon inclusion or exon exclusion in one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is use of Compound (I) to increase exon inclusion in one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is use of Compound (I) to increase exon exclusion in one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is use of Compound (I) to modulate exon inclusion or exon exclusion in one or more mature RNA isoforms from a gene transcript in a cell comprising, contacting the cell in vivo or in vitro with Compound (I), wherein the gene transcript is transcribed from a gene selected from Tables 1, 2, 3, 4, 5, 6, 7, 8, 9a, 10, 11, 12 or 13.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene transcript is transcribed from a gene selected from Tables 1, 2, 3, 4, 5, 6, 7, 8, 9a, 10, 11, 12 or 13.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene transcript is transcribed from a gene selected from Tables 1, 2, 3, 4, 5, 6, 7, 8, 9a, 10, 11, 12 or 13.

Another aspect described herein is use of Compound (I) to treat a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene transcript is transcribed from a gene selected from Tables 1, 2, 3, 4, 5, 6, 7, 8, 9a, 10, 11, 12 or 13.

### Compound (I) Use in a Method

One aspect described herein is Compound (I) for use in a method for modulating the production of one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a gene transcript in a cell, wherein the method comprises, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is Compound (I) for use in a method for modulating the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is Compound (I) for use in a method for modulating exon inclusion or exon exclusion in one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in a method for increasing exon inclusion in one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in a method for increasing exon exclusion in one or more mature RNA isoforms from a gene transcript in a cell, wherein the method comprises, contacting the cell with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in a method for modulating exon inclusion or exon exclusion in one or more mature RNA isoforms from a gene transcript in a cell, wherein the method comprises, contacting the cell in vivo or in vitro with Compound (I), and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Tables 1, 2, 3, 4, 5, 6, 7, 8, 9a, 10, 11, 12 or 13.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

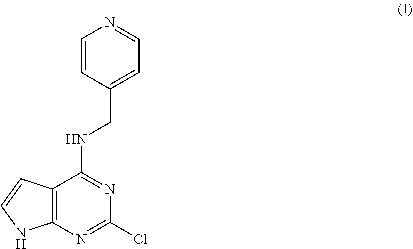 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Tables 1, 2, 3, 4, 5, 6, 7, 8, 9a, 10, 11, 12 or 13.

Another aspect described herein is Compound (I) for use in the method, wherein a gene having the mutated transcript is selected from Tables 1, 2, 3, 4, 5, 6, 7, 8, 9a, 10, 11, 12 or 13. METHODS FOR MODULATING ISOFORM PRODUCTION

One aspect described herein is a method for modulating the production of one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a gene transcript in a cell comprising, contacting the cell with Compound (I), wherein the gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Tables 1, 2, 3, 4, 5, 6, 7, 8, 9a, 10, 11, 12 or 13.

Another aspect described herein is a method for modulating the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Tables 1, 2, 3, 4, 5, 6, 7, 8, 9a, 10, 11, 12 or 13.

One aspect described herein is a method for modulating exon inclusion or exon exclusion in one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is a method for increasing exon inclusion in one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is a method for increasing exon exclusion in one or more mature RNA isoforms from a gene transcript in a cell comprising, contacting the cell with Compound (I), wherein the gene transcript is transcribed from a gene selected from Tables 1, 2, 3, 4, 5, 6, 7, 8, 9a, 10, 11, 12 or 13.

Another aspect described herein is a method for modulating exon inclusion or exon exclusion in one or more mature RNA isoforms from a gene transcript in a cell comprising, contacting the cell in vivo or in vitro with Compound (I), wherein the gene transcript is transcribed from a gene selected from Tables 1, 2, 3, 4, 5, 6, 7, 8, 9a, 10, 11, 12 or 13.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the gene transcript is transcribed from a gene selected from Tables 1, 2, 3, 4, 5, 6, 7, 8, 9a, 10, 11, 12 or 13.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the gene transcript is transcribed from a gene selected from Tables 1, 2, 3, 4, 5, 6, 7, 8, 9a, 10, 11, 12 or 13.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the gene is selected from Tables 1, 2, 3, 4, 5, 6, 7, 8, 9a, 10, 11, 12 or 13.

Another aspect described herein is a method for treating a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the gene is selected from Tables 1, 2, 3, 4, 5, 6, 7, 8, 9a, 10, 11, 12 or 13.

### Methods for Treating a Disease

One aspect described herein is Compound (I) for use in a method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the disease is selected from Table 14 or 15.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human, wherein the disease is selected from Table 14 or 15.

Another aspect described herein is Compound (I) for use in the method, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein, wherein the disease is selected from Table 14 or 15.

Another aspect described herein is a pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I):

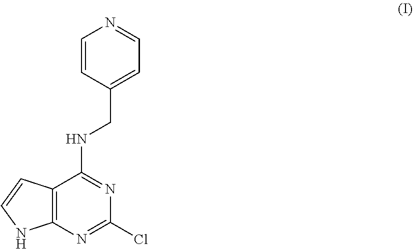 [SMILES: Clc1nc(NCc2ccncc2)c2cc[nH]c2n1]

wherein exon inclusion or exon exclusion is modulated in one or more mature RNA isoforms produced from a gene transcript in a cell, and wherein the gene transcript is transcribed from a gene selected from Table 14 or 15.

Another aspect described herein is Compound (I) for use in the method, wherein a gene having the mutated transcript is selected from Table 14 or 15.

One aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the disease is selected from Table 14 or 15.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human, wherein the disease is selected from Table 14 or 15.

Another aspect described herein is use of Compound (I) to treat a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein, wherein the disease is selected from Table 14 or 15.

Another aspect described herein is use of Compound (I) to treat a disease associated with or mediated by a mutation in a gene transcript in a subject in need thereof comprising, modulating the production of one or more mature RNA isoforms from a gene transcript by administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the disease is selected from Table 14 or 15.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein the subject is human, and wherein the disease is selected from Table 14 or 15.

Another aspect described herein is a method for treating a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, wherein one or more of the mature RNA isoforms produce a functional protein, and wherein the disease is selected from Table 14 or 15.

Another aspect described herein is a method for treating a disease associated with or mediated by a mutation in a gene transcript in a subject in need thereof comprising, administering to the subject a pharmaceutical composition of Compound (I) and a pharmaceutically acceptable carrier, excipient, or diluent, wherein exon inclusion or exon exclusion is modulated in one or more mature RNA isoforms produced from the gene transcript, and wherein the disease is selected from Table 14 or 15.

Table 14 lists diseases associated with genes having a mutated transcript identified by Clin VAR SpliceAI scores and the CNN Model, as described herein, that are predicted to be modulated by Compound (I) toward either or both exon exclusion and exon inclusion.

TABLE 14

| Gene | Disease |
|---|---|
| ABCA4 | Stargardt disease 1 |
| ABCC9 | Hypertrichotic osteochondrodysplasia; |
| | Dilated cardiomyopathy 1O |
| ACADSB | Deficiency of 2-methylbutyryl-CoA dehydrogenase |
| ADAM10 | Reticulate acropigmentation of Kitamura |
| AGK | Inborn genetic diseases; Sengers syndrome; |
| | Cataract, autosomal recessive congenital 5 |
| ALDH3A2 | Sjögren-Larsson syndrome |
| ALMS1 | not provided |
| ANTXR2 | Hyaline fibromatosis syndrome |
| APC | Hereditary cancer-predisposing syndrome; |
| | Familial adenomatous polyposis 1 |
| ARMC9 | Joubert Syndrome 30; ARMC9-related |
| | Joubert syndrome |
| ASAH1 | Farber disease |
| ASPM | Microcephaly; Primary autosomal |
| | recessive microcephaly 5 |
| ATM | Ataxia-telangiectasia syndrome; Familial cancer |
| | of breast; Hereditary cancer-predisposing syndrome |
| ATRX | ATR-X syndrome |
| BBS4 | Bardet-Biedl syndrome |
| BFSP1 | Cataract 33, multiple types |
| BMPR2 | Primary pulmonary hypertension |
| BRCA1 | Hereditary breast and ovarian cancer |
| | syndrome; Hereditary cancer-predisposing |
| | syndrome; Breast-ovarian cancer, familial 1 |
| BRCA2 | Tracheoesophageal fistula; Medulloblastoma; |
| | Malignant tumor of prostate; |
| | Hereditary breast and ovarian cancer |
| | syndrome; Familial cancer of breast; |
| | Hereditary cancer-predisposing syndrome; |
| | Fanconi anemia, complementation |
| | group D1; Breast-ovarian cancer, familial |
| | 2; Breast and/or ovarian cancer; |
| | Glioma susceptibility 3; Pancreatic |
| | cancer 2; Wilms tumor 1 |
| BRIP1 | Hereditary cancer-predisposing syndrome; |
| | Familial cancer of breast; Fanconi |
| | anemia, complementation group J |
| CA5A | Carbonic anhydrase VA deficiency, |
| | hyperammonemia due to |
| CAPN3 | Muscle weakness; Muscular Diseases; |
| | Absent Achilles reflex |
| CD3D | Immunodeficiency 19 |
| CDAN1 | Congenital dyserythropoietic anemia, type I |
| CDH1 | Hereditary cancer-predisposing syndrome |
| CDH23 | Usher syndrome, type 1D |
| CERKL | Retinitis pigmentosa; Retinitis pigmentosa 26 |
| CFTR | Inborn genetic diseases; Cystic fibrosis; |
| | Hereditary pancreatitis |
| CHD7 | Inborn genetic diseases |
| CLCN1 | Congenital myotonia, autosomal recessive |
| | form; Congenital myotonia, |
| | autosomal dominant form |
| CLMP | Intestinal pseudo-obstruction |
| CLN3 | Juvenile neuronal ceroid lipofuscinosis |
| CNGB3 | Achromatopsia 3 |
| COG6 | Congenital disorder of glycosylation type 2L |
| COL11A1 | Connective tissue disorder |
| COL3A1 | Ehlers-Danlos syndrome, type 4 |
| COL4A3 | Alport syndrome, autosomal recessive |
| COL4A5 | Alport syndrome 1, X-linked recessive |
| COL5A2 | Ehlers-Danlos syndrome, classic type |
| COL6A1 | Bethlem myopathy 1 |
| COL7A1 | Transient bullous dermolysis of the newborn |
| CSTB | Unverricht-Lundborg syndrome |
| CTNS | Nephropathic cystinosis |
| CTSK | Pyknodysostosis |
| CTU2 | Microcephaly, facial dysmorphism, renal |
| | agenesis, and ambiguous genitalia syndrome |
| CUBN | Megaloblastic anemia due to inborn |
| | errors of metabolism |
| CWC27 | Retinitis pigmentosa with or without |
| | skeletal anomalies |
| CYBB | Chronic granulomatous disease, X-linked |
| DCX | Heterotopia |
| DGKE | Hemolytic-uremic syndrome |
| DGUOK | not provided |
| DMD | Becker muscular dystrophy |
| DNAH5 | Ciliary dyskinesia |
| DNAH9 | Ciliary dyskinesia, primary, 40 |
| DYNC2H1 | Jeune thoracic dystrophy; Short-rib thoracic |
| | dysplasia 3 with or without polydactyly |
| DYNC2LI1 | Short-rib thoracic dysplasia 15 with polydactyly |
| EBF3 | Hypotonia, ataxia, and delayed |
| | development syndrome |
| EBP | Chondrodysplasia punctata 2 X-linked |
| | dominant; MEND syndrome |
| EP300 | Rubinstein-Taybi syndrome 2 |
| ERCC6 | Cockayne syndrome B |
| F10 | Factor X deficiency |
| F13A1 | Factor XIII subunit A deficiency |
| F8 | Hereditary factor VIII deficiency disease |
| FBN1 | Marfan syndrome; Cardiovascular phenotype |
| FIG4 | Polymicrogyria, Amyotrophic lateral |
| | sclerosis 11, Charcot-Marie-Tooth |
| | disease, type 4J, Yunis-Varon syndrome |
| FLT3 | Acute myeloid leukemia |
| FUT8 | Congenital disorder of glycosylation |
| | with defective fucosylation 1 |
| G6PC | Glycogen storage disease type 1A |
| GAA | Glycogen storage disease, type II |
| GABRG2 | Rolandic epilepsy |
| GCK | Maturity-onset diabetes of the young, type 2 |
| GLA | Fabry disease; Fabry disease, cardiac variant |
| GNB5 | Intellectual developmental disorder |
| | with cardiac arrhythmia |
| GNPTAB | Pseudo-Hurler polydystrophy; I cell disease; |
| | Mucolipidosis III alpha/beta, atypical |
| GPR143 | Ocular albinism, type I |
| GPX4 | Spondylometaphyseal dysplasia Sedaghatian type |
| GRHPR | Nephrocalcinosis; Nephrolithiasis |
| GRN | Frontotemporal dementia |
| GUCY2C | Meconium ileus |
| GYPA | BLOOD GROUP ERIK |
| HBB | beta Thalassemia |
| HFE | Hemochromatosis type 1 |
| IDH1 | Hepatocellular carcinoma; Medulloblastoma; |
| | Acute myeloid leukemia; |
| | Transitional cell carcinoma of the bladder; |
| | Multiple myeloma; Astrocytoma; |
| | Brainstem glioma; Lung adenocarcinoma; |
| | Neoplasm of brain; Neoplasm of the |
| | breast; Neoplasm of the large intestine; |
| | Malignant melanoma of skin; Adenoid |
| | cystic carcinoma; Glioblastoma; Myelodysplastic |
| | syndrome; Adenocarcinoma of prostate |
| IFT57 | Orofaciodigital Syndrome XVIII |
| IL36RN | Pustular psoriasis, generalized |
| KDSR | Erythrokeratodermia variabilis et progressiva 4 |
| KIAA0586 | Short-rib thoracic dysplasia 14 with polydactyly |
| KIAA1109 | Alkuraya-Kucinskas Syndrome |
| KIF14 | Microcephaly 20, primary, autosomal recessive |
| KIT | Partial albinism |
| KMT2D | Kabuki syndrome 1 |
| KRIT1 | not provided |
| LAMB3 | Adult junctional epidermolysis bullosa |
| LDLR | Familial hypercholesterolemia |
| LHCGR | Leydig cell agenesis |
| LIPA | Lysosomal acid lipase deficiency |
| LMNA | Cardiovascular phenotype; Benign scapuloperoneal |
| | muscular dystrophy with cardiomyopathy |
| MAPT | Frontotemporal dementia |
| MCFD2 | Factor v and factor viii, combined deficiency of, 2 |
| MLH1 | Hereditary nonpolyposis colon cancer; |
| | Hereditary cancer-predisposing |
| | syndrome; Lynch syndrome |
| MMAB | Methylmalonic aciduria cblB type |
| MPC1 | Mitochondrial pyruvate carrier deficiency |
| MSH2 | Hereditary cancer-predisposing syndrome |
| MTM1 | Severe X-linked myotubular myopathy |
| MYBPC3 | Hypertrophic cardiomyopathy |
| MYO7A | Usher syndrome, type 1 |
| NF1 | Multiple cafe-au-lait spots; Hereditary |
| | cancer-predisposing syndrome; |
| | Neurofibromatosis, type 1 |
| NIPBL | Cornelia de Lange syndrome 1 |
| NPHP1 | Nephronophthisis; Nephronophthisis 1 |
| NR3C2 | Pseudohypoaldosteronism type 1 autosomal dominant |
| NSD1 | Beckwith-Wiedemann syndrome |
| OCA2 | Tyrosinase-positive oculocutaneous albinism |
| OFD1 | Oral-facial-digital syndrome |
| OGT | Mental retardation, X-Linked 106 |
| ORC6 | Meier-Gorlin syndrome 3 |
| OTC | not provided |
| OTOGL | Deafness, autosomal recessive 84b |
| PAFAH1B1 | Lissencephaly 1 |
| PAH | Phenylketonuria |
| PARN | Dyskeratosis congenita, autosomal recessive 6 |
| PDCD10 | Cerebral cavernous malformations 3 |
| PIGN | Multiple congenital anomalies- |
| | hypotonia-seizures syndrome 1 |
| PMS2 | Hereditary nonpolyposis colon cancer; |
| | Hereditary cancer-predisposing |
| | syndrome; Lynch syndrome; Turcot syndrome |
| POLG | Seizures; Progressive sclerosing poliodystrophy |
| POMGNT1 | Congenital muscular dystrophy- |
| | dystroglycanopathy with brain and eye |
| | anomalies, type A3; Muscle eye brain disease |
| PPT1 | Ceroid lipofuscinosis neuronal 1 |
| PRMT7 | Short stature, brachydactyly, intellectual |
| | developmental disability, and seizures |
| PTEN | Hereditary cancer-predisposing syndrome; PTEN |
| | hamartoma tumor syndrome; Cowden syndrome 1 |
| RAD51B | Hereditary breast and ovarian cancer syndrome; |
| | Hereditary cancer-predisposing syndrome |
| RAD51C | Hereditary cancer-predisposing syndrome; |
| | Fanconi anemia, complementation |
| | group O; Breast-ovarian cancer, familial 3 |
| RB1 | Hereditary cancer-predisposing syndrome; |
| | Retinoblastoma |
| RHAG | Rh-null, regulator type |
| RNF216 | Hypogonadotropic hypogonadism 7 with |
| | or without anosmia; Leukodystrophy |
| RTTN | Congenital microcephaly; Microcephaly, |
| | short stature, and polymicrogyria with |
| | or without seizures |
| SCN1A | Severe myoclonic epilepsy in infancy |
| SCN5A | Brugada syndrome 1 |
| SDCCAG8 | Senior-Loken syndrome 7 |
| SDHD | Pheochromocytoma; Hereditary cancer- |
| | predisposing syndrome; Paraganglioma |
| | and gastric stromal sarcoma; Paragangliomas |
| | 1; Cowden syndrome 3 |
| SLC10A7 | Short stature, amelogenesis imperfecta, |
| | and skeletal dysplasia with scoliosis |
| SLC12A1 | Bartter syndrome, type 1, antenatal |
| SLC4A11 | Corneal endothelial dystrophy |
| SMS | Snyder Robinson syndrome |
| SNX10 | Osteopetrosis, autosomal recessive 8 |
| SPAST | Spastic paraplegia 4, autosomal dominant |
| SPG11 | Spastic paraplegia 11, autosomal recessive |
| SPTA1 | Congenital hemolytic anemia; Hereditary |
| | pyropoikilocytosis; Spherocytosis type 3 |
| SPTB | Spherocytosis type 2 |
| SRD5A2 | 3-Oxo-5 alpha-steroid delta |
| | 4-dehydrogenase deficiency |
| STK11 | Peutz-Jeghers syndrome |
| STXBP1 | Inborn genetic diseases |
| SYNGAP1 | Mental retardation, autosomal dominant 5 |
| TCIRG1 | Osteopetrosis autosomal recessive 1 |
| TECTA | Nonsyndromic hearing loss and deafness |
| TFR2 | Hemochromatosis type 3 |
| TGFBR2 | Loeys-Dietz syndrome 2 |
| TJP2 | Progressive familial intrahepatic cholestasis 4 |
| TMEM138 | Joubert syndrome 16 |
| TMPRSS6 | Microcytic anemia |
| TP53 | Hereditary cancer-predisposing |
| | syndrome; Li-Fraumeni syndrome |
| TPO | Deficiency of iodide peroxidase |
| TRAPPC2 | Spondyloepiphyseal dysplasia tarda |
| TRIM37 | Mulibrey nanism syndrome |
| TYR | Tyrosinase-negative oculocutaneous albinism |
| UROD | Familial porphyria cutanea tarda |
| VMA21 | Inborn genetic diseases; Myopathy, |
| | X-linked, with excessive autophagy |
| VPS13B | not provided |
| WDR35 | Short rib polydactyly syndrome 5 |
| WDR73 | Galloway-Mowat syndrome 1 |
| XPC | Xeroderma pigmentosum, group C |

Table 15 lists diseases associated with mutated genes that may be modulated toward exon inclusion, as disclosed in International Publication No. WO2016/115434.

TABLE 15

| | | | GeneBank |
|---|---|---|---|
| Gene | Gene Name | Associated Diseases | Acc. No. |
| ARFGEF2 | ADP- | Periventricular | NG 011490.1 |
| | ribosylation | heterotopia | |
| | factor guanine | with microcephaly | |
| | nucleotide- | | |
| | exchange | | |
| | factor 2 | | |
| | (brefeldin | | |
| | A-inhibited) | | |
| ARHGEF6 | RHO guanine | Mental retardation, | NG 008873.1 |
| | nucleotide | X-linked 46 | |
| | exchange | | |
| | factor 6 | | |
| CACNA1S | Calcium | Hypokalemic | NG 009816.1 |
| | channel, | periodic paralysis, | |
| | voltage- | type 1; Malignant | |
| | dependent, | hyperthermia | |
| | L Type, alpha- | susceptibility 5; | |
| | IS subunit | Thyrotoxic periodic | |
| | | paralysis, | |
| | | susceptibility to, 1 | |
| CC2D2A | Coiled-coil | COACH syndrome; | NG 013035.1 |
| | and C2 | Joubert syndrome | |
| | domain- | 9; Meckel syndrome 6 | |
| | containing | | |
| | protein 2A | | |
| CDKL5 | Cyclin- | Angelman syndrome-like; | NG 008475.1 |
| | dependent | Epileptic encephalopathy, | |
| | kinase-like 5 | early infantile, 2 | |
| CHD2 | Chromodomain | Epileptic encephalopathy, | NG 012826.1 |
| | helicase DNA- | childhood-onset | |
| | binding protein 2 | | |
| CHD7 | Chromodomain | CHARGE syndrome; | NG 007009.1 |
| | helicase DNA- | Hypogonadotropic | |
| | binding protein 7 | hypogonadism 5 with | |
| | | or without anosmia; | |
| | | Scoliosis, idiopathic 3 | |
| CHD8 | Chromodomain | Autism, susceptibility | NG 021249.1 |
| | helicase DNA- | | |
| | binding protein 8 | | |
| CHRNA4 | Cholinergic | Epilepsy, nocturnal | NG 011931.1 |
| | receptor, | frontal lobe, 1; Nicotine | |
| | neuronal | addiction, susceptibility to | |
| | nicotinic, alpha | | |
| | polypeptide 4 | | |
| Cl9orfl2 | Chromosome | Spastic paraplegia 43, | NG 031970.1 |
| | 10 open | autosornal recessive; | |
| | reading frame 12 | Neurodegeneration with | |
| | | brain iron accumulation 4 | |
| COL6A3 | Collagen type | Bethlem myopathy; | NG 008676.1 |
| | VI, alpha-3 | Ullrich congenital | |
| | | muscular dystrophy | |
| CUL4B | Cullin 4b | Mental retardation, | NG 009388.1 |
| | | X-linked, | |
| | | syndromic 15 | |
| | | (Cabezas type) | |
| DEPDC5 | DEP domain- | Epilepsy, familial focal, | NG 034067.1 |
| | containing | with variable foci | |
| | protein 5 | | |
| DES | Desmin | Muscular dystrophy, | NG 008043.1 |
| | | limb-girdle, type 2R; | |
| | | Cardiomyopathy, | |
| | | dilated, 11; Myopathy, | |
| | | myofibrillar, 1; | |
| | | Scapuloperoneal | |
| | | syndrome, | |
| | | neurogenic, Kaeser type | |
| DMD | Dystrophin | Becker muscular | NG 012232.1 |
| | | dystrophy; | |
| | | Cardiomyopathy, | |
| | | dilated, 3B; Duchenne | |
| | | muscular dystrophy | |
| DNAJC6 | DNAJ/HSP40 | Parkinson disease 19, | NG 033843.1 |
| | homology | juvenile-onset | |
| | subfamily | | |
| | C member6 | | |
| DYNC2H1 | Dynein, | Short-rib thoracic | NG 016423.1 |
| | cytoplasmic 2 | dysplasia 3 with or | |
| | heavy chain 1 | without polvdactvlv | |
| FBN1 | Fibrillin 1 | Acromicric | NG 008805.2 |
| | | dysplasia; Aortic | |
| | | aneurysm, ascending, | |
| | | and dissection; Ectopia | |
| | | lentis, familial; Marfan | |
| | | syndrome; | |
| | | MASS syndrome; | |
| | | Stiff skin syndrome; | |
| | | Weill-Marchesani | |
| | | syndrome | |
| | | 2, dominant | |
| FIG4 | SAC domain- | Polymicrogyria, bilateral | NG 007977.1 |
| | containing | temporooccipital, | |
| | inositol | Amyotrophic lateral | |
| | phosphatase 3 | sclerosis 11, | |
| | | Charcot-Marie-Tooth | |
| | | disease, type 4J, | |
| | | Yunis-Varon syndrome | |
| FKTN | Fukutin | Cardiomyopathy, dilated, | NG 008754.1 |
| | | IX; Muscular dystrophy- | |
| | | dystroglycanopathy | |
| | | (congenital with brain | |
| | | and eye anomalies), | |
| | | type A4, B4 and C4 | |
| FMR1 | Fragile X mental | Fragile X syndrome; | NG 007529.1 |
| | retardation | Fragile X | |
| | protein | tremor/ataxia syndrome; | |
| | | Premature ovarian | |
| | | failure 1 | |
| GOSR2 | Golgi SNAP | Epilepsy, progressive | NG 031806.1 |
| | receptor | myoclonic 6 | |
| | complex | | |
| | member 2 | | |
| GRIN2A | Glutamate | Epilepsy, focal, | NG 011812.1 |
| | receptor, | with speech disorder | |
| | ionotropic, | and with or without | |
| | N-methyl- | mental retardation | |
| | D-aspartate, | | |
| | subunit 2A | | |
| HDAC8 | Histone | Cornelia de Lange | NG 015851.1 |
| | deacetylase 8 | syndrome 5; | |
| | | Wilson-Turner svndrome | |
| IGHMBP2 | Immunoglobin 2 | Charcot-Marie-Tooth | NG 007976.1 |
| | MU-binding | disease, axonal, type 2S; | |
| | protein2 | Neuronopathy, distal | |
| | | hereditary motor, type VI | |
| IKBKAP | Inhibitor of | Dysautonomia, familial | NG 008788.1 |
| | kappa light | | |
| | polypeptide gene | | |
| | enhancer | | |
| | in B cells, | | |
| | kinase complex- | | |
| | associated | | |
| | protein | | |
| KDM5C | Lysine-specific | Mental retardation, | NG 008085.1 |
| | demethylase 5C | X-linked, syndromic, | |
| | | Claes-Jensen type | |
| LAMA2 | Laminin alpha-2 | Muscular dystrophy, | NG 008678.1 |
| | | congenital | |
| | | merosin-deficient; | |
| | | Muscular dystrophy, | |
| | | congenital, due | |
| | | to partial | |
| | | LAMA2 deficiency | |
| LRRK2 | Leucine-rich | Parkinson disease 8 | NG 011709.1 |
| | repeat kinase 2 | | |
| LRSAM1 | Leucine-rich | Charcot-Marie-Toothe | NG 032008.1 |
| | repeat-and | disease, axonal, type 2P | |
| | sterile | | |
| | alpha motif- | | |
| | containing 1 | | |
| MBD5 | Methyl-CpG- | Mental retardation, | NG 017003.1 |
| | binding | autosomal dominant 1 | |
| | domain protein 5 | | |
| MECP2 | Methyl-CpG- | Angelman syndrome; | NG 007107.2 |
| | binding | Encephalopathy, | |
| | protein 2 | neonatal severe; | |
| | | Mental retardation, | |
| | | X-linked syndromic, | |
| | | Lubs type; Mental | |
| | | retardation, X-linked, | |
| | | syndromic 13; Rett | |
| | | syndrome; Rett syndrome, | |
| | | preserved speech | |
| | | variant; Autism | |
| | | susceptibility, X-linked 3 | |
| MICU1 | Mitochondrial | Myopathy with | NG 033179.1 |
| | calcium | extrapyramidal signs | |
| | uptake protein 1 | | |
| MTM1 | Myotubularin 1 | Myotubular mvopathv, | NG 008199.1 |
| | | X-linked | |
| NEB | Nebulin | Nemaline myopathy 2, | NG 009382.2 |
| | | autosomal recessive | |
| OPHN1 | Oligophreninl | Mental retardation, | NG 008960.1 |
| | | X-linked, with | |
| | | cerebellar hypoplasia and | |
| | | distinctive facial | |
| | | appearance | |
| PGAP1 | Post-GPI | Mental retardation, | NC 000002.12 |
| | attachment to | autosomal | Range: |
| | proteins 1 | recessive 42 | 196833004 |
| | | | 19692 6995 |
| PLEC | Plectin | Epidermolysis bullosa | NG 012492.1 |
| | | simplex with | |
| | | pyloric atresia; | |
| | | Epidermolysis bullosa | |
| | | simplex, Ogna type; | |
| | | Muscular dystrophy with | |
| | | epidermolysis | |
| | | bullosa simplex; | |
| | | Muscular dystrophy, | |
| | | limb-girdle, type 2Q | |
| RBI | Retinoblastoma | Bladder cancer, | NG 009009.1 |
| | 1 | somatic; Osteosarcoma, | |
| | | somatic; Retinoblastoma; | |
| | | Retinoblastoma, | |
| | | trilateral; Small cell | |
| | | cancer of the | |
| | | lung, somatic | |
| RYR1 | Ryanodine | Central core disease; | NG 008866.1 |
| | receptor 1 | King-Denbo rough | |
| | | syndrome; Minicore | |
| | | myopathy with external | |
| | | ophthalmoplegia; | |
| | | Neuromuscular disease, | |
| | | congenital, with uniform | |
| | | type 1 fiber; | |
| | | Malignant hyperthermia | |
| | | susceptibility 1 | |
| SBF2 | SET-binding | Charcot-Marie-Tooth | NG 008074.1 |
| | factor 2 | disease, type 4B2 | |
| SCN9A | Sodium channel, | Epilepsy, generalized, | NG 012798.1 |
| | voltage- | with febrile seizures | |
| | gated, type | plus, type 7; | |
| | IX, alpha subunit | Erythermalgia, | |
| | | primary; Febrile seizures, | |
| | | familial, 3B; HSAN2D, | |
| | | autosomal recessive; | |
| | | Paroxysmal extreme | |
| | | pain disorder, Small | |
| | | fiber neuropathy; Dravet | |
| | | syndrome, modifier of | |
| SCN1A | Sodium channel, | Dravet syndrome; | NG 011906.1 |
| | neuronal type 1, | Epilepsy, generalized, | |
| | alpha subunit | with febrile seizures | |
| | | plus, type 2; | |
| | | Febrile seizures, | |
| | | familial, 3A; Migraine, | |
| | | familial hemiplegic, 3 | |
| SLC35A3 | Solute carrier | Arthrogryposis, mental | NG 033857.1 |
| | family 35 | retardation, and seizures | |
| | (UDP-N- | | |
| | acetylglucos- | | |
| | amine | | |
| | transporter) | | |
| | member 3 | | |
| SLC6A8 | Solute carrier | Cerebral creatine | NG 012016.1 |
| | family 6 | deficiency syndrome 1 | |
| | (neurotransmitter | | |
| | transporter | | |
| | creatine) | | |
| | member 8 | | |
| SLC9A9 | Solute carrier | Autism susceptibility | NG 017077.1 |
| | family 9 | | |
| | (sodium/ | | |
| | hydrogen | | |
| | exchanger) | | |
| | member 9 | | |
| SMCHD1 | Structural | Fascioscapulohumer al | NG 031972.1 |
| | maintenance of | muscular | |
| | chromosomes | dystrophy 2, digenic | |
| | flexible hinge | | |
| | domain- | | |
| | containing | | |
| | protein 1 | | |
| STXBP1 | Syntaxin-binding | Epileptic encephalopathy, | NG 016623.1 |
| | protein 1 | early infantile, 4 | |
| SYNGAP1 | Synaptic RAS- | Mental retardation, | NG 016137.1 |
| | GTPase- | autosomal dominant 5 | |
| | activating | | |
| | protein 1 | | |
| SZT2 | Seizure | Epileptic encephalopathy, | NG 029091.1 |
| | threshold 2 | early infantile, 18 | |
| TSC2 | Tuberin | Lymphangioleiomyo | NG 005895.1 |
| | | matosis, somatic; | |
| | | Tuberous sclerosis-2 | |
| TSC1 | Hamartin | Focal cortical dysplasia, | NG 012386.1 |
| | | Taylor balloon cell type; | |
| | | Lymphangioleiomyo | |
| | | matosis; | |
| | | Tuberous sclerosis-I | |
| WDR45 | WD40 repeat- | Neurodegeneration with | NG 033004.1 |
| | containing | brain iron accumulation 5 | |
| | protein 45 | | |

Another aspect provided herein is a disease associated with or mediated by a mutation in a gene, wherein the disease is selected from Table 14 or 15.

Another aspect provided herein is a disease associated with or mediated by a mutation in a gene, wherein the disease is selected from Table 14.

Another aspect provided herein is a disease associated with or mediated by a mutation in a gene, wherein the disease is selected from Table 15.

Another aspect provided herein is a disease associated with or mediated by a mutation in a gene, wherein the disease is other than a disease selected from Table 14 or 15.

Another aspect provided herein is a disease associated with or mediated by a mutation in a gene, wherein the disease is other than a disease selected from Table 14.

Another aspect provided herein is a disease associated with or mediated by a mutation in a gene, wherein the disease is other than a disease selected from Table 15.

Another aspect provided herein is a disease associated with or mediated by a mutation in a gene, wherein the disease is selected from the group consisting of: 3-Oxo-5 alpha-steroid delta 4-dehydrogenase deficiency, Absent Achilles reflex, Achromatopsia 3, Acute myeloid leukemia, Adenocarcinoma of prostate, Adenoid cystic carcinoma, Adult junctional epidermolysis bullosa, Alkuraya-kucinskas syndrome, Alport syndrome (Autosomal recessive), Alport syndrome 1 (X-linked recessive), Ambiguous genitalia syndrome, Amelogenesis imperfecta (Short stature and skeletal dysplasia with scoliosis), Amyotrophic lateral sclerosis, Astrocytoma, Ataxia, Ataxia-telangiectasia syndrome, ATR-X syndrome, Autism spectrum disorders, Autism, Bardet-Biedl syndrome, Bartter syndrome (Type 1, antenatal), Becker muscular dystrophy, Beckwith-Wiedemann syndrome, Benign scapuloperoneal muscular dystrophy with cardiomyopathy, Beta thalassemia, Bethlem myopathy 1, Blood group ERIK, Brainstem glioma, Breast cancer (familial), Breast-ovarian cancer (familial 1), Breast-ovarian cancer (familial 2), Breast-ovarian cancer (Familial 3), Breast-ovarian cancer, Brugada syndrome 1, Carbonic anhydrase VA deficiency (hyperammonemia due to), Cardiovascular phenotype, Cataract (autosomal recessive congenital 5), Cataract 33 (multiple types), Cerebral cavernous malformations 3, Ceroid lipofuscinosis neuronal 1, Charcot-Marie-Tooth disease, CHARGE syndrome, Chondrodysplasia punctata 2 (X-linked dominant), Chronic granulomatous disease (X-linked), Ciliary dyskinesia (Primary 40), Ciliary dyskinesia, Cockayne syndrome B, Congenital disorder of glycosylation (Type 2L), Congenital disorder of glycosylation with defective fucosylation, Congenital dyserythropoietic anemia (Type I), Congenital hemolytic anemia, Congenital microcephaly, Congenital muscular dystrophy-dystroglycanopathy with brain and eye anomalies (Type A3), Congenital myotonia (Autosomal dominant form), Congenital myotonia (Autosomal recessive form), Connective tissue disorder, Corneal endothelial dystrophy, Cornelia de Lange syndrome 1, Cowden syndrome 1, Cowden syndrome 3, Cystic fibrosis (Atypical), Cystic fibrosis, Deafness (Autosomal recessive 84b), Deficiency of 2-methylbutyryl-CoA dehydrogenase, Deficiency of iodide peroxidase, Delayed development syndrome, Dementia, Dilated cardiomyopathy 10, Dyskeratosis congenita (Autosomal recessive 6), Ehlers-Danlos syndrome (Classic type), Ehlers-Danlos syndrome (Type 4), Epilepsy, Epileptic encephalopathy, Erythrokeratodermia variabilis et progressiva 4, Fabry disease (Cardiac variant), Fabry disease, Facial dysmorphism, Factor v and Factor viii (combined deficiency of, 2), Factor X deficiency, Factor XIII subunit A deficiency, Familial adenomatous polyposis 1, Familial cancer of breast, Familial dysautonomia, Familial hypercholesterolemia, Familial isolated growth hormone deficiency type II, Familial porphyria cutanea tarda, Fanconi anemia (complementation group D1), Fanconi anemia (complementation group J), Fanconi anemia (Complementation group O), Farber disease, Frasier syndrome, Frontotemporal dementia, Galloway-Mowat syndrome 1, Glioblastoma, Glioma susceptibility 3, Glycogen storage disease (Type 1A), Glycogen storage disease (Type II), Hemochromatosis (Type 1), Hemochromatosis (Type 3), Hemolytic-uremic syndrome, Hepatocellular carcinoma, Hereditary breast and ovarian cancer syndrome, Hereditary cancer-predisposing syndrome, Hereditary factor VIII deficiency disease, Hereditary nonpolyposis colon cancer, Hereditary pancreatitis, Hereditary pyropoikilocytosis, Heterotopia, Huntington's disease, Hyaline fibromatosis syndrome, Hypertrichotic osteochondrodysplasia, Hypertrophic cardiomyopathy, Hypogonadotropic hypogonadism 7 with anosmia, Hypogonadotropic hypogonadism 7 without anosmia, Hypotonia, I cell disease, Immunodeficiency 19, Inborn genetic diseases, Intellectual developmental disability (with short stature, brachydactyly and seizures), Intellectual developmental disorder (Cardiac arrhythmia), Intestinal pseudo-obstruction, Jeune thoracic dystrophy, Joubert syndrome (Armc9-related), Joubert syndrome 16, Joubert syndrome 30, Juvenile neuronal ceroid lipofuscinosis, Kabuki syndrome 1, Leukodystrophy, Leydig cell agenesis, Li-Fraumeni syndrome, Lissencephaly 1, Loeys-Dietz syndrome 2, Lung adenocarcinoma, Lynch syndrome, Lysosomal acid lipase deficiency, Malignant melanoma of skin, Malignant tumor of prostate, Marfan syndrome (Cardiovascular phenotype), Marfan syndrome, Maturity-onset diabetes of the young (Type 2), Meconium ileus, Medulloblastoma, Megaloblastic anemia due to inborn errors of metabolism, Meier-Gorlin syndrome 3, MEND syndrome, Menkes disease, Mental retardation (Autosomal dominant 5), Mental retardation (X-linked 106), Methylmalonic aciduria (cblB type), Microcephaly (short stature), Microcephaly 20 (Primary, autosomal recessive), Microcephaly 5 (Primary autosomal recessive), Microcephaly, Microcytic anemia, Mitochondrial DNA-depletion syndrome 3, Mitochondrial pyruvate carrier deficiency, Mucolipidosis III alpha/beta (Atypical), Mulibrey nanism syndrome, Multiple cafe-au-lait spots, Multiple congenital anomalies-hypotonia-seizures syndrome 1, Multiple myeloma, Muscle eye brain disease, Muscle weakness, Muscular diseases, Muscular dystrophies, Myelodysplastic syndrome, Myopathies, Myopathy (X-linked, with excessive autophagy) Myotonic dystrophy (Type 1), Myotonic dystrophy (Type 2), Neoplasm of the brain, Neoplasm of the breast, Neoplasm of the large intestine, Nephrocalcinosis, Nephrolithiasis, Nephronophthisis 1, Nephronophthisis, Nephropathic cystinosis, Neurofibromatosis (Peripheral), Neurofibromatosis (Type 1), Neurofibromatosis (Von Recklinghausen), Nonsyndromic hearing loss and deafness, Occipital horn syndrome, Ocular albinism (Type I), Oral-facial-digital syndrome, Orofaciodigital syndrome XVIII, Osteopetrosis (Autosomal recessive 8), Osteopetrosis (Autosomal recessive 1), Ovarian cancer, Pancreatic cancer 2, Paraganglioma and gastric stromal sarcoma, Paragangliomas 1, Parkinson's disease linked to Chromosome 17, Parkinson's disease, Partial albinism, Peutz-Jeghers syndrome, Phenylketonuria, Pheochromocytoma, Polymicrogyria with seizures, Polymicrogyria without seizures, Primary pulmonary hypertension, Progressive familial intrahepatic cholestasis 4, Progressive sclerosing poliodystrophy, Pseudo-Hurler polydystrophy, Pseudohypoaldosteronism type 1 (Autosomal dominant), PTEN hamartoma tumor syndrome, Pustular psoriasis (Generalized), Pyknodysostosis, Renal agenesis, Reticulate acropigmentation of Kitamura, Retinitis pigmentosa 26, Retinitis pigmentosa with skeletal anomalies, Retinitis pigmentosa without skeletal anomalies, Retinitis pigmentosa, Retinoblastoma, Rh-null (Regulator type), Rolandic epilepsy, Rubinstein-Taybi syndrome 2, Schizophrenia, Seizures, Sengers syndrome, Senior-Loken syndrome 7, Severe myoclonic epilepsy (Infancy), Severe X-linked myotubular myopathy, Short rib polydactyly syndrome 5, Short-rib thoracic dysplasia 14 with polydactyly, Short-rib thoracic dysplasia 15 with polydactyly, Short-rib thoracic dysplasia 3 with polydactyly, Short-rib thoracic dysplasia 3 without polydactyly, Sj√∂gren-Larsson syndrome, Snyder Robinson syndrome, Spastic paraplegia 11 (Autosomal recessive), Spastic paraplegia 4 (Autosomal dominant), Spherocytosis type 2, Spherocytosis type 3, Spondyloepiphyseal dysplasia tarda, Spondylometaphyseal dysplasia (Sedaghatian type), Stargardt disease 1, Tracheoesophageal fistula, Transient bullous dermolysis of the newborn, Transitional cell carcinoma of the bladder, Tuberous sclerosis, Turcot syndrome, Tyrosinase-negative oculocutaneous albinism, Tyrosinase-positive oculocutaneous albinism, Unverricht-Lundborg syndrome, Usher syndrome (Type 1), Usher syndrome (Type 1D), Wilms tumor 1, and Xeroderma pigmentosum (Group C), and wherein the gene is selected from Tables 1, 2, 3, 4, 5, 6, 7, 8, 9a, 10, 11, 12 or 13.

Another aspect provided herein is a disease associated with or mediated by a mutation in a gene, wherein the disease is selected from: Autism, Blood group ERIK, Breast cancer, Ovarian cancer, Dementia, Epilepsy, Mental retardation, Muscular dystrophies, Myopathies, Neurofibromatosis 1, Parkinson's disease, or Schizophrenia, and wherein the gene is selected from Tables 1, 2, 3, 4, 5, 6, 7, 8, 9a, 10, 11, 12 or 13.

Another aspect provided herein is a disease associated with or mediated by a mutation in a gene, wherein the disease is selected from: 3-Oxo-5 alpha-steroid delta 4-dehydrogenase deficiency, Amyotrophic lateral sclerosis, Ataxia-telangiectasia syndrome, Atypical cystic fibrosis, Autism, Autism spectrum disorders, Beckwith-Wiedemann syndrome, Beta thalassemia, Blood group ERIK, Breast cancer, Ovarian cancer, Carbonic anhydrase VA deficiency, Charcot-Marie-Tooth disease, CHARGE syndrome, Ciliary dyskinesia, Congenital hemolytic anemia, Deficiency of iodide peroxidase, Dementia, Dyskeratosis congenita, Epilepsy, Epileptic encephalopathy, Fabry disease, Familial dysautonomia, Familial isolated growth hormone deficiency type II, Frasier syndrome, Frontotemporal dementia, Huntington's disease, Lysosomal acid lipase deficiency, Marfan syndrome, Meier-Gorlin syndrome 3, Menkes Disease, Mental retardation, Mitochondrial DNA-depletion syndrome 3, Multiple congenital anomalies-hypotonia-seizures syndrome 1, Muscular dystrophies, Myopathies, Myotonic dystrophy type 1, Myotonic dystrophy type 2, Neurofibromatosis 1, Neurofibromatosis (Peripheral), Neurofibromatosis (Von Recklinghausen), Occipital horn syndrome, Osteopetrosis autosomal recessive 1, Parkinson's disease, Parkinson's linked to Chromosome 17, Progressive sclerosing poliodystrophy, Pustular psoriasis, Retinoblastoma, Schizophrenia, Spondylometaphyseal dysplasia, or Tuberous sclerosis, and wherein the gene is selected from Tables 1, 2, 3, 4, 5, 6, 7, 8, 9a, 10, 11, 12 or 13.

Another aspect provided herein is a disease associated with or mediated by a mutation in a gene, wherein the disease is selected from: 3-Oxo-5 alpha-steroid delta 4-dehydrogenase deficiency, Amyotrophic lateral sclerosis, Ataxia-telangiectasia syndrome, Atypical cystic fibrosis, Autism spectrum disorders, Beckwith-Wiedemann syndrome, Beta thalassemia, Carbonic anhydrase VA deficiency, Charcot-Marie-Tooth disease, CHARGE syndrome, Ciliary dyskinesia, Congenital hemolytic anemia, Deficiency of iodide peroxidase, Dyskeratosis congenita, Epileptic encephalopathy, Fabry disease, Familial dysautonomia, Familial isolated growth hormone deficiency type II, Frasier syndrome, Frontotemporal dementia, Huntington's disease, Lysosomal acid lipase deficiency, Marfan syndrome, Meier-Gorlin syndrome 3, Menkes Disease, Mitochondrial DNA-depletion syndrome 3, Multiple congenital anomalies-hypotonia-seizures syndrome 1, Myotonic dystrophy type 1, Myotonic dystrophy type 2, Neurofibromatosis (Peripheral), Neurofibromatosis (Von Recklinghausen), Occipital horn syndrome, Osteopetrosis autosomal recessive 1, Parkinson's linked to Chromosome 17, Progressive sclerosing poliodystrophy, Pustular psoriasis, Retinoblastoma, Spondylometaphyseal dysplasia, or Tuberous sclerosis.

Another aspect provided herein is a disease associated with or mediated by a mutation in a gene, wherein the disease is selected from Table 14 or 15.

Another aspect provided herein is a disease associated with or mediated by a mutation in a gene, wherein the disease is selected from Table 14.

Another aspect provided herein is a disease associated with or mediated by a mutation in a gene, wherein the disease is other than a disease selected from Table 14.

Another aspect provided herein is a disease associated with or mediated by a mutation in a gene, wherein the disease is selected from Table 15.

Another aspect provided herein is a disease associated with or mediated by a mutation in a gene, wherein the disease is other than a disease selected from Table 15.

### In Vitro and In Vivo Assays of RNA Transcripts

In one aspect, the cell(s) contacted or cultured with Compound (I) is from a cell line. In another aspect, the cell(s) contacted or cultured with Compound (I) is a cell line derived from a subject with a disease (e.g., a disease identified herein). In another aspect, the cell(s) contacted or cultured with Compound (I) is from a cell line known to have aberrant RNA transcript levels transcribed from a particular gene(s). In another aspect, the cell(s) contacted or cultured with Compound (I) is from a cell line derived from a subject with a disease known to have aberrant RNA transcript levels transcribed from a particular gene(s). In another aspect, the cell(s) contacted or cultured with Compound (I) is a cancer cell line. In another aspect, the cell(s) contacted or cultured with Compound (I) is from a cell line derived from a subject with a disease known to have an aberrant amount of an RNA isoform(s) transcribed from a particular gene(s), an aberrant amount of a protein isoform(s) produced from the particular gene(s), or both. Non-limiting examples of cell lines include 293, 3T3, 4T1, 721, 9L, A2780, A172, A20, A253, A431, A-549, ALC, B16, B35, BCP-1, BEAS-2B, bEnd.3, BHK, BR 293, BT20, BT483, BxPC3, C2C12, C3H-10T1/2, C6/36, C6, Cal-27, CHO, COR-L23, COS, COV-434, CML T1, CMT, CRL7030, CT26, D17, DH82, DU145, DuCaP, EL4, EM2, EM3, EMT6, FM3, H1299, H69, H1B54, H1B55, HCA2, HEK-293, HeLa, Hepalclc7, HL-60, HMEC, Hs578T, HsS78Bst, HT-29, HTB2, HUVEC, Jurkat, J558L, JY, K562, Ku812, KCL22, KG1, KYO1, LNCap, Ma-Mel, MC-38, MCF-7, MCF-10A, MDA-MB-231, MDA-MB-468, MDA-MB-435, MDCK, MG63, MOR/0.2R, MONO-MAC 6, MRC5, MTD-1A, NCI-H69, NIH-3T3, NALM-1, NSO, NW-145, OPCN, OPCT, PNT-1A, PNT-2, Raji, RBL, RenCa, RIN-5F, RMA, Saos-2, Sf21, Sf9, SiHa, SKBR3, SKOV-3, T2, T-47D, T84, THP1, U373, U87, U937, VCaP, Vero, VERY, W138, WM39, WT-49, X63, YAC-1, and YAR cells. In another aspect, the cells are fibroblasts differentiated from PSC (pluripotent stem cells). In another aspect, the cells are human fibroblasts. In another aspect, the cells are from a patient.

In another aspect, provided herein is a method for determining whether Compound (I) modulates the amount of an RNA transcript (e.g., an mRNA transcript), comprising: (a) contacting a cell or tissue sample with Compound (I); and (b) determining the amount of the RNA transcript produced by the tissue sample, wherein an alteration in the amount of the RNA transcript in the presence of the compound relative to the amount of the RNA transcript in the absence of the compound or the presence of a negative control (e.g., a vehicle control such as PBS or DMSO) indicates that Compound (I) modulates the amount of the RNA transcript.

In another aspect, provided herein is a method for determining whether Compound (I) modulates the amount of an RNA transcript (e.g., an mRNA transcript), comprising: (a) contacting a first cell or a first tissue sample with Compound (I), (b) contacting a second cell or a second tissue sample with a negative control (e.g., a vehicle control, such as PBS or DMSO); and (c) determining the amount of the RNA transcript produced by the first cell or first tissue sample and the second cell or second tissue sample; and (d) comparing the amount of the RNA transcript produced by the first cell or first tissue sample to the amount of the RNA transcript produced by the second cell or second tissue sample, wherein an alteration in the amount of the RNA transcript produced by the first cell or first tissue sample relative to the amount of the RNA transcript produced by the second cell or second tissue sample indicates that Compound (I) modulates the amount of the RNA transcript. Any tissue sample containing cells may be used in the accordance with these methods. In another aspect, the tissue sample is a blood sample, a skin sample, a muscle sample, or a tumor sample. Techniques known to one skilled in the art may be used to obtain a tissue sample from a subject.

In another aspect, a dose-response assay is performed. In another aspect, the dose response assay comprises: (a) contacting a cell(s) with a concentration of Compound (I); (b) determining the amount of the RNA transcript produced by the cell(s), wherein an alteration in the amount of the RNA transcript in the presence of the compound relative to the amount of the RNA transcript in the absence of the compound or the presence of a negative control (e.g., a vehicle control such as PBS or DMSO) indicates that Compound (I) modulates the amount of the RNA transcript; (c) repeating steps (a) and (b), wherein the only experimental variable changed is the concentration of the compound; and (d) comparing the amount of the RNA transcript produced at the different concentrations of the compound. In another aspect, the dose response assay comprises: (a) culturing a cell(s) in the presence of Compound (I), (b) isolating the RNA transcript from the cell(s) after a certain period of time; (c) determining the amount of the RNA transcript produced by the cell(s), wherein an alteration in the amount of the RNA transcript in the presence of the compound relative to the amount of the RNA transcript in the absence of the compound or the presence of a negative control (e.g., a vehicle control such as PBS or DMSO) indicates that Compound (I) modulates the amount of the RNA transcript; (d) repeating steps (a), (b), and (c), wherein the only experimental variable changed is the concentration of the compound; and (e) comparing the amount of the RNA transcript produced at the different concentrations of the compound. In another aspect, the dose-response assay comprises: (a) contacting each well of a microtiter plate containing cells with a different concentration of Compound (I); (b) determining the amount of an RNA transcript produced by cells in each well; and (c) assessing the change of the amount of the RNA transcript at the different concentrations of the compound or form thereof.

In another aspect, the dose response assay comprises: (a) contacting cells with a concentration of Compound (I), wherein the cells are within the wells of a tissue culture container (e.g., a 96-well plate) at about the same density within each well, and wherein the cells are contacted with different concentrations of Compound (I) in different wells; (b) isolating the RNA from said cells in each well; (c) determining the amount of the RNA transcript produced by the cell(s) in each well; and (d) assessing change in the amount of the RNA transcript in the presence of one or more concentrations of compound relative to the amount of the RNA transcript in the presence of a different concentration of the compound or the absence of Compound (I) or the presence of a negative control (e.g., a vehicle control such as PBS or DMSO).

In another aspect, contacting of the cell(s) with the compound occurs in cell culture. In another aspect, contacting of the cell(s) with the compound occurs in a subject, such as a non-human animal subject.

In another aspect described herein, the cell(s) is contacted or cultured with Compound (I), or a tissue sample is contacted with Compound (I), or a negative control for a period of 15 minutes, 30 minutes, 45 minutes, 1 hour, 2 hours, 3 hours, 4 hours, 5 hours, 6 hours, 8 hours, 12 hours, 18 hours, 24 hours, 48 hours, 72 hours or more. In another aspect described herein, the cell(s) is contacted or cultured with Compound (I), or a tissue sample is contacted with Compound (I), or a negative control for a period of 15 minutes to 1 hour, 1 to 2 hours, 2 to 4 hours, 6 to 12 hours, 12 to 18 hours, 12 to 24 hours, 28 to 24 hours, 24 to 48 hours, 48 to 72 hours.

In another aspect described herein, the cell(s) is contacted or cultured with a certain concentration of Compound (I), or a tissue sample is contacted with a certain concentration of Compound (I), wherein the certain concentration is 0.01 μM, 0.05 μM, 1 μM, 2 μM, 5 μM, 10 μM, 15 μM, 20 μM, 25 μM, 50 μM, 60 μM, 75 μM, 100 μM, or 150 μM. In one aspect described herein, the cell(s) is contacted or cultured with certain concentration of Compound (I), or a tissue sample is contacted with a certain concentration of Compound (I), wherein the certain concentration is 175 μM, 200 μM, 250 μM, 275 μM, 300 μM, 350 μM, 400 μM, 450 μM, 500 μM, 550 μM 600 μM, 650 μM, 700 μM, 750 μM, 800 μM, 850 μM, 900 μM, 950 μM or 1 mM. In another aspect described herein, the cell(s) is contacted or cultured with certain concentration of Compound (I), or a tissue sample is contacted with a certain concentration of Compound (I), wherein the certain concentration is 5 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, or 950 nM. In another aspect described herein, the cell(s) is contacted or cultured with certain concentration of Compound (I), or a tissue sample is contacted with a certain concentration of Compound (I), wherein the certain concentration is between 0.01 μM to 0.1 μM, 0.1 μM to 1 μM, 1 μM to 50 μM, 50 μM to 100 μM, 100 μM to 500 μM, 500 μM to 1 nM, 1 nM to 10 nM, 10 nM to 50 nM, 50 nM to 100 nM, 100 nM to 500 nM, 500 nM to 1000 nM.

In another aspect, provided herein is a method for determining whether Compound (I) modulates the amount of an RNA transcript (e.g., an mRNA transcript), comprising: (a) administering Compound (I) to a subject (in another aspect, a non-human animal); and (b) determining the amount of the RNA transcript in a sample obtained from the subject, wherein an alteration in the amount of the RNA transcript measured in the sample from the subject administered the compound or form thereof relative to the amount of the RNA transcript in a sample from the subject prior to administration of the compound or form thereof or a sample from a different subject from the same species not administered the compound or form thereof indicates that Compound (I) modulates the amount of the RNA transcript. In another aspect, provided herein is a method for determining whether Compound (I) modulates the amount of an RNA transcript (e.g., an mRNA transcript), comprising: (a) administering Compound (I) to a first subject (in another aspect, a non-human animal); (b) administering a negative control (e.g., a pharmaceutical carrier) to a second subject (in another aspect, a non-human animal) of the same species as the first subject; and (c) determining the amount of the RNA transcript in a first tissue sample from the first subject and the amount of the RNA transcript in the second tissue sample from the second subject; and (d) comparing the amount of the RNA transcript in the first tissue sample to the amount of the RNA transcript in the second tissue sample, wherein an alteration in the amount of the RNA transcript in the first tissue sample relative to the amount of the RNA transcript in the second tissue sample indicates that Compound (I) modulates the amount of the RNA transcript.

In another aspect, Compound (I) or form thereof is administered to a subject at a dose of about 0.001 mg/kg/day to about 500 mg/kg/day. In another aspect, a single dose of Compound (I) is administered to a subject in accordance with the methods described herein. In another aspect, 2, 3, 4, 5 or more doses of Compound (I) is administered to a subject in accordance with the methods described herein. In another aspect, Compound (I) is administered in a subject in a pharmaceutically acceptable carrier, excipient or diluent.

In another aspect, provided herein is a method for determining whether Compound (I) modulates the splicing of an RNA transcript (e.g., an mRNA transcript), comprising: (a) administering Compound (I) to a subject (in another aspect, a non-human animal); and (b) determining the amount of two or more RNA transcript splice variants in a sample obtained from the subject, wherein an alteration in the amount of the two or more RNA transcript splice variants measured in the sample from the subject administered the compound or form thereof relative to the amount of the two or more RNA transcript splice variants in a sample from the subject prior to administration of the compound or form thereof or a sample from a different subject from the same species not administered the compound or form thereof indicates that Compound (I) modulates the splicing of the RNA transcript. In another aspect, provided herein is a method for determining whether Compound (I) modulates the splicing of an RNA transcript (e.g., an mRNA transcript), comprising: (a) administering Compound (I) to a first subject (in another aspect, a non-human animal); (b) administering a negative control (e.g., a pharmaceutical carrier) to a second subject (in another aspect, a non-human animal) of the same species as the first subject; and (c) determining the amount of two or more RNA transcript splice variants in a first tissue sample from the first subject and the amount of two or more RNA transcript splice variants in the second tissue sample from the second subject; and (d) comparing the amount of the two or more RNA transcript splice variants in the first tissue sample to the amount of the two or more RNA transcript splice variants in the second tissue sample, wherein an alteration in the amount of the two or more RNA transcript splice variants in the first tissue sample relative to the amount of the two or more RNA transcript splice variants in the second tissue sample indicates that Compound (I) modulates the splicing of the RNA transcript. In another aspect, Compound (I) or form thereof is administered to a subject at a dose of about 0.001 mg/kg/day to about 500 mg/kg/day. In another aspect, a single dose of Compound (I) is administered to a subject in accordance with the methods described herein. In another aspect, 2, 3, 4, 5 or more doses of Compound (I) is administered to a subject in accordance with the methods described herein. In another aspect, Compound (I) is administered in a subject in a pharmaceutically acceptable carrier, excipient or diluent. In another aspect, Compound (I) that is contacted or cultured with a cell(s) or a tissue sample or administered to a subject is a compound described herein.

Techniques known to one skilled in the art may be used to determine the amount of an RNA transcript(s). In one aspect, the amount of one, two, three or more RNA transcripts is measured using deep sequencing, such as ILLUMIINA® RNASeq, ILLUMIINA® next generation sequencing (NGS), ION TORRENT™ RNA next generation sequencing, 454™ pyrosequencing, or Sequencing by Oligo Ligation Detection (SOLID™). In another aspect, the amount of RNA transcripts is measured using an exon array, such as the GENECHIP® human exon array. In another aspect, the amount of one, two, three or more RNA transcripts is determined by RT-PCR. In another aspect, the amount of one, two, three or more RNA transcripts is measured by RT-qPCR. Techniques for conducting these assays are known to one skilled in the art.

In another aspect, the stability of one or more RNA transcripts is determined by serial analysis of gene expression (SAGE), differential display analysis (DD), RNA arbitrarily primer (RAP)-PCR, restriction endonuclease-lytic analysis of differentially expressed sequences (READS), amplified restriction fragment-length polymorphism (ALFP), total gene expression analysis (TOGA), RT-PCR, RT-qPCR, high-density cDNA filter hybridization analysis (HDFCA), suppression subtractive hybridization (SSH), differential screening (DS), cDNA arrays, oligonucleotide chips, or tissue microarrays. In another aspect, the stability of one or more RNA transcripts is determined by Northern blots, RNase protection, or slot blots.

In another aspect, the transcription in a cell(s) or tissue sample is inhibited before (e.g., 5 minutes, 10 minutes, 30 minutes, 1 hour, 2 hours, 4 hours, 6 hours, 8 hours, 12 hours, 18 hours, 24 hours, 36 hours, 48 hours, or 72 hours before) or after (e.g., 5 minutes, 10 minutes, 30 minutes, 1 hour, 2 hours, 4 hours, 6 hours, 8 hours, 12 hours, 18 hours, 24 hours, 36 hours, 48 hours, or 72 hours after) the cell or the tissue sample is contacted or cultured with an inhibitor of transcription, such as α-amanitin, DRB, flavopiridol, triptolide, or actinomycin-D. In another aspect, the transcription in a cell(s) or tissue sample is inhibited with an inhibitor of transcription, such as α-amanitin, DRB, flavopiridol, triptolide, or actinomycin-D, while the cell(s) or tissue sample is contacted or cultured with Compound (I).

In another aspect, the level of transcription of one or more RNA transcripts is determined by nuclear run-on assay or an in vitro transcription initiation and elongation assay. In another aspect, the detection of transcription is based on measuring radioactivity or fluorescence. In another aspect, a PCR-based amplification step is used.

In one aspect, the amount of alternatively spliced forms of the RNA transcripts of a particular gene are measured to see if there is an alteration in the amount of one, two or more alternatively spliced forms of the RNA transcripts of the gene. In another aspect, the amount of an isoform(s) encoded by a particular gene is measured to see if there is an alteration in the amount of the isoform(s). In another aspect, the levels of spliced forms of RNA are quantified by RT-PCR, RT-qPCR, or northern blotting. In another aspect, sequence-specific techniques may be used to detect the levels of an individual spliceoform. In another aspect, splicing is measured in vitro using nuclear extracts. In another aspect, detection is based on measuring radioactivity or fluorescence. Techniques known to one skilled in the art may be used to measure alterations in the amount of alternatively spliced forms of an RNA transcript of a gene and alterations in the amount of an isoform encoded by a gene.

### Modulating Isoform Splicing

In some aspects, analysis is performed on data derived from the assay to measure the magnitude of splicing to determine the amount of exons spliced into an mRNA transcript that is produced in the presence of Compound (I) relative to the amount in the absence of Compound (I) or presence of a negative control. In a preferred aspect, the method utilized is calculation of change in Percent Spliced In (ΔPSI). The method utilizes read data from RNAseq (or any other method that can distinguish mRNA splice isoforms) to calculate the ratio (percentage) between reads that either demonstrate inclusion (junctions between the upstream exon and the exon of interest) or exclusion (junction between the upstream and downstream exons, excluding the exon of interest), to demonstrate whether the presence of Compound (I) affects the amount of exon inclusion relative to the amount of inclusion in the absence of Compound (I) or the presence of a negative control. The ΔPSI value is derived from the formula:

ΔPSI (%)=(C−U)×100

Where “U” represents the Percent Spliced In value for splicing to occur in the absence of Compound (I):

U=(a+b)/2/[(a+b)/2+c]

Where “C” represents the Percent Spliced In value for splicing to occur in the presence of Compound (I).

C=(a+b)/2/[(a+b)/2+c]

The values for “a” and “b” represent the number of reads supporting the probability for potential inclusion or exclusion of an exon in an RNA transcript. The “a” value is derived from the number of reads within an exon triplet covering, in 5′ to 3′ order: the last few bases (>1 nucleotide) of an upstream exon operably linked to the first few bases (>1 nucleotide) of a middle exon. The “b” value is derived from the number of reads within an exon triplet covering, in 5′ to 3′ order: the last few bases (>1 nucleotide) of the middle exon operably linked to the first few bases (>1 nucleotide) of a downstream exon. The number of reads will indicate whether the splice junction of a middle exon has been identified as potentially having a CNN Motif(s) by the CNN Model described herein, wherein the inclusion or exclusion of the middle exon may be modulated in the presence of Compound (I) to produce one or more mature RNA isoforms from the gene transcript. The value for “c” represents the number of reads supporting the exclusion of the middle exon.

Accordingly, an alteration in the CNN Motifs may affect the splicing toward one Class or another (inclusion or exclusion) of the middle exon in the presence of Compound (I), thus making the PSI value for “C” in the presence of Compound (I) different from the PSI value for “U” in the absence of Compound (I). The statistically significant value for the probability of inclusion or exclusion may be obtained according to statistical or probability analysis methods known to those of ordinary skill in the art.

In some aspects, a statistical analysis or other probability analysis is performed on data from the assay utilized to measure an RNA transcript. In certain aspects, for example, a Fisher's Exact Test statistical analysis is performed by comparing the total number of reads for the inclusion and exclusion of a middle exon based on data from one or more assays used to measure whether the amount of an RNA transcript is modulated in the presence of Compound (I) relative to the amount in the absence of Compound (I) or presence of a negative control. In specific aspects, the statistical analysis results in a confidence value for those modulated RNA transcripts of 10%, 5%, 4%, 3%, 2%, %, 0.5%, 0.1%, 0.01%, 0.001% or 0.0001%. In some specific aspects, the confidence value is a p value for those modulated RNA transcripts of 10%, 5%, 4%, 3%, 2%, 10%, 0.5%, 0.1%, 0.010%, 0.0010% or 0.00010%. In certain specific aspects, an exact test, student t-test or p value for those modulated RNA transcripts is 10%, 5%, 4%, 3%, 2%, 1%, 0.5% or 0.1% and 10%, 5%, 4%, 3%, 2%, 1%, 0.5%, 0.1%, 0.01%, 0.001% or 0.0001%, respectively.

[In certain aspects, a further analysis is performed to determine how Compound (I) is changing the amount of an RNA transcript(s). In specific aspects, a further analysis is performed to determine if modulation in the amount of an RNA transcript(s) in the presence of Compound (I) relative the amount of the RNA transcript(s) in the absence of Compound (I) or a form thereof, or the presence of a negative control is due to changes in transcription, splicing, and/or stability of the RNA transcript(s). Techniques known to one skilled in the art may be used to determine whether Compound (I) changes, e.g., the transcription, splicing and/or stability of an RNA transcript(s).

In certain aspects, the stability of one or more RNA transcripts is determined by serial analysis of gene expression (SAGE), differential display analysis (DD), RNA arbitrary primer (RAP)-PCR, restriction endonuclease-lytic analysis of differentially expressed sequences (READS), amplified restriction fragment-length polymorphism (ALFP), total gene expression analysis (TOGA), RT-PCR, RT-RPA (recombinase polymerase amplification), RT-qPCR, RNA-Seq, digital color-coded barcode technology, high-density cDNA filter hybridization analysis (HDFCA), suppression subtractive hybridization (SSH), differential screening (DS), cDNA arrays, oligonucleotide chips, or tissue microarrays. In other aspects, the stability of one or more RNA transcripts is determined by Northern blot, RNase protection, or slot blot.

In some aspects, the transcription in a cell(s) or tissue sample is inhibited before (e.g., 5 minutes, 10 minutes, 30 minutes, 1 hour, 2 hours, 4 hours, 6 hours, 8 hours, 12 hours, 18 hours, 24 hours, 36 hours, 48 hours, or 72 hours before) or after (e.g., 5 minutes, 10 minutes, 30 minutes, 1 hour, 2 hours, 4 hours, 6 hours, 8 hours, 12 hours, 18 hours, 24 hours, 36 hours, 48 hours, or 72 hours) the cell or the tissue sample is contacted or cultured with an inhibitor of transcription, such as α-amanitin, DRB, flavopiridol, triptolide, or actinomycin-D. In other aspects, the transcription in a cell(s) or tissue sample is inhibited with an inhibitor of transcription, such as α-amanitin, DRB, flavopiridol, triptolide, or actinomycin-D, while the cell(s) or tissue sample is contacted or cultured with Compound (I).

In certain aspects, the level of transcription of one or more RNA transcripts is determined by nuclear run-on assay or an in vitro transcription initiation and elongation assay. In some aspects, the detection of transcription is based on measuring radioactivity or fluorescence. In some aspects, a PCR-based amplification step is used.

In specific aspects, the amount of alternatively spliced forms of the RNA transcripts of a particular gene are measured to see if there is modulation in the amount of one, two or more alternatively spliced forms of the RNA transcripts of the gene. In some aspects, the amount of an isoform(s) encoded by a particular gene is measured to see if there is modulation in the amount of the isoform(s). In certain aspects, the levels of spliced forms of RNA are quantified by RT-PCR, RT-qPCR, RNA-Seq, digital color-coded barcode technology, or Northern blot. In other aspects, sequence-specific techniques may be used to detect the levels of an individual spliceoform. In certain aspects, splicing is measured in vitro using nuclear extracts. In some aspects, detection is based on measuring radioactivity or fluorescence. Techniques known to one skilled in the art may be used to measure modulation in the amount of alternatively spliced forms of an RNA transcript of a gene and modulation in the amount of an isoform encoded by a gene.

### Examples

To describe in more detail and assist in understanding the present description, the following non-limiting examples are offered to more fully illustrate the scope of the description and are not to be construed as specifically limiting the scope thereof. Such variations of the present description that may be now known or later developed, which would be within the purview of one skilled in the art to ascertain, are considered to fall within the scope of the present description and as hereinafter claimed. The examples below illustrate the usefulness of the CNN Model and the 39 CNN Motifs identified herein.

### Identification of Genes Affected by Compound (I) Splicing Modulation Using a Convolutional Neural Network (CNN) Model

A specific deep learning convolutional neural network (CNN) was applied and optimized to discover nucleotide motifs likely to be responsive to splicing modulation by a small molecule splicing modulator Compound (I). As described herein, motifs likely to be responsive in 171 additional disease-associated genes using OMIM and ClinVar were identified. As further described herein, in vitro validation demonstrated that the CNN Model successfully predicted Compound (I) splice modulation in minigene assays and in patient-specific cell lines. This approach suggests that the integration of genomic datasets, clinical annotation of disease associated variants, and deep learning techniques have significant potential to predict therapeutic targeting for precision medicine.

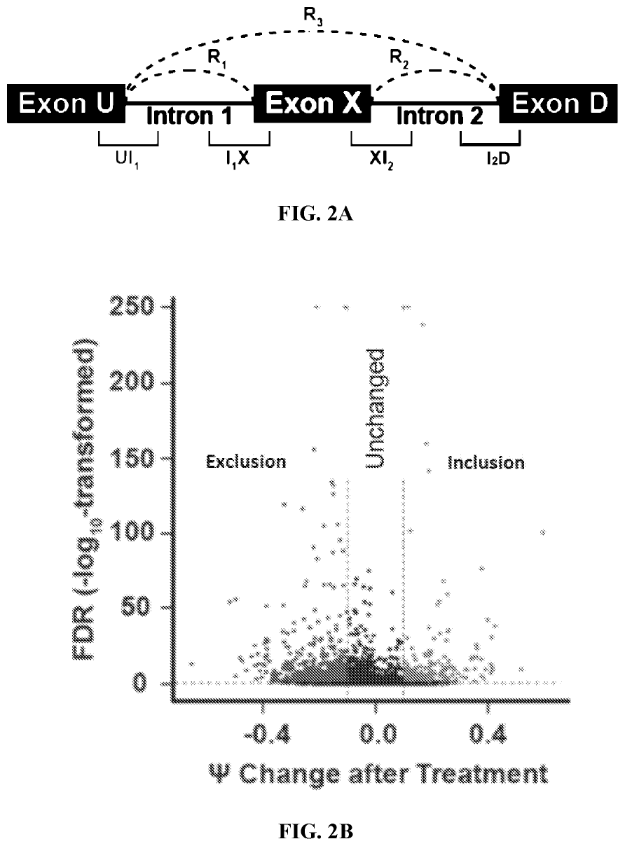

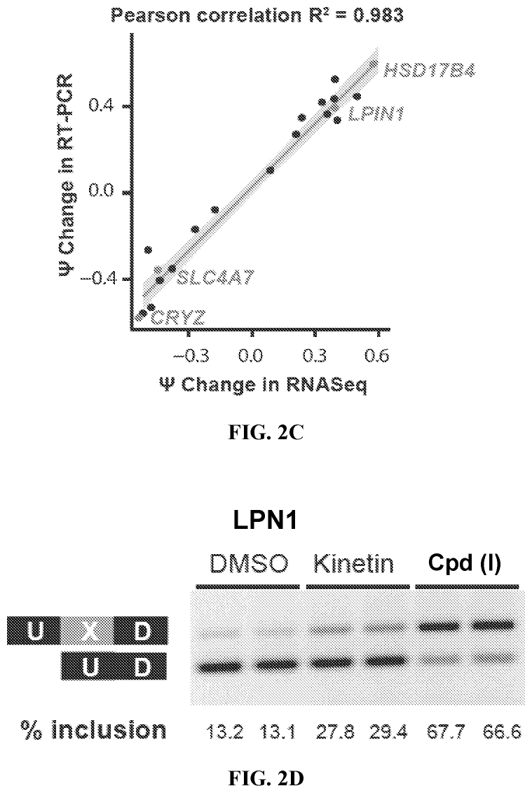

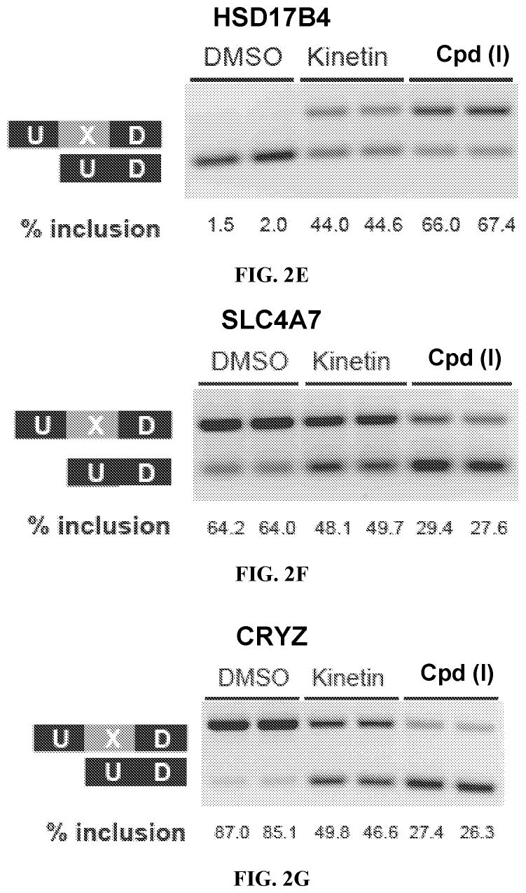

The effect on splicing modulation and corresponding splice differences toward exon inclusion or exclusion was determined by counting the RNASeq reads covering two splice junctions of exon triplets (three consecutive exons: Exon U, Exon X, Exon D) and the corresponding intervening introns, then comparing the change in Percent Spliced In (ΔPSI or Δψ) of the middle exon (Exon X) after treatment (as shown in FIG. 2A)^{52,53}. 934 exon triplets were identified showing differential middle exon inclusion or exclusion in response to Compound (I) treatment. Of these, 254 were exon inclusion events (Δψ≥0.1 and FDR<0.1) and 680 were exon exclusion events (Δψ≤−0.1 and FDR<0.1) (as shown in FIG. 2B). Compound (I) exhibited selective splicing modulation activity for splicing changes in 0.58% of all expressed triplets (934 out of 161,097 expressed triplets). The effect of Compound (I) treatment on genes, as shown in FIG. 2C, demonstrated by reproducible PSI changes (Δψ) determined by RNA-seq, was confirmed. The splicing results were evaluated by RT-PCR in independent replicate experiments, confirming all the calculated Δψ values. These results suggest that Compound (I) targets a specific subset of exons for splicing modulation.

Pre-mRNA splicing is driven by sequence elements throughout exons and introns. These sequences govern interaction with the spliceosome and splicing factors and regulate the fate of exon recognition and inclusion^{2,54}. These sequence signatures within the exon triplets are the key determinate of small molecule splicing modulator compound responsiveness. To identify such sequences, a convolutional neural network (CNN) model was trained using the inclusion-response set (254 exon triplets), exclusion-response set (680 exon triplets) and the unchanged-response set (382 exon triplets, Δψ≤0.01) identified by RNASeq (see Table 16).

### Gene Modulation Using Compound (I)

The effect of Compound (I) on transcriptome-wide splicing as measured by RNA sequence (RNASeq) analysis is provided in Table 16 (where Intron 1 is upstream of the affected Exon and Intron 2 is downstream; the Effect shown is Exc, Inc, or Unc representing Exclusion, Inclusion and Unchanged, respectively; C-PSI, T-PSI and ΔPSI are the average Percent Spliced In values for the initial PSI, Treated-PSI and ΔPSI, respectively and the associated p value), in which six different wildtype (WT) human fibroblast cell lines were treated with Compound (I) and vehicle (DMSO) for seven days.

TABLE 16

| Gene | Intron1 | Exon | Intron2 | Effect | C-PSI | T-PSI | ΔPSI | p value |
|---|---|---|---|---|---|---|---|---|
| A1BG-AS1 | 58859211- | 58864687- | 58864841- | Exc | 0.729 | 0.361 | −0.368 | 5.73E−04 |
| | 58864686 | 58864840 | 58865079 | | | | | |
| AAAS | 53708226- | 53708535- | 53708634- | Exc | 0.811 | 0.645 | −0.166 | 9.73E−10 |
| | 53708534 | 53708633 | 53708877 | | | | | |
| AAMDC | 77532288- | 77552065- | 77552107- | Unc | 0.465 | 0.460 | −0.005 | 8.72E−01 |
| | 77552064 | 77552106 | 77553524 | | | | | |
| ABCA1 | 107645439- | 107646708- | 107646850- | Exc | 0.895 | 0.724 | −0.172 | 4.32E−06 |
| | 107646707 | 107646849 | 107651382 | | | | | |
| ABCA2 | 139917505- | 139918290- | 139918293- | Unc | 0.900 | 0.902 | 0.003 | 9.85E−01 |
| | 139918289 | 139918292 | 139918594 | | | | | |
| ABCA6 | 67085684- | 67087284- | 67087404- | Exc | 0.991 | 0.852 | −0.139 | 6.61E−06 |
| | 67087283 | 67087403 | 67092380 | | | | | |
| ABCA9 | 66992156- | 67003900- | 67004014- | Exc | 0.993 | 0.553 | −0.439 | 6.55E−22 |
| | 67003899 | 67004013 | 67004202 | | | | | |
| ABCB6 | 220079805- | 220080719- | 220080903- | Exc | 0.979 | 0.874 | −0.104 | 4.77E−04 |
| | 220080718 | 220080902 | 220081085 | | | | | |
| ABCC3 | 48752838- | 48752992- | 48753137- | Exc | 0.968 | 0.817 | −0.150 | 1.13E−16 |
| | 48752991 | 48753136 | 48753243 | | | | | |
| ABCC3 | 48736730- | 48738284- | 48738476- | Unc | 0.856 | 0.850 | −0.006 | 9.15E−01 |
| | 48738283 | 48738475 | 48741041 | | | | | |
| ABCD4 | 74766379- | 74766853- | 74766972- | Unc | 0.844 | 0.849 | 0.005 | 9.14E−01 |
| | 74766852 | 74766971 | 74769577 | | | | | |
| ABHD14A- | 52018175- | 52019223- | 52019288- | Exc | 0.950 | 0.825 | −0.125 | 1.20E−04 |
| ACY1 | 52019222 | 52019287 | 52019376 | | | | | |
| ABHD16A | 31668806- | 31669851- | 31669908- | Unc | 0.820 | 0.826 | 0.006 | 9.16E−01 |
| | 31669850 | 31669907 | 31670926 | | | | | |
| ABI1 | 27059275- | 27060004- | 27060019- | Inc | 0.559 | 0.699 | 0.141 | 8.08E−08 |
| | 27060003 | 27060018 | 27065993 | | | | | |
| ABI2 | 204267458- | 204276008- | 204276095- | Inc | 0.475 | 0.634 | 0.158 | 8.84E−08 |
| | 204276007 | 204276094 | 204281630 | | | | | |
| ABI3BP | 100581230- | 100581682- | 100581754- | Exc | 0.791 | 0.629 | −0.162 | 7.19E−09 |
| | 100581681 | 100581753 | 100582845 | | | | | |
| ABI3BP | 100581230- | 100582846- | 100582921- | Exc | 0.566 | 0.361 | −0.205 | 3.25E−05 |
| | 100582845 | 100582920 | 100583676 | | | | | |
| ABI3BP | 100581230- | 100581682- | 100581754- | Exc | 0.404 | 0.162 | −0.241 | 2.59E−09 |
| | 100581681 | 100581753 | 100583676 | | | | | |
| ABI3BP | 100499054- | 100506572- | 100506647- | Unc | 0.797 | 0.792 | −0.005 | 1.77E−01 |
| | 100506571 | 100506646 | 100508297 | | | | | |
| AC024560.3 | 197348740- | 197349058- | 197349202- | Exc | 0.827 | 0.651 | −0.176 | 5.59E−04 |
| | 197349057 | 197349201 | 197350090 | | | | | |
| AC027612.6 | 91825060- | 91842946- | 91843024- | Inc | 0.380 | 0.557 | 0.177 | 1.59E−05 |
| | 91842945 | 91843023 | 91843393 | | | | | |
| AC037459.4 | 22447255- | 22449064- | 22449182- | Exc | 0.864 | 0.542 | −0.322 | 9.15E−124 |
| | 22449063 | 22449181 | 22451245 | | | | | |
| ACAA1 | 38170880- | 38173087- | 38173130- | Exc | 0.935 | 0.787 | −0.148 | 1.21E−14 |
| | 38173086 | 38173129 | 38173416 | | | | | |
| ACACA | 35564715- | 35567381- | 35567405- | Exc | 0.816 | 0.625 | −0.191 | 3.02E−08 |
| | 35567380 | 35567404 | 35578644 | | | | | |
| ACAD10 | 112147489- | 112150302- | 112150462- | Exc | 0.960 | 0.786 | −0.173 | 7.85E−04 |
| | 112150301 | 112150461 | 112153624 | | | | | |
| ACAD10 | 112147489- | 112148077- | 112148170- | Unc | 0.245 | 0.239 | −0.006 | 8.44E−01 |
| | 112148076 | 112148169 | 112150301 | | | | | |
| ACAP3 | 1230009- | 1230098- | 1230197- | Unc | 0.867 | 0.859 | −0.008 | 8.80E−01 |
| | 1230097 | 1230196 | 1230826 | | | | | |
| ACBD4 | 43214144- | 43214386- | 43214507- | Exc | 0.816 | 0.628 | −0.187 | 3.62E−04 |
| | 43214385 | 43214506 | 43214734 | | | | | |
| ACBD5 | 27508787- | 27512136- | 27512169- | Inc | 0.127 | 0.253 | 0.126 | 5.61E−05 |
| | 27512135 | 27512168 | 27512266 | | | | | |
| ACCS | 44102871- | 44103615- | 44103743- | Unc | 0.118 | 0.126 | 0.008 | 9.66E−01 |
| | 44103614 | 44103742 | 44104718 | | | | | |
| ACIN1 | 23551046- | 23559191- | 23559311- | Inc | 0.504 | 0.611 | 0.107 | 5.87E−05 |
| | 23559190 | 23559310 | 23559730 | | | | | |
| ACLY | 40049428- | 40052873- | 40052903- | Inc | 0.610 | 0.792 | 0.181 | 1.68E−22 |
| | 40052872 | 40052902 | 40054001 | | | | | |
| ACOT9 | 23751335- | 23754036- | 23754134- | Unc | 0.891 | 0.887 | −0.005 | 9.58E−01 |
| | 23754035 | 23754133 | 23761241 | | | | | |
| ACOX1 | 73953648- | 73956296- | 73956457- | Inc | 0.642 | 0.791 | 0.149 | 1.57E−04 |
| | 73956295 | 73956456 | 73969705 | | | | | |
| ACSL3 | 223752607- | 223765392- | 223765499- | Inc | 0.342 | 0.507 | 0.164 | 1.87E−10 |
| | 223765391 | 223765498 | 223773450 | | | | | |
| ACSL4 | 108926896- | 108939373- | 108939426- | Exc | 0.896 | 0.729 | −0.167 | 5.02E−04 |
| | 108939372 | 108939425 | 108976367 | | | | | |
| ACTN1 | 69349310- | 69349590- | 69349773- | Exc | 0.978 | 0.863 | −0.115 | 1.11E−92 |
| | 69349589 | 69349772 | 69350884 | | | | | |
| ACTN1 | 69343958- | 69345175- | 69345241- | Unc | 0.102 | 0.098 | −0.003 | 1.48E−01 |
| | 69345174 | 69345240 | 69345705 | | | | | |
| ACY1 | 52018175- | 52019223- | 52019288- | Exc | 0.950 | 0.825 | −0.125 | 1.20E−04 |
| | 52019222 | 52019287 | 52019376 | | | | | |
| AD000671.6 | 36235323- | 36235527- | 36235640- | Exc | 0.519 | 0.093 | −0.426 | 1.77E−15 |
| | 36235526 | 36235639 | 36236025 | | | | | |
| ADA | 43249789- | 43251229- | 43251294- | Exc | 0.943 | 0.813 | −0.131 | 1.85E−05 |
| | 43251228 | 43251293 | 43251469 | | | | | |
| ADAM15 | 155032820- | 155033239- | 155033309- | Unc | 0.797 | 0.805 | 0.008 | 8.01E−01 |
| | 155033238 | 155033308 | 155034720 | | | | | |
| ADAM33 | 3655341- | 3655420- | 3655497- | Exc | 0.970 | 0.861 | −0.108 | 6.77E−10 |
| | 3655419 | 3655496 | 3655673 | | | | | |
| ADAMTSL4 | 150521976- | 150522298- | 150522372- | Exc | 0.900 | 0.716 | −0.183 | 3.37E−07 |
| | 150522297 | 150522371 | 150524680 | | | | | |
| ADAMTSL4 | 150528843- | 150529097- | 150529270- | Exc | 0.956 | 0.724 | −0.231 | 2.64E−30 |
| | 150529096 | 150529269 | 150529414 | | | | | |
| ADARB1 | 46603426- | 46604389- | 46604509- | Exc | 0.761 | 0.649 | −0.113 | 9.54E−05 |
| | 46604388 | 46604508 | 46604837 | | | | | |
| ADK | 76158338- | 76285014- | 76285185- | Exc | 0.974 | 0.833 | −0.141 | 7.08E−16 |
| | 76285013 | 76285184 | 76349039 | | | | | |
| AFMID | 76198833- | 76200909- | 76200982- | Inc | 0.128 | 0.647 | 0.519 | 8.70E−13 |
| | 76200908 | 76200981 | 76202026 | | | | | |
| AFTPH | 64800203- | 64806620- | 64806681- | Unc | 0.889 | 0.889 | −0.001 | 9.61E−01 |
| | 64806619 | 64806680 | 64812555 | | | | | |
| AFTPH | 64800203- | 64806620- | 64808408- | Unc | 0.838 | 0.832 | −0.006 | 9.12E−01 |
| | 64806619 | 64808407 | 64812555 | | | | | |
| AHI1 | 135813430- | 135816952- | 135817037- | Inc | 0.370 | 0.584 | 0.214 | 7.37E−04 |
| | 135816951 | 135817036 | 135818325 | | | | | |
| AK4 | 65613349- | 65613961- | 65614236- | Unc | 0.694 | 0.703 | 0.010 | 8.91E−01 |
| | 65613960 | 65614235 | 65656392 | | | | | |
| AKAP11 | 42869879- | 42871184- | 42871319- | Exc | 0.944 | 0.786 | −0.158 | 2.49E−07 |
| | 42871183 | 42871318 | 42872668 | | | | | |
| AKAP13 | 86199019- | 86201768- | 86201822- | Inc | 0.740 | 0.874 | 0.134 | 3.44E−04 |
| | 86201767 | 86201821 | 86207793 | | | | | |
| AKIP1 | 8933219- | 8934000- | 8934081- | Exc | 0.528 | 0.339 | −0.189 | 1.79E−06 |
| | 8933999 | 8934080 | 8936372 | | | | | |
| AKIP1 | 8936478- | 8938834- | 8938915- | Exc | 0.983 | 0.769 | −0.214 | 1.28E−34 |
| | 8938833 | 8938914 | 8940883 | | | | | |
| AKR1A1 | 46033850- | 46034157- | 46034357- | Exc | 0.941 | 0.687 | −0.254 | 3.92E−47 |
| | 46034156 | 46034356 | 46034598 | | | | | |
| AL163636.6 | 21152918- | 21161706- | 21161810- | Inc | 0.172 | 0.283 | 0.112 | 1.19E−06 |
| | 21161705 | 21161809 | 21167513 | | | | | |
| ALDH3B1 | 67782930- | 67785997- | 67786107- | Exc | 0.932 | 0.792 | −0.140 | 1.96E−08 |
| | 67785996 | 67786106 | 67786241 | | | | | |
| ALDH4A1 | 19212123- | 19212958- | 19213006- | Exc | 0.958 | 0.792 | −0.166 | 4.30E−04 |
| | 19212957 | 19213005 | 19215855 | | | | | |
| ALKBH1 | 78146314- | 78161081- | 78161244- | Exc | 0.967 | 0.828 | −0.139 | 3.85E−04 |
| | 78161080 | 78161243 | 78170711 | | | | | |
| ALKBH3 | 43911379- | 43923066- | 43923276- | Exc | 0.950 | 0.802 | −0.148 | 2.58E−06 |
| | 43923065 | 43923275 | 43940587 | | | | | |
| ALKBH3 | 43911379- | 43913591- | 43913680- | Exc | 0.858 | 0.549 | −0.309 | 8.23E−17 |
| | 43913590 | 43913679 | 43923065 | | | | | |
| AMBRA1 | 46529921- | 46534277- | 46534364- | Unc | 0.637 | 0.640 | 0.003 | 6.56E−01 |
| | 46534276 | 46534363 | 46563494 | | | | | |
| AMDHD2 | 2571125- | 2577562- | 2577617- | Unc | 0.810 | 0.813 | 0.003 | 9.67E−01 |
| | 2577561 | 2577616 | 2577773 | | | | | |
| AMIGO2 | 47472849- | 47472925- | 47473032- | Unc | 0.540 | 0.534 | −0.006 | 8.12E−01 |
| | 47472924 | 47473031 | 47473244 | | | | | |
| AMMECR1L | 128631847- | 128641777- | 128641887- | Exc | 0.970 | 0.829 | −0.142 | 1.23E−05 |
| | 128641776 | 128641886 | 128643383 | | | | | |
| AMOTL1 | 94501727- | 94528177- | 94528327- | Unc | 0.757 | 0.757 | −0.001 | 9.49E−01 |
| | 94528176 | 94528326 | 94532555 | | | | | |
| AMPD2 | 110162901- | 110167925- | 110168056- | Exc | 0.844 | 0.690 | −0.154 | 3.82E−04 |
| | 110167924 | 110168055 | 110168283 | | | | | |
| AMZ2P1 | 62969046- | 62969360- | 62969636- | Unc | 0.832 | 0.836 | 0.004 | 8.86E−01 |
| | 62969359 | 62969635 | 62970672 | | | | | |
| ANAPC11 | 79849710- | 79851428- | 79851491- | Unc | 0.705 | 0.708 | 0.003 | 9.31E−01 |
| | 79851427 | 79851490 | 79852342 | | | | | |
| ANK2 | 114290962- | 114293689- | 114293782- | Exc | 0.671 | 0.422 | −0.249 | 4.15E−06 |
| | 114293688 | 114293781 | 114294245 | | | | | |
| ANKRD10 | 111545611- | 111552877- | 111553009- | Exc | 0.380 | 0.100 | −0.280 | 4.11E−21 |
| | 111552876 | 111553008 | 111558379 | | | | | |
| ANKRD10 | 1115456Il- | 111552877- | 111553042- | Exc | 0.390 | 0.088 | −0.302 | 4.37E−24 |
| | li 1552876 | 111553041 | 111558379 | | | | | |
| ANKRD11 | 89357592- | 89358089- | 89358186- | Inc | 0.347 | 0.523 | 0.176 | 3.41E−09 |
| | 89358088 | 89358185 | 89371613 | | | | | |
| ANKRD17 | 74000980- | 74005248- | 74006001- | Unc | 0.859 | 0.862 | 0.003 | 9.24E−01 |
| | 74005247 | 74006000 | 74007457 | | | | | |
| ANKRD36 | 97875447- | 97875537- | 97875610- | Exc | 0.301 | 0.066 | −0.235 | 1.47E−04 |
| | 97875536 | 97875609 | 97877282 | | | | | |
| ANO6 | 45610175- | 45695797- | 45695877- | Exc | 0.882 | 0.765 | −0.117 | 1.59E−10 |
| | 45695796 | 45695876 | 45725077 | | | | | |
| ANO6 | 45610175- | 45664245- | 45664308- | Unc | 0.124 | 0.133 | 0.009 | 6.17E−01 |
| | 45664244 | 45664307 | 45695796 | | | | | |
| ANXA2 | 60678286- | 60689457- | 60689538- | Inc | 0.054 | 0.154 | 0.100 | 0.00E+00 |
| | 60689456 | 60689537 | 60690141 | | | | | |
| AOX1 | 201474138- | 201476107- | 201476217- | Unc | 0.904 | 0.896 | −0.008 | 4.94E−01 |
| | 201476106 | 201476216 | 201477331 | | | | | |
| AP1G1 | 71799488- | 71801780- | 71801789- | Inc | 0.251 | 0.489 | 0.238 | 4.95E−16 |
| | 71801779 | 71801788 | 71803525 | | | | | |
| AP2M1 | 183898040- | 183898433- | 183898530- | Exc | 0.423 | 0.261 | −0.162 | 1.38E−70 |
| | 183898432 | 183898529 | 183898636 | | | | | |
| AP2M1 | 183898040- | 183898433- | 183898439- | Exc | 0.605 | 0.423 | −0.182 | 3.32E−109 |
| | 183898432 | 183898438 | 183898636 | | | | | |
| APBB2 | 41035322- | 41067593- | 41067691- | Exc | 0.698 | 0.538 | −0.161 | 9.66E−05 |
| | 41067592 | 41067690 | 41102655 | | | | | |
| APOL1 | 36651046- | 36653129- | 36653183- | Exc | 0.862 | 0.753 | −0.109 | 1.35E−06 |
| | 36653128 | 36653182 | 36653364 | | | | | |
| APP | 27354791- | 27369675- | 27369732- | Exc | 0.739 | 0.590 | −0.149 | 8.80E−131 |
| | 27369674 | 27369731 | 27372329 | | | | | |
| ARHGAP12 | 32120729- | 32128565- | 32128640- | Inc | 0.554 | 0.896 | 0.342 | 4.84E−20 |
| | 32128564 | 32128639 | 32132388 | | | | | |
| ARHGAP12 | 32141526- | 32142994- | 32143135- | Inc | 0.788 | 0.929 | 0.140 | 1.26E−10 |
| | 32142993 | 32143134 | 32150322 | | | | | |
| ARHGAP23 | 36654069- | 36654656- | 36654753- | Exc | 0.821 | 0.687 | −0.134 | 6.60E−20 |
| | 36654655 | 36654752 | 36656838 | | | | | |
| ARHGEF10 | 1824901- | 1828214- | 1828331- | Inc | 0.536 | 0.725 | 0.190 | 4.24E−10 |
| | 1828213 | 1828330 | 1830800 | | | | | |
| ARHGEF10 | 1772280- | 1791519- | 1791603- | Unc | 0.859 | 0.860 | 0.002 | 9.61E−01 |
| | 1791518 | 1791602 | 1806125 | | | | | |
| ARHGEF10L | 17958962- | 17961043- | 17961058- | Exc | 0.874 | 0.744 | −0.130 | 1.03E−03 |
| | 17961042 | 17961057 | 17961329 | | | | | |
| ARHGEF12 | 120300227- | 120300421- | 120300541- | Exc | 0.810 | 0.709 | −0.102 | 2.31E−06 |
| | 120300420 | 120300540 | 120302479 | | | | | |
| ARHGEF25 | 58008801- | 58009020- | 58009098- | Exc | 0.959 | 0.856 | −0.103 | 8.02E−06 |
| | 58009019 | 58009097 | 58009294 | | | | | |
| ARID4B | 235359431- | 235377084- | 235377342- | Exc | 0.662 | 0.512 | −0.150 | 2.83E−04 |
| | 235377083 | 235377341 | 235383107 | | | | | |
| ARID5A | 97213255- | 97215058- | 97215197- | Unc | 0.520 | 0.523 | 0.004 | 7.80E−01 |
| | 97215057 | 97215196 | 97215489 | | | | | |
| ARIH2 | 48960245- | 48962151- | 48962405- | Exc | 0.530 | 0.371 | −0.159 | 1.77E−04 |
| | 48962150 | 48962404 | 48964894 | | | | | |
| ARIH2 | 48960245- | 48962151- | 48962273- | Exc | 0.646 | 0.470 | −0.175 | 3.49E−06 |
| | 48962150 | 48962272 | 48964894 | | | | | |
| ARIH2 | 48960245- | 48982569- | 48982615- | Unc | 0.897 | 0.906 | 0.009 | 9.17E−01 |
| | 48982568 | 48982614 | 48999044 | | | | | |
| ARIH2 | 48965247- | 48982569- | 48982615- | Unc | 0.289 | 0.292 | 0.002 | 9.70E−01 |
| | 48982568 | 48982614 | 48999044 | | | | | |
| ARMC10 | 102727212- | 102732924- | 102733101- | Unc | 0.780 | 0.781 | 0.002 | 9.84E−01 |
| | 102732923 | 102733100 | 102737723 | | | | | |
| ARSJ | 114824832- | 114827821- | 114827902- | Unc | 0.103 | 0.103 | 0.000 | 9.59E−01 |
| | 114827820 | 114827901 | 114899592 | | | | | |
| ARSK | 94903754- | 94916596- | 94916647- | Inc | 0.039 | 0.219 | 0.180 | 1.16E−06 |
| | 94916595 | 94916646 | 94918619 | | | | | |
| ASAP2 | 9528676- | 9531191- | 9531326- | Inc | 0.700 | 0.900 | 0.200 | 4.73E−06 |
| | 9531190 | 9531325 | 9533610 | | | | | |
| ASCC2 | 30221247- | 30221611- | 30221770- | Exc | 0.863 | 0.730 | −0.133 | 4.24E−05 |
| | 30221610 | 30221769 | 30228233 | | | | | |
| ASCC2 | 30228332- | 30230478- | 30230540- | Unc | 0.605 | 0.615 | 0.009 | 9.35E−01 |
| | 30230477 | 30230539 | 30234166 | | | | | |
| ASL | 65551650- | 65551731- | 65551809- | Exc | 0.850 | 0.744 | −0.106 | 3.00E−04 |
| | 65551730 | 65551808 | 65552320 | | | | | |
| ASPH | 62555993- | 62556504- | 62556561- | Exc | 0.839 | 0.726 | −0.113 | 8.37E−70 |
| | 62556503 | 62556560 | 62557158 | | | | | |
| ASPH | 62557192- | 62559309- | 62559438- | Inc | 0.786 | 0.955 | 0.169 | 1.59E−243 |
| | 62559308 | 62559437 | 62563608 | | | | | |
| ASUN | 27067062- | 27067341- | 27067512- | Exc | 0.834 | 0.705 | −0.128 | 1.87E−04 |
| | 27067340 | 27067511 | 27068934 | | | | | |
| ASXL1 | 31017235- | 31017704- | 31017857- | Exc | 0.851 | 0.696 | −0.155 | 9.56E−04 |
| | 31017703 | 31017856 | 31019123 | | | | | |
| ATG10 | 81474407- | 81548381- | 81548479- | Inc | 0.811 | 0.970 | 0.159 | 1.16E−03 |
| | 81548380 | 81548478 | 81571963 | | | | | |
| ATG12 | 115173462- | 115176194- | 115176310- | Unc | 0.624 | 0.633 | 0.009 | 8.94E−01 |
| | 115176193 | 115176309 | 115176514 | | | | | |
| ATG13 | 46639926- | 46651595- | 46651651- | Unc | 0.845 | 0.854 | 0.010 | 7.41E−01 |
| | 46651594 | 46651650 | 46665828 | | | | | |
| ATG13 | 46639441- | 46639875- | 46639926- | Unc | 0.224 | 0.228 | 0.003 | 9.18E−01 |
| | 46639874 | 46639925 | 46651594 | | | | | |
| ATG16L1 | 234181699- | 234182367- | 234182424- | Exc | 0.763 | 0.487 | −0.277 | 9.35E−10 |
| | 234182366 | 234182423 | 234183321 | | | | | |
| ATG4B | 242590751- | 242592722- | 242592785- | Unc | 0.109 | 0.110 | 0.001 | 9.93E−01 |
| | 242592721 | 242592784 | 242592926 | | | | | |
| ATG7 | 11340330- | 11340836- | 11340891- | Exc | 0.993 | 0.892 | −0.101 | 4.09E−07 |
| | 11340835 | 11340890 | 11348416 | | | | | |
| ATG9A | 220079805- | 220080719- | 220080903- | Exc | 0.979 | 0.874 | −0.104 | 4.77E−04 |
| | 220080718 | 220080902 | 220081085 | | | | | |
| ATP2C1 | 130613182- | 130613575- | 130613620- | Inc | 0.666 | 0.818 | 0.152 | 3.71E−07 |
| | 130613574 | 130613619 | 130649259 | | | | | |
| ATP2C1 | 130613182- | 130613552- | 130613620- | Inc | 0.677 | 0.824 | 0.146 | 4.82E−07 |
| | 130613551 | 130613619 | 130649259 | | | | | |
| ATP2C1 | 130613182- | 130613434- | 130613620- | Inc | 0.684 | 0.826 | 0.142 | 7.77E−07 |
| | 130613433 | 130613619 | 130649259 | | | | | |
| ATP5C1 | 7844818- | 7848937- | 7848974- | Unc | 0.215 | 0.207 | −0.007 | 7.79E−01 |
| | 7848936 | 7848973 | 7849621 | | | | | |
| ATP5SL | 41939579- | 41942297- | 41942378- | Inc | 0.850 | 0.964 | 0.114 | 2.08E−09 |
| | 41942296 | 41942377 | 41944122 | | | | | |
| ATP6C | 2571125- | 2577562- | 2577617- | Unc | 0.810 | 0.813 | 0.003 | 9.67E−01 |
| | 2577561 | 2577616 | 2577773 | | | | | |
| ATPAF1 | 47118301- | 47119489- | 47119540- | Exc | 0.964 | 0.745 | −0.219 | 3.04E−29 |
| | 47119488 | 47119539 | 47123798 | | | | | |
| ATXN1 | 16658133- | 16753464- | 16753579- | Exc | 0.820 | 0.688 | −0.132 | 3.78E−04 |
| | 16753463 | 16753578 | 16761528 | | | | | |
| ATXN3 | 92559663- | 92560090- | 92560176- | Exc | 0.950 | 0.785 | −0.165 | 1.73E−07 |
| | 92560089 | 92560175 | 92562436 | | | | | |
| ATXN3 | 92559663- | 92560090- | 92560195- | Exc | 0.908 | 0.636 | −0.271 | 5.26E−07 |
| | 92560089 | 92560194 | 92562436 | | | | | |
| AUP1 | 74755617- | 74755878- | 74756063- | Exc | 0.928 | 0.813 | −0.114 | 4.48E−17 |
| | 74755877 | 74756062 | 74756258 | | | | | |
| AUTS2 | 70236631- | 70239014- | 70239086- | Exc | 0.901 | 0.758 | −0.143 | 1.11E−03 |
| | 70239013 | 70239085 | 70240342 | | | | | |
| AVL9 | 32615685- | 32619831- | 32619885- | Exc | 0.972 | 0.592 | −0.379 | 4.43E−19 |
| | 32619830 | 32619884 | 32620413 | | | | | |
| B3GALNT1 | 160804577- | 160818927- | 160819022- | Inc | 0.700 | 0.864 | 0.164 | 6.43E−04 |
| | 160818926 | 160819021 | 160821214 | | | | | |
| B3GALNT1 | 160804577- | 160807732- | 160807851- | Unc | 0.150 | 0.156 | 0.007 | 8.87E−01 |
| | 160807731 | 160807850 | 160818926 | | | | | |
| BAG6 | 31607004- | 31607277- | 31607424- | Exc | 0.597 | 0.458 | −0.139 | 2.29E−11 |
| | 31607276 | 31607423 | 31607975 | | | | | |
| BANP | 88017866- | 88037901- | 88038018- | Inc | 0.825 | 0.985 | 0.160 | 5.85E−04 |
| | 88037900 | 88038017 | 88039695 | | | | | |
| BAX | 49458220- | 49458805- | 49458857- | Exc | 0.937 | 0.832 | −0.105 | 1.22E−06 |
| | 49458804 | 49458856 | 49459454 | | | | | |
| BAZ1A | 35255208- | 35255332- | 35255428- | Exc | 0.877 | 0.692 | −0.185 | 9.82E−04 |
| | 35255331 | 35255427 | 35261980 | | | | | |
| BAZ2A | 57011317- | 57024560- | 57024650- | Unc | 0.551 | 0.549 | −0.002 | 9.42E−01 |
| | 57024559 | 57024649 | 57029965 | | | | | |
| BBS4 | 73004649- | 73007632- | 73007744- | Exc | 0.982 | 0.750 | −0.232 | 1.36E−11 |
| | 73007631 | 73007743 | 73009118 | | | | | |
| BBS5 | 170349520- | 170350251- | 170350347- | Exc | 1.000 | 0.824 | −0.176 | 7.16E−07 |
| | 170350250 | 170350346 | 170355995 | | | | | |
| BBX | 107497367- | 107508634- | 107508724- | Inc | 0.640 | 0.757 | 0.117 | 5.10E−05 |
| | 107508633 | 107508723 | 107510086 | | | | | |
| BCAR1 | 75269885- | 75271081- | 75271243- | Exc | 0.908 | 0.761 | −0.147 | 5.95E−05 |
| | 75271080 | 75271242 | 75276367 | | | | | |
| BCAR1 | 75269885- | 75270780- | 75270897- | Exc | 0.982 | 0.797 | −0.185 | 3.94E−21 |
| | 75270779 | 75270896 | 75271080 | | | | | |
| BCAR1 | 75269885- | 75270726- | 75270897- | Exc | 0.959 | 0.642 | −0.317 | 2.84E−18 |
| | 75270725 | 75270896 | 75271080 | | | | | |
| BCL2L2 | 23777409- | 23778025- | 23778142- | Unc | 0.905 | 0.898 | −0.007 | 9.53E−01 |
| | 23778024 | 23778141 | 23780181 | | | | | |
| BCOR | 39930413- | 39930890- | 39930944- | Exc | 0.661 | 0.452 | −0.209 | 8.56E−04 |
| | 39930889 | 39930943 | 39931601 | | | | | |
| BEND6 | 56857354- | 56879260- | 56879324- | Unc | 0.101 | 0.100 | 0.000 | 9.69E−01 |
| | 56879259 | 56879323 | 56879930 | | | | | |
| BIN1 | 127808489- | 127808730- | 127808820- | Exc | 0.878 | 0.726 | −0.152 | 1.40E−04 |
| | 127808729 | 127808819 | 127815048 | | | | | |
| BIN1 | 127808489- | 127808730- | 127808820- | Exc | 0.884 | 0.712 | −0.172 | 6.43E−05 |
| | 127808729 | 127808819 | 127816586 | | | | | |
| BIRC6 | 32658873- | 32660564- | 32660654- | Exc | 0.787 | 0.552 | −0.235 | 3.99E−06 |
| | 32660563 | 32660653 | 32661120 | | | | | |
| BIRC6 | 32800434- | 32815873- | 32816046- | Unc | 0.108 | 0.108 | 0.000 | 8.86E−01 |
| | 32815872 | 32816045 | 32818981 | | | | | |
| BLOC1S6 | 45879724- | 45884333- | 45884475- | Exc | 0.724 | 0.597 | −0.127 | 5.48E−04 |
| | 45884332 | 45884474 | 45897625 | | | | | |
| BLOC1S6 | 45879724- | 45884333- | 45884475- | Exc | 0.779 | 0.516 | −0.263 | 3.98E−23 |
| | 45884332 | 45884474 | 45895297 | | | | | |
| BLOC1S6 | 45879724- | 45895298- | 45895386- | Unc | 0.834 | 0.841 | 0.007 | 7.47E−01 |
| | 45895297 | 45895385 | 45897625 | | | | | |
| BMP2K | 79800046- | 79808328- | 79808439- | Exc | 0.554 | 0.274 | −0.281 | 2.14E−11 |
| | 79808327 | 79808438 | 79831763 | | | | | |
| BOK | 242501892- | 242509540- | 242509704- | Exc | 0.874 | 0.748 | −0.126 | 4.30E−04 |
| | 242509539 | 242509703 | 242511711 | | | | | |
| BPTF | 65955992- | 65959449- | 65959623- | Exc | 0.585 | 0.199 | −0.386 | 5.43E−23 |
| | 65959448 | 65959622 | 65960327 | | | | | |
| BPTF | 65871861- | 65882244- | 65882433- | Inc | 0.665 | 0.832 | 0.167 | 2.66E−04 |
| | 65882243 | 65882432 | 65887959 | | | | | |
| BRD8 | 137492957- | 137495244- | 137495289- | Exc | 0.610 | 0.401 | −0.208 | 1.57E−11 |
| | 137495243 | 137495288 | 137495757 | | | | | |
| BRD8 | 137503768- | 137504159- | 137504378- | Unc | 0.735 | 0.739 | 0.004 | 9.45E−01 |
| | 137504158 | 137504377 | 137504910 | | | | | |
| BRD8 | 137501798- | 137502207- | 137502417- | Unc | 0.729 | 0.722 | −0.007 | 8.43E−01 |
| | 137502206 | 137502416 | 137503622 | | | | | |
| BROX | 222886285- | 222889026- | 222889143- | Inc | 0.863 | 0.968 | 0.105 | 5.17E−07 |
| | 222889025 | 222889142 | 222892266 | | | | | |
| BTBD19 | 45274579- | 45275885- | 45276099- | Exc | 0.992 | 0.721 | −0.270 | 1.50E−07 |
| | 45275884 | 45276098 | 45276225 | | | | | |
| BTBD19 | 45274579- | 45275885- | 45275922- | Exc | 0.988 | 0.668 | −0.320 | 3.77E−06 |
| | 45275884 | 45275921 | 45276225 | | | | | |
| BTBD3 | 11871603- | 11898426- | 11898660- | Unc | 0.230 | 0.232 | 0.001 | 9.69E−01 |
| | 11898425 | 11898659 | 11898981 | | | | | |
| BTF3L4 | 52522052- | 52525507- | 52525574- | Unc | 0.893 | 0.902 | 0.008 | 5.65E−01 |
| | 52525506 | 52525573 | 52530496 | | | | | |
| C10orf118 | 115923089- | 115932900- | 115933025- | Unc | 0.238 | 0.228 | −0.010 | 9.66E−01 |
| | 115932899 | 115933024 | 115933773 | | | | | |
| C11orf30 | 76169403- | 76170980- | 76171130- | Inc | 0.629 | 0.903 | 0.274 | 3.48E−04 |
| | 76170979 | 76171129 | 76174864 | | | | | |
| C11orf30 | 76169403- | 76170977- | 76171130- | Inc | 0.729 | 0.936 | 0.207 | 7.07E−05 |
| | 76170976 | 76171129 | 76174864 | | | | | |
| C11orf30 | 76248995- | 76250643- | 76250685- | Unc | 0.754 | 0.753 | −0.001 | 8.84E−01 |
| | 76250642 | 76250684 | 76253259 | | | | | |
| C11orf57 | 111945083- | 111945611- | 111945641- | Unc | 0.395 | 0.390 | −0.005 | 9.74E−01 |
| | 111945610 | 111945640 | 111946290 | | | | | |
| C11orf70 | 101929687- | 101937216- | 101937383- | Exc | 0.973 | 0.843 | −0.130 | 1.77E−04 |
| | 101937215 | 101937382 | 101946603 | | | | | |
| C12orf29 | 88429516- | 88433925- | 88434043- | Inc | 0.745 | 0.897 | 0.152 | 1.32E−05 |
| | 88433924 | 88434042 | 88436601 | | | | | |
| C14orf159 | 91580628- | 91611565- | 91611658- | Unc | 0.121 | 0.124 | 0.003 | 9.48E−01 |
| | 91611564 | 91611657 | 91623982 | | | | | |
| C16orf13 | 684798- | 685518- | 685775- | Exc | 0.760 | 0.447 | −0.313 | 4.99E−04 |
| | 685517 | 685774 | 686093 | | | | | |
| C1orf85 | 156264357- | 156264550- | 156264808- | Exc | 0.844 | 0.698 | −0.147 | 4.56E−15 |
| | 156264549 | 156264807 | 156265316 | | | | | |
| C1RL | 7254684- | 7260847- | 7261076- | Exc | 0.865 | 0.689 | −0.176 | 6.02E−11 |
| | 7260846 | 7261075 | 7261705 | | | | | |
| C2CD5 | 22610096- | 22612426- | 22612477- | Inc | 0.523 | 0.770 | 0.247 | 3.77E−06 |
| | 22612425 | 22612476 | 22622642 | | | | | |
| C2CD5 | 22610096- | 22611418- | 22611490- | Inc | 0.854 | 1.000 | 0.146 | 1.04E−06 |
| | 22611417 | 22611489 | 22612425 | | | | | |
| C2CD5 | 22610096- | 22611418- | 22611520- | Inc | 0.881 | 1.000 | 0.119 | 3.44E−07 |
| | 22611417 | 22611519 | 22612425 | | | | | |
| C2orf76 | 120075080- | 120078730- | 120078781- | Unc | 0.865 | 0.858 | −0.008 | 9.03E−01 |
| | 120078729 | 120078780 | 120097402 | | | | | |
| C3orf18 | 50599179- | 50602897- | 50603293- | Inc | 0.427 | 0.627 | 0.200 | 5.28E−04 |
| | 50602896 | 50603292 | 50604893 | | | | | |
| C5orf42 | 37162669- | 37164375- | 37164430- | Exc | 0.973 | 0.869 | −0.103 | 7.10E−04 |
| | 37164374 | 37164429 | 37165640 | | | | | |
| C5orf45 | 179275067- | 179280197- | 179280277- | Inc | 0.132 | 0.390 | 0.258 | 3.00E−06 |
| | 179280196 | 179280276 | 179280377 | | | | | |
| C8orf59 | 86129732- | 86131552- | 86131593- | Inc | 0.127 | 0.317 | 0.191 | 1.66E−25 |
| | 86131551 | 86131592 | 86132534 | | | | | |
| C8orf59 | 86129732- | 86131465- | 86131593- | Inc | 0.105 | 0.225 | 0.119 | 9.55E−14 |
| | 86131464 | 86131592 | 86132534 | | | | | |
| C9orf156 | 100672899- | 100675155- | 100675258- | Inc | 0.130 | 0.274 | 0.145 | 1.03E−03 |
| | 100675154 | 100675257 | 100675682 | | | | | |
| C9orf156 | 100675841- | 100678446- | 100678621- | Unc | 0.758 | 0.762 | 0.004 | 8.93E−01 |
| | 100678445 | 100678620 | 100684699 | | | | | |
| C9orf85 | 74562029- | 74586421- | 74586535- | Exc | 0.609 | 0.362 | −0.247 | 1.04E−04 |
| | 74586420 | 74586534 | 74597572 | | | | | |
| CA5BP1 | 15706982- | 15711086- | 15711182- | Inc | 0.727 | 0.908 | 0.181 | 1.00E−03 |
| | 15711085 | 15711181 | 15720904 | | | | | |
| CACNA1C | 2721180- | 2742795- | 2742879- | Unc | 0.201 | 0.192 | −0.009 | 9.98E−01 |
| | 2742794 | 2742878 | 2743462 | | | | | |
| CALU | 128388859- | 128394316- | 128394510- | Inc | 0.625 | 0.807 | 0.182 | 2.09E−164 |
| | 128394315 | 128394509 | 128394582 | | | | | |
| CAMK2D | 114375672- | 114376882- | 114376978- | Exc | 0.251 | 0.079 | −0.173 | 8.44E−18 |
| | 114376881 | 114376977 | 114378490 | | | | | |
| CAMK2G | 75583843- | 75585037- | 75585106- | Unc | 0.881 | 0.890 | 0.009 | 9.67E−01 |
| | 75585036 | 75585105 | 75597225 | | | | | |
| CAMSAP1 | 138742308- | 138754313- | 138754455- | Unc | 0.817 | 0.816 | −0.001 | 9.89E−01 |
| | 138754312 | 138754454 | 138758301 | | | | | |
| CAMTA1 | 6845636- | 6880241- | 6880311- | Inc | 0.732 | 0.895 | 0.163 | 4.82E−15 |
| | 6880240 | 6880310 | 6885151 | | | | | |
| CAPN10 | 241535939- | 241536098- | 241536360- | Unc | 0.895 | 0.887 | −0.008 | 9.33E−01 |
| | 241536097 | 241536359 | 241537304 | | | | | |
| CAPN7 | 15283761- | 15287033- | 15287179- | Exc | 0.963 | 0.863 | −0.101 | 2.98E−08 |
| | 15287032 | 15287178 | 15288238 | | | | | |
| CAPN7 | 15288965- | 15292482- | 15292531- | Exc | 0.969 | 0.852 | −0.117 | 1.75E−06 |
| | 15292481 | 15292530 | 15292622 | | | | | |
| CAPRIN2 | 30869611- | 30872013- | 30873850- | Exc | 0.835 | 0.625 | −0.210 | 9.09E−11 |
| | 30872012 | 30873849 | 30876192 | | | | | |
| CAPRIN2 | 30869611- | 30872013- | 30872160- | Exc | 0.768 | 0.538 | −0.230 | 2.34E−08 |
| | 30872012 | 30872159 | 30876192 | | | | | |
| CAPRIN2 | 30869611- | 30873745- | 30873850- | Exc | 0.722 | 0.471 | −0.251 | 4.48E−09 |
| | 30873744 | 30873849 | 30876192 | | | | | |
| CAPRIN2 | 30884445- | 30886563- | 30886646- | Exc | 0.473 | 0.150 | −0.323 | 6.22E−16 |
| | 30886562 | 30886645 | 30887901 | | | | | |
| CARD8 | 48737801- | 48741640- | 48741790- | Exc | 0.667 | 0.426 | −0.242 | 6.35E−05 |
| | 48741639 | 48741789 | 48744218 | | | | | |
| CARD8 | 48737801- | 48741640- | 48741784- | Exc | 0.477 | 0.185 | −0.293 | 7.65E−05 |
| | 48741639 | 48741783 | 48744218 | | | | | |
| CARF | 203782767- | 203806583- | 203806704- | Unc | 0.801 | 0.806 | 0.005 | 9.25E−01 |
| | 203806582 | 203806703 | 203817281 | | | | | |
| CARKD | 111276627- | 111277537- | 111277626- | Exc | 0.962 | 0.771 | −0.191 | 4.83E−12 |
| | 111277536 | 111277625 | 111279785 | | | | | |
| CASP3 | 185559623- | 185569619- | 185569786- | Inc | 0.330 | 0.471 | 0.141 | 7.63E−05 |
| | 185569618 | 185569785 | 185570548 | | | | | |
| CAST | 96058403- | 96062458- | 96062564- | Exc | 0.278 | 0.161 | −0.117 | 4.69E−21 |
| | 96062457 | 96062563 | 96063192 | | | | | |
| CAST | 96058403- | 96062498- | 96062564- | Exc | 0.425 | 0.268 | −0.156 | 1.16E−32 |
| | 96062497 | 96062563 | 96063192 | | | | | |
| CCBL2 | 89435151- | 89453935- | 89454035- | Inc | 0.399 | 0.517 | 0.119 | 3.36E−04 |
| | 89453934 | 89454034 | 89458267 | | | | | |
| CCDC126 | 23637671- | 23643705- | 23643899- | Unc | 0.337 | 0.340 | 0.003 | 9.87E−01 |
| | 23643704 | 23643898 | 23650789 | | | | | |
| CCDC136 | 128445956- | 128446291- | 128446450- | Unc | 0.872 | 0.871 | −0.001 | 9.71E−01 |
| | 128446290 | 128446449 | 128446741 | | | | | |
| CCDC25 | 27605797- | 27606012- | 27606116- | Exc | 0.871 | 0.699 | −0.171 | 2.16E−10 |
| | 27606011 | 27606115 | 27610028 | | | | | |
| CCDC25 | 27610105- | 27614236- | 27614288- | Unc | 0.813 | 0.813 | 0.000 | 9.07E−01 |
| | 27614235 | 27614287 | 27619935 | | | | | |
| CCDC90B | 82985784- | 82989769- | 82989873- | Exc | 0.857 | 0.745 | −0.113 | 1.89E−11 |
| | 82989768 | 82989872 | 82991183 | | | | | |
| CCNDBP1 | 43477806- | 43478018- | 43478078- | Exc | 0.923 | 0.767 | −0.156 | 9.47E−08 |
| | 43478017 | 43478077 | 43481396 | | | | | |
| CCNL1 | 156868171- | 156869518- | 156869720- | Exc | 0.326 | 0.204 | −0.123 | 2.22E−04 |
| | 156869517 | 156869719 | 156869965 | | | | | |
| CCNL2 | 1326246- | 1326677- | 1326956- | Unc | 0.890 | 0.889 | −0.001 | 9.31E−01 |
| | 1326676 | 1326955 | 1328058 | | | | | |
| CCNT1 | 49089613- | 49089782- | 49089946- | Unc | 0.876 | 0.872 | −0.005 | 9.13E−01 |
| | 49089781 | 49089945 | 49091920 | | | | | |
| CCNYL1 | 208591601- | 208598671- | 208598772- | Unc | 0.795 | 0.792 | −0.002 | 4.41E−01 |
| | 208598670 | 208598771 | 208602134 | | | | | |
| CD27-AS1 | 6557904- | 6560036- | 6560147- | Unc | 0.432 | 0.429 | −0.003 | 9.65E−01 |
| | 6560035 | 6560146 | 6560634 | | | | | |
| CD27-AS1 | 6557904- | 6559507- | 6560147- | Unc | 0.533 | 0.525 | −0.008 | 8.65E−01 |
| | 6559506 | 6560146 | 6560634 | | | | | |
| CD44 | 35211520- | 35232793- | 35232997- | Inc | 0.595 | 0.873 | 0.277 | 5.56E−09 |
| | 35232792 | 35232996 | 35236398 | | | | | |
| CD46 | 207940541- | 207941124- | 207941169- | Exc | 0.653 | 0.522 | −0.131 | 9.35E−20 |
| | 207941123 | 207941168 | 207943665 | | | | | |
| CD46 | 207959028- | 207963598- | 207963691- | Inc | 0.527 | 0.748 | 0.221 | 6.66E−57 |
| | 207963597 | 207963690 | 207966863 | | | | | |
| CD55 | 207512763- | 207513736- | 207513854- | Exc | 0.278 | 0.162 | −0.116 | 4.30E−09 |
| | 207513735 | 207513853 | 207532890 | | | | | |
| CD99P1 | 2541495- | 2544638- | 2544684- | Exc | 0.966 | 0.694 | −0.272 | 3.51E−05 |
| | 2544637 | 2544683 | 2556014 | | | | | |
| CD99P1 | 2537539- | 2540731- | 2540776- | Exc | 0.599 | 0.141 | −0.458 | 3.02E−12 |
| | 2540730 | 2540775 | 2541425 | | | | | |
| CDC14B | 99272072- | 99277931- | 99278075- | Exc | 0.458 | 0.228 | −0.230 | 1.57E−09 |
| | 99277930 | 99278074 | 99284787 | | | | | |
| CDC14B | 99266072- | 99271955- | 99272072- | Inc | 0.547 | 0.795 | 0.248 | 1.82E−08 |
| | 99271954 | 99272071 | 99277930 | | | | | |
| CDC16 | 115000608- | 115002120- | 115002175- | Exc | 0.870 | 0.559 | −0.311 | 2.24E−37 |
| | 115002119 | 115002174 | 115002273 | | | | | |
| CDC42BPA | 227300124- | 227300372- | 227300615- | Unc | 0.779 | 0.775 | −0.004 | 9.88E−01 |
| | 227300371 | 227300614 | 227307504 | | | | | |
| CDCA7L | 21948126- | 21951235- | 21951370- | Unc | 0.738 | 0.747 | 0.009 | 8.31E−01 |
| | 21951234 | 21951369 | 21956371 | | | | | |
| CDK16 | 47078064- | 47081672- | 47081742- | Inc | 0.286 | 0.566 | 0.280 | 8.90E−04 |
| | 47081671 | 47081741 | 47082950 | | | | | |
| CDK16 | 47078505- | 47081660- | 47081742- | Inc | 0.094 | 0.246 | 0.152 | 2.82E−06 |
| | 47081659 | 47081741 | 47082950 | | | | | |
| CDK16 | 47078505- | 47081660- | 47081780- | Inc | 0.056 | 0.169 | 0.113 | 3.41E−05 |
| | 47081659 | 47081779 | 47082950 | | | | | |
| CDK5RAP2 | 123220901- | 123222850- | 123223026- | Unc | 0.858 | 0.858 | 0.000 | 9.52E−01 |
| | 123222849 | 123223025 | 123230137 | | | | | |
| CEP164 | 117252585- | 117253512- | 117253659- | Unc | 0.235 | 0.229 | −0.006 | 9.69E−01 |
| | 117253511 | 117253658 | 117257918 | | | | | |
| CEP290 | 88447524- | 88448117- | 88448191- | Inc | 0.761 | 0.941 | 0.180 | 1.02E−07 |
| | 88448116 | 88448190 | 88449352 | | | | | |
| CHD3 | 7810807- | 7810919- | 7811021- | Exc | 0.533 | 0.342 | −0.191 | 6.58E−16 |
| | 7810918 | 7811020 | 7811211 | | | | | |
| CHEK2 | 29095926- | 29099493- | 29099555- | Exc | 0.613 | 0.260 | −0.353 | 6.09E−08 |
| | 29099492 | 29099554 | 29105993 | | | | | |
| CHKB | 51018232- | 51018486- | 51018512- | Exc | 0.958 | 0.848 | −0.110 | 5.70E−05 |
| | 51018485 | 51018511 | 51018618 | | | | | |
| CIRBP | 1272051- | 1273600- | 1273715- | Unc | 0.128 | 0.129 | 0.001 | 9.31E−01 |
| | 1273599 | 1273714 | 1274305 | | | | | |
| CIRBP | 1272051- | 1273493- | 1273715- | Unc | 0.152 | 0.144 | −0.007 | 8.95E−01 |
| | 1273492 | 1273714 | 1274305 | | | | | |
| CKLF | 66586697- | 66592093- | 66592252- | Exc | 0.541 | 0.438 | −0.103 | 5.18E−04 |
| | 66592092 | 66592251 | 66597024 | | | | | |
| CLASP1 | 122165292- | 122166600- | 122166624- | Inc | 0.165 | 0.429 | 0.264 | 5.96E−14 |
| | 122166599 | 122166623 | 122168441 | | | | | |
| CLASP2 | 33592888- | 33600617- | 33600668- | Unc | 0.637 | 0.628 | −0.009 | 9.35E−01 |
| | 33600616 | 33600667 | 33600798 | | | | | |
| CLCN6 | 11888277- | 11888515- | 11888682- | Exc | 0.604 | 0.333 | −0.271 | 5.43E−06 |
| | 11888514 | 11888681 | 11889252 | | | | | |
| CLK1 | 201724470- | 201724848- | 201724939- | Inc | 0.451 | 0.655 | 0.204 | 2.02E−11 |
| | 201724847 | 201724938 | 201725960 | | | | | |
| CMC1 | 28283304- | 28304782- | 28304872- | Inc | 0.748 | 0.878 | 0.130 | 1.98E−04 |
| | 28304781 | 28304871 | 28357823 | | | | | |
| CNOT10 | 32804356- | 32805947- | 32805983- | Exc | 1.000 | 0.899 | −0.101 | 2.05E−05 |
| | 32805946 | 32805982 | 32806173 | | | | | |
| COL12A1 | 75875496- | 75884754- | 75885027- | Inc | 0.758 | 0.876 | 0.118 | 0.00E+00 |
| | 75884753 | 75885026 | 75887378 | | | | | |
| COL16A1 | 32145287- | 32145405- | 32145453- | Exc | 0.543 | 0.400 | −0.143 | 1.15E−23 |
| | 32145404 | 32145452 | 32145642 | | | | | |
| COL16A1 | 32134457- | 32136157- | 32136248- | Exc | 0.349 | 0.163 | −0.186 | 5.75E−27 |
| | 32136156 | 32136247 | 32137215 | | | | | |
| COL16A1 | 32134457- | 32136203- | 32136248- | Exc | 0.508 | 0.277 | −0.231 | 2.46E−41 |
| | 32136202 | 32136247 | 32137215 | | | | | |
| COL6A3 | 238296828- | 238303230- | 238303848- | Exc | 0.394 | 0.175 | −0.219 | 9.35E−95 |
| | 238303229 | 238303847 | 238305369 | | | | | |
| COL6A3 | 238290143- | 238296225- | 238296828- | Inc | 0.310 | 0.501 | 0.191 | 2.58E−146 |
| | 238296224 | 238296827 | 238305369 | | | | | |
| COPB2 | 139102278- | 139103846- | 139104043- | Inc | 0.457 | 0.696 | 0.239 | 4.28E−04 |
| | 139103845 | 139104042 | 139108310 | | | | | |
| COPS7A | 6833985- | 6837092- | 6837168- | Exc | 0.870 | 0.743 | −0.127 | 1.13E−17 |
| | 6837091 | 6837167 | 6837388 | | | | | |
| COPS8 | 237994686- | 237995375- | 237995440- | Inc | 0.080 | 0.311 | 0.230 | 1.16E−37 |
| | 237995374 | 237995439 | 237995769 | | | | | |
| COQ6 | 74425928- | 74426118- | 74426226- | Exc | 0.943 | 0.796 | −0.147 | 1.71E−06 |
| | 74426117 | 74426225 | 74427875 | | | | | |
| COX20 | 244999059- | 245005246- | 245005361- | Exc | 0.649 | 0.446 | −0.203 | 1.39E−13 |
| | 245005245 | 245005360 | 245005496 | | | | | |
| CPEB2 | 15034836- | 15042088- | 15042112- | Exc | 0.257 | 0.132 | −0.125 | 8.15E−04 |
| | 15042087 | 15042111 | 15054037 | | | | | |
| CPNE1 | 34220846- | 34246852- | 34246937- | Unc | 0.361 | 0.358 | −0.003 | 9.59E−01 |
| | 34246851 | 34246936 | 34252681 | | | | | |
| CPSF7 | 61188046- | 61188664- | 61188730- | Inc | 0.065 | 0.211 | 0.146 | 3.45E−14 |
| | 61188663 | 61188729 | 61188861 | | | | | |
| CPT1C | 50194598- | 50195078- | 50195211- | Exc | 0.503 | 0.145 | −0.358 | 3.72E−05 |
| | 50195077 | 50195210 | 50195495 | | | | | |
| CPT1C | 50194598- | 50195078- | 50195147- | Exc | 0.651 | 0.285 | −0.366 | 5.73E−06 |
| | 50195077 | 50195146 | 50195495 | | | | | |
| CRAT | 131870357- | 131871458- | 131871557- | Exc | 0.574 | 0.424 | −0.150 | 5.12E−05 |
| | 131871457 | 131871556 | 131872761 | | | | | |
| CREBBP | 3824695- | 3827614- | 3827659- | Inc | 0.836 | 0.969 | 0.133 | 2.33E−10 |
| | 3827613 | 3827658 | 3828011 | | | | | |
| CRLS1 | 5990559- | 5996007- | 5996137- | Exc | 0.992 | 0.889 | −0.103 | 1.14E−06 |
| | 5996006 | 5996136 | 6012657 | | | | | |
| CRNDE | 54953122- | 54954210- | 54954251- | Exc | 0.973 | 0.867 | −0.106 | 2.55E−09 |
| | 54954209 | 54954250 | 54957496 | | | | | |
| CRYZ | 75190519- | 75196066- | 75196105- | Exc | 0.196 | 0.091 | −0.105 | 1.56E−04 |
| | 75196065 | 75196104 | 75198639 | | | | | |
| CRYZ | 75172679- | 75172787- | 75172889- | Exc | 0.838 | 0.322 | −0.516 | 1.73E−58 |
| | 75172786 | 75172888 | 75175781 | | | | | |
| CSAD | 53554629- | 53554911- | 53554976- | Exc | 0.398 | 0.224 | −0.174 | 6.03E−04 |
| | 53554910 | 53554975 | 53555056 | | | | | |
| CSDE1 | 115279477- | 115280092- | 115280185- | Unc | 0.104 | 0.096 | −0.008 | 2.11E−01 |
| | 115280091 | 115280184 | 115280583 | | | | | |
| CSNK1A1 | 148892773- | 148897357- | 148897441- | Unc | 0.113 | 0.116 | 0.003 | 7.55E−01 |
| | 148897356 | 148897440 | 148899852 | | | | | |
| CTDSPL | 37988703- | 37998602- | 37998635- | Exc | 0.596 | 0.336 | −0.261 | 1.76E−10 |
| | 37998601 | 37998634 | 38006061 | | | | | |
| CTIF | 46065684- | 46066129- | 46066203- | Unc | 0.235 | 0.241 | 0.006 | 9.23E−01 |
| | 46066128 | 46066202 | 46145908 | | | | | |
| CTSB | 11710989- | 11721885- | 11721973- | Unc | 0.220 | 0.219 | −0.001 | 9.29E−01 |
| | 11721884 | 11721972 | 11725509 | | | | | |
| CUTC | 101507148- | 101510126- | 101510154- | Exc | 0.509 | 0.240 | −0.269 | 5.92E−10 |
| | 101510125 | 101510153 | 101514285 | | | | | |
| CWC25 | 36966019- | 36966529- | 36966654- | Exc | 0.181 | 0.024 | −0.157 | 6.59E−08 |
| | 36966528 | 36966653 | 36966720 | | | | | |
| CYB561D2 | 50388992- | 50389440- | 50389478- | Exc | 0.959 | 0.823 | −0.136 | 2.79E−05 |
| | 50389439 | 50389477 | 50390671 | | | | | |
| CYLD | 50776753- | 50778641- | 50778778- | Inc | 0.441 | 0.856 | 0.415 | 6.01E−15 |
| | 50778640 | 50778777 | 50783486 | | | | | |
| CYP20A1 | 204131405- | 204143296- | 204143412- | Exc | 0.974 | 0.830 | −0.145 | 1.01E−04 |
| | 204143295 | 204143411 | 204144781 | | | | | |
| CYP20A1 | 204103858- | 204110569- | 204110619- | Exc | 0.700 | 0.410 | −0.290 | 2.73E−05 |
| | 204110568 | 204110618 | 204116689 | | | | | |
| DAB2 | 39394524- | 39417291- | 39417387- | Inc | 0.138 | 0.334 | 0.196 | 1.02E−04 |
| | 39417290 | 39417386 | 39418282 | | | | | |
| DBT | 100671858- | 100672001- | 100672193- | Exc | 0.982 | 0.880 | −0.103 | 2.36E−04 |
| | 100672000 | 100672192 | 100676249 | | | | | |
| DCAF10 | 37819399- | 37842086- | 37842284- | Exc | 0.661 | 0.533 | −0.128 | 5.29E−04 |
| | 37842085 | 37842283 | 37854776 | | | | | |
| DCAF11 | 24586603- | 24586877- | 24586942- | Exc | 0.982 | 0.828 | −0.154 | 1.63E−15 |
| | 24586876 | 24586941 | 24587263 | | | | | |
| DCAF17 | 172325541- | 172330376- | 172330486- | Exc | 0.962 | 0.826 | −0.135 | 1.29E−03 |
| | 172330375 | 172330485 | 172333369 | | | | | |
| DCAF8 | 160213825- | 160231075- | 160231149- | Inc | 0.762 | 0.864 | 0.102 | 2.04E−04 |
| | 160231074 | 160231148 | 160231906 | | | | | |
| DCAF8 | 160210161- | 160213750- | 160213825- | Unc | 0.907 | 0.899 | −0.009 | 5.77E−01 |
| | 160213749 | 160213824 | 160231074 | | | | | |
| DCP1A | 53326858- | 53338207- | 53338321- | Inc | 0.890 | 0.990 | 0.100 | 3.80E−04 |
| | 53338206 | 53338320 | 53346270 | | | | | |
| DCTD | 183836729- | 183837572- | 183837693- | Unc | 0.232 | 0.235 | 0.003 | 9.85E−01 |
| | 183837571 | 183837692 | 183838463 | | | | | |
| DCTD | 183836729- | 183837572- | 183837693- | Unc | 0.126 | 0.122 | −0.004 | 9.67E−01 |
| | 183837571 | 183837692 | 183838440 | | | | | |
| DCUN1D5 | 102937297- | 102953477- | 102953569- | Inc | 0.856 | 0.993 | 0.137 | 8.56E−23 |
| | 102953476 | 102953568 | 102953984 | | | | | |
| DDB2 | 47256224- | 47256308- | 47256486- | Exc | 0.923 | 0.772 | −0.151 | 9.72E−09 |
| | 47256307 | 47256485 | 47256820 | | | | | |
| DGKA | 56331837- | 56332295- | 56332345- | Exc | 0.999 | 0.873 | −0.126 | 8.04E−15 |
| | 56332294 | 56332344 | 56332698 | | | | | |
| DGUOK | 74154180- | 74166037- | 74166150- | Unc | 0.285 | 0.287 | 0.002 | 9.97E−01 |
| | 74166036 | 74166149 | 74173845 | | | | | |
| DHRSX | 2209645- | 2310395- | 2310516- | Exc | 0.127 | 0.024 | −0.103 | 2.86E−11 |
| | 2310394 | 2310515 | 2326785 | | | | | |
| DIMT1 | 61686810- | 61687998- | 61688062- | Exc | 0.991 | 0.851 | −0.140 | 4.42E−19 |
| | 61687997 | 61688061 | 61688752 | | | | | |
| DIS3 | 73355142- | 73355427- | 73355495- | Exc | 0.338 | 0.170 | −0.168 | 3.13E−04 |
| | 73355426 | 73355494 | 73355742 | | | | | |
| DIS3 | 73352519- | 73354984- | 73355142- | Inc | 0.537 | 0.720 | 0.183 | 8.07E−05 |
| | 73354983 | 73355141 | 73355426 | | | | | |
| DLG1 | 196876668- | 196888511- | 196888610- | Exc | 0.613 | 0.500 | −0.113 | 3.68E−05 |
| | 196888510 | 196888609 | 196921295 | | | | | |
| DLGAP1-AS1 | 3594483- | 3596577- | 3596674- | Unc | 0.832 | 0.834 | 0.002 | 8.24E−01 |
| | 3596576 | 3596673 | 3597173 | | | | | |
| DMD | 31191722- | 31196049- | 31196088- | Exc | 0.495 | 0.086 | −0.409 | 9.35E−12 |
| | 31196048 | 31196087 | 31196785 | | | | | |
| DMKN | 35990932- | 35991280- | 35991322- | Unc | 0.224 | 0.230 | 0.006 | 7.69E−01 |
| | 35991279 | 35991321 | 35991434 | | | | | |
| DMTF1 | 86781872- | 86783706- | 86783845- | Exc | 0.219 | 0.076 | −0.144 | 3.88E−04 |
| | 86783705 | 86783844 | 86792810 | | | | | |
| DMTF1 | 86781872- | 86792555- | 86792649- | Exc | 0.254 | 0.098 | −0.157 | 5.81E−05 |
| | 86792554 | 86792648 | 86792810 | | | | | |
| DMTF1 | 86823419- | 86824000- | 86824145- | Exc | 0.944 | 0.779 | −0.165 | 1.51E−14 |
| | 86823999 | 86824144 | 86824346 | | | | | |
| DMWD | 46287549- | 46287899- | 46287974- | Unc | 0.648 | 0.642 | −0.006 | 9.50E−01 |
| | 46287898 | 46287973 | 46288851 | | | | | |
| DNAAF2 | 50092767- | 50094730- | 50094874- | Unc | 0.680 | 0.673 | −0.007 | 9.48E−01 |
| | 50094729 | 50094873 | 50100004 | | | | | |
| DNAJC14 | 56222499- | 56223273- | 56223421- | Unc | 0.764 | 0.767 | 0.003 | 9.33E−01 |
| | 56223272 | 56223420 | 56224479 | | | | | |
| DNAJC19 | 180703785- | 180704786- | 180704811- | Inc | 0.676 | 0.811 | 0.135 | 1.53E−05 |
| | 180704785 | 180704810 | 180705810 | | | | | |
| DNAJC19 | 180703785- | 180704731- | 180704811- | Inc | 0.808 | 0.910 | 0.102 | 1.73E−08 |
| | 180704730 | 180704810 | 180705810 | | | | | |
| DNAJC2 | 102967132- | 102967779- | 102967826- | Exc | 0.152 | 0.051 | −0.102 | 1.95E−05 |
| | 102967778 | 102967825 | 102968102 | | | | | |
| DNAJC24 | 31392407- | 31436358- | 31436497- | Unc | 0.904 | 0.896 | −0.008 | 9.12E−01 |
| | 31436357 | 31436496 | 31447833 | | | | | |
| DNM1L | 32890096- | 32891198- | 32891231- | Exc | 0.271 | 0.050 | −0.221 | 4.56E−18 |
| | 32891197 | 32891230 | 32892997 | | | | | |
| DNM1L | 32890877- | 32891198- | 32891231- | Exc | 0.547 | 0.180 | −0.366 | 2.76E−09 |
| | 32891197 | 32891230 | 32892997 | | | | | |
| DNM1L | 32890096- | 32890799- | 32890877- | Inc | 0.545 | 0.788 | 0.243 | 2.25E−04 |
| | 32890798 | 32890876 | 32891197 | | | | | |
| DNMT1 | 10288044- | 10290863- | 10290911- | Exc | 0.279 | 0.138 | −0.141 | 3.31E−04 |
| | 10290862 | 10290910 | 10291025 | | | | | |
| DOCK5 | 25240295- | 25246325- | 25246458- | Inc | 0.085 | 0.336 | 0.251 | 2.26E−12 |
| | 25246324 | 25246457 | 25246606 | | | | | |
| DOCK7 | 62943497- | 62953069- | 62953084- | Exc | 0.912 | 0.691 | −0.221 | 1.17E−04 |
| | 62953068 | 62953083 | 62954604 | | | | | |
| DPH3 | 16302337- | 16305662- | 16305737- | Inc | 0.511 | 0.757 | 0.247 | 1.39E−21 |
| | 16305661 | 16305736 | 16306275 | | | | | |
| DPH7 | 140470855- | 140471922- | 140472056- | Unc | 0.854 | 0.850 | −0.004 | 9.52E−01 |
| | 140471921 | 140472055 | 140473076 | | | | | |
| DPH7 | 140470620- | 140471922- | 140472056- | Unc | 0.724 | 0.718 | −0.006 | 8.33E−01 |
| | 140471921 | 140472055 | 140473076 | | | | | |
| DPM1 | 49552800- | 49557402- | 49557493- | Unc | 0.894 | 0.893 | −0.001 | 9.37E−01 |
| | 49557401 | 49557492 | 49558567 | | | | | |
| DPY19L4 | 95732243- | 95738559- | 95738670- | Exc | 0.975 | 0.874 | −0.101 | 4.43E−04 |
| | 95738558 | 95738669 | 95750596 | | | | | |
| DRAM2 | 111663316- | 111667364- | 111667504- | Exc | 0.924 | 0.755 | −0.169 | 2. ISE−17 |
| | 111667363 | 111667503 | 111668848 | | | | | |
| DRAM2 | 111680209- | 1116822lO- | 111682334- | Inc | 0.466 | 0.668 | 0.202 | 5.23E−07 |
| | 111682209 | ll 1682333 | 111682659 | | | | | |
| DSCR3 | 38604753- | 38605663- | 38605744- | Exc | 0.922 | 0.634 | −0.288 | 9.92E−45 |
| | 38605662 | 38605743 | 38610760 | | | | | |
| DTNBP1 | 15638036- | 15651544- | 15651595- | Exc | 0.963 | 0.855 | −0.108 | 2.61E−04 |
| | 15651543 | 15651594 | 15652317 | | | | | |
| DTNBP1 | 15638036- | 15651544- | 15651640- | Exc | 0.927 | 0.751 | −0.176 | 9.68E−04 |
| | 15651543 | 15651639 | 15652317 | | | | | |
| DUS2 | 68057255- | 68059318- | 68059398- | Unc | 0.869 | 0.860 | −0.009 | 9.16E−01 |
| | 68059317 | 68059397 | 68071908 | | | | | |
| DUSP11 | 73994031- | 73994288- | 73994335- | Exc | 0.981 | 0.659 | −0.322 | 9.71E−32 |
| | 73994287 | 73994334 | 73996391 | | | | | |
| DUSP22 | 311963- | 335114- | 335164- | Exc | 0.729 | 0.479 | −0.250 | 9.80E−08 |
| | 335113 | 335163 | 345853 | | | | | |
| DYNC2H1 | 103027500- | 103029406- | 103029539- | Exc | 0.988 | 0.828 | −0.160 | 3.48E−06 |
| | 103029405 | 103029538 | 103029638 | | | | | |
| EBPL | 50237332- | 50243913- | 50243983- | Exc | 0.861 | 0.680 | −0.181 | 4.60E−04 |
| | 50243912 | 50243982 | 50265389 | | | | | |
| EBPL | 50235345- | 50243913- | 50243983- | Exc | 0.743 | 0.441 | −0.302 | 1.74E−04 |
| | 50243912 | 50243982 | 50265389 | | | | | |
| ECHDC2 | 53370506- | 53370706- | 53370763- | Unc | 0.889 | 0.882 | −0.007 | 9.98E−01 |
| | 53370705 | 53370762 | 53373539 | | | | | |
| EDC3 | 74967484- | 74979432- | 74979521- | Exc | 0.181 | 0.069 | −0.112 | 6.98E−05 |
| | 74979431 | 74979520 | 74988220 | | | | | |
| EDEM2 | 33719587- | 33721908- | 33721959- | Inc | 0.134 | 0.409 | 0.276 | 6.98E−09 |
| | 33721907 | 33721958 | 33722540 | | | | | |
| EDRF1 | 127414408- | 127417572- | 127417674- | Exc | 0.713 | 0.348 | −0.365 | 4.46E−06 |
| | 127417571 | 127417673 | 127417926 | | | | | |
| EFEMP1 | 56149583- | 56150034- | 56150075- | Inc | 0.322 | 0.447 | 0.125 | 4.08E−106 |
| | 56150033 | 56150074 | 56150845 | | | | | |
| EHBP1 | 63206471- | 63215066- | 63215174- | Exc | 0.236 | 0.133 | −0.103 | 3.49E−13 |
| | 63215065 | 63215173 | 63217850 | | | | | |
| EHBP1L1 | 65349437- | 65351063- | 65351237- | Unc | 0.844 | 0.841 | −0.004 | 8.14E−01 |
| | 65351062 | 65351236 | 65351711 | | | | | |
| EHMT2 | 31856525- | 31856746- | 31856848- | Exc | 0.894 | 0.761 | −0.133 | 1.07E−04 |
| | 31856745 | 31856847 | 31857004 | | | | | |
| EIF4A2 | 186502486- | 186502751- | 186502891- | Exc | 0.760 | 0.622 | −0.138 | 4.02E−43 |
| | 186502750 | 186502890 | 186503671 | | | | | |
| EIF4E2 | 233421241- | 233422594- | 233422729- | Exc | 0.969 | 0.858 | −0.111 | 1.51E−25 |
| | 233422593 | 233422728 | 233428956 | | | | | |
| EIF4G2 | 10822635- | 10823208- | 10823322- | Unc | 0.881 | 0.889 | 0.009 | 7.64E−02 |
| | 10823207 | 10823321 | 10823595 | | | | | |
| EIF4H | 73604249- | 73604577- | 73604637- | Inc | 0.082 | 0.194 | 0.113 | 1.21E−34 |
| | 73604576 | 73604636 | 73609070 | | | | | |
| ELK1 | 47500875- | 47509320- | 47509426- | Exc | 0.470 | 0.261 | −0.209 | 2.18E−09 |
| | 47509319 | 47509425 | 47509821 | | | | | |
| ELMOD3 | 85598333- | 85598563- | 85598686- | Unc | 0.688 | 0.679 | −0.009 | 9.08E−01 |
| | 85598562 | 85598685 | 85604466 | | | | | |
| ELMOD3 | 85582294- | 85582678- | 85582840- | Unc | 0.747 | 0.738 | −0.010 | 9.67E−01 |
| | 85582677 | 85582839 | 85584089 | | | | | |
| ELN | 73471044- | 73471970- | 73472027- | Exc | 0.682 | 0.531 | −0.151 | 2.07E−13 |
| | 73471969 | 73472026 | 73474215 | | | | | |
| ELN | 73474378- | 73474470- | 73474515- | Exc | 0.989 | 0.821 | −0.167 | 4.96E−48 |
| | 73474469 | 73474514 | 73474705 | | | | | |
| ELOVL1 | 43830680- | 43830857- | 43831048- | Exc | 0.954 | 0.841 | −0.113 | 6.36E−25 |
| | 43830856 | 43831047 | 43831234 | | | | | |
| EMC4 | 34520048- | 34520630- | 34520791- | Unc | 0.808 | 0.806 | −0.001 | 9.68E−01 |
| | 34520629 | 34520790 | 34521953 | | | | | |
| EMC4 | 34520048- | 34520638- | 34520791- | Unc | 0.656 | 0.655 | −0.002 | 9.81E−01 |
| | 34520637 | 34520790 | 34521953 | | | | | |
| ENC1 | 73932324- | 73933390- | 73933527- | Inc | 0.108 | 0.356 | 0.248 | 7.14E−06 |
| | 73933389 | 73933526 | 73936131 | | | | | |
| ENC1 | 73932324- | 73933319- | 73933444- | Unc | 0.133 | 0.128 | −0.006 | 7.41E−01 |
| | 73933318 | 73933443 | 73936131 | | | | | |
| ENOSF1 | 677873- | 678696- | 678738- | Exc | 0.942 | 0.804 | −0.138 | 2.00E−05 |
| | 678695 | 678737 | 683245 | | | | | |
| EP400 | 132446500- | 132448077- | 132448188- | Unc | 0.220 | 0.214 | −0.006 | 9.02E−01 |
| | 132448076 | 132448187 | 132464241 | | | | | |
| EPB41L1 | 34806885- | 34807683- | 34807767- | Exc | 0.939 | 0.839 | −0.101 | 6.57E−07 |
| | 34807682 | 34807766 | 34809785 | | | | | |
| EPB41L1 | 34802363- | 34807683- | 34807767- | Exc | 0.878 | 0.759 | −0.119 | 4.31E−04 |
| | 34807682 | 34807766 | 34809785 | | | | | |
| EPB41L2 | 131191267- | 131191468- | 131191522- | Inc | 0.302 | 0.713 | 0.411 | 1.34E−16 |
| | 131191467 | 131191521 | 131197813 | | | | | |
| EPS15L1 | 16472796- | 16487933- | 16488066- | Unc | 0.858 | 0.852 | −0.006 | 9.33E−01 |
| | 16487932 | 16488065 | 16495939 | | | | | |
| ERBB2IP | 65350780- | 65370852- | 65371059- | Exc | 0.434 | 0.245 | −0.190 | 2.48E−22 |
| | 65370851 | 65371058 | 65372143 | | | | | |
| ERLEC1 | 54028980- | 54035436- | 54035598- | Exc | 0.858 | 0.745 | −0.112 | 5.82E−15 |
| | 54035435 | 54035597 | 54036350 | | | | | |
| ERMAP | 43304593- | 43305704- | 43305725- | Exc | 0.981 | 0.830 | −0.151 | 1.04E−08 |
| | 43305703 | 43305724 | 43305822 | | | | | |
| ERMARD | 170151760- | 170153383- | 170153469- | Unc | 0.103 | 0.108 | 0.005 | 8.37E−01 |
| | 170153382 | 170153468 | 170153959 | | | | | |
| ERMARD | 170151760- | 170153413- | 170153469- | Unc | 0.103 | 0.108 | 0.005 | 8.37E−01 |
| | 170153412 | 170153468 | 170153959 | | | | | |
| ETHE1 | 44015719- | 44030667- | 44030812- | Exc | 0.990 | 0.882 | −0.108 | 1.26E−06 |
| | 44030666 | 44030811 | 44031248 | | | | | |
| ETHE1 | 44015719- | 44030353- | 44030502- | Exc | 0.853 | 0.637 | −0.216 | 4.42E−20 |
| | 44030352 | 44030501 | 44030666 | | | | | |
| EVA1A | 75749614- | 75753238- | 75753326- | Unc | 0.538 | 0.533 | −0.005 | 4.61E−01 |
| | 75753237 | 75753325 | 75787805 | | | | | |
| EVC | 5815890- | 5819938- | 5819982- | Exc | 0.872 | 0.376 | −0.496 | 1.54E−60 |
| | 5819937 | 5819981 | 5825343 | | | | | |
| EVI5L | 7920955- | 7921978- | 7922011- | Exc | 0.676 | 0.479 | −0.197 | 1.73E−05 |
| | 7921977 | 7922010 | 7923076 | | | | | |
| FAM104A | 71205908- | 71208817- | 71208880- | Exc | 0.310 | 0.197 | −0.113 | 1.25E−03 |
| | 71208816 | 71208879 | 71223303 | | | | | |
| FAM111A | 58910778- | 58910970- | 58913330- | Exc | 0.900 | 0.699 | −0.201 | 4.46E−04 |
| | 58910969 | 58913329 | 58916308 | | | | | |
| FAM114A1 | 38870168- | 38879692- | 38880048- | Unc | 0.898 | 0.906 | 0.008 | 6.74E−01 |
| | 38879691 | 38880047 | 38893363 | | | | | |
| FAM134C | 40738910- | 40739852- | 40739883- | Exc | 0.979 | 0.878 | −0.101 | 1.32E−06 |
| | 40739851 | 40739882 | 40744073 | | | | | |
| FAM13B | 137354204- | 137354644- | 137354836- | Unc | 0.861 | 0.861 | 0.001 | 1.00E+00 |
| | 137354643 | 137354835 | 137356719 | | | | | |
| FAM13B | 137281687- | 137281917- | 137282001- | Unc | 0.874 | 0.874 | 0.000 | 9.76E−01 |
| | 137281916 | 137282000 | 137284643 | | | | | |
| FAM149B1 | 74994699- | 74994951- | 74995077- | Exc | 0.927 | 0.795 | −0.133 | 2.48E−04 |
| | 74994950 | 74995076 | 74999069 | | | | | |
| FAM160B2 | 21946810- | 21947272- | 21947366- | Unc | 0.101 | 0.108 | 0.007 | 8.30E−01 |
| | 21947271 | 21947365 | 21951950 | | | | | |
| FAM172A | 93300229- | 93386436- | 93386537- | Exc | 0.824 | 0.677 | −0.147 | 1.92E−05 |
| | 93386435 | 93386536 | 93388830 | | | | | |
| FAM172A | 93388933- | 93410351- | 93410475- | Inc | 0.644 | 0.790 | 0.146 | 5.58E−05 |
| | 93410350 | 93410474 | 93447215 | | | | | |
| FAM173A | 771942- | 772084- | 772135- | Unc | 0.795 | 0.796 | 0.002 | 7.41E−01 |
| | 772083 | 772134 | 772308 | | | | | |
| FAM175A | 84390305- | 84391356- | 84391550- | Unc | 0.888 | 0.892 | 0.005 | 8.51E−01 |
| | 84391355 | 84391549 | 84397795 | | | | | |
| FAM175A | 84403398- | 84405145- | 84405320- | Unc | 0.119 | 0.113 | −0.005 | 8.75E−01 |
| | 84405144 | 84405319 | 84406138 | | | | | |
| FAM204A | 120095936- | 120101239- | 120101440- | Exc | 0.583 | 0.449 | −0.134 | 1.38E−05 |
| | 120101238 | 120101439 | 120101781 | | | | | |
| FAM208B | 5773167- | 5777267- | 5777510- | Unc | 0.816 | 0.807 | −0.009 | 7.29E−01 |
| | 5777266 | 5777509 | 5781580 | | | | | |
| FAM211A- | 16342729- | 16342974- | 16343018- | Exc | 0.279 | 0.155 | −0.124 | 3.11E−100 |
| AS1 | 16342973 | 16343017 | 16343498 | | | | | |
| FAM211A- | 16342729- | 16342895- | 16343018- | Exc | 0.313 | 0.180 | −0.133 | 1.77E−110 |
| AS1 | 16342894 | 16343017 | 16343498 | | | | | |
| FAM211A- | 16342729- | 16342842- | 16343018- | Exc | 0.328 | 0.179 | −0.149 | 2.05E−136 |
| AS1 | 16342841 | 16343017 | 16343498 | | | | | |
| FAM21C | 46268807- | 46272721- | 46272874- | Inc | 0.421 | 0.663 | 0.243 | 1.76E−04 |
| | 46272720 | 46272873 | 46274400 | | | | | |
| FAM35A | 88930732- | 88935646- | 88935853- | Unc | 0.737 | 0.743 | 0.006 | 9.25E−01 |
| | 88935645 | 88935852 | 88939831 | | | | | |
| FAM45A | 120863710- | 120864276- | 120864535- | Unc | 0.655 | 0.664 | 0.009 | 8.17E−01 |
| | 120864275 | 120864534 | 120864822 | | | | | |
| FAP | 163054251- | 163054689- | 163054809- | Exc | 0.561 | 0.303 | −0.258 | 4.88E−06 |
| | 163054688 | 163054808 | 163055266 | | | | | |
| FBXL12 | 9922394- | 9923907- | 9923951- | Exc | 0.170 | 0.025 | −0.145 | 5.57E−07 |
| | 9923906 | 9923950 | 9929220 | | | | | |
| FBXL3 | 77592858- | 77595792- | 77595997- | Inc | 0.757 | 0.926 | 0.169 | 4.58E−04 |
| | 77595791 | 77595996 | 77600961 | | | | | |
| FBXO25 | 381445- | 382886- | 382936- | Exc | 0.840 | 0.715 | −0.125 | 8.60E−05 |
| | 382885 | 382935 | 385614 | | | | | |
| FBXW11 | 171337802- | 171341347- | 171341410- | Exc | 0.474 | 0.318 | −0.156 | 4.86E−05 |
| | 171341346 | 171341409 | 171433461 | | | | | |
| FBXW11 | 171337802- | 171341347- | 171341410- | Exc | 0.775 | 0.569 | −0.206 | 2.10E−04 |
| | 171341346 | 171341409 | 171423893 | | | | | |
| FDPS | 155278757- | 155279544- | 155279757- | Unc | 0.113 | 0.122 | 0.008 | 9.95E−01 |
| | 155279543 | 155279756 | 155279833 | | | | | |
| FGF5 | 81188334- | 81196063- | 81196167- | Exc | 0.766 | 0.541 | −0.225 | 8.66E−07 |
| | 81196062 | 81196166 | 81207478 | | | | | |
| FGFR1 | 38286909- | 38287200- | 38287467- | Unc | 0.906 | 0.899 | −0.007 | 8.46E−01 |
| | 38287199 | 38287466 | 38314873 | | | | | |
| FGFR1OP | 167424382- | 167426996- | 167427056- | Inc | 0.506 | 0.802 | 0.295 | 3.12E−10 |
| | 167426995 | 167427055 | 167435896 | | | | | |
| FHL2 | 106002998- | 106013104- | 106013155- | Exc | 0.550 | 0.449 | −0.101 | 1.22E−10 |
| | 106013103 | 106013154 | 106015298 | | | | | |
| FHOD3 | 33935609- | 33952643- | 33952708- | Unc | 0.902 | 0.893 | −0.009 | 9.47E−01 |
| | 33952642 | 33952707 | 34081894 | | | | | |
| FIP1L1 | 54244091- | 54245240- | 54245285- | Exc | 0.467 | 0.329 | −0.138 | 4.26E−06 |
| | 54245239 | 54245284 | 54245391 | | | | | |
| FIP1L1 | 54294351- | 54306749- | 54306776- | Exc | 0.450 | 0.228 | −0.223 | 2.87E−18 |
| | 54306748 | 54306775 | 54308819 | | | | | |
| FIP1L1 | 54257307- | 54257597- | 54257666- | Inc | 0.426 | 0.529 | 0.104 | 5.19E−05 |
| | 54257596 | 54257665 | 54265896 | | | | | |
| FLAD1 | 154961326- | 154962036- | 154962184- | Exc | 0.922 | 0.744 | −0.177 | 7.38E−05 |
| | 154962035 | 154962183 | 154962634 | | | | | |
| FLNA | 153583441- | 153585619- | 153585643- | Exc | 0.631 | 0.481 | −0.150 | 1.98E−91 |
| | 153585618 | 153585642 | 153585801 | | | | | |
| FLNB | 58124257- | 58127585- | 58127657- | Unc | 0.788 | 0.791 | 0.003 | 9.83E−01 |
| | 58127584 | 58127656 | 58128376 | | | | | |
| FLNB | 58124257- | 58127585- | 58127624- | Unc | 0.710 | 0.709 | −0.001 | 9.04E−01 |
| | 58127584 | 58127623 | 58128376 | | | | | |
| FN1 | 216244041- | 216245534- | 216245804- | Exc | 0.330 | 0.226 | −0.104 | 0.00E+00 |
| | 216245533 | 216245803 | 216246934 | | | | | |
| FNBP1 | 132671279- | 132678245- | 132678260- | Unc | 0.306 | 0.316 | 0.009 | 7.86E−01 |
| | 132678244 | 132678259 | 132686122 | | | | | |
| FNBP4 | 47746331- | 47747289- | 47747389- | Inc | 0.151 | 0.431 | 0.280 | 8.28E−14 |
| | 47747288 | 47747388 | 47752925 | | | | | |
| FNIP2 | 159754781- | 159754953- | 159755043- | Exc | 0.722 | 0.580 | −0.142 | 4.97E−06 |
| | 159754952 | 159755042 | 159756556 | | | | | |
| FOSL1 | 65660768- | 65661485- | 65661593- | Unc | 0.889 | 0.887 | −0.001 | 9.75E−01 |
| | 65661484 | 65661592 | 65664279 | | | | | |
| FOXN3 | 89647151- | 89656731- | 89656794- | Exc | 0.301 | 0.077 | −0.224 | 8.32E−15 |
| | 89656730 | 89656793 | 89747293 | | | | | |
| FOXN3 | 89647151- | 89656728- | 89656794- | Exc | 0.347 | 0.097 | −0.250 | 1.19E−16 |
| | 89656727 | 89656793 | 89747293 | | | | | |
| FOXRED2 | 36897455- | 36900145- | 36900415- | Unc | 0.857 | 0.865 | 0.008 | 9.89E−01 |
| | 36900144 | 36900414 | 36900561 | | | | | |
| FRS2 | 69925837- | 69931696- | 69931764- | Unc | 0.100 | 0.095 | −0.006 | 9.70E−01 |
| | 69931695 | 69931763 | 69955959 | | | | | |
| FRYL | 48503751- | 48504845- | 48504863- | Inc | 0.035 | 0.222 | 0.187 | 4.68E−08 |
| | 48504844 | 48504862 | 48507563 | | | | | |
| FUT8 | 66083092- | 66083808- | 66083910- | Inc | 0.006 | 0.234 | 0.228 | 4.47E−59 |
| | 66083807 | 66083909 | 66096209 | | | | | |
| FXR2 | 7495244- | 7495389- | 7495476- | Inc | 0.020 | 0.152 | 0.132 | 1.28E−12 |
| | 7495388 | 7495475 | 7495571 | | | | | |
| FYN | 112101839- | 112167792- | 112167833- | Exc | 0.877 | 0.741 | −0.136 | 3.57E−07 |
| | 112167791 | 112167832 | 112194170 | | | | | |
| GAB1 | 144361536- | 144378833- | 144378923- | Exc | 0.400 | 0.213 | −0.187 | 2.06E−04 |
| | 144378832 | 144378922 | 144380537 | | | | | |
| GABPB2 | 151070479- | 151076038- | 151076152- | Exc | 0.702 | 0.490 | −0.213 | 2.46E−04 |
| | 151076037 | 151076151 | 151079512 | | | | | |
| GABRE | 151128449- | 151129755- | 151129838- | Exc | 0.122 | 0.006 | −0.116 | 4.60E−05 |
| | 151129754 | 151129837 | 151130894 | | | | | |
| GALNS | 88902676- | 88904030- | 88904174- | Exc | 0.989 | 0.869 | −0.120 | 2.92E−09 |
| | 88904029 | 88904173 | 88907399 | | | | | |
| GALNS | 88908380- | 88909114- | 88909238- | Exc | 0.935 | 0.666 | −0.269 | 2.89E−11 |
| | 88909113 | 88909237 | 88923165 | | | | | |
| GAS7 | 9862573- | 9872994- | 9873080- | Unc | 0.871 | 0.876 | 0.005 | 4.60E−01 |
| | 9872993 | 9873079 | 9885120 | | | | | |
| GBP3 | 89481098- | 89485809- | 89485895- | Unc | 0.358 | 0.367 | 0.009 | 5.52E−01 |
| | 89485808 | 89485894 | 89486214 | | | | | |
| GBP3 | 89481098- | 89485813- | 89485895- | Unc | 0.234 | 0.225 | −0.009 | 7.69E−01 |
| | 89485812 | 89485894 | 89486214 | | | | | |
| GEMIN8 | 14039631- | 14044171- | 14044341- | Unc | 0.696 | 0.687 | −0.009 | 9.24E−01 |
| | 14044170 | 14044340 | 14047895 | | | | | |
| GFPT2 | 179728609- | 179729423- | 179729585- | Exc | 0.912 | 0.756 | −0.156 | 9.65E−06 |
| | 179729422 | 179729584 | 179731771 | | | | | |
| GGCT | 30538555- | 30540152- | 30540298- | Inc | 0.755 | 0.964 | 0.209 | 2.33E−17 |
| | 30540151 | 30540297 | 30544184 | | | | | |
| GIT2 | 110383155- | 110385061- | 110385310- | Exc | 0.977 | 0.876 | −0.101 | 4.89E−06 |
| | 110385060 | 110385309 | 110388971 | | | | | |
| GIT2 | 110385310- | 110388972- | 110389122- | Inc | 0.822 | 0.946 | 0.124 | 1.00E−08 |
| | 110388971 | 110389121 | 110390896 | | | | | |
| GK | 30714801- | 30715849- | 30715867- | Exc | 0.606 | 0.246 | −0.360 | 1.00E−04 |
| | 30715848 | 30715866 | 30718530 | | | | | |
| GLIS3 | 4125942- | 4286038- | 4286524- | Unc | 0.807 | 0.808 | 0.001 | 8.26E−01 |
| | 4286037 | 4286523 | 4299420 | | | | | |
| GLIS3 | 4118882- | 4125734- | 4125942- | Unc | 0.614 | 0.605 | −0.008 | 9.77E−01 |
| | 4125733 | 4125941 | 4286037 | | | | | |
| GLRB | 158065112- | 158073870- | 158074163- | Unc | 0.754 | 0.752 | −0.002 | 6.97E−01 |
| | 158073869 | 158074162 | 158091583 | | | | | |
| GLS | 191778091- | 191784941- | 191784975- | Exc | 0.710 | 0.295 | −0.414 | 1.11E−16 |
| | 191784940 | 191784974 | 191785749 | | | | | |
| GLS | 191775048- | 191777919- | 191778091- | Inc | 0.699 | 0.876 | 0.177 | 1.35E−06 |
| | 191777918 | 191778090 | 191784940 | | | | | |
| GLT8D1 | 52734513- | 52738740- | 52738969- | Unc | 0.449 | 0.441 | −0.009 | 5.89E−01 |
| | 52738739 | 52738968 | 52739462 | | | | | |
| GNAS | 57470740- | 57473996- | 57474041- | Unc | 0.314 | 0.316 | 0.003 | 7.51E−01 |
| | 57473995 | 57474040 | 57478582 | | | | | |
| GNAS | 57470740- | 57473996- | 57474041- | Unc | 0.602 | 0.595 | −0.006 | 4.76E−01 |
| | 57473995 | 57474040 | 57478585 | | | | | |
| GOLGA4 | 37396679- | 37402734- | 37402797- | Exc | 0.445 | 0.313 | −0.132 | 4.72E−06 |
| | 37402733 | 37402796 | 37407570 | | | | | |
| GOLGB1 | 121437349- | 121438493- | 121438616- | Exc | 0.523 | 0.340 | −0.183 | 4.28E−05 |
| | 121438492 | 121438615 | 121441108 | | | | | |
| GOLGB1 | 121437349- | 121438493- | 121438601- | Exc | 0.519 | 0.326 | −0.193 | 1.78E−05 |
| | 121438492 | 121438600 | 121441108 | | | | | |
| GOLT1B | 21654883- | 21661317- | 21661496- | Exc | 0.977 | 0.875 | −0.101 | 1.30E−06 |
| | 21661316 | 21661495 | 21665228 | | | | | |
| GPATCH8 | 42513914- | 42516039- | 42516084- | Unc | 0.137 | 0.136 | −0.002 | 9.47E−01 |
| | 42516038 | 42516083 | 42541839 | | | | | |
| GPBP1 | 56531860- | 56532940- | 56533000- | Unc | 0.223 | 0.231 | 0.008 | 6.31E−01 |
| | 56532939 | 56532999 | 56542126 | | | | | |
| GPR133 | 131621560- | 131622189- | 131622775- | Exc | 0.972 | 0.837 | −0.136 | 1.65E−07 |
| | 131622188 | 131622774 | 131623712 | | | | | |
| GPR180 | 95264645- | 95271404- | 95271585- | Exc | 0.676 | 0.385 | −0.291 | 1.84E−08 |
| | 95271403 | 95271584 | 95271721 | | | | | |
| GRB10 | 50771606- | 50778572- | 50778688- | Unc | 0.239 | 0.231 | −0.007 | 9.10E−01 |
| | 50778571 | 50778687 | 50823583 | | | | | |
| GRTPAP1 | 48853770- | 48854566- | 48854593- | Exc | 0.992 | 0.878 | −0.114 | 2.78E−07 |
| | 48854565 | 48854592 | 48855649 | | | | | |
| GTF2I | 74157860- | 74158478- | 74158590- | Inc | 0.407 | 0.799 | 0.392 | 8.75E−13 |
| | 74158477 | 74158589 | 74159096 | | | | | |
| GTF2I | 74131271- | 74133198- | 74133261- | Inc | 0.309 | 0.435 | 0.126 | 5.68E−11 |
| | 74133197 | 74133260 | 74143123 | | | | | |
| GTF3A | 28004759- | 28006868- | 28006942- | Exc | 0.818 | 0.713 | −0.105 | 5.18E−10 |
| | 28006867 | 28006941 | 28008275 | | | | | |
| GUF1 | 44696531- | 44697632- | 44697752- | Exc | 0.965 | 0.859 | −0.107 | 1.21E−04 |
| | 44697631 | 44697751 | 44699424 | | | | | |
| GUSB | 65439692- | 65439906- | 65440059- | Exc | 0.567 | 0.435 | −0.132 | 1.21E−04 |
| | 65439905 | 65440058 | 65441001 | | | | | |
| HACL1 | 15624497- | 15626755- | 15626850- | Exc | 0.868 | 0.550 | −0.318 | 7.12E−06 |
| | 15626754 | 15626849 | 15628031 | | | | | |
| HAUS7 | 152719967- | 152720335- | 152720512- | Exc | 0.465 | 0.347 | −0.119 | 9.05E−04 |
| | 152720334 | 152720511 | 152720999 | | | | | |
| HCFC1R1 | 3073363- | 3073475- | 3073532- | Exc | 0.922 | 0.794 | −0.128 | 8.08E−07 |
| | 3073474 | 3073531 | 3073847 | | | | | |
| HDAC10 | 50685396- | 50686121- | 50686206- | Exc | 0.741 | 0.534 | −0.207 | 7.53E−05 |
| | 50686120 | 50686205 | 50686318 | | | | | |
| HDAC7 | 48189551- | 48189689- | 48189800- | Exc | 0.465 | 0.218 | −0.247 | 1.91E−13 |
| | 48189688 | 48189799 | 48189989 | | | | | |
| HDAC9 | 18633653- | 18668973- | 18669105- | Unc | 0.509 | 0.518 | 0.009 | 9.69E−01 |
| | 18668972 | 18669104 | 18674249 | | | | | |
| HECTD3 | 45475356- | 45475658- | 45475794- | Exc | 0.929 | 0.801 | −0.128 | 8.69E−06 |
| | 45475657 | 45475793 | 45475872 | | | | | |
| HER3 | 89591404- | 89597369- | 89597393- | Exc | 0.804 | 0.430 | −0.374 | 8.53E−19 |
| | 89597368 | 89597392 | 89597484 | | | | | |
| HIPK3 | 33369560- | 33369712- | 33369775- | Exc | 0.190 | 0.084 | −0.105 | 3.69E−06 |
| | 33369711 | 33369774 | 33370067 | | | | | |
| HMCN1 | 186143775- | 186147548- | 186147899- | Unc | 0.589 | 0.585 | −0.003 | 8.17E−01 |
| | 186147547 | 186147898 | 186151299 | | | | | |
| HMGXB4 | 35659868- | 35659975- | 35660090- | Exc | 0.335 | 0.200 | −0.135 | 4.79E−04 |
| | 35659974 | 35660089 | 35660640 | | | | | |
| HNRNPA2B1 | 26230081- | 26230613- | 26230749- | Inc | 0.761 | 0.874 | 0.113 | 3.95E−08 |
| | 26230612 | 26230748 | 26232114 | | | | | |
| HNRNPD | 83276555- | 83277690- | 83277837- | Exc | 0.452 | 0.346 | −0.106 | 7.97E−15 |
| | 83277689 | 83277836 | 83277948 | | | | | |
| HNRNPDL | 83346037- | 83346716- | 83346821- | Inc | 0.187 | 0.302 | 0.115 | 2.19E−16 |
| | 83346715 | 83346820 | 83347189 | | | | | |
| HNRNPK | 86588315- | 86588817- | 86588889- | Unc | 0.894 | 0.893 | −0.001 | 8.03E−01 |
| | 86588816 | 86588888 | 86589431 | | | | | |
| HPS1 | 100193849- | 100195029- | 100195172- | Exc | 0.900 | 0.795 | −0.105 | 1.72E−08 |
| | 100195028 | 100195171 | 100195391 | | | | | |
| HSCB | 29139967- | 29140603- | 29140698- | Unc | 0.869 | 0.864 | −0.006 | 9.85E−01 |
| | 29140602 | 29140697 | 29147228 | | | | | |
| HSD17B4 | 118792064- | 118807331- | 118807396- | Inc | 0.022 | 0.619 | 0.597 | 5.07E−105 |
| | 118807330 | 118807395 | 118809602 | | | | | |
| HSF2 | 122744832- | 122749048- | 122749102- | Inc | 0.256 | 0.583 | 0.328 | 1.59E−12 |
| | 122749047 | 122749101 | 122752574 | | | | | |
| HUWE1 | 53652220- | 53652747- | 53652990- | Exc | 0.384 | 0.270 | −0.114 | 3.10E−05 |
| | 53652746 | 53652989 | 53654360 | | | | | |
| IFT88 | 21157159- | 21163950- | 21164007- | Exc | 0.845 | 0.579 | −0.266 | 4.98E−06 |
| | 21163949 | 21164006 | 21165105 | | | | | |
| IGF2BP2 | 185376198- | 185390329- | 185390458- | Exc | 0.643 | 0.384 | −0.258 | 1.12E−16 |
| | 185390328 | 185390457 | 185393083 | | | | | |
| IL15RA | 6002531- | 6005706- | 6005802- | Inc | 0.493 | 0.782 | 0.289 | 1.07E−03 |
| | 6005705 | 6005801 | 6008107 | | | | | |
| 1L15RA | 6002531- | 6005706- | 6005805- | Inc | 0.571 | 0.825 | 0.254 | 7.29E−04 |
| | 6005705 | 6005804 | 6008107 | | | | | |
| IL17RA | 17586493- | 17586743- | 17586845- | Unc | 0.834 | 0.841 | 0.008 | 7.57E−01 |
| | 17586742 | 17586844 | 17588616 | | | | | |
| IL17RC | 9974388- | 9974504- | 9974543- | Unc | 0.575 | 0.582 | 0.007 | 9.92E−01 |
| | 9974503 | 9974542 | 9974636 | | | | | |
| IL6 | 22766901- | 22767132- | 22767254- | Unc | 0.899 | 0.895 | −0.003 | 6.25E−01 |
| | 22767131 | 22767253 | 22768311 | | | | | |
| INF2 | 105181194- | 105181621- | 105181678- | Inc | 0.599 | 0.732 | 0.133 | 3.41E−05 |
| | 105181620 | 105181677 | 105185131 | | | | | |
| ING4 | 6760552- | 6761437- | 6761476- | Exc | 0.993 | 0.866 | −0.128 | 1.40E−09 |
| | 6761436 | 6761475 | 6761827 | | | | | |
| INO80E | 30012362- | 30012786- | 30012852- | Inc | 0.539 | 0.828 | 0.289 | 8.16E−06 |
| | 30012785 | 30012851 | 30016541 | | | | | |
| INO80E | 30012362- | 30012533- | 30015979- | Inc | 0.562 | 0.819 | 0.258 | 1.08E−04 |
| | 30012532 | 30015978 | 30016541 | | | | | |
| INO80E | 30012362- | 30012533- | 30012852- | Inc | 0.702 | 0.902 | 0.201 | 4.35E−06 |
| | 30012532 | 30012851 | 30016541 | | | | | |
| INO80E | 30012362- | 30012735- | 30012852- | Inc | 0.794 | 0.942 | 0.148 | 2.03E−07 |
| | 30012734 | 30012851 | 30016541 | | | | | |
| INPP1 | 191208688- | 191224372- | 191224425- | Inc | 0.091 | 0.224 | 0.133 | 5.46E−04 |
| | 191224371 | 191224424 | 191224764 | | | | | |
| INTS9 | 28695294- | 28704264- | 28704327- | Unc | 0.832 | 0.830 | −0.002 | 9.06E−01 |
| | 28704263 | 28704326 | 28707729 | | | | | |
| IP6K2 | 48731674- | 48731892- | 48731959- | Inc | 0.134 | 0.552 | 0.419 | 6.60E−13 |
| | 48731891 | 48731958 | 48732522 | | | | | |
| IP6K2 | 48731674- | 48732127- | 48732258- | Unc | 0.290 | 0.296 | 0.006 | 8.95E−01 |
| | 48732126 | 48732257 | 48732522 | | | | | |
| IPOS | 30833573- | 30834593- | 30834752- | Exc | 0.993 | 0.892 | −0.101 | 9.08E−09 |
| | 30834592 | 30834751 | 30837234 | | | | | |
| IQCB1 | 121545028- | 121547317- | 121547480- | Inc | 0.705 | 0.868 | 0.163 | 7.90E−04 |
| | 121547316 | 121547479 | 121547707 | | | | | |
| IRAK4 | 44162076- | 44165023- | 44165169- | Exc | 0.554 | 0.239 | −0.315 | 4.03E−08 |
| | 44165022 | 44165168 | 44165982 | | | | | |
| ISOC2 | 55966698- | 55967003- | 55967213- | Exc | 0.875 | 0.761 | −0.115 | 1.01E−06 |
| | 55967002 | 55967212 | 55967715 | | | | | |
| IST1 | 71956584- | 71957191- | 71957284- | Exc | 0.951 | 0.805 | −0.146 | 5.81E−27 |
| | 71957190 | 71957283 | 71958671 | | | | | |
| ITGB1BP1 | 9558862- | 9560119- | 9560230- | Inc | 0.310 | 0.522 | 0.212 | 3.37E−10 |
| | 9560118 | 9560229 | 9562607 | | | | | |
| KANSL3 | 97285282- | 97285409- | 97285500- | Unc | 0.889 | 0.892 | 0.003 | 9.79E−01 |
| | 97285408 | 97285499 | 97297048 | | | | | |
| KAT6A | 41906821- | 41907138- | 41907226- | Unc | 0.182 | 0.186 | 0.005 | 9.37E−01 |
| | 41907137 | 41907225 | 41909418 | | | | | |
| KATNBL1 | 34455892- | 34470022- | 34470201- | Inc | 0.077 | 0.219 | 0.142 | 3.89E−05 |
| | 34470021 | 34470200 | 34502151 | | | | | |
| KCTD20 | 36438035- | 36442566- | 36442840- | Unc | 0.899 | 0.893 | −0.006 | 6.98E−01 |
| | 36442565 | 36442839 | 36446897 | | | | | |
| KDM4C | 7103871- | 7105402- | 7105501- | Exc | 0.174 | 0.022 | −0.152 | 7.90E−05 |
| | 7105401 | 7105500 | 7128065 | | | | | |
| KDM5A | 394829- | 395292- | 395374- | Inc | 0.301 | 0.443 | 0.143 | 3.33E−04 |
| | 395291 | 395373 | 401924 | | | | | |
| KIAA0100 | 26960770- | 26960913- | 26961105- | Exc | 0.975 | 0.850 | −0.125 | 1.28E−22 |
| | 26960912 | 26961104 | 26961534 | | | | | |
| KIAA0232 | 6873410- | 6878387- | 6878485- | Unc | 0.894 | 0.885 | −0.009 | 6.15E−01 |
| | 6878386 | 6878484 | 6882513 | | | | | |
| KIAA0368 | 114213836- | 114235177- | 114235281- | Unc | 0.809 | 0.813 | 0.003 | 8.91E−01 |
| | 114235176 | 114235280 | 114246264 | | | | | |
| KIAA0753 | 6526892- | 6528075- | 6528182- | Unc | 0.857 | 0.858 | 0.001 | 8.05E−01 |
| | 6528074 | 6528181 | 6531436 | | | | | |
| KIAA1551 | 32113806- | 32123123- | 32123291- | Unc | 0.893 | 0.898 | 0.005 | 9.29E−01 |
| | 32123122 | 32123290 | 32133811 | | | | | |
| KIAA1586 | 56912195- | 56915572- | 56915653- | Unc | 0.866 | 0.863 | −0.003 | 9.48E−01 |
| | 56915571 | 56915652 | 56917483 | | | | | |
| KIAA1731 | 93454579- | 93455120- | 93455217- | Unc | 0.833 | 0.838 | 0.005 | 9.89E−01 |
| | 93455119 | 93455216 | 93456206 | | | | | |
| KIF3A | 132039312- | 132042143- | 132042152- | Exc | 0.525 | 0.260 | −0.265 | 3.02E−06 |
| | 132042142 | 132042151 | 132046650 | | | | | |
| KIFC3 | 57806201- | 57819376- | 57819579- | Unc | 0.168 | 0.165 | −0.003 | 9.58E−01 |
| | 57819375 | 57819578 | 57828910 | | | | | |
| KIFC3 | 57806201- | 57824842- | 57824979- | Unc | 0.109 | 0.104 | −0.005 | 9.03E−01 |
| | 57824841 | 57824978 | 57828910 | | | | | |
| KITLG | 88900915- | 88909311- | 88909395- | Exc | 0.985 | 0.884 | −0.101 | 2.32E−16 |
| | 88909310 | 88909394 | 88910110 | | | | | |
| KLC1 | 104145883- | 104151323- | 104151374- | Exc | 0.286 | 0.176 | −0.110 | 1.43E−06 |
| | 104151322 | 104151373 | 104153417 | | | | | |
| KLC1 | 104153549- | 104158696- | 104158763- | Inc | 0.330 | 0.755 | 0.425 | 1.93E−42 |
| | 104158695 | 104158762 | 104166991 | | | | | |
| KLHL12 | 202861788- | 202862367- | 202862554- | Unc | 0.855 | 0.860 | 0.005 | 9.23E−01 |
| | 202862366 | 202862553 | 202863311 | | | | | |
| KTN1 | 56128331- | 56130673- | 56130760- | Inc | 0.268 | 0.525 | 0.257 | 7.38E−64 |
| | 56130672 | 56130759 | 56133958 | | | | | |
| KTN1 | 56139731- | 56139890- | 56139974- | Inc | 0.387 | 0.495 | 0.108 | 4.17E−12 |
| | 56139889 | 56139973 | 56142552 | | | | | |
| KTN1 | 56047073- | 56068475- | 56068599- | Unc | 0.410 | 0.406 | −0.004 | 9.19E−01 |
| | 56068474 | 56068598 | 56078736 | | | | | |
| L3HYPDH | 59939809- | 59941157- | 59941245- | Inc | 0.197 | 0.344 | 0.147 | 4.42E−06 |
| | 59941156 | 59941244 | 59942586 | | | | | |
| LACC1 | 44463119- | 44464250- | 44464411- | Exc | 0.925 | 0.789 | −0.136 | 1.91E−05 |
| | 44464249 | 44464410 | 44465584 | | | | | |
| LAMTOR3 | 100813192- | 100815112- | 100815158- | Exc | 0.890 | 0.750 | −0.140 | 9.62E−12 |
| | 100815111 | 100815157 | 100815491 | | | | | |
| LAS1L | 64744143- | 64744444- | 64744495- | Exc | 0.214 | 0.004 | −0.210 | 4.86E−12 |
| | 64744443 | 64744494 | 64744844 | | | | | |
| LBR | 225594535- | 225597993- | 225598119- | Unc | 0.885 | 0.885 | 0.000 | 9.73E−01 |
| | 225597992 | 225598118 | 225599038 | | | | | |
| LDB2 | 16504497- | 16507463- | 16507606- | Unc | 0.623 | 0.618 | −0.006 | 8.46E−01 |
| | 16507462 | 16507605 | 16510157 | | | | | |
| LENG8 | 54963932- | 54964725- | 54964836- | Exc | 0.899 | 0.779 | −0.120 | 1.85E−19 |
| | 54964724 | 54964835 | 54965608 | | | | | |
| LETMD1 | 51442969- | 51445875- | 51445991- | Exc | 0.439 | 0.207 | −0.232 | 2.76E−10 |
| | 51445874 | 51445990 | 51450132 | | | | | |
| LETMD1 | 51442969- | 51449927- | 51450029- | Exc | 0.516 | 0.214 | −0.302 | 1.21E−17 |
| | 51449926 | 51450028 | 51450132 | | | | | |
| LETMD1 | 51442969- | 51445875- | 51445991- | Exc | 0.872 | 0.524 | −0.348 | 4.96E−10 |
| | 51445874 | 51445990 | 51449617 | | | | | |
| LETMD1 | 51442969- | 51447561- | 51447644- | Exc | 0.904 | 0.512 | −0.392 | 1.12E−15 |
| | 51447560 | 51447643 | 51449617 | | | | | |
| LETMD1 | 51442969- | 51447595- | 51447644- | Exc | 0.909 | 0.511 | −0.398 | 1.35E−16 |
| | 51447594 | 51447643 | 51449617 | | | | | |
| LGALS8 | 236706088- | 236706215- | 236706341- | Inc | 0.243 | 0.408 | 0.165 | 7.61E−08 |
| | 236706214 | 236706340 | 236706859 | | | | | |
| LINC00963 | 132251579- | 132255695- | 132255875- | Exc | 0.837 | 0.709 | −0.128 | 8.09E−04 |
| | 132255694 | 132255874 | 132265601 | | | | | |
| LMAN2L | 97377763- | 97399256- | 97399339- | Exc | 0.956 | 0.849 | −0.108 | 1.64E−05 |
| | 97399255 | 97399338 | 97400145 | | | | | |
| LMAN2L | 97400264- | 97402874- | 97402955- | Inc | 0.049 | 0.228 | 0.179 | 2.48E−06 |
| | 97402873 | 97402954 | 97403685 | | | | | |
| LMO7 | 76210863- | 76287318- | 76287389- | Exc | 0.761 | 0.597 | −0.164 | 5.14E−38 |
| | 76287317 | 76287388 | 76301164 | | | | | |
| LONRF1 | 12594650- | 12594781- | 12594870- | Inc | 0.005 | 0.243 | 0.239 | 7.33E−11 |
| | 12594780 | 12594869 | 12595503 | | | | | |
| LOXL3 | 74763599- | 74763836- | 74764056- | Exc | 0.907 | 0.751 | −0.156 | 1.95E−05 |
| | 74763835 | 74764055 | 74776495 | | | | | |
| LPHN2 | 82451040- | 82452973- | 82453026- | Exc | 0.469 | 0.362 | −0.107 | 4.04E−04 |
| | 82452972 | 82453025 | 82456074 | | | | | |
| LPHN2 | 82451040- | 82452944- | 82453026- | Exc | 0.413 | 0.261 | −0.152 | 2.47E−06 |
| | 82452943 | 82453025 | 82456074 | | | | | |
| LPHN2 | 82417827- | 82418671- | 82418710- | Inc | 0.193 | 0.455 | 0.263 | 1.56E−19 |
| | 82418670 | 82418709 | 82421560 | | | | | |
| LPIN1 | 11913872- | 11916212- | 11916320- | Inc | 0.118 | 0.532 | 0.414 | 4.79E−35 |
| | 11916211 | 11916319 | 11919644 | | | | | |
| LRCH3 | 197581317- | 197585705- | 197585777- | Inc | 0.352 | 0.633 | 0.281 | 1.38E−12 |
| | 197585704 | 197585776 | 197592293 | | | | | |
| LRP12 | 105521303- | 105544135- | 105544192- | Unc | 0.738 | 0.739 | 0.002 | 9.98E−01 |
| | 105544134 | 105544191 | 105601046 | | | | | |
| LRRC28 | 99796331- | 99816781- | 99816822- | Exc | 0.764 | 0.347 | −0.417 | 1.41E−23 |
| | 99816780 | 99816821 | 99827461 | | | | | |
| LRRC32 | 76369079- | 76370627- | 76372553- | Exc | 0.939 | 0.838 | −0.101 | 2.33E−04 |
| | 76370626 | 76372552 | 76376914 | | | | | |
| LSM1 | 38021359- | 38027320- | 38027436- | Inc | 0.827 | 0.957 | 0.130 | 2.13E−27 |
| | 38027319 | 38027435 | 38029482 | | | | | |
| LTA4H | 96396843- | 96397616- | 96397760- | Exc | 0.864 | 0.762 | −0.103 | 6.37E−05 |
| | 96397615 | 96397759 | 96400091 | | | | | |
| LTBP1 | 33540337- | 33567905- | 33568031- | Exc | 0.947 | 0.834 | −0.114 | 1.40E−16 |
| | 33567904 | 33568030 | 33572433 | | | | | |
| LTBP3 | 65307353- | 65307484- | 65307625- | Exc | 0.663 | 0.325 | −0.338 | 3.24E−05 |
| | 65307483 | 65307624 | 65307715 | | | | | |
| LTBP4 | 41128573- | 41128855- | 41128927- | Exc | 0.869 | 0.723 | −0.147 | 5.23E−04 |
| | 41128854 | 41128926 | 41129508 | | | | | |
| LUC7L | 258188- | 258600- | 258664- | Exc | 0.196 | 0.001 | −0.195 | 5.16E−33 |
| | 258599 | 258663 | 270647 | | | | | |
| LUC7L | 277336- | 278332- | 278402- | Exc | 0.388 | 0.003 | −0.385 | 4.67E−27 |
| | 278331 | 278401 | 279277 | | | | | |
| LYPLAL1 | 219352589- | 219366424- | 219366594- | Exc | 0.949 | 0.848 | −0.101 | 5.35E−04 |
| | 219366423 | 219366593 | 219383873 | | | | | |
| LYRM7 | 130517993- | 130522721- | 130522803- | Exc | 0.934 | 0.750 | −0.184 | 5.86E−05 |
| | 130522720 | 130522802 | 130535223 | | | | | |
| LZTFL1 | 45877277- | 45879419- | 45879544- | Exc | 0.856 | 0.727 | −0.129 | 1.28E−03 |
| | 45879418 | 45879543 | 45883480 | | | | | |
| MACF1 | 39929359- | 39930767- | 39930785- | Exc | 0.611 | 0.503 | −0.108 | 1.71E−07 |
| | 39930766 | 39930784 | 39934286 | | | | | |
| MACF1 | 39715776- | 39717518- | 39717614- | Inc | 0.005 | 0.126 | 0.121 | 3.68E−08 |
| | 39717517 | 39717613 | 39719969 | | | | | |
| MADD | 47291302- | 47295378- | 47295528- | Exc | 0.989 | 0.788 | −0.201 | 1.89E−04 |
| | 47295377 | 47295527 | 47296113 | | | | | |
| MADD | 47346129- | 47348289- | 47348359- | Unc | 0.883 | 0.887 | 0.005 | 9.39E−01 |
| | 47348288 | 47348358 | 47350208 | | | | | |
| MAGOHB | 10763280- | 10765239- | 10765578- | Inc | 0.098 | 0.210 | 0.111 | 6.40E−04 |
| | 10765238 | 10765577 | 10766037 | | | | | |
| MAGOHB | 10760536- | 10761697- | 10761983- | Inc | 0.140 | 0.249 | 0.109 | 4.64E−06 |
| | 10761696 | 10761982 | 10762429 | | | | | |
| MAN2C1 | 75654795- | 75654983- | 75655090- | Exc | 0.899 | 0.652 | −0.247 | 3.24E−12 |
| | 75654982 | 75655089 | 75656828 | | | | | |
| MAP2K5 | 67995747- | 68020254- | 68020284- | Unc | 0.835 | 0.842 | 0.007 | 7.64E−01 |
| | 68020253 | 68020283 | 68040568 | | | | | |
| MAP3K3 | 61769223- | 61769602- | 61769780- | Exc | 0.933 | 0.803 | −0.130 | 2.79E−11 |
| | 61769601 | 61769779 | 61770908 | | | | | |
| MAP4K4 | 102486878- | 102487956- | 102488148- | Exc | 0.453 | 0.327 | −0.126 | 1.29E−18 |
| | 102487955 | 102488147 | 102490108 | | | | | |
| MAP7D1 | 36639080- | 36640499- | 36640610- | Unc | 0.666 | 0.668 | 0.002 | 9.54E−01 |
| | 36640498 | 36640609 | 36641799 | | | | | |
| MAPK11 | 50705912- | 50705998- | 50706057- | Exc | 0.972 | 0.797 | −0.175 | 6.33E−04 |
| | 50705997 | 50706056 | 50706248 | | | | | |
| MAPK12 | 50685396- | 50686121- | 50686206- | Exc | 0.741 | 0.534 | −0.207 | 7.53E−05 |
| | 50686120 | 50686205 | 50686318 | | | | | |
| MAPK9 | 179707609- | 179713975- | 179714068- | Unc | 0.198 | 0.200 | 0.001 | 9.40E−01 |
| | 179713974 | 179714067 | 179718847 | | | | | |
| MAPKAP1 | 128246863- | 128268589- | 128268697- | Inc | 0.571 | 0.683 | 0.112 | 9.36E−08 |
| | 128268588 | 128268696 | 128305337 | | | | | |
| MARK3 | 103964866- | 103966493- | 103966538- | Inc | 0.538 | 0.737 | 0.199 | 3.24E−16 |
| | 103966492 | 103966537 | 103969218 | | | | | |
| MARK3 | 103958372- | 103966493- | 103966538- | Inc | 0.758 | 0.880 | 0.122 | 1.67E−04 |
| | 103966492 | 103966537 | 103969218 | | | | | |
| MAZ | 29820063- | 29820861- | 29821086- | Unc | 0.160 | 0.165 | 0.005 | 8.90E−01 |
| | 29820860 | 29821085 | 29821397 | | | | | |
| MBD1 | 47799326- | 47799704- | 47799842- | Unc | 0.848 | 0.858 | 0.010 | 7.89E−01 |
| | 47799703 | 47799841 | 47799933 | | | | | |
| MBD5 | 148779254- | 148811960- | 148812019- | Exc | 0.267 | 0.017 | −0.250 | 2.05E−06 |
| | 148811959 | 148812018 | 148936268 | | | | | |
| MBNL1 | 152165563- | 152173331- | 152173367- | Inc | 0.612 | 0.771 | 0.159 | 6.22E−15 |
| | 152173330 | 152173366 | 152174055 | | | | | |
| MBOAT2 | 9000895- | 9002720- | 9002853- | Exc | 0.993 | 0.819 | −0.174 | 3.07E−22 |
| | 9002719 | 9002852 | 9004299 | | | | | |
| MBOAT2 | 9000895- | 9002401- | 9002453- | Exc | 0.949 | 0.296 | −0.653 | 6.20E−17 |
| | 9002400 | 9002452 | 9004299 | | | | | |
| MCCC2 | 70900296- | 70922467- | 70922581- | Unc | 0.850 | 0.845 | −0.005 | 9.93E−01 |
| | 70922466 | 70922580 | 70927947 | | | | | |
| ME3 | 86161441- | 86168408- | 86168802- | Inc | 0.222 | 0.453 | 0.231 | 1.78E−04 |
| | 86168407 | 86168801 | 86176132 | | | | | |
| MECR | 29543198- | 29547350- | 29547434- | Unc | 0.195 | 0.195 | 0.000 | 9.67E−01 |
| | 29547349 | 29547433 | 29557242 | | | | | |
| MED13 | 60032929- | 60033041- | 60033200- | Exc | 0.975 | 0.819 | −0.156 | 1.34E−14 |
| | 60033040 | 60033199 | 60033699 | | | | | |
| MED15 | 20922919- | 20929400- | 20929520- | Exc | 0.596 | 0.429 | −0.167 | 7.19E−05 |
| | 20929399 | 20929519 | 20936897 | | | | | |
| MED23 | 131931387- | 131936464- | 131936482- | Unc | 0.210 | 0.217 | 0.007 | 9.45E−01 |
| | 131936463 | 131936481 | 131937046 | | | | | |
| MEG3 | 101297872- | 101298849- | 101298979- | Exc | 0.273 | 0.138 | −0.135 | 1.15E−09 |
| | 101298848 | 101298978 | 101302503 | | | | | |
| MEG3 | 101296087- | 101298849- | 101298979- | Exc | 0.654 | 0.482 | −0.172 | 6.45E−05 |
| | 101298848 | 101298978 | 101302503 | | | | | |
| MEG3 | 101297872- | 101302076- | 101302217- | Exc | 0.660 | 0.431 | −0.229 | 5.34E−23 |
| | 101302075 | 101302216 | 101302503 | | | | | |
| MEG3 | 101297872- | 101302073- | 101302217- | Exc | 0.659 | 0.428 | −0.232 | 2.36E−23 |
| | 101302072 | 101302216 | 101302503 | | | | | |
| MEG3 | 101296087- | 101297758- | 101297872- | Inc | 0.606 | 0.798 | 0.192 | 3.43E−14 |
| | 101297757 | 101297871 | 101298848 | | | | | |
| MEGF6 | 3415391- | 3415702- | 3415831- | Exc | 0.752 | 0.480 | −0.271 | 1.38E−06 |
| | 3415701 | 3415830 | 3416151 | | | | | |
| MEGF6 | 3413348- | 3413552- | 3413684- | Exc | 0.622 | 0.264 | −0.358 | 3.37E−12 |
| | 3413551 | 3413683 | 3413796 | | | | | |
| MEGF6 | 3407524- | 3409203- | 3409332- | Unc | 0.852 | 0.860 | 0.008 | 3.46E−01 |
| | 3409202 | 3409331 | 3410334 | | | | | |
| MEIS1 | 66795889- | 66796182- | 66796278- | Unc | 0.654 | 0.655 | 0.002 | 3.87E−01 |
| | 66796181 | 66796277 | 66798377 | | | | | |
| METAP1 | 99917019- | 99926920- | 99927018- | Inc | 0.022 | 0.202 | 0.179 | 7.38E−14 |
| | 99926919 | 99927017 | 99950017 | | | | | |
| METAP1 | 99917019- | 99926920- | 99927022- | Inc | 0.011 | 0.126 | 0.114 | 4.21E−09 |
| | 99926919 | 99927021 | 99950017 | | | | | |
| METTL10 | 126449073- | 126450869- | 126451128- | Unc | 0.492 | 0.482 | −0.010 | 8.27E−01 |
| | 126450868 | 126451127 | 126453960 | | | | | |
| METTL14 | 119625207- | 119626766- | 119626977- | Exc | 0.896 | 0.795 | −0.102 | 8.30E−04 |
| | 119626765 | 119626976 | 119631152 | | | | | |
| METTL21A | 208478168- | 208485365- | 208485419- | Unc | 0.142 | 0.135 | −0.007 | 8.61E−01 |
| | 208485364 | 208485418 | 208486529 | | | | | |
| MFF | 228205097- | 228207461- | 228207536- | Exc | 0.326 | 0.185 | −0.141 | 1.63E−11 |
| | 228207460 | 228207535 | 228211941 | | | | | |
| MFF | 228190144- | 228195342- | 228195563- | Exc | 0.902 | 0.752 | −0.150 | 4.68E−17 |
| | 228195341 | 228195562 | 228197134 | | | | | |
| MFF | 228205097- | 228211942- | 228212101- | Exc | 0.851 | 0.689 | −0.162 | 1.35E−17 |
| | 228211941 | 228212100 | 228217229 | | | | | |
| MFF | 228190144- | 228193394- | 228195563- | Exc | 0.914 | 0.743 | −0.171 | 3.75E−23 |
| | 228193393 | 228195562 | 228197134 | | | | | |
| MFF | 228205097- | 228207461- | 228207536- | Exc | 0.378 | 0.171 | −0.206 | 1.50E−12 |
| | 228207460 | 228207535 | 228220392 | | | | | |
| MFF | 228190144- | 228193394- | 228193506- | Exc | 0.836 | 0.448 | −0.387 | 1.16E−33 |
| | 228193393 | 228193505 | 228195341 | | | | | |
| MFF | 228193506- | 228194322- | 228194500- | Inc | 0.091 | 0.196 | 0.104 | 3.48E−05 |
| | 228194321 | 228194499 | 228195341 | | | | | |
| MFSD12 | 3546167- | 3546253- | 3546424- | Exc | 0.952 | 0.836 | −0.117 | 1.26E−06 |
| | 3546252 | 3546423 | 3547269 | | | | | |
| MGEA5 | 103552701- | 103553670- | 103553756- | Exc | 0.921 | 0.814 | −0.107 | 2.90E−10 |
| | 103553669 | 103553755 | 103557736 | | | | | |
| MGLL | 127414034- | 127429419- | 127429509- | Unc | 0.893 | 0.892 | −0.002 | 8.74E−01 |
| | 127429418 | 127429508 | 127439895 | | | | | |
| MGST2 | 140587232- | 140599697- | 140599797- | Exc | 0.942 | 0.729 | −0.213 | 1.98E−06 |
| | 140599696 | 140599796 | 140616350 | | | | | |
| MICU3 | 16921747- | 16927197- | 16927229- | Unc | 0.814 | 0.806 | −0.008 | 8.50E−01 |
| | 16927196 | 16927228 | 16935291 | | | | | |
| MINK1 | 4795530- | 4795697- | 4795808- | Exc | 0.424 | 0.289 | −0.134 | 7.35E−05 |
| | 4795696 | 4795807 | 4795950 | | | | | |
| MIR22HG | 1617309- | 1617665- | 1617748- | Exc | 0.817 | 0.649 | −0.168 | 3.76E−05 |
| | 1617664 | 1617747 | 1619423 | | | | | |
| MKNK2 | 2037829- | 2039630- | 2039856- | Unc | 0.552 | 0.545 | −0.007 | 4.92E−01 |
| | 2039629 | 2039855 | 2040132 | | | | | |
| MKS1 | 56291749- | 56292102- | 56292200- | Exc | 0.973 | 0.797 | −0.176 | 4.57E−04 |
| | 56292101 | 56292199 | 56293448 | | | | | |
| MLF1 | 158289181- | 158310223- | 158310371- | Exc | 1.000 | 0.824 | −0.176 | 2.77E−04 |
| | 158310222 | 158310370 | 158314650 | | | | | |
| MLH1 | 37053591- | 37055923- | 37056036- | Exc | 1.000 | 0.890 | −0.110 | 1.29E−10 |
| | 37055922 | 37056035 | 37058996 | | | | | |
| MLH1 | 37053591- | 37055923- | 37056036- | Exc | 0.982 | 0.802 | −0.179 | 1.88E−07 |
| | 37055922 | 37056035 | 37061800 | | | | | |
| MLLT10 | 21827842- | 21846550- | 21846754- | Unc | 0.212 | 0.204 | −0.008 | 9.92E−01 |
| | 21846549 | 21846753 | 21875222 | | | | | |
| MLLT6 | 36877001- | 36878131- | 36878481- | Exc | 0.838 | 0.636 | −0.202 | 3.61E−04 |
| | 36878130 | 36878480 | 36878930 | | | | | |
| MLST8 | 2257347- | 2258211- | 2258336- | Exc | 0.881 | 0.640 | −0.241 | 1.01E−18 |
| | 2258210 | 2258335 | 2258450 | | | | | |
| MMP19 | 56234667- | 56234890- | 56235021- | Exc | 0.952 | 0.743 | −0.209 | 2.66E−07 |
| | 56234889 | 56235020 | 56236136 | | | | | |
| MMP19 | 56234667- | 56234890- | 56234992- | Exc | 0.920 | 0.626 | −0.293 | 2.11E−06 |
| | 56234889 | 56234991 | 56236136 | | | | | |
| MORF4L2 | 102933529- | 102939609- | 102939658- | Exc | 0.154 | 0.053 | −0.101 | 8.00E−52 |
| | 102939608 | 102939657 | 102940098 | | | | | |
| MORF4L2 | 102933580- | 102939609- | 102939658- | Exc | 0.385 | 0.203 | −0.183 | 8.78E−18 |
| | 102939608 | 102939657 | 102940098 | | | | | |
| MORF4L2 | 102933549- | 102939609- | 102939658- | Exc | 0.597 | 0.281 | −0.316 | 1.77E−28 |
| | 102939608 | 102939657 | 102940098 | | | | | |
| MOSPD1 | 134031065- | 134033120- | 134033196- | Exc | 0.971 | 0.832 | −0.139 | 1.45E−09 |
| | 134033119 | 134033195 | 134033309 | | | | | |
| MOSPD1 | 134023223- | 134025509- | 134025671- | Inc | 0.625 | 0.832 | 0.207 | 1.93E−09 |
| | 134025508 | 134025670 | 134030846 | | | | | |
| MPDZ | 13183585- | 13186269- | 13186386- | Exc | 0.894 | 0.770 | −0.124 | 4.45E−07 |
| | 13186268 | 13186385 | 13188782 | | | | | |
| MPI | 75188667- | 75189352- | 75189561- | Exc | 0.908 | 0.800 | −0.107 | 1.24E−04 |
| | 75189351 | 75189560 | 75189852 | | | | | |
| MPPE1 | 11897356- | 11897525- | 11897620- | Unc | 0.416 | 0.424 | 0.008 | 9.19E−01 |
| | 11897524 | 11897619 | 11906201 | | | | | |
| MPV17 | 27535640- | 27535861- | 27536022- | Exc | 0.916 | 0.811 | −0.106 | 2.99E−07 |
| | 27535860 | 27536021 | 27545314 | | | | | |
| MRI1 | 13875924- | 13876768- | 13876944- | Unc | 0.692 | 0.700 | 0.008 | 9.25E−01 |
| | 13876767 | 13876943 | 13879172 | | | | | |
| MRI1 | 13875924- | 13876768- | 13876880- | Unc | 0.604 | 0.611 | 0.007 | 9.69E−01 |
| | 13876767 | 13876879 | 13879172 | | | | | |
| MRPL33 | 27995560- | 27997291- | 27997398- | Exc | 0.447 | 0.291 | −0.156 | 3.59E−28 |
| | 27997290 | 27997397 | 28002299 | | | | | |
| MRPL55 | 228296020- | 228296138- | 228296176- | Unc | 0.278 | 0.270 | −0.008 | 9.35E−01 |
| | 228296137 | 228296175 | 228296655 | | | | | |
| MRPL55 | 228296020- | 228296138- | 228296210- | Unc | 0.243 | 0.235 | −0.008 | 9.55E−01 |
| | 228296137 | 228296209 | 228296655 | | | | | |
| MSRB3 | 65672646- | 65702309- | 65702436- | Exc | 0.521 | 0.420 | −0.101 | 1.46E−04 |
| | 65702308 | 65702435 | 65720605 | | | | | |
| MSRB3 | 65672646- | 65700643- | 65700807- | Inc | 0.140 | 0.290 | 0.149 | 1.45E−04 |
| | 65700642 | 65700806 | 65702308 | | | | | |
| MTCH2 | 47656287- | 47657097- | 47657124- | Exc | 0.915 | 0.813 | −0.102 | 2.02E−16 |
| | 47657096 | 47657123 | 47660250 | | | | | |
| MTIF2 | 55491002- | 55493517- | 55493655- | Unc | 0.163 | 0.155 | −0.008 | 6.61E−01 |
| | 55493516 | 55493654 | 55494704 | | | | | |
| MTMR2 | 95598841- | 95620776- | 95620849- | Exc | 0.262 | 0.122 | −0.141 | 3.54E−05 |
| | 95620775 | 95620848 | 95621319 | | | | | |
| MTMR2 | 95621426- | 95647406- | 95647477- | Inc | 0.506 | 0.679 | 0.173 | 8.49E−04 |
| | 95647405 | 95647476 | 95657038 | | | | | |
| MTMR3 | 30416874- | 30418018- | 30418129- | Exc | 0.810 | 0.641 | −0.169 | 6.23E−06 |
| | 30418017 | 30418128 | 30418597 | | | | | |
| MTMR3 | 30418687- | 30419446- | 30419473- | Exc | 0.384 | 0.214 | −0.169 | 3.78E−06 |
| | 30419445 | 30419472 | 30421618 | | | | | |
| MTMR6 | 25823631- | 25825787- | 25825914- | Exc | 0.934 | 0.675 | −0.259 | 7.28E−27 |
| | 25825786 | 25825913 | 25825990 | | | | | |
| MTRR | 7871037- | 7873486- | 7873640- | Exc | 0.770 | 0.468 | −0.302 | 2.38E−12 |
| | 7873485 | 7873639 | 7875370 | | | | | |
| MTRR | 7871037- | 7873486- | 7873626- | Exc | 0.625 | 0.288 | −0.336 | 1.13E−09 |
| | 7873485 | 7873625 | 7875370 | | | | | |
| MTSS1L | 70712313- | 70713218- | 70713227- | Exc | 0.308 | 0.193 | −0.115 | 6.64E−12 |
| | 70713217 | 70713226 | 70713532 | | | | | |
| MXI1 | 111988080- | 112004586- | 112004632- | Unc | 0.892 | 0.898 | 0.006 | 9.46E−01 |
| | 112004585 | 112004631 | 112038937 | | | | | |
| MXRA7 | 74676962- | 74679929- | 74680010- | Unc | 0.158 | 0.165 | 0.007 | 3.84E−01 |
| | 74679928 | 74680009 | 74681153 | | | | | |
| MYBL1 | 67477061- | 67478299- | 67478479- | Exc | 0.243 | 0.053 | −0.190 | 9.27E−05 |
| | 67478298 | 67478478 | 67478918 | | | | | |
| MYCBP2 | 77699604- | 77700453- | 77700678- | Exc | 0.898 | 0.773 | −0.125 | 4.67E−06 |
| | 77700452 | 77700677 | 77713330 | | | | | |
| MYCBP2 | 77673149- | 77692475- | 77692655- | Unc | 0.215 | 0.207 | −0.007 | 8.24E−01 |
| | 77692474 | 77692654 | 77695507 | | | | | |
| MYEF2 | 48441569- | 48443297- | 48443369- | Inc | 0.369 | 0.722 | 0.353 | 1.36E−04 |
| | 48443296 | 48443368 | 48443669 | | | | | |
| MYLK | 123444926- | 123451743- | 123451950- | Exc | 0.802 | 0.611 | −0.191 | 4.65E−34 |
| | 123451742 | 123451949 | 123452533 | | | | | |
| MYLK | 123550518- | 123554715- | 123554783- | Inc | 0.244 | 0.589 | 0.345 | 1.49E−21 |
| | 123554714 | 123554782 | 123595405 | | | | | |
| MYLK | 123550518- | 123595406- | 123595465- | Inc | 0.739 | 0.897 | 0.158 | 1.79E−05 |
| | 123595405 | 123595464 | 123603052 | | | | | |
| MYNN | 169500432- | 169501265- | 169501349- | Exc | 0.805 | 0.625 | −0.181 | 2.38E−05 |
| | 169501264 | 169501348 | 169502409 | | | | | |
| MYNN | 169491251- | 169491819- | 169491886- | Inc | 0.186 | 0.328 | 0.142 | 7.51E−05 |
| | 169491818 | 169491885 | 169492052 | | | | | |
| MYO18A | 27442859- | 27443462- | 27443474- | Inc | 0.106 | 0.359 | 0.253 | 2.29E−07 |
| | 27443461 | 27443473 | 27445062 | | | | | |
| MYO18A | 27449272- | 27464034- | 27464103- | Unc | 0.324 | 0.330 | 0.005 | 7.92E−01 |
| | 27464033 | 27464102 | 27467357 | | | | | |
| MYO19 | 34870392- | 34870956- | 34871026- | Exc | 0.980 | 0.870 | −0.109 | 9.03E−04 |
| | 34870955 | 34871025 | 34871700 | | | | | |
| MYO1B | 192265195- | 192267358- | 192267445- | Inc | 0.293 | 0.483 | 0.190 | 3.03E−41 |
| | 192267357 | 192267444 | 192272840 | | | | | |
| MYO1B | 192265195- | 192265475- | 192265562- | Inc | 0.169 | 0.296 | 0.128 | 2.28E−11 |
| | 192265474 | 192265561 | 192267357 | | | | | |
| MYO5A | 52628754- | 52630007- | 52630082- | Exc | 0.180 | 0.063 | −0.118 | 5.42E−05 |
| | 52630006 | 52630081 | 52632392 | | | | | |
| MYO5A | 52638659- | 52641015- | 52641024- | Inc | 0.823 | 0.953 | 0.130 | 1.78E−04 |
| | 52641014 | 52641023 | 52643450 | | | | | |
| MYO5A | 52632592- | 52635314- | 52635395- | Unc | 0.610 | 0.617 | 0.007 | 9.78E−01 |
| | 52635313 | 52635394 | 52638557 | | | | | |
| N6AMT2 | 21306261- | 21311862- | 21311945- | Unc | 0.745 | 0.755 | 0.010 | 9.61E−01 |
| | 21311861 | 21311944 | 21331593 | | | | | |
| NABP1 | 192546744- | 192547218- | 192547322- | Inc | 0.531 | 0.729 | 0.198 | 2.10E−25 |
| | 192547217 | 192547321 | 192548454 | | | | | |
| NADK2 | 36207368- | 36208727- | 36208793- | Exc | 0.107 | 0.005 | −0.102 | 2.64E−11 |
| | 36208726 | 36208792 | 36211945 | | | | | |
| NAE1 | 66860487- | 66860580- | 66860684- | Exc | 0.974 | 0.859 | −0.115 | 2.65E−09 |
| | 66860579 | 66860683 | 66864749 | | | | | |
| NAE1 | 66860487- | 66860571- | 66860684- | Exc | 0.912 | 0.596 | −0.315 | 1.31E−08 |
| | 66860570 | 66860683 | 66864749 | | | | | |
| NBN | 90993752- | 90994181- | 90994231- | Exc | 0.173 | 0.017 | −0.156 | 9.26E−10 |
| | 90994180 | 90994230 | 90994949 | | | | | |
| NCK2 | 106471746- | 106497784- | 106498506- | Unc | 0.595 | 0.586 | −0.008 | 9.98E−01 |
| | 106497783 | 106498505 | 106509437 | | | | | |
| NCOR2 | 124810917- | 124811955- | 124812180- | Unc | 0.657 | 0.657 | 0.000 | 9.08E−01 |
| | 124811954 | 124812179 | 124815390 | | | | | |
| NCOR2 | 124810917- | 124812093- | 124812180- | Unc | 0.682 | 0.681 | −0.001 | 9.02E−01 |
| | 124812092 | 124812179 | 124815390 | | | | | |
| NEDD1 | 97301403- | 97301480- | 97301635- | Unc | 0.870 | 0.878 | 0.008 | 9.75E−01 |
| | 97301479 | 97301634 | 97303529 | | | | | |
| NEDD1 | 97301079- | 97301382- | 97301635- | Unc | 0.829 | 0.826 | −0.003 | 9.29E−01 |
| | 97301381 | 97301634 | 97303529 | | | | | |
| NEK1 | 170506701- | 170508710- | 170508765- | Exc | 0.982 | 0.830 | −0.152 | 4.60E−07 |
| | 170508709 | 170508764 | 170509799 | | | | | |
| NEO1 | 73566347- | 73567033- | 73567066- | Inc | 0.029 | 0.149 | 0.120 | 6.18E−09 |
| | 73567032 | 73567065 | 73570471 | | | | | |
| NEXN | 78383959- | 78390873- | 78390915- | Exc | 0.792 | 0.654 | −0.138 | 1.27E−08 |
| | 78390872 | 78390914 | 78392098 | | | | | |
| NF2 | 30077591- | 30079009- | 30079069- | Inc | 0.564 | 0.807 | 0.244 | 4.92E−09 |
| | 30079008 | 30079068 | 30090740 | | | | | |
| NF2 | 30077591- | 30079009- | 30079054- | Inc | 0.733 | 0.900 | 0.167 | 1.66E−10 |
| | 30079008 | 30079053 | 30090740 | | | | | |
| NFE2L1 | 46133961- | 46134394- | 46134484- | Exc | 0.483 | 0.327 | −0.156 | 1.37E−34 |
| | 46134393 | 46134483 | 46134705 | | | | | |
| NIN | 51230683- | 51233025- | 51233115- | Unc | 0.888 | 0.896 | 0.008 | 8.80E−01 |
| | 51233024 | 51233114 | 51233497 | | | | | |
| NIPA2 | 23021430- | 23033278- | 23033414- | Exc | 0.771 | 0.608 | −0.163 | 9.71E−04 |
| | 23033277 | 23033413 | 23033893 | | | | | |
| NIPA2 | 23021430- | 23027801- | 23027923- | Exc | 0.511 | 0.326 | −0.185 | 1.06E−05 |
| | 23027800 | 23027922 | 23033277 | | | | | |
| NLRC5 | 57108596- | 57110731- | 57110815- | Exc | 0.979 | 0.857 | −0.122 | 1.51E−05 |
| | 57110730 | 57110814 | 57111207 | | | | | |
| NLRX1 | 119042183- | 119043065- | 119043135- | Exc | 0.885 | 0.742 | −0.143 | 1.09E−03 |
| | 119043064 | 119043134 | 119043609 | | | | | |
| NPEPPS | 45646861- | 45654447- | 45654527- | Inc | 0.036 | 0.172 | 0.135 | 4.25E−07 |
| | 45654446 | 45654526 | 45656755 | | | | | |
| NPR2 | 35802605- | 35802729- | 35802801- | Exc | 0.979 | 0.878 | −0.101 | 2.79E−11 |
| | 35802728 | 35802800 | 35805507 | | | | | |
| NPRL3 | 162775- | 167300- | 167375- | Exc | 0.852 | 0.697 | −0.155 | 1.74E−05 |
| | 167299 | 167374 | 169124 | | | | | |
| NPRL3 | 169255- | 174936- | 175073- | Unc | 0.201 | 0.206 | 0.006 | 9.15E−01 |
| | 174935 | 175072 | 180520 | | | | | |
| NPRL3 | 162775- | 180521- | 180591- | Unc | 0.856 | 0.858 | 0.003 | 9.12E−01 |
| | 180520 | 180590 | 188148 | | | | | |
| NRG1 | 32617925- | 32620736- | 32620878- | Inc | 0.328 | 0.510 | 0.182 | 1.86E−07 |
| | 32620735 | 32620877 | 32621265 | | | | | |
| NSFL1C | 1435778- | 1436359- | 1436516- | Exc | 0.337 | 0.214 | −0.123 | 3.38E−07 |
| | 1436358 | 1436515 | 1438844 | | | | | |
| NSFL1C | 1435778- | 1436359- | 1436365- | Exc | 0.498 | 0.345 | −0.153 | 1.50E−10 |
| | 1436358 | 1436364 | 1438844 | | | | | |
| NSMCE2 | 126163520- | 126168242- | 126168341- | Inc | 0.029 | 0.162 | 0.133 | 3.75E−10 |
| | 126168241 | 126168340 | 126194344 | | | | | |
| NSUN4 | 46810817- | 46812593- | 46812748- | Exc | 0.941 | 0.817 | −0.124 | 8.10E−04 |
| | 46812592 | 46812747 | 46818539 | | | | | |
| NT5C2 | 104865559- | 104871502- | 104871563- | Exc | 0.657 | 0.351 | −0.306 | 4.94E−05 |
| | 104871501 | 104871562 | 104899162 | | | | | |
| NTMT1 | 132388575- | 132394929- | 132395081- | Unc | 0.837 | 0.828 | −0.009 | 8.12E−01 |
| | 132394928 | 132395080 | 132396332 | | | | | |
| NTPCR | 233086491- | 233091303- | 233091466- | Unc | 0.776 | 0.785 | 0.009 | 8.51E−01 |
| | 233091302 | 233091465 | 233092092 | | | | | |
| NUB1 | 151042553- | 151046159- | 151046327- | Exc | 0.984 | 0.858 | −0.126 | 3.03E−19 |
| | 151046158 | 151046326 | 151048509 | | | | | |
| NUBP2 | 1836657- | 1836758- | 1836957- | Unc | 0.569 | 0.575 | 0.006 | 7.28E−01 |
| | 1836757 | 1836956 | 1837677 | | | | | |
| NUMB | 73822475- | 73833605- | 73833690- | Unc | 0.700 | 0.697 | −0.003 | 8.55E−01 |
| | 73833604 | 73833689 | 73876644 | | | | | |
| NUP43 | 150057759- | 150059779- | 150059915- | Exc | 0.983 | 0.865 | −0.118 | 7.07E−08 |
| | 150059778 | 150059914 | 150063525 | | | | | |
| NUP62 | 50413142- | 50430951- | 50431106- | Inc | 0.662 | 0.787 | 0.124 | 1.01E−03 |
| | 50430950 | 50431105 | 50432582 | | | | | |
| NUP98 | 3704672- | 3707294- | 3707425- | Unc | 0.872 | 0.862 | −0.009 | 8.71E−01 |
| | 3707293 | 3707424 | 3712576 | | | | | |
| NUPL2 | 23226766- | 23235458- | 23235535- | Exc | 0.846 | 0.602 | −0.244 | 4.56E−09 |
| | 23235457 | 23235534 | 23236298 | | | | | |
| NUTF2 | 67880889- | 67881181- | 67881360- | Unc | 0.107 | 0.104 | −0.003 | 9.22E−01 |
| | 67881180 | 67881359 | 67899004 | | | | | |
| NUTM2A-AS1 | 89048253- | 89067714- | 89067818- | Exc | 0.220 | 0.100 | −0.120 | 1.51E−07 |
| | 89067713 | 89067817 | 89086386 | | | | | |
| OARD1 | 41035177- | 41036580- | 41036693- | Exc | 0.830 | 0.563 | −0.267 | 6.53E−07 |
| | 41036579 | 41036692 | 41037814 | | | | | |
| OCRL | 128710530- | 128718321- | 128718345- | Inc | 0.104 | 0.364 | 0.260 | 1.97E−18 |
| | 128718320 | 128718344 | 128720978 | | | | | |
| ODF2 | 131223290- | 131231462- | 131231633- | Exc | 0.974 | 0.872 | −0.102 | 3.16E−04 |
| | 131231461 | 131231632 | 131233586 | | | | | |
| ODF2L | 86820543- | 86822127- | 86822286- | Exc | 0.786 | 0.514 | −0.272 | 2.12E−07 |
| | 86822126 | 86822285 | 86824469 | | | | | |
| OFD1 | 13780564- | 13781864- | 13781975- | Inc | 0.850 | 0.981 | 0.131 | 7.32E−06 |
| | 13781863 | 13781974 | 13785245 | | | | | |
| OPN3 | 241761300- | 241767722- | 241767882- | Unc | 0.441 | 0.437 | −0.004 | 9.18E−01 |
| | 241767721 | 241767881 | 241803183 | | | | | |
| OPTN | 13150290- | 13150962- | 13151016- | Exc | 0.341 | 0.121 | −0.220 | 4.83E−19 |
| | 13150961 | 13151015 | 13151111 | | | | | |
| OPTN | 13142303- | 13150138- | 13150290- | Inc | 0.210 | 0.387 | 0.176 | 8.72E−24 |
| | 13150137 | 13150289 | 13151111 | | | | | |
| ORMDL1 | 190647329- | 190647740- | 190647850- | Exc | 0.702 | 0.407 | −0.295 | 6.70E−15 |
| | 190647739 | 190647849 | 190648994 | | | | | |
| OS9 | 58112966- | 58113882- | 58114047- | Exc | 0.525 | 0.344 | −0.181 | 3.42E−70 |
| | 58113881 | 58114046 | 58114188 | | | | | |
| OSBPL8 | 76844760- | 76853639- | 76853676- | Inc | 0.096 | 0.296 | 0.200 | 5.73E−12 |
| | 76853638 | 76853675 | 76881289 | | | | | |
| OSBPL8 | 76844769- | 76853639- | 76853676- | Inc | 0.078 | 0.269 | 0.191 | 2.58E−22 |
| | 76853638 | 76853675 | 76881289 | | | | | |
| OSER1-AS1 | 42843644- | 42846007- | 42846323- | Unc | 0.153 | 0.153 | 0.000 | 8.79E−01 |
| | 42846006 | 42846322 | 42853460 | | | | | |
| P4HA2 | 131554338- | 131562390- | 131562915- | Unc | 0.162 | 0.155 | −0.007 | 7.05E−01 |
| | 131562389 | 131562914 | 131563482 | | | | | |
| P4HA2 | 131554338- | 131562625- | 131562915- | Unc | 0.167 | 0.160 | −0.007 | 7.10E−01 |
| | 131562624 | 131562914 | 131563482 | | | | | |
| PACRGL | 20715163- | 20726431- | 20726512- | Exc | 0.216 | 0.095 | −0.120 | 7.77E−05 |
| | 20726430 | 20726511 | 20728907 | | | | | |
| PACRGL | 20706438- | 20709426- | 20709494- | Exc | 0.845 | 0.666 | −0.179 | 1.22E−04 |
| | 20709425 | 20709493 | 20714410 | | | | | |
| PACRGL | 20709494- | 20711306- | 20711397- | Exc | 0.805 | 0.563 | −0.242 | 1.38E−05 |
| | 20711305 | 20711396 | 20714410 | | | | | |
| PACRGL | 20706438- | 20711306- | 20711397- | Exc | 0.767 | 0.462 | −0.305 | 8.76E−06 |
| | 20711305 | 20711396 | 20714410 | | | | | |
| PAM | 102296934- | 102309820- | 102310141- | Exc | 0.601 | 0.478 | −0.123 | 4.07E−33 |
| | 102309819 | 102310140 | 102325975 | | | | | |
| PARD3 | 34690846- | 34739245- | 34739377- | Exc | 0.749 | 0.584 | −0.165 | 4.33E−06 |
| | 34739244 | 34739376 | 34759012 | | | | | |
| PARD3 | 34620273- | 34625127- | 34625172- | Unc | 0.809 | 0.805 | −0.003 | 8.88E−01 |
| | 34625126 | 34625171 | 34626205 | | | | | |
| PARL | 183551378- | 183551512- | 183551614- | Exc | 0.937 | 0.819 | −0.118 | 1.42E−10 |
| | 183551511 | 183551613 | 183558357 | | | | | |
| PARP11 | 3935400- | 3938076- | 3938197- | Exc | 0.495 | 0.250 | −0.245 | 8.16E−04 |
| | 3938075 | 3938196 | 3939055 | | | | | |
| PARP6 | 72542434- | 72543186- | 72543299- | Inc | 0.705 | 0.870 | 0.164 | 2.78E−06 |
| | 72543185 | 72543298 | 72543547 | | | | | |
| PARP6 | 72542434- | 72543186- | 72543296- | Inc | 0.754 | 0.902 | 0.148 | 1.76E−07 |
| | 72543185 | 72543295 | 72543547 | | | | | |
| PATL1 | 59423214- | 59423429- | 59423519- | Exc | 0.878 | 0.710 | −0.168 | 2.80E−12 |
| | 59423428 | 59423518 | 59423971 | | | | | |
| PCBP2 | 53861078- | 53861589- | 53861628- | Exc | 0.649 | 0.431 | −0.218 | 2.54E−160 |
| | 53861588 | 53861627 | 53862560 | | | | | |
| PCID2 | 113839875- | 113845186- | 113845290- | Exc | 0.973 | 0.757 | −0.217 | 1.52E−31 |
| | 113845185 | 113845289 | 113849384 | | | | | |
| PCM1 | 17830197- | 17838100- | 17838265- | Exc | 0.579 | 0.444 | −0.135 | 4.45E−05 |
| | 17838099 | 17838264 | 17842955 | | | | | |
| PCNXL2 | 233344436- | 233353645- | 233353678- | Exc | 1.000 | 0.899 | −0.101 | 1.52E−05 |
| | 233353644 | 233353677 | 233353776 | | | | | |
| PCNXL2 | 233152901- | 233160892- | 233161146- | Unc | 0.882 | 0.891 | 0.009 | 9.04E−01 |
| | 233160891 | 233161145 | 233190013 | | | | | |
| PCNXL4 | 60559138- | 60574304- | 60575046- | Exc | 0.648 | 0.498 | −0.151 | 3.73E−06 |
| | 60574303 | 60575045 | 60581417 | | | | | |
| PCYT2 | 79863031- | 79863258- | 79863324- | Exc | 0.954 | 0.804 | −0.150 | 4.38E−10 |
| | 79863257 | 79863323 | 79863545 | | | | | |
| PDE4DIP | 144857729- | 144859759- | 144859999- | Inc | 0.682 | 0.821 | 0.140 | 6.83E−07 |
| | 144859758 | 144859998 | 144863317 | | | | | |
| PDGFC | 157771569- | 157782581- | 157782642- | Unc | 0.135 | 0.141 | 0.006 | 9.73E−01 |
| | 157782580 | 157782641 | 157891937 | | | | | |
| PDLIM2 | 22447255- | 22449064- | 22449182- | Exc | 0.864 | 0.542 | −0.322 | 9. ISE−124 |
| | 22449063 | 22449181 | 22451245 | | | | | |
| PDP1 | 94929308- | 94930139- | 94930174- | Unc | 0.185 | 0.184 | −0.002 | 8.92E−01 |
| | 94930138 | 94930173 | 94934243 | | | | | |
| PDPR | 70148344- | 70148773- | 70148834- | Unc | 0.272 | 0.268 | −0.005 | 9.18E−01 |
| | 70148772 | 70148833 | 70154363 | | | | | |
| PDXDC2P | 70010636- | 70010714- | 70011907- | Unc | 0.250 | 0.246 | −0.004 | 9.01E−01 |
| | 70010713 | 70011906 | 70011979 | | | | | |
| PDZD11 | 69509205- | 69509372- | 69509444- | Inc | 0.160 | 0.417 | 0.257 | 8.46E−39 |
| | 69509371 | 69509443 | 69509709 | | | | | |
| PEAK1 | 77544869- | 77576225- | 77576373- | Exc | 0.371 | 0.201 | −0.170 | 7.16E−08 |
| | 77576224 | 77576372 | 77577299 | | | | | |
| PEAK1 | 77578847- | 77657505- | 77657568- | Unc | 0.807 | 0.806 | −0.001 | 9.36E−01 |
| | 77657504 | 77657567 | 77712347 | | | | | |
| PEX1 | 92136441- | 92138643- | 92138726- | Exc | 0.785 | 0.444 | −0.341 | 1.47E−05 |
| | 92138642 | 92138725 | 92140257 | | | | | |
| PEX11A | 90227180- | 90229662- | 90229778- | Exc | 0.719 | 0.316 | −0.403 | 1.16E−07 |
| | 90229661 | 90229777 | 90233807 | | | | | |
| PEX5 | 7354438- | 7354837- | 7354948- | Exc | 0.442 | 0.279 | −0.163 | 9.51E−06 |
| | 7354836 | 7354947 | 7355207 | | | | | |
| PFDN1 | 139661119- | 139680001- | 139680168- | Exc | 0.784 | 0.458 | −0.326 | 1.30E−56 |
| | 139680000 | 139680167 | 139682625 | | | | | |
| PFDN5 | 53689424- | 53690214- | 53690336- | Unc | 0.167 | 0.175 | 0.008 | 9.25E−01 |
| | 53690213 | 53690335 | 53691633 | | | | | |
| PFDN5 | 53689424- | 53690238- | 53690336- | Unc | 0.173 | 0.180 | 0.007 | 9.26E−01 |
| | 53690237 | 53690335 | 53691633 | | | | | |
| PFDN6 | 33258022- | 33258103- | 33258228- | Exc | 0.964 | 0.847 | −0.117 | 1.39E−14 |
| | 33258102 | 33258227 | 33258492 | | | | | |
| PFKM | 48528822- | 48529074- | 48529167- | Exc | 0.683 | 0.563 | −0.120 | 1.32E−05 |
| | 48529073 | 48529166 | 48531503 | | | | | |
| PHC3 | 169840533- | 169846128- | 169846176- | Inc | 0.225 | 0.373 | 0.148 | 6.06E−04 |
| | 169846127 | 169846175 | 169846471 | | | | | |
| PHKG2 | 30764879- | 30767503- | 30767594- | Exc | 0.976 | 0.799 | −0.177 | 1.74E−07 |
| | 30767502 | 30767593 | 30767687 | | | | | |
| PHLDB1 | 118478415- | 118484009- | 118484166- | Inc | 0.095 | 0.239 | 0.143 | 5.84E−04 |
| | 118484008 | 118484165 | 118484530 | | | | | |
| PHLPP2 | 71715809- | 71718379- | 71718505- | Unc | 0.866 | 0.867 | 0.001 | 9.68E−01 |
| | 71718378 | 71718504 | 71724421 | | | | | |
| PI4KB | 151280278- | 151282687- | 151282732- | Exc | 0.296 | 0.168 | −0.129 | 2.68E−07 |
| | 151282686 | 151282731 | 151288048 | | | | | |
| PI4KB | 151288986- | 151298648- | 151298850- | Exc | 0.509 | 0.314 | −0.195 | 1.63E−06 |
| | 151298647 | 151298849 | 151299746 | | | | | |
| PIGF | 46808731- | 46815214- | 46815319- | Unc | 0.142 | 0.139 | −0.004 | 9.38E−01 |
| | 46815213 | 46815318 | 46819613 | | | | | |
| PIGG | 521020- | 524225- | 524535- | Exc | 0.946 | 0.784 | −0.162 | 3.14E−07 |
| | 524224 | 524534 | 527606 | | | | | |
| PIGN | 59829563- | 59830764- | 59830890- | Inc | 0.622 | 0.945 | 0.323 | 5.16E−07 |
| | 59830763 | 59830889 | 59854076 | | | | | |
| PIGP | 38441925- | 38444445- | 38444611- | Unc | 0.161 | 0.163 | 0.001 | 9.58E−01 |
| | 38444444 | 38444610 | 38444733 | | | | | |
| PIGQ | 630973- | 632248- | 632310- | Unc | 0.785 | 0.790 | 0.005 | 6.73E−01 |
| | 632247 | 632309 | 632882 | | | | | |
| PIGT | 44044984- | 44045157- | 44045295- | Exc | 0.917 | 0.752 | −0.165 | 6.18E−17 |
| | 44045156 | 44045294 | 44047934 | | | | | |
| PIGT | 44044984- | 44045157- | 44045335- | Exc | 0.918 | 0.751 | −0.167 | 3.18E−17 |
| | 44045156 | 44045334 | 44047934 | | | | | |
| PIK3C2A | 17167490- | 17169064- | 17169176- | Exc | 0.982 | 0.871 | −0.111 | 1.27E−08 |
| | 17169063 | 17169175 | 17170213 | | | | | |
| PIKFYVE | 209138458- | 209138742- | 209138778- | Exc | 0.499 | 0.309 | −0.190 | 6.54E−04 |
| | 209138741 | 209138777 | 209141435 | | | | | |
| PILRB | 99950747- | 99950833- | 99950894- | Exc | 0.952 | 0.688 | −0.264 | 9.36E−10 |
| | 99950832 | 99950893 | 99952765 | | | | | |
| PIN1 | 9946079- | 9947499- | 9947552- | Inc | 0.025 | 0.131 | 0.107 | 4.07E−07 |
| | 9947498 | 9947551 | 9949111 | | | | | |
| PINX1 | 10623427- | 10677703- | 10677780- | Unc | 0.726 | 0.736 | 0.010 | 8.30E−01 |
| | 10677702 | 10677779 | 10683661 | | | | | |
| PKIG | 43160620- | 43211226- | 43211373- | Exc | 0.569 | 0.417 | −0.152 | 1.06E−06 |
| | 43211225 | 43211372 | 43218437 | | | | | |
| PLA2G12A | 110638870- | 110639839- | 110639916- | Exc | 0.977 | 0.669 | −0.308 | 3.05E−28 |
| | 110639838 | 110639915 | 110650757 | | | | | |
| PLA2G12A | 110638870- | 110639845- | 110639916- | Exc | 0.940 | 0.460 | −0.480 | 4.91E−21 |
| | 110639844 | 110639915 | 110650757 | | | | | |
| PLA2R1 | 160832737- | 160833196- | 160833232- | Exc | 0.953 | 0.831 | −0.122 | 2.11E−08 |
| | 160833195 | 160833231 | 160833794 | | | | | |
| PLAGL1 | 144290116- | 144306272- | 144306312- | Exc | 0.234 | 0.047 | −0.187 | 2.90E−06 |
| | 144306271 | 144306311 | 144329226 | | | | | |
| PLAGL1 | 144285956- | 144287295- | 144287336- | Exc | 0.939 | 0.713 | −0.225 | 1.52E−10 |
| | 144287294 | 144287335 | 144290043 | | | | | |
| PLAGL1 | 144285956- | 144287295- | 144287350- | Exc | 0.918 | 0.657 | −0.261 | 1.80E−09 |
| | 144287294 | 144287349 | 144290043 | | | | | |
| PLAGL1 | 144285956- | 144287295- | 144287368- | Exc | 0.909 | 0.637 | −0.272 | 3.25E−09 |
| | 144287294 | 144287367 | 144290043 | | | | | |
| PLBD2 | 113822071- | 113822656- | 113822752- | Exc | 0.988 | 0.840 | −0.148 | 1.86E−68 |
| | 113822655 | 113822751 | 113823016 | | | | | |
| PLD3 | 40854632- | 40871460- | 40871838- | Unc | 0.890 | 0.883 | −0.007 | 5.77E−01 |
| | 40871459 | 40871837 | 40872325 | | | | | |
| PLD3 | 40854676- | 40871625- | 40871838- | Unc | 0.798 | 0.790 | −0.008 | 9.05E−01 |
| | 40871624 | 40871837 | 40872290 | | | | | |
| PLSCR4 | 145941565- | 145968344- | 145968425- | Unc | 0.700 | 0.691 | −0.009 | 6.67E−01 |
| | 145968343 | 145968424 | 145968746 | | | | | |
| PMS2P3 | 75140437- | 75141633- | 75141746- | Exc | 0.932 | 0.700 | −0.232 | 2.63E−04 |
| | 75141632 | 75141745 | 75141912 | | | | | |
| PNPLA8 | 108156019- | 108161920- | 108161966- | Inc | 0.701 | 0.871 | 0.170 | 9.72E−05 |
| | 108161919 | 108161965 | 108166472 | | | | | |
| PODNL1 | 14044807- | 14046793- | 14046858- | Exc | 0.645 | 0.491 | −0.154 | 2.46E−06 |
| | 14046792 | 14046857 | 14047179 | | | | | |
| POLR3GL | 145457605- | 145457935- | 145458004- | Exc | 0.976 | 0.858 | −0.118 | 1.04E−12 |
| | 145457934 | 145458003 | 145459651 | | | | | |
| PPAP2A | 54763978- | 54786788- | 54786943- | Exc | 0.736 | 0.379 | −0.356 | 4.31E−08 |
| | 54786787 | 54786942 | 54830399 | | | | | |
| PPFIBP1 | 27826761- | 27827125- | 27827155- | Exc | 0.858 | 0.757 | −0.100 | 3.19E−05 |
| | 27827124 | 27827154 | 27829360 | | | | | |
| PPIL3 | 201746212- | 201747065- | 201747159- | Exc | 0.868 | 0.767 | −0.101 | 4.82E−04 |
| | 201747064 | 201747158 | 201750420 | | | | | |
| PPIL3 | 201746212- | 201747065- | 201747106- | Exc | 0.800 | 0.666 | −0.134 | 8.74E−04 |
| | 201747064 | 201747105 | 201750420 | | | | | |
| PPIP5K2 | 102515890- | 102518935- | 102519109- | Exc | 0.381 | 0.132 | −0.249 | 5.73E−16 |
| | 102518934 | 102519108 | 102520372 | | | | | |
| PPM1M | 52280829- | 52280990- | 52281245- | Exc | 0.844 | 0.454 | −0.390 | 1.59E−21 |
| | 52280989 | 52281244 | 52281697 | | | | | |
| PPP1R12A | 80199549- | 80199946- | 80200114- | Exc | 0.738 | 0.616 | −0.122 | 1.02E−06 |
| | 80199945 | 80200113 | 80201005 | | | | | |
| PPP1R12A | 80199549- | 80199946- | 80200078- | Exc | 0.642 | 0.515 | −0.128 | 2.82E−05 |
| | 80199945 | 80200077 | 80201005 | | | | | |
| PPP1R18 | 30647167- | 30652185- | 30653824- | Unc | 0.868 | 0.877 | 0.009 | 7.54E−01 |
| | 30652184 | 30653823 | 30654890 | | | | | |
| PPP1R21 | 48725875- | 48732703- | 48732736- | Exc | 0.918 | 0.799 | −0.119 | 9.11E−04 |
| | 48732702 | 48732735 | 48734407 | | | | | |
| PPP2R3C | 35568591- | 35576510- | 35576581- | Exc | 0.987 | 0.858 | −0.129 | 4.10E−13 |
| | 35576509 | 35576580 | 35577344 | | | | | |
| PPP3CB | 75198179- | 75199630- | 75199660- | Inc | 0.238 | 0.638 | 0.401 | 6.54E−47 |
| | 75199629 | 75199659 | 75204482 | | | | | |
| PPP3CC | 22390532- | 22396982- | 22397012- | Inc | 0.100 | 0.299 | 0.199 | 1.62E−08 |
| | 22396981 | 22397011 | 22398127 | | | | | |
| PPP4C | 30087797- | 30092580- | 30092632- | Exc | 0.957 | 0.851 | −0.105 | 2.62E−16 |
| | 30092579 | 30092631 | 30093804 | | | | | |
| PPP4C | 30087797- | 30092521- | 30092632- | Exc | 0.926 | 0.767 | −0.159 | 4.27E−15 |
| | 30092520 | 30092631 | 30093804 | | | | | |
| PPP6R2 | 50874882- | 50875417- | 50875498- | Exc | 0.490 | 0.339 | −0.151 | 9.91E−07 |
| | 50875416 | 50875497 | 50875934 | | | | | |
| PPP6R3 | 68228296- | 68272613- | 68272740- | Unc | 0.419 | 0.422 | 0.003 | 9.79E−01 |
| | 68272612 | 68272739 | 68286968 | | | | | |
| PPP6R3 | 68228296- | 68272613- | 68272718- | Unc | 0.248 | 0.240 | −0.008 | 9.10E−01 |
| | 68272612 | 68272717 | 68286968 | | | | | |
| PPRC1 | 103897796- | 103898376- | 103898523- | Exc | 0.976 | 0.852 | −0.124 | 8.23E−06 |
| | 103898375 | 103898522 | 103898635 | | | | | |
| PRDM5 | 121737730- | 121737987- | 121738080- | Unc | 0.855 | 0.846 | −0.010 | 8.96E−01 |
| | 121737986 | 121738079 | 121739507 | | | | | |
| PREB | 27354377- | 27354542- | 27354700- | Exc | 0.971 | 0.867 | −0.104 | 2.68E−05 |
| | 27354541 | 27354699 | 27354877 | | | | | |
| PREPL | 44573530- | 44586636- | 44587178- | Inc | 0.142 | 0.262 | 0.119 | 1.92E−04 |
| | 44586635 | 44587177 | 44588518 | | | | | |
| PREPL | 44573530- | 44586636- | 44586890- | Inc | 0.140 | 0.248 | 0.108 | 6.19E−04 |
| | 44586635 | 44586889 | 44588518 | | | | | |
| PRKAG1 | 49399327- | 49399526- | 49399636- | Exc | 0.940 | 0.814 | −0.126 | 2.16E−17 |
| | 49399525 | 49399635 | 49406844 | | | | | |
| PRKAG1 | 49399327- | 49399526- | 49399665- | Exc | 0.899 | 0.703 | −0.196 | 5.74E−17 |
| | 49399525 | 49399664 | 49406844 | | | | | |
| PRKAG1 | 49397706- | 49398290- | 49398417- | Exc | 0.977 | 0.763 | −0.214 | 3.87E−53 |
| | 49398289 | 49398416 | 49398746 | | | | | |
| PRKAG1 | 49397074- | 49397341- | 49397379- | Exc | 0.985 | 0.741 | −0.244 | 7.28E−73 |
| | 49397340 | 49397378 | 49397539 | | | | | |
| PRMT2 | 48069652- | 48081712- | 48081849- | Exc | 0.981 | 0.783 | −0.199 | 1.98E−09 |
| | 48081711 | 48081848 | 48083294 | | | | | |
| PRPF39 | 45565432- | 45565627- | 45565962- | Inc | 0.472 | 0.663 | 0.190 | 7.61E−04 |
| | 45565626 | 45565961 | 45566089 | | | | | |
| PRPF40B | 50026664- | 50026797- | 50026908- | Exc | 0.888 | 0.566 | −0.322 | 1.00E−05 |
| | 50026796 | 50026907 | 50027209 | | | | | |
| PRR16 | 119952888- | 119954137- | 119954261- | Exc | 0.759 | 0.541 | −0.218 | 2.87E−04 |
| | 119954136 | 119954260 | 120021648 | | | | | |
| PRR4 | 11126321- | 11187381- | 11187485- | Exc | 0.455 | 0.165 | −0.290 | 5.30E−09 |
| | 11187380 | 11187484 | 11199618 | | | | | |
| PRRC2B | 134349112- | 134349841- | 134351923- | Unc | 0.770 | 0.776 | 0.006 | 8.86E−01 |
| | 134349840 | 134351922 | 134353130 | | | | | |
| PRRX1 | 170695543- | 170699418- | 170699490- | Inc | 0.538 | 0.681 | 0.143 | 8.34E−20 |
| | 170699417 | 170699489 | 170705188 | | | | | |
| PRUNE | 150990381- | 150991033- | 150991146- | Inc | 0.727 | 0.932 | 0.205 | 2.28E−05 |
| | 150991032 | 150991145 | 150997086 | | | | | |
| PRUNE | 150990381- | 150990943- | 150991146- | Inc | 0.774 | 0.944 | 0.171 | 4.97E−05 |
| | 150990942 | 150991145 | 150997086 | | | | | |
| PRUNE | 150999804- | 151001262- | 151001421- | Unc | 0.759 | 0.768 | 0.009 | 9.56E−01 |
| | 151001261 | 151001420 | 151006281 | | | | | |
| PSME4 | 54164678- | 54167093- | 54167138- | Exc | 0.991 | 0.868 | −0.123 | 6.57E−10 |
| | 54167092 | 54167137 | 54175557 | | | | | |
| PTAR1 | 72333601- | 72338242- | 72338547- | Unc | 0.873 | 0.866 | −0.006 | 9.96E−01 |
| | 72338241 | 72338546 | 72347054 | | | | | |
| PTAR1 | 72356775- | 72365697- | 72365867- | Unc | 0.903 | 0.896 | −0.008 | 9.79E−01 |
| | 72365696 | 72365866 | 72374768 | | | | | |
| PTPN13 | 87672278- | 87674162- | 87674219- | Exc | 0.423 | 0.293 | −0.131 | 2.96E−05 |
| | 87674161 | 87674218 | 87679412 | | | | | |
| PTPN21 | 88962837- | 88963547- | 88963635- | Exc | 0.997 | 0.889 | −0.108 | 2.48E−10 |
| | 88963546 | 88963634 | 88967126 | | | | | |
| PTPN23 | 47437708- | 47446142- | 47446270- | Exc | 0.846 | 0.653 | −0.193 | 1.09E−07 |
| | 47446141 | 47446269 | 47446494 | | | | | |
| PTPN4 | 120635119- | 120639362- | 120639407- | Exc | 0.945 | 0.633 | −0.312 | 7.64E−08 |
| | 120639361 | 120639406 | 120639672 | | | | | |
| PTPRA | 2903932- | 2928628- | 2928671- | Exc | 0.733 | 0.548 | −0.185 | 8.06E−12 |
| | 2928627 | 2928670 | 2944917 | | | | | |
| PTPRG | 62204658- | 62216899- | 62216986- | Unc | 0.176 | 0.180 | 0.004 | 8.55E−01 |
| | 62216898 | 62216985 | 62229480 | | | | | |
| PTPRS | 5218544- | 5218798- | 5218810- | Unc | 0.810 | 0.816 | 0.006 | 8.40E−01 |
| | 5218797 | 5218809 | 5219320 | | | | | |
| PUF60 | 144900705- | 144902836- | 144902887- | Unc | 0.702 | 0.706 | 0.004 | 8.39E−01 |
| | 144902835 | 144902886 | 144903766 | | | | | |
| PUM2 | 20518407- | 20526022- | 20526147- | Unc | 0.127 | 0.122 | −0.005 | 9.57E−01 |
| | 20526021 | 20526146 | 20527070 | | | | | |
| PVR | 45161179- | 45162010- | 45162169- | Exc | 0.822 | 0.704 | −0.118 | 1.90E−08 |
| | 45162009 | 45162168 | 45164558 | | | | | |
| PVR | 45161179- | 45162010- | 45162034- | Exc | 0.765 | 0.627 | −0.138 | 3.54E−08 |
| | 45162009 | 45162033 | 45164558 | | | | | |
| PXDN | 1687924- | 1691404- | 1691476- | Exc | 0.818 | 0.560 | −0.258 | 9.68E−121 |
| | 1691403 | 1691475 | 1695699 | | | | | |
| QTRTD1 | 113775712- | 113775846- | 113775954- | Inc | 0.328 | 0.715 | 0.387 | 9.51E−07 |
| | 113775845 | 113775953 | 113784083 | | | | | |
| R3HCC1L | 99923155- | 99946224- | 99946321- | Inc | 0.181 | 0.373 | 0.191 | 9.16E−04 |
| | 99946223 | 99946320 | 99967857 | | | | | |
| R3HDM1 | 136362587- | 136374238- | 136374328- | Unc | 0.194 | 0.189 | −0.004 | 9.92E−01 |
| | 136374237 | 136374327 | 136379063 | | | | | |
| R3HDM4 | 900953- | 901420- | 901547- | Exc | 0.959 | 0.838 | −0.121 | 5.37E−07 |
| | 901419 | 901546 | 901975 | | | | | |
| RAB11FIP2 | 119774622- | 119793493- | 119793553- | Exc | 0.147 | 0.036 | −0.110 | 2.48E−14 |
| | 119793492 | 119793552 | 119798482 | | | | | |
| RABEPK | 127970001- | 127982818- | 127982980- | Exc | 0.824 | 0.631 | −0.192 | 1.69E−06 |
| | 127982817 | 127982979 | 127990188 | | | | | |
| RAD1 | 34911918- | 34913575- | 34913684- | Exc | 0.630 | 0.518 | −0.112 | 4.26E−06 |
| | 34913574 | 34913683 | 34914799 | | | | | |
| RAD51D | 33433501- | 33434385- | 33434467- | Unc | 0.181 | 0.182 | 0.001 | 9.34E−01 |
| | 33434384 | 33434466 | 33446129 | | | | | |
| RAF1 | 12645789- | 12647700- | 12647799- | Unc | 0.877 | 0.872 | −0.005 | 7.91E−01 |
| | 12647699 | 12647798 | 12650264 | | | | | |
| RALGPS2 | 178858832- | 178861365- | 178861443- | Exc | 0.299 | 0.071 | −0.228 | 7.32E−14 |
| | 178861364 | 178861442 | 178863053 | | | | | |
| RALY | 32661442- | 32661625- | 32661673- | Inc | 0.721 | 0.832 | 0.111 | 9.14E−12 |
| | 32661624 | 32661672 | 32663679 | | | | | |
| RAPH1 | 204326649- | 204334962- | 204335037- | Unc | 0.185 | 0.191 | 0.005 | 6.97E−01 |
| | 204334961 | 204335036 | 204354306 | | | | | |
| RBBP9 | 18474708- | 18476482- | 18476525- | Inc | 0.845 | 0.961 | 0.116 | 3.55E−05 |
| | 18476481 | 18476524 | 18477712 | | | | | |
| RBCK1 | 400376- | 401515- | 401651- | Exc | 0.986 | 0.770 | −0.216 | 8.18E−04 |
| | 401514 | 401650 | 402770 | | | | | |
| RBM10 | 47028898- | 47030427- | 47030658- | Unc | 0.773 | 0.764 | −0.009 | 9.45E−01 |
| | 47030426 | 47030657 | 47032526 | | | | | |
| RBM27 | 145616996- | 145631274- | 145631439- | Unc | 0.697 | 0.699 | 0.003 | 9.86E−01 |
| | 145631273 | 145631438 | 145634505 | | | | | |
| RBM4 | 66407595- | 66410921- | 66411612- | Unc | 0.792 | 0.800 | 0.009 | 7.93E−01 |
| | 66410920 | 66411611 | 66413497 | | | | | |
| RBM41 | 106332070- | 106356627- | 106356699- | Inc | 0.206 | 0.467 | 0.262 | 1.16E−06 |
| | 106356626 | 106356698 | 106358581 | | | | | |
| RBM5 | 50137485- | 50137965- | 50138039- | Unc | 0.901 | 0.892 | −0.009 | 7.37E−01 |
| | 50137964 | 50138038 | 50140515 | | | | | |
| RCC1 | 28856452- | 28857035- | 28857128- | Unc | 0.211 | 0.215 | 0.004 | 9.95E−01 |
| | 28857034 | 28857127 | 28858314 | | | | | |
| RCOR3 | 211477483- | 211485697- | 211485830- | Exc | 0.148 | 0.048 | −0.101 | 3.06E−05 |
| | 211485696 | 211485829 | 211486061 | | | | | |
| REPS1 | 139242262- | 139247538- | 139247619- | Exc | 0.328 | 0.187 | −0.141 | 5.65E−04 |
| | 139247537 | 139247618 | 139251113 | | | | | |
| REV3L | 111737676- | 111793216- | 111793344- | Inc | 0.508 | 0.738 | 0.230 | 1.57E−08 |
| | 111793215 | 111793343 | 111803952 | | | | | |
| RFC2 | 73657577- | 73660992- | 73661094- | Unc | 0.898 | 0.892 | −0.006 | 8.01E−01 |
| | 73660991 | 73661093 | 73663341 | | | | | |
| RFWD2 | 176104223- | 176105624- | 176105684- | Inc | 0.782 | 0.934 | 0.152 | 7.36E−10 |
| | 176105623 | 176105683 | 176118141 | | | | | |
| RGL1 | 183605637- | 183666538- | 183666648- | Unc | 0.290 | 0.297 | 0.008 | 9.66E−01 |
| | 183666537 | 183666647 | 183711260 | | | | | |
| RGL2 | 33264893- | 33266232- | 33266429- | Exc | 0.872 | 0.647 | −0.225 | 2.35E−04 |
| | 33266231 | 33266428 | 33266646 | | | | | |
| RGN | 46944000- | 46951077- | 46951209- | Exc | 0.924 | 0.580 | −0.345 | 2.79E−05 |
| | 46951076 | 46951208 | 46951459 | | | | | |
| RHBDD2 | 75508579- | 75510683- | 75510805- | Exc | 0.388 | 0.279 | −0.110 | 2.25E−04 |
| | 75510682 | 75510804 | 75511146 | | | | | |
| RHOBTB1 | 62631410- | 62631801- | 62632049- | Exc | 0.969 | 0.828 | −0.141 | 1.82E−05 |
| | 62631800 | 62632048 | 62634711 | | | | | |
| RHOT1 | 30535329- | 30538135- | 30538258- | Exc | 0.379 | 0.258 | −0.121 | 1.89E−04 |
| | 30538134 | 30538257 | 30551634 | | | | | |
| RHOT1 | 30535329- | 30536369- | 30536465- | Exc | 0.205 | 0.065 | −0.140 | 2.04E−07 |
| | 30536368 | 30536464 | 30551634 | | | | | |
| RHOT1 | 30535329- | 30536369- | 30536465- | Exc | 0.284 | 0.096 | −0.188 | 4.46E−05 |
| | 30536368 | 30536464 | 30538134 | | | | | |
| RIF1 | 152276894- | 152279464- | 152279557- | Exc | 0.995 | 0.814 | −0.182 | 7.06E−08 |
| | 152279463 | 152279556 | 152285297 | | | | | |
| RIF1 | 152324661- | 152324988- | 152325066- | Unc | 0.593 | 0.600 | 0.006 | 8.92E−01 |
| | 152324987 | 152325065 | 152325154 | | | | | |
| RILP | 1549914- | 1550200- | 1550284- | Unc | 0.641 | 0.641 | 0.000 | 5.97E−01 |
| | 1550199 | 1550283 | 1551128 | | | | | |
| RILPL2 | 123907705- | 123915055- | 123915207- | Unc | 0.850 | 0.850 | 0.001 | 9.24E−01 |
| | 123915054 | 123915206 | 123920628 | | | | | |
| RIPK2 | 90770462- | 90775057- | 90775211- | Exc | 0.705 | 0.527 | −0.178 | 1.29E−03 |
| | 90775056 | 90775210 | 90777568 | | | | | |
| RNASE4 | 21152918- | 21161706- | 21161810- | Inc | 0.172 | 0.283 | 0.112 | 1.19E−06 |
| | 21161705 | 21161809 | 21167513 | | | | | |
| RNF14 | 141353308- | 141354369- | 141354521- | Unc | 0.819 | 0.812 | −0.007 | 8.30E−01 |
| | 141354368 | 141354520 | 141357867 | | | | | |
| RNF146 | 127601486- | 127607195- | 127607323- | Exc | 0.726 | 0.487 | −0.239 | 2.13E−14 |
| | 127607194 | 127607322 | 127607760 | | | | | |
| RNF170 | 42716999- | 42720559- | 42720633- | Exc | 0.801 | 0.683 | −0.118 | 6.72E−05 |
| | 42720558 | 42720632 | 42725146 | | | | | |
| RNF214 | 117153253- | 117153462- | 117153567- | Exc | 0.986 | 0.818 | −0.167 | 1.57E−08 |
| | 117153461 | 117153566 | 117153830 | | | | | |
| RNF34 | 121838022- | 121840545- | 121840611- | Inc | 0.031 | 0.154 | 0.123 | 9.16E−04 |
| | 121840544 | 121840610 | 121853961 | | | | | |
| RNF8 | 373448JO- | 37348926- | 37349131- | Unc | 0.898 | 0.893 | −0.005 | 9.04E−01 |
| | 37348925 | 37349130 | 37358517 | | | | | |
| RNFT1 | 58035806- | 58039901- | 58039978- | Exc | 0.621 | 0.199 | −0.422 | 2.53E−06 |
| | 58039900 | 58039977 | 58040187 | | | | | |
| RNGTT | 89479594- | 89511283- | 89511352- | Unc | 0.908 | 0.900 | −0.008 | 9.70E−01 |
| | 89511282 | 89511351 | 89554075 | | | | | |
| ROBO1 | 78695341- | 78696779- | 78696806- | Exc | 0.288 | 0.177 | −0.110 | 9.96E−04 |
| | 78696778 | 78696805 | 78700881 | | | | | |
| RP11- | 79412708- | 79413764- | 79413857- | Exc | 0.430 | 0.173 | −0.257 | 5.68E−04 |
| 1055B8.7 | 79413763 | 79413856 | 79414061 | | | | | |
| RP11-14N7.2 | 148932921- | 148933291- | 148933369- | Inc | 0.244 | 0.470 | 0.226 | 9.63E−19 |
| | 148933290 | 148933368 | 148951244 | | | | | |
| RP11- | 128214846- | 128218018- | 128218088- | Inc | 0.564 | 0.975 | 0.411 | 1.21E−10 |
| 274B21.1 | 128218017 | 128218087 | 128218943 | | | | | |
| RP11-33B1.1 | 120415679- | 120418966- | 120419059- | Exc | 0.958 | 0.767 | −0.190 | 1.45E−05 |
| | 120418965 | 120419058 | 120420715 | | | | | |
| RP11- | 72875284- | 72885773- | 72885895- | Exc | 0.366 | 0.205 | −0.162 | 2.32E−04 |
| 383H13.1 | 72885772 | 72885894 | 72964773 | | | | | |
| RP11- | 72875284- | 72877644- | 72877770- | Exc | 0.714 | 0.322 | −0.392 | 8.26E−31 |
| 383H13.1 | 72877643 | 72877769 | 72964773 | | | | | |
| RP11- | 9808576- | 9809164- | 9809294- | Unc | 0.108 | 0.115 | 0.007 | 9.25E−01 |
| 705C15.2 | 9809163 | 9809293 | 9809516 | | | | | |
| RP11- | 9808576- | 9809164- | 9809246- | Unc | 0.131 | 0.137 | 0.007 | 9.76E−01 |
| 705C15.2 | 9809163 | 9809245 | 9809516 | | | | | |
| RP11- | 81565879- | 81568515- | 81568589- | Exc | 0.886 | 0.668 | −0.218 | 3.45E−15 |
| 773D16.1 | 81568514 | 81568588 | 81574368 | | | | | |
| RP11- | 81565879- | 81568519- | 81568720- | Exc | 0.504 | 0.147 | −0.357 | 5.56E−10 |
| 773D16.1 | 81568518 | 81568719 | 81574368 | | | | | |
| RP1-178F15.4 | 153604717- | 153605512- | 153605634- | Unc | 0.221 | 0.228 | 0.008 | 5.93E−01 |
| | 153605511 | 153605633 | 153606433 | | | | | |
| RP13- | 19212123- | 19212958- | 19213006- | Exc | 0.958 | 0.792 | −0.166 | 4.30E−04 |
| 279N23.2 | 19212957 | 19213005 | 19215855 | | | | | |
| RPAIN | 5326150- | 5329556- | 5329620- | Exc | 0.369 | 0.208 | −0.160 | 5.41E−05 |
| | 5329555 | 5329619 | 5335861 | | | | | |
| RPAIN | 5329403- | 5329556- | 5329620- | Exc | 0.889 | 0.597 | −0.293 | 2.20E−13 |
| | 5329555 | 5329619 | 5335861 | | | | | |
| RPAIN | 5329403- | 5331391- | 5331532- | Exc | 0.808 | 0.420 | −0.388 | 3.96E−11 |
| | 5331390 | 5331531 | 5335861 | | | | | |
| RPAIN | 5326150- | 5329291- | 5329403- | Inc | 0.773 | 0.916 | 0.143 | 3.51E−08 |
| | 5329290 | 5329402 | 5329555 | | | | | |
| RPAIN | 5326150- | 5329291- | 5329620- | Unc | 0.544 | 0.540 | −0.004 | 9.53E−01 |
| | 5329290 | 5329619 | 5335861 | | | | | |
| RPAIN | 5329620- | 5331391- | 5331532- | Unc | 0.686 | 0.678 | −0.008 | 9.72E−01 |
| | 5331390 | 5331531 | 5335861 | | | | | |
| RPL7L1 | 42847690- | 42848599- | 42848705- | Exc | 0.836 | 0.697 | −0.139 | 3.61E−12 |
| | 42848598 | 42848704 | 42851188 | | | | | |
| RPL7L1 | 42847690- | 42848599- | 42848821- | Exc | 0.724 | 0.545 | −0.179 | 7.34E−10 |
| | 42848598 | 42848820 | 42851188 | | | | | |
| RPP14 | 58292145- | 58296017- | 58296134- | Exc | 0.532 | 0.269 | −0.263 | 4.78E−05 |
| | 58296016 | 58296133 | 58296233 | | | | | |
| RPP14 | 58292145- | 58296036- | 58296134- | Exc | 0.638 | 0.346 | −0.291 | 8.98E−07 |
| | 58296035 | 58296133 | 58296233 | | | | | |
| RPP38 | 15139409- | 15144217- | 15144336- | Unc | 0.547 | 0.545 | −0.001 | 9.89E−01 |
| | 15144216 | 15144335 | 15145303 | | | | | |
| RPS24 | 79797063- | 79799962- | 79799984- | Unc | 0.887 | 0.887 | 0.000 | 8.72E−01 |
| | 79799961 | 79799983 | 79800372 | | | | | |
| RPS24 | 79797063- | 79799959- | 79799984- | Unc | 0.788 | 0.787 | −0.001 | 8.72E−01 |
| | 79799958 | 79799983 | 79800372 | | | | | |
| RPS6KB2 | 67196095- | 67196453- | 67196494- | Exc | 0.977 | 0.869 | −0.108 | 3.73E−04 |
| | 67196452 | 67196493 | 67196590 | | | | | |
| RPS6KB2 | 67196095- | 67196384- | 67196494- | Exc | 0.965 | 0.797 | −0.167 | 3.88E−04 |
| | 67196383 | 67196493 | 67196590 | | | | | |
| RPUSD1 | 836929- | 837068- | 837180- | Unc | 0.884 | 0.885 | 0.001 | 9.88E−01 |
| | 837067 | 837179 | 837353 | | | | | |
| RRBP1 | 17641174- | 17660644- | 17660721- | Inc | 0.417 | 0.538 | 0.121 | 8.20E−09 |
| | 17660643 | 17660720 | 17662672 | | | | | |
| RRNAD1 | 156703313- | 156703801- | 156703845- | Unc | 0.517 | 0.523 | 0.006 | 9.83E−01 |
| | 156703800 | 156703844 | 156705516 | | | | | |
| RSU1 | 16824084- | 16858972- | 16859084- | Exc | 0.911 | 0.802 | −0.109 | 6.95E−22 |
| | 16858971 | 16859083 | 16859313 | | | | | |
| RTEL1 | 62325842- | 62326094- | 62326328- | Exc | 0.969 | 0.771 | −0.197 | 2.00E−04 |
| | 62326093 | 62326327 | 62326418 | | | | | |
| RTEL1- | 62325842- | 62326094- | 62326328- | Exc | 0.969 | 0.771 | −0.197 | 2.00E−04 |
| TNFRSF6B | 62326093 | 62326327 | 62326418 | | | | | |
| RTN2 | 45992812- | 45996418- | 45996637- | Unc | 0.584 | 0.577 | −0.008 | 9.89E−01 |
| | 45996417 | 45996636 | 45997423 | | | | | |
| RUFY2 | 70154209- | 70156537- | 70156639- | Exc | 0.884 | 0.699 | −0.185 | 1.17E−07 |
| | 70156536 | 70156638 | 70161376 | | | | | |
| RUNX1 | 36171760- | 36206707- | 36206899- | Unc | 0.804 | 0.812 | 0.008 | 7.25E−01 |
| | 36206706 | 36206898 | 36231770 | | | | | |
| RWDD1 | 116892819- | 116895221- | 116895335- | Inc | 0.291 | 0.554 | 0.263 | 7.98E−40 |
| | 116895220 | 116895334 | 116901457 | | | | | |
| RWDD2B | 30380445- | 30380561- | 30380629- | Exc | 0.853 | 0.706 | −0.147 | 1.60E−06 |
| | 30380560 | 30380628 | 30380715 | | | | | |
| RWDD4 | 184572285- | 184572371- | 184572481- | Exc | 0.976 | 0.815 | −0.162 | 1.52E−17 |
| | 184572370 | 184572480 | 184577033 | | | | | |
| RWDD4 | 184572285- | 184572371- | 184572477- | Exc | 0.963 | 0.742 | −0.221 | 1.04E−15 |
| | 184572370 | 184572476 | 184577033 | | | | | |
| SBF1 | 50895103- | 50895463- | 50895541- | Exc | 0.669 | 0.512 | −0.156 | 5.40E−05 |
| | 50895462 | 50895540 | 50897683 | | | | | |
| SCMH1 | 41617357- | 41625397- | 41625606- | Exc | 0.591 | 0.412 | −0.180 | 7.57E−04 |
| | 41625396 | 41625605 | 41626546 | | | | | |
| SCRN3 | 175260546- | 175262064- | 175262133- | Unc | 0.107 | 0.112 | 0.005 | 9.46E−01 |
| | 175262063 | 175262132 | 175263002 | | | | | |
| SDCCAG8 | 243456522- | 243456701- | 243456797- | Inc | 0.154 | 0.388 | 0.234 | 1.02E−09 |
| | 243456700 | 243456796 | 243468014 | | | | | |
| SEC13 | 10359779- | 10360823- | 10360857- | Unc | 0.268 | 0.273 | 0.005 | 7.94E−01 |
| | 10360822 | 10360856 | 10362192 | | | | | |
| SEC31A | 83778918- | 83783687- | 83783726- | Unc | 0.738 | 0.735 | −0.003 | 8.76E−01 |
| | 83783686 | 83783725 | 83784470 | | | | | |
| SEC31A | 83803094- | 83819142- | 83819216- | Unc | 0.873 | 0.868 | −0.004 | 8.31E−01 |
| | 83819141 | 83819215 | 83821229 | | | | | |
| SEC31B | 102249519- | 102249765- | 102250080- | Exc | 0.783 | 0.464 | −0.319 | 1.99E−05 |
| | 102249764 | 102250079 | 102250462 | | | | | |
| SECISBP2L | 49309826- | 49311615- | 49311750- | Exc | 0.734 | 0.533 | −0.201 | 1.87E−10 |
| | 49311614 | 49311749 | 49319561 | | | | | |
| SEMA4F | 74884752- | 74884980- | 74885079- | Exc | 0.302 | 0.016 | −0.286 | 8.40E−07 |
| | 74884979 | 74885078 | 74889858 | | | | | |
| SENP1 | 48459464- | 48460710- | 48460749- | Exc | 0.959 | 0.742 | −0.218 | 4.11E−09 |
| | 48460709 | 48460748 | 48465449 | | | | | |
| SENP6 | 76331342- | 76332467- | 76332575- | Exc | 0.227 | 0.126 | −0.101 | 1.01E−04 |
| | 76332466 | 76332574 | 76333615 | | | | | |
| SENP6 | 76344528- | 76350403- | 76350421- | Exc | 0.717 | 0.594 | −0.123 | 1.33E−04 |
| | 76350402 | 76350420 | 76357446 | | | | | |
| SENP6 | 76344528- | 76350400- | 76350421- | Exc | 0.708 | 0.568 | −0.140 | 1.63E−05 |
| | 76350399 | 76350420 | 76357446 | | | | | |
| SERAC1 | 158571622- | 158576347- | 158576384- | Exc | 0.783 | 0.575 | −0.208 | 7.72E−05 |
| | 158576346 | 158576383 | 158579304 | | | | | |
| SETD2 | 47108609- | 47122406- | 47122574- | Unc | 0.111 | 0.109 | −0.002 | 9.11E−01 |
| | 47122405 | 47122573 | 47125209 | | | | | |
| SETD5 | 9476170- | 9476274- | 9476315- | Exc | 0.328 | 0.138 | −0.190 | 2.82E−11 |
| | 9476273 | 9476314 | 9476507 | | | | | |
| SETX | 135140373- | 135144790- | 135144877- | Inc | 0.316 | 0.466 | 0.150 | 3.99E−06 |
| | 135144789 | 135144876 | 135145001 | | | | | |
| SEZ6L2 | 29883848- | 29884026- | 29884065- | Unc | 0.572 | 0.582 | 0.010 | 7.00E−01 |
| | 29884025 | 29884064 | 29884560 | | | | | |
| SFXN4 | 120920482- | 120920567- | 120920594- | Exc | 1.000 | 0.812 | −0.188 | 2.01E−06 |
| | 120920566 | 120920593 | 120921851 | | | | | |
| SGSM2 | 2268636- | 2270565- | 2270700- | Unc | 0.311 | 0.317 | 0.005 | 9.14E−01 |
| | 2270564 | 2270699 | 2274555 | | | | | |
| SGSM3 | 40796818- | 40797597- | 40797680- | Exc | 0.784 | 0.494 | −0.290 | 3.39E−19 |
| | 40797596 | 40797679 | 40798142 | | | | | |
| SH3D19 | 152065203- | 152065372- | 152065441- | Inc | 0.477 | 0.620 | 0.143 | 5.12E−10 |
| | 152065371 | 152065440 | 152069073 | | | | | |
| SHMT2 | 57624784- | 57625264- | 57625344- | Exc | 0.892 | 0.754 | −0.138 | 3.03E−40 |
| | 57625263 | 57625343 | 57625495 | | | | | |
| SIKE1 | 115321906- | 115322731- | 115322837- | Unc | 0.896 | 0.891 | −0.005 | 9.28E−01 |
| | 115322730 | 115322836 | 115323069 | | | | | |
| SIPA1L1 | 71787899- | 71867511- | 71867636- | Exc | 0.241 | 0.000 | −0.241 | 8.40E−12 |
| | 71867510 | 71867635 | 71979459 | | | | | |
| SIPA1L1 | 71867636- | 71880665- | 71880732- | Inc | 0.609 | 0.986 | 0.377 | 2.94E−08 |
| | 71880664 | 71880731 | 71979459 | | | | | |
| SLAIN2 | 48385802- | 48396593- | 48396671- | Unc | 0.726 | 0.717 | −0.009 | 7.85E−01 |
| | 48396592 | 48396670 | 48422141 | | | | | |
| SLC15A4 | 129294019- | 129294488- | 129294599- | Inc | 0.869 | 0.982 | 0.113 | 5.60E−08 |
| | 129294487 | 129294598 | 129299319 | | | | | |
| SLC22A17 | 23816422- | 23816723- | 23816941- | Exc | 0.855 | 0.684 | −0.171 | 3.27E−04 |
| | 23816722 | 23816940 | 23817369 | | | | | |
| SLC25A17 | 41175130- | 41188529- | 41188681- | Exc | 0.969 | 0.772 | −0.197 | 1.14E−05 |
| | 41188528 | 41188680 | 41195026 | | | | | |
| SLC25A17 | 41175130- | 41190518- | 41190585- | Exc | 0.966 | 0.769 | −0.197 | 4.59E−05 |
| | 41190517 | 41190584 | 41195026 | | | | | |
| SLC25A32 | 104415553- | 104417004- | 104417090- | Exc | 0.935 | 0.801 | −0.134 | 4.51E−09 |
| | 104417003 | 104417089 | 104419861 | | | | | |
| SLC25A36 | 140682070- | 140685842- | 140685879- | Exc | 0.273 | 0.073 | −0.201 | 3.17E−11 |
| | 140685841 | 140685878 | 140689768 | | | | | |
| SLC25A36 | 140682070- | 140685776- | 140685879- | Exc | 0.326 | 0.096 | −0.230 | 7.87E−13 |
| | 140685775 | 140685878 | 140689768 | | | | | |
| SLC30A6 | 32419053- | 32422413- | 32422462- | Unc | 0.781 | 0.790 | 0.009 | 9.62E−01 |
| | 32422412 | 32422461 | 32422775 | | | | | |
| SLC35A1 | 88182738- | 88187080- | 88187258- | Exc | 0.954 | 0.760 | −0.194 | 7.27E−08 |
| | 88187079 | 88187257 | 88210875 | | | | | |
| SLC35A3 | 100435719- | 100440527- | 100440626- | Inc | 0.377 | 0.613 | 0.237 | 4.81E−04 |
| | 100440526 | 100440625 | 100459092 | | | | | |
| SLC35D2 | 99083624- | 99084280- | 99084363- | Exc | 0.977 | 0.841 | −0.136 | 4.48E−07 |
| | 99084279 | 99084362 | 99086372 | | | | | |
| SLC37A3 | 140037150- | 140045669- | 140045771- | Exc | 0.790 | 0.548 | −0.241 | 8.26E−05 |
| | 140045668 | 140045770 | 140048425 | | | | | |
| SLC38A2 | 46761124- | 46764295- | 46764411- | Exc | 0.902 | 0.748 | −0.154 | 4.38E−139 |
| | 46764294 | 46764410 | 46764558 | | | | | |
| SLC38A6 | 61509931- | 61510168- | 61510222- | Exc | 0.865 | 0.653 | −0.212 | 4.01E−06 |
| | 61510167 | 61510221 | 61512063 | | | | | |
| SLC38A6 | 61512886- | 61517230- | 61517355- | Exc | 0.988 | 0.717 | −0.271 | 2.20E−16 |
| | 61517229 | 61517354 | 61518504 | | | | | |
| SLC38A6 | 61512886- | 61514869- | 61515016- | Inc | 0.033 | 0.190 | 0.157 | 1.74E−04 |
| | 61514868 | 61515015 | 61517229 | | | | | |
| SLC38A9 | 54993821- | 54998567- | 54998723- | Unc | 0.125 | 0.116 | −0.009 | 9.71E−01 |
| | 54998566 | 54998722 | 55007279 | | | | | |
| SLC39A9 | 69890920- | 69908786- | 69908984- | Unc | 0.879 | 0.883 | 0.004 | 8.49E−01 |
| | 69908785 | 69908983 | 69919957 | | | | | |
| SLC4A7 | 27446455- | 27450847- | 27451022- | Exc | 0.804 | 0.378 | −0.426 | 1.96E−29 |
| | 27450846 | 27451021 | 27453132 | | | | | |
| SLC5A6 | 27428962- | 27429351- | 27429403- | Exc | 0.938 | 0.763 | −0.174 | 5.73E−04 |
| | 27429350 | 27429402 | 27429744 | | | | | |
| SLC7A6 | 68300625- | 68307725- | 68307808- | Exc | 0.429 | 0.224 | −0.205 | 6.86E−07 |
| | 68307724 | 68307807 | 68308593 | | | | | |
| SLC7A6 | 68300625- | 68307721- | 68307808- | Exc | 0.542 | 0.312 | −0.230 | 6.22E−09 |
| | 68307720 | 68307807 | 68308593 | | | | | |
| SLC9B2 | 103947749- | 103949904- | 103950041- | Exc | 0.875 | 0.553 | −0.322 | 5.93E−13 |
| | 103949903 | 103950040 | 103952856 | | | | | |
| SLFN11 | 33690846- | 33693966- | 33694083- | Unc | 0.843 | 0.836 | −0.006 | 9.73E−01 |
| | 33693965 | 33694082 | 33700492 | | | | | |
| SLFN11 | 33690846- | 33693966- | 33694063- | Unc | 0.843 | 0.836 | −0.007 | 9.49E−01 |
| | 33693965 | 33694062 | 33700492 | | | | | |
| SLIT2 | 20547723- | 20550111- | 20550183- | Exc | 0.912 | 0.748 | −0.163 | 2.81E−17 |
| | 20550110 | 20550182 | 20550679 | | | | | |
| SLMAP | 57850444- | 57857363- | 57857426- | Exc | 0.606 | 0.339 | −0.267 | 2.38E−05 |
| | 57857362 | 57857425 | 57875767 | | | | | |
| SLMAP | 57882660- | 57893611- | 57893734- | Unc | 0.899 | 0.899 | 0.000 | 9.81E−01 |
| | 57893610 | 57893733 | 57894802 | | | | | |
| SLMO1 | 12421629- | 12427040- | 12427111- | Exc | 0.984 | 0.854 | −0.130 | 5.35E−06 |
| | 12427039 | 12427110 | 12427219 | | | | | |
| SLTM | 59193487- | 59204762- | 59204810- | Inc | 0.825 | 0.928 | 0.104 | 9.23E−05 |
| | 59204761 | 59204809 | 59209133 | | | | | |
| SLTM | 59191052- | 59191668- | 59192083- | Unc | 0.730 | 0.731 | 0.001 | 9.55E−01 |
| | 59191667 | 59192082 | 59193458 | | | | | |
| SLTM | 59191052- | 59191668- | 59192137- | Unc | 0.770 | 0.763 | −0.007 | 7.30E−01 |
| | 59191667 | 59192136 | 59193458 | | | | | |
| SMAD5 | 135468652- | 135483521- | 135483596- | Exc | 0.787 | 0.590 | −0.197 | 6.32E−07 |
| | 135483520 | 135483595 | 135489280 | | | | | |
| SMARCA1 | 128626071- | 128627017- | 128627053- | Inc | 0.618 | 0.870 | 0.253 | 3.54E−33 |
| | 128627016 | 128627052 | 128630726 | | | | | |
| SMARCD1 | 50480662- | 50481146- | 50481269- | Exc | 0.800 | 0.548 | −0.252 | 3.33E−18 |
| | 50481145 | 50481268 | 50482303 | | | | | |
| SMC5 | 72879362- | 72882839- | 72882892- | Exc | 0.979 | 0.854 | −0.125 | 5.98E−08 |
| | 72882838 | 72882891 | 72892225 | | | | | |
| SMEK2 | 55795500- | 55800755- | 55800914- | Exc | 0.955 | 0.819 | −0.136 | 1.39E−13 |
| | 55800754 | 55800913 | 55804450 | | | | | |
| SMEK2 | 55804493- | 55805383- | 55805479- | Unc | 0.450 | 0.445 | −0.005 | 9.40E−01 |
| | 55805382 | 55805478 | 55806814 | | | | | |
| SMG7 | 183441785- | 183471388- | 183471527- | Unc | 0.206 | 0.200 | −0.006 | 9.36E−01 |
| | 183471387 | 183471526 | 183481971 | | | | | |
| SMPDL3A | 123110604- | 123116822- | 123117036- | Exc | 0.855 | 0.702 | −0.152 | 1.92E−04 |
| | 123116821 | 123117035 | 123117968 | | | | | |
| SMURF2 | 62594609- | 62602720- | 62602759- | Exc | 0.892 | 0.742 | −0.150 | 9.59E−05 |
| | 62602719 | 62602758 | 62657946 | | | | | |
| SMURF2 | 62589692- | 62590109- | 62590223- | Unc | 0.111 | 0.111 | −0.001 | 8.17E−01 |
| | 62590108 | 62590222 | 62594499 | | | | | |
| SNAPC5 | 66786891- | 66787668- | 66787758- | Exc | 0.779 | 0.646 | −0.133 | 1.46E−04 |
| | 66787667 | 66787757 | 66789979 | | | | | |
| SNED1 | 241992744- | 242002208- | 242002322- | Exc | 0.880 | 0.660 | −0.220 | 9.72E−05 |
| | 242002207 | 242002321 | 242003003 | | | | | |
| SNHG14 | 25357042- | 25360446- | 25360804- | Exc | 0.979 | 0.860 | −0.119 | 1.90E−05 |
| | 25360445 | 25360803 | 25362556 | | | | | |
| SNHG14 | 25328675- | 25330337- | 25330470- | Unc | 0.851 | 0.852 | 0.001 | 9.66E−01 |
| | 25330336 | 25330469 | 25332613 | | | | | |
| SNHG15 | 45023646- | 45023956- | 45024026- | Exc | 0.960 | 0.832 | −0.127 | 1.03E−05 |
| | 45023955 | 45024025 | 45025619 | | | | | |
| SNRNP70 | 49604729- | 49605371- | 49606845- | Inc | 0.210 | 0.325 | 0.114 | 5.34E−16 |
| | 49605370 | 49606844 | 49607890 | | | | | |
| SNRNP70 | 49604729- | 49605371- | 49605431- | Inc | 0.201 | 0.302 | 0.102 | 2.96E−13 |
| | 49605370 | 49605430 | 49607890 | | | | | |
| SNRNP70 | 49604729- | 49605371- | 49605443- | Inc | 0.199 | 0.299 | 0.100 | 6.96E−13 |
| | 49605370 | 49605442 | 49607890 | | | | | |
| SNRPA1 | 101826007- | 101826419- | 101826499- | Exc | 0.708 | 0.499 | −0.209 | 1.53E−11 |
| | 101826418 | 101826498 | 101827112 | | | | | |
| SNRPG | 70515325- | 70516482- | 70516505- | Exc | 0.856 | 0.696 | −0.160 | 1.18E−28 |
| | 70516481 | 70516504 | 70520749 | | | | | |
| SNX14 | 86246643- | 86248556- | 86248583- | Exc | 0.829 | 0.668 | −0.160 | 6.31E−15 |
| | 86248555 | 86248582 | 86251702 | | | | | |
| SNX21 | 44463756- | 44468961- | 44469098- | Exc | 0.314 | 0.167 | −0.148 | 2.05E−04 |
| | 44468960 | 44469097 | 44469277 | | | | | |
| SNX21 | 44463756- | 44469087- | 44469098- | Exc | 0.423 | 0.263 | −0.160 | 6.59E−05 |
| | 44469086 | 44469097 | 44469277 | | | | | |
| SPATA20 | 48626548- | 48626647- | 48626849- | Exc | 0.979 | 0.856 | −0.123 | 9.61E−18 |
| | 48626646 | 48626848 | 48627345 | | | | | |
| SPATA20 | 48624647- | 48625026- | 48625129- | Exc | 0.948 | 0.815 | −0.133 | 1.65E−08 |
| | 48625025 | 48625128 | 48625643 | | | | | |
| SPATA20 | 48624647- | 48625081- | 48625129- | Exc | 0.943 | 0.792 | −0.151 | 1.04E−08 |
| | 48625080 | 48625128 | 48625643 | | | | | |
| SPATA20 | 48624647- | 48625026- | 48625316- | Exc | 0.790 | 0.531 | −0.259 | 8.43E−05 |
| | 48625025 | 48625315 | 48625643 | | | | | |
| SPATA20 | 48624647- | 48625081- | 48625316- | Exc | 0.647 | 0.347 | −0.300 | 9.22E−04 |
| | 48625080 | 48625315 | 48625643 | | | | | |
| SPATA7 | 88857800- | 88859737- | 88859833- | Inc | 0.670 | 0.925 | 0.255 | 4.37E−05 |
| | 88859736 | 88859832 | 88862499 | | | | | |
| SPDL1 | 169015580- | 169017757- | 169017834- | Inc | 0.107 | 0.355 | 0.248 | 7.98E−13 |
| | 169017756 | 169017833 | 169018051 | | | | | |
| SPDL1 | 169015580- | 169017760- | 169017834- | Inc | 0.104 | 0.345 | 0.241 | 1.54E−12 |
| | 169017759 | 169017833 | 169018051 | | | | | |
| SPEN | 16199632- | 16202697- | 16203174- | Unc | 0.859 | 0.861 | 0.002 | 9.99E−01 |
| | 16202696 | 16203173 | 16235815 | | | | | |
| SPIDR | 48206620- | 48320423- | 48320524- | Unc | 0.885 | 0.878 | −0.007 | 9.47E−01 |
| | 48320422 | 48320523 | 48352884 | | | | | |
| SPPL2A | 51017521- | 51018270- | 51018324- | Inc | 0.176 | 0.554 | 0.378 | 4.89E−81 |
| | 51018269 | 51018323 | 51018517 | | | | | |
| SPTAN1 | 131353905- | 131355262- | 131355322- | Exc | 0.289 | 0.040 | −0.249 | 1.83E−69 |
| | 131355261 | 131355321 | 131356453 | | | | | |
| SRSF2 | 74731241- | 74731854- | 74731958- | Unc | 0.112 | 0.113 | 0.001 | 9.79E−01 |
| | 74731853 | 74731957 | 74732235 | | | | | |
| ST6GALNAC6 | 130658612- | 130660235- | 130660290- | Inc | 0.529 | 0.659 | 0.130 | 3.79E−04 |
| | 130660234 | 130660289 | 130661781 | | | | | |
| ST7 | 116849992- | 116859138- | 116859231- | Exc | 0.994 | 0.857 | −0.137 | 3.24E−05 |
| | 116859137 | 116859230 | 116869815 | | | | | |
| ST7 | 116849992- | 116861977- | 116862117- | Exc | 0.989 | 0.771 | −0.218 | 7.60E−05 |
| | 116861976 | 116862116 | 116869815 | | | | | |
| STAG1 | 136261038- | 136287607- | 136287704- | Unc | 0.878 | 0.885 | 0.007 | 7.91E−01 |
| | 136287606 | 136287703 | 136323150 | | | | | |
| STAG2 | 123094717- | 123155217- | 123155282- | Unc | 0.278 | 0.279 | 0.002 | 9.96E−01 |
| | 123155216 | 123155281 | 123156380 | | | | | |
| STARD3 | 37814776- | 37814962- | 37815074- | Unc | 0.862 | 0.871 | 0.009 | 8.06E−01 |
| | 37814961 | 37815073 | 37815303 | | | | | |
| STARD3NL | 38218023- | 38218692- | 38218748- | Inc | 0.087 | 0.195 | 0.108 | 1.74E−04 |
| | 38218691 | 38218747 | 38247047 | | | | | |
| STARD4 | 110836815- | 110837660- | 110837787- | Unc | 0.769 | 0.760 | −0.008 | 9.68E−01 |
| | 110837659 | 110837786 | 110842027 | | | | | |
| STARD5 | 81614882- | 81615240- | 81615290- | Exc | 0.949 | 0.780 | −0.169 | 1.97E−04 |
| | 81615239 | 81615289 | 81616142 | | | | | |
| STAT6 | 57501098- | 57501442- | 57501527- | Exc | 0.856 | 0.732 | −0.124 | 1.48E−08 |
| | 57501441 | 57501526 | 57501945 | | | | | |
| STK16 | 220111599- | 220111835- | 220111969- | Exc | 0.919 | 0.703 | −0.216 | 4.52E−20 |
| | 220111834 | 220111968 | 220112136 | | | | | |
| STK19 | 31946776- | 31947191- | 31947331- | Exc | 0.943 | 0.773 | −0.171 | 2.51E−11 |
| | 31947190 | 31947330 | 31948227 | | | | | |
| STK38L | 27455122- | 27461272- | 27461395- | Unc | 0.907 | 0.898 | −0.008 | 9.94E−01 |
| | 27461271 | 27461394 | 27462046 | | | | | |
| STK40 | 36826942- | 36833453- | 36833686- | Inc | 0.242 | 0.551 | 0.309 | 7.98E−08 |
| | 36833452 | 36833685 | 36851323 | | | | | |
| STK40 | 36826942- | 36833449- | 36833686- | Inc | 0.137 | 0.358 | 0.221 | 1.21E−04 |
| | 36833448 | 36833685 | 36851323 | | | | | |
| STOML1 | 74281144- | 74281371- | 74281599- | Unc | 0.817 | 0.818 | 0.001 | 8.95E−01 |
| | 74281370 | 74281598 | 74282691 | | | | | |
| STOML1 | 74281144- | 74281449- | 74281599- | Unc | 0.901 | 0.898 | −0.004 | 8.91E−01 |
| | 74281448 | 74281598 | 74282691 | | | | | |
| STRA13 | 79977258- | 79977386- | 79977571- | Exc | 0.368 | 0.142 | −0.226 | 1.53E−08 |
| | 79977385 | 79977570 | 79977733 | | | | | |
| STRA13 | 79977258- | 79977517- | 79977571- | Exc | 0.510 | 0.249 | −0.261 | 8.77E−11 |
| | 79977516 | 79977570 | 79977733 | | | | | |
| STRADA | 61784100- | 61784607- | 61784779- | Exc | 0.961 | 0.833 | −0.129 | 8.80E−06 |
| | 61784606 | 61784778 | 61787850 | | | | | |
| STRN3 | 31382864- | 31388172- | 31388313- | Inc | 0.077 | 0.187 | 0.110 | 1.98E−05 |
| | 31388171 | 31388312 | 31404368 | | | | | |
| STX16 | 57243184- | 57244347- | 57244510- | Exc | 0.822 | 0.495 | −0.327 | 9.78E−39 |
| | 57244346 | 57244509 | 57245567 | | | | | |
| STX16 | 57243184- | 57244358- | 57244510- | Exc | 0.715 | 0.335 | −0.380 | 1.01E−32 |
| | 57244357 | 57244509 | 57245567 | | | | | |
| STX16- | 57243184- | 57244347- | 57244510- | Exc | 0.822 | 0.495 | −0.327 | 9.78E−39 |
| NPEPL1 | 57244346 | 57244509 | 57245567 | | | | | |
| STX3 | 59562956- | 59564756- | 59564870- | Exc | 0.868 | 0.629 | −0.239 | 1.40E−09 |
| | 59564755 | 59564869 | 59568327 | | | | | |
| STXBP3 | 109319046- | 109321908- | 109322033- | Exc | 0.990 | 0.862 | −0.128 | 6.63E−12 |
| | 109321907 | 109322032 | 109325043 | | | | | |
| STXBP5 | 147660375- | 147660473- | 147660521- | Unc | 0.829 | 0.827 | −0.002 | 9.55E−01 |
| | 147660472 | 147660520 | 147674458 | | | | | |
| STYXL1 | 75634723- | 75643060- | 75643206- | Exc | 0.749 | 0.572 | −0.177 | 1.03E−06 |
| | 75643059 | 75643205 | 75651168 | | | | | |
| SUCO | 172520767- | 172522400- | 172522511- | Inc | 0.605 | 0.967 | 0.362 | 2.47E−27 |
| | 172522399 | 172522510 | 172525008 | | | | | |
| SUGP2 | 19101959- | 19104457- | 19104550- | Inc | 0.791 | 0.902 | 0.111 | 1.27E−03 |
| | 19104456 | 19104549 | 19105174 | | | | | |
| SUGP2 | 19101959- | 19104447- | 19104550- | Inc | 0.802 | 0.908 | 0.106 | 9.46E−04 |
| | 19104446 | 19104549 | 19105174 | | | | | |
| SUGT1 | 53233385- | 53235610- | 53235706- | Exc | 0.411 | 0.285 | −0.126 | 1.95E−07 |
| | 53235609 | 53235705 | 53236783 | | | | | |
| SULF1 | 70379186- | 70408000- | 70408162- | Exc | 0.652 | 0.446 | −0.206 | 1.38E−87 |
| | 70407999 | 70408161 | 70414108 | | | | | |
| SULF1 | 70541915- | 70550737- | 70550880- | Exc | 0.911 | 0.702 | −0.209 | 0.00E+00 |
| | 70550736 | 70550879 | 70550969 | | | | | |
| SUN2 | 39148671- | 39150647- | 39150712- | Exc | 0.353 | 0.221 | −0.132 | 1.12E−03 |
| | 39150646 | 39150711 | 39151767 | | | | | |
| SUOX | 56391124- | 56391399- | 56391508- | Unc | 0.631 | 0.626 | −0.005 | 9.30E−01 |
| | 56391398 | 56391507 | 56395995 | | | | | |
| SUPT20H | 37622074- | 37622701- | 37622737- | Exc | 0.766 | 0.609 | −0.157 | 4.61E−05 |
| | 37622700 | 37622736 | 37625624 | | | | | |
| SUPT20H | 37583947- | 37584689- | 37584793- | Inc | 0.420 | 0.779 | 0.359 | 3.13E−21 |
| | 37584688 | 37584792 | 37586328 | | | | | |
| SYNE1 | 152615263- | 152621776- | 152621917- | Unc | 0.784 | 0.778 | −0.006 | 6.13E−01 |
| | 152621775 | 152621916 | 152623003 | | | | | |
| SYNJ2BP | 70842489- | 70855187- | 70855324- | Unc | 0.876 | 0.869 | −0.007 | 8.71E−01 |
| | 70855186 | 70855323 | 70883616 | | | | | |
| SYNRG | 35879175- | 35880282- | 35880318- | Inc | 0.460 | 0.836 | 0.377 | 2.57E−20 |
| | 35880281 | 35880317 | 35880640 | | | | | |
| TAF1 | 70678217- | 70679000- | 70679102- | Exc | 0.253 | 0.082 | −0.171 | 1.03E−07 |
| | 70678999 | 70679101 | 70679401 | | | | | |
| TAF2 | 120756634- | 120757121- | 120757277- | Inc | 0.069 | 0.176 | 0.107 | 1.20E−04 |
| | 120757120 | 120757276 | 120758944 | | | | | |
| TAMM41 | 11871339- | 11874477- | 11874626- | Unc | 0.124 | 0.122 | −0.002 | 9.01E−01 |
| | 11874476 | 11874625 | 11880695 | | | | | |
| TANGO2 | 20043537- | 20049053- | 20049230- | Exc | 0.985 | 0.855 | −0.130 | 2.86E−05 |
| | 20049052 | 20049229 | 20050860 | | | | | |
| TANGO2 | 20040108- | 20040883- | 20041075- | Exc | 0.456 | 0.293 | −0.163 | 4.78E−05 |
| | 20040882 | 20041074 | 20043465 | | | | | |
| TANGO2 | 20040108- | 20040960- | 20041075- | Exc | 0.585 | 0.412 | −0.173 | 2.36E−06 |
| | 20040959 | 20041074 | 20043465 | | | | | |
| TARBP2 | 53898600- | 53898919- | 53899047- | Exc | 0.810 | 0.650 | −0.160 | 1.16E−04 |
| | 53898918 | 53899046 | 53899432 | | | | | |
| TAS2R14 | 11126321- | 11187381- | 11187485- | Exc | 0.455 | 0.165 | −0.290 | 5.30E−09 |
| | 11187380 | 11187484 | 11199618 | | | | | |
| TAZ | 153647963- | 153648044- | 153648086- | Exc | 0.874 | 0.620 | −0.254 | 1.12E−05 |
| | 153648043 | 153648085 | 153648370 | | | | | |
| TBC1D14 | 7008454- | 7011604- | 7011676- | Exc | 0.857 | 0.729 | −0.128 | 2.05E−06 |
| | 7011603 | 7011675 | 7012379 | | | | | |
| TBC1D17 | 50386150- | 50386230- | 50386334- | Exc | 0.994 | 0.892 | −0.102 | 3.08E−11 |
| | 50386229 | 50386333 | 50386907 | | | | | |
| TBC1D25 | 48399831- | 48403257- | 48403412- | Exc | 0.691 | 0.459 | −0.232 | 3.35E−04 |
| | 48403256 | 48403411 | 48417284 | | | | | |
| TBC1D32 | 121482203- | 121491551- | 121491674- | Unc | 0.890 | 0.885 | −0.006 | 9.13E−01 |
| | 121491550 | 121491673 | 121526220 | | | | | |
| TBC1D5 | 17550098- | 17665341- | 17665406- | Exc | 0.806 | 0.498 | −0.308 | 3.67E−13 |
| | 17665340 | 17665405 | 17783972 | | | | | |
| TBCE | 235543465- | 235564818- | 235564903- | Unc | 0.805 | 0.814 | 0.009 | 9.68E−01 |
| | 235564817 | 235564902 | 235577747 | | | | | |
| TBCEL | 120918377- | 120924260- | 120924442- | Unc | 0.813 | 0.805 | −0.008 | 7.85E−01 |
| | 120924259 | 120924441 | 120925760 | | | | | |
| TBCK | 107114928- | 107115875- | 107115912- | Exc | 0.936 | 0.549 | −0.387 | 6.40E−56 |
| | 107115874 | 107115911 | 107133906 | | | | | |
| TBCK | 107173165- | 107176104- | 107176205- | Inc | 0.047 | 0.406 | 0.360 | 4.86E−30 |
| | 107176103 | 107176204 | 107181593 | | | | | |
| TBPL1 | 134273869- | 134274323- | 134274564- | Inc | 0.633 | 0.887 | 0.253 | 9.06E−04 |
| | 134274322 | 134274563 | 134301219 | | | | | |
| TBX15 | 119466227- | 119467269- | 119467441- | Exc | 0.972 | 0.815 | −0.157 | 3.14E−06 |
| | 119467268 | 119467440 | 119469132 | | | | | |
| TCERG1 | 145888809- | 145889630- | 145889724- | Exc | 0.227 | 0.120 | −0.107 | 9.87E−04 |
| | 145889629 | 145889723 | 145890003 | | | | | |
| TCF20 | 42557365- | 42564615- | 42564743- | Unc | 0.243 | 0.233 | −0.010 | 9.77E−01 |
| | 42564614 | 42564742 | 42565852 | | | | | |
| TCF7L2 | 114917829- | 114918426- | 114918477- | Unc | 0.770 | 0.780 | 0.010 | 7.78E−01 |
| | 114918425 | 114918476 | 114920377 | | | | | |
| TCF7L2 | 114917829- | 114919679- | 114919752- | Unc | 0.516 | 0.525 | 0.009 | 9.75E−01 |
| | 114919678 | 114919751 | 114920377 | | | | | |
| TCF7L2 | 114917829- | 114919679- | 114919752- | Unc | 0.338 | 0.334 | −0.004 | 6.21E−01 |
| | 114919678 | 114919751 | 114925313 | | | | | |
| TEAD2 | 49858677- | 49859216- | 49859228- | Exc | 0.413 | 0.183 | −0.230 | 6.40E−11 |
| | 49859215 | 49859227 | 49860508 | | | | | |
| TENM2 | 167182198- | 167302991- | 167303201- | Unc | 0.873 | 0.878 | 0.005 | 6.28E−01 |
| | 167302990 | 167303200 | 167379592 | | | | | |
| TENM2 | 167631627- | 167638739- | 167638760- | Unc | 0.143 | 0.138 | −0.005 | 8.43E−01 |
| | 167638738 | 167638759 | 167642044 | | | | | |
| TEP1 | 20849561- | 20849712- | 20849846- | Exc | 0.602 | 0.343 | −0.258 | 1.09E−05 |
| | 20849711 | 20849845 | 20850071 | | | | | |
| TEP1 | 20839792- | 20840892- | 20841017- | Exc | 0.758 | 0.466 | −0.293 | 1.63E−09 |
| | 20840891 | 20841016 | 20841169 | | | | | |
| TET2 | 106068137- | 106111517- | 106111663- | Unc | 0.732 | 0.724 | −0.008 | 9.36E−01 |
| | 106111516 | 106111662 | 106155053 | | | | | |
| TFB2M | 246714605- | 246719875- | 246720024- | Unc | 0.873 | 0.864 | −0.009 | 9.05E−01 |
| | 246719874 | 246720023 | 246727647 | | | | | |
| TFDP1 | 114277602- | 114285938- | 114286060- | Exc | 0.923 | 0.820 | −0.103 | 5.57E−17 |
| | 114285937 | 114286059 | 114287434 | | | | | |
| TFDP1 | 114277602- | 114285938- | 114286221- | Exc | 0.853 | 0.685 | −0.168 | 1.24E−15 |
| | 114285937 | 114286220 | 114287434 | | | | | |
| TFDP2 | 141724387- | 141772957- | 141772992- | Exc | 0.260 | 0.116 | −0.144 | 1.48E−04 |
| | 141772956 | 141772991 | 141811902 | | | | | |
| TFDP2 | 141697525- | 141712380- | 141712428- | Unc | 0.864 | 0.869 | 0.005 | 9.77E−01 |
| | 141712379 | 141712427 | 141724282 | | | | | |
| TFDP2 | 141697525- | 141713862- | 141713984- | Unc | 0.857 | 0.847 | −0.010 | 6.43E−01 |
| | 141713861 | 141713983 | 141724282 | | | | | |
| TFPI | 188368498- | 188394091- | 188394213- | Exc | 0.214 | 0.058 | −0.156 | 1.45E−10 |
| | 188394090 | 188394212 | 188418926 | | | | | |
| TGFBR2 | 30648470- | 30664691- | 30664766- | Inc | 0.247 | 0.361 | 0.114 | 1.28E−15 |
| | 30664690 | 30664765 | 30686238 | | | | | |
| TGIF1 | 3450069- | 3450323- | 3450504- | Unc | 0.727 | 0.736 | 0.009 | 6.27E−01 |
| | 3450322 | 3450503 | 3456351 | | | | | |
| THADA | 43625279- | 43657352- | 43657442- | Unc | 0.858 | 0.859 | 0.000 | 9.41E−01 |
| | 43657351 | 43657441 | 43712372 | | | | | |
| THAP6 | 76442190- | 76446946- | 76447072- | Exc | 0.949 | 0.794 | −0.155 | 2.70E−04 |
| | 76446945 | 76447071 | 76452169 | | | | | |
| THAP9-AS1 | 83816928- | 83819142- | 83819216- | Inc | 0.865 | 0.971 | 0.106 | 2.38E−05 |
| | 83819141 | 83819215 | 83821229 | | | | | |
| THBS3 | 155173097- | 155173304- | 155173331- | Exc | 0.984 | 0.861 | −0.123 | 1.84E−05 |
| | 155173303 | 155173330 | 155174850 | | | | | |
| THBS3 | 155169905- | 155170242- | 155170402- | Exc | 0.930 | 0.744 | −0.186 | 2.81E−24 |
| | 155170241 | 155170401 | 155170687 | | | | | |
| THOC2 | 122757135- | 122757495- | 122757561- | Exc | 0.188 | 0.077 | −0.111 | 1.23E−03 |
| | 122757494 | 122757560 | 122757637 | | | | | |
| THTPA | 24025553- | 24025952- | 24026249- | Unc | 0.291 | 0.297 | 0.005 | 9.10E−01 |
| | 24025951 | 24026248 | 24027903 | | | | | |
| THTPA | 24025553- | 24025952- | 24026244- | Unc | 0.270 | 0.263 | −0.008 | 9.94E−01 |
| | 24025951 | 24026243 | 24027903 | | | | | |
| TIA1 | 70454955- | 70455476- | 70455595- | Inc | 0.210 | 0.532 | 0.322 | 1.88E−12 |
| | 70455475 | 70455594 | 70456190 | | | | | |
| TIA1 | 70451762- | 70452460- | 70452526- | Inc | 0.123 | 0.374 | 0.251 | 2.51E−14 |
| | 70452459 | 70452525 | 70454866 | | | | | |
| TIAL1 | 121339523- | 121339983- | 121340359- | Inc | 0.068 | 0.278 | 0.210 | 5.41E−34 |
| | 121339982 | 121340358 | 121341433 | | | | | |
| TIAL1 | 121339523- | 121339983- | 121340051- | Inc | 0.072 | 0.178 | 0.106 | 8.55E−12 |
| | 121339982 | 121340050 | 121341433 | | | | | |
| TIMM17B | 48752385- | 48752635- | 48752785- | Inc | 0.105 | 0.216 | 0.111 | 1.53E−05 |
| | 48752634 | 48752784 | 48754041 | | | | | |
| TIMM21 | 71816345- | 71822380- | 71822443- | Exc | 1.000 | 0.836 | −0.164 | 2.93E−14 |
| | 71822379 | 71822442 | 71822540 | | | | | |
| TJAP1 | 43445971- | 43446397- | 43446457- | Unc | 0.181 | 0.180 | −0.001 | 9.49E−01 |
| | 43446396 | 43446456 | 43465618 | | | | | |
| TJP1 | 30011343- | 30011981- | 30012221- | Unc | 0.317 | 0.310 | −0.007 | 9.42E−01 |
| | 30011980 | 30012220 | 30012561 | | | | | |
| TLK2 | 60600539- | 60601597- | 60601693- | Unc | 0.413 | 0.423 | 0.010 | 9.00E−01 |
| | 60601596 | 60601692 | 60613530 | | | | | |
| TM7SF3 | 27128592- | 27129193- | 27129291- | Inc | 0.836 | 0.966 | 0.129 | 2.07E−09 |
| | 27129192 | 27129290 | 27133498 | | | | | |
| TMCO4 | 20067435- | 20072025- | 20072145- | Exc | 0.711 | 0.348 | −0.364 | 6.18E−05 |
| | 20072024 | 20072144 | 20072948 | | | | | |
| TMEM11 | 21102154- | 21114249- | 21114541- | Exc | 0.327 | 0.206 | −0.121 | 1.46E−05 |
| | 21114248 | 21114540 | 21117403 | | | | | |
| TMEM119 | 108986174- | 108987940- | 108988322- | Unc | 0.306 | 0.302 | −0.004 | 6.73E−01 |
| | 108987939 | 108988321 | 108991745 | | | | | |
| TMEM120A | 75616747- | 75616856- | 75616921- | Exc | 0.963 | 0.574 | −0.389 | 1.44E−31 |
| | 75616855 | 75616920 | 75617035 | | | | | |
| TMEM126B | 85339733- | 85342189- | 85342361- | Inc | 0.622 | 0.779 | 0.157 | 7.27E−10 |
| | 85342188 | 85342360 | 85342730 | | | | | |
| TMEM126B | 85339733- | 85340176- | 85340307- | Inc | 0.053 | 0.198 | 0.145 | 1.01E−05 |
| | 85340175 | 85340306 | 85342730 | | | | | |
| TMEM161B | 87524346- | 87536630- | 87536734- | Unc | 0.824 | 0.818 | −0.006 | 8.70E−01 |
| | 87536629 | 87536733 | 87564537 | | | | | |
| TMEM175 | 926329- | 941497- | 941943- | Unc | 0.531 | 0.525 | −0.007 | 9.38E−01 |
| | 941496 | 941942 | 944208 | | | | | |
| TMEM18 | 675631- | 675758- | 676239- | Inc | 0.104 | 0.246 | 0.143 | 1.52E−06 |
| | 675757 | 676238 | 677288 | | | | | |
| TMEM194A | 57458523- | 57463017- | 57463090- | Exc | 0.855 | 0.568 | −0.287 | 1.77E−04 |
| | 57463016 | 57463089 | 57464602 | | | | | |
| TMEM230 | 5090092- | 5092146- | 5092252- | Unc | 0.508 | 0.505 | −0.003 | 9.86E−01 |
| | 5092145 | 5092251 | 5093388 | | | | | |
| TMEM234 | 32682953- | 32683038- | 32683179- | Inc | 0.094 | 0.210 | 0.116 | 9.14E−05 |
| | 32683037 | 32683178 | 32686731 | | | | | |
| TMEM260 | 57085482- | 57088249- | 57088421- | Exc | 0.884 | 0.727 | −0.157 | 9.42E−04 |
| | 57088248 | 57088420 | 57092099 | | | | | |
| TMEM260 | 57082746- | 57083901- | 57084016- | Exc | 0.964 | 0.804 | −0.161 | 1.21E−05 |
| | 57083900 | 57084015 | 57085311 | | | | | |
| TMEM39B | 32541424- | 32542765- | 32542920- | Exc | 0.772 | 0.276 | −0.496 | 2.72E−08 |
| | 32542764 | 32542919 | 32557275 | | | | | |
| TMEM62 | 43461876- | 43470805- | 43470910- | Exc | 0.978 | 0.786 | −0.192 | 7.18E−07 |
| | 43470804 | 43470909 | 43473378 | | | | | |
| TMOD2 | 52073372- | 52074918- | 52075026- | Exc | 0.993 | 0.862 | −0.131 | 1.39E−04 |
| | 52074917 | 52075025 | 52090393 | | | | | |
| TMUB2 | 42264478- | 42265044- | 42265112- | Exc | 0.898 | 0.675 | −0.223 | 4.21E−06 |
| | 42265043 | 42265111 | 42265274 | | | | | |
| TMUB2 | 42266668- | 42266779- | 42266957- | Unc | 0.846 | 0.849 | 0.004 | 9.41E−01 |
| | 42266778 | 42266956 | 42267868 | | | | | |
| TMUB2 | 42266668- | 42266792- | 42266957- | Unc | 0.844 | 0.848 | 0.003 | 9.51E−01 |
| | 42266791 | 42266956 | 42267868 | | | | | |
| TMX3 | 66367723- | 66368982- | 66369028- | Exc | 0.967 | 0.821 | −0.146 | 5.98E−29 |
| | 66368981 | 66369027 | 66377257 | | | | | |
| TOM1 | 35713955- | 35717952- | 35718013- | Exc | 0.931 | 0.817 | −0.114 | 2.97E−10 |
| | 35717951 | 35718012 | 35719020 | | | | | |
| TOR1AIP2 | 179821947- | 179834571- | 179834990- | Unc | 0.810 | 0.814 | 0.005 | 9.93E−01 |
| | 179834570 | 179834989 | 179846373 | | | | | |
| TOR3A | 179052189- | 179054763- | 179055029- | Unc | 0.886 | 0.890 | 0.004 | 9.77E−01 |
| | 179054762 | 179055028 | 179057045 | | | | | |
| TP53BP2 | 224002056- | 224005908- | 224006040- | Unc | 0.108 | 0.118 | 0.010 | 8.29E−01 |
| | 224005907 | 224006039 | 224008921 | | | | | |
| TP53INP1 | 95942957- | 95944298- | 95944336- | Exc | 0.573 | 0.361 | −0.212 | 1.18E−09 |
| | 95944297 | 95944335 | 95952087 | | | | | |
| TP53TG1 | 86971001- | 86974359- | 86974550- | Exc | 0.583 | 0.415 | −0.168 | 1.27E−04 |
| | 86974358 | 86974549 | 86974620 | | | | | |
| TPD52L1 | 125569530- | 125578244- | 125578305- | Inc | 0.338 | 0.440 | 0.102 | 1.32E−04 |
| | 125578243 | 125578304 | 125583979 | | | | | |
| TPD52L2 | 62505170- | 62507169- | 62507229- | Exc | 0.868 | 0.741 | −0.126 | 3.05E−32 |
| | 62507168 | 62507228 | 62514071 | | | | | |
| TPRA1 | 127294349- | 127294592- | 127294653- | Exc | 0.909 | 0.621 | −0.288 | 4.41E−39 |
| | 127294591 | 127294652 | 127294782 | | | | | |
| TPT1-AS1 | 45957371- | 45957895- | 45957974- | Inc | 0.259 | 0.514 | 0.255 | 6.01E−04 |
| | 45957894 | 45957973 | 45963869 | | | | | |
| TPT1-AS1 | 45963956- | 45964893- | 45965038- | Inc | 0.792 | 0.941 | 0.149 | 2.04E−04 |
| | 45964892 | 45965037 | 45965166 | | | | | |
| TPT1-AS1 | 45963956- | 45964849- | 45965038- | Inc | 0.835 | 0.950 | 0.116 | 4.16E−04 |
| | 45964848 | 45965037 | 45965166 | | | | | |
| TRA2A | 23561460- | 23561740- | 23562052- | Unc | 0.101 | 0.104 | 0.003 | 9.31E−01 |
| | 23561739 | 23562051 | 23571407 | | | | | |
| TRAPPC12 | 3469467- | 3481463- | 3481567- | Exc | 0.973 | 0.858 | −0.115 | 6.99E−05 |
| | 3481462 | 3481566 | 3482616 | | | | | |
| TREX2 | 152719967- | 152720335- | 152720512- | Exc | 0.465 | 0.347 | −0.119 | 9.05E−04 |
| | 152720334 | 152720511 | 152720999 | | | | | |
| TRIM16 | 15546131- | 15554405- | 15555261- | Inc | 0.791 | 0.955 | 0.164 | 1.23E−05 |
| | 15554404 | 15555260 | 15580489 | | | | | |
| TRIM37 | 57076821- | 57078959- | 57079103- | Unc | 0.841 | 0.835 | −0.005 | 9.69E−01 |
| | 57078958 | 57079102 | 57089688 | | | | | |
| TRIM65 | 73887429- | 73887894- | 73887960- | Inc | 0.430 | 0.645 | 0.214 | 1.65E−04 |
| | 73887893 | 73887959 | 73888091 | | | | | |
| TRIO | 14502767- | 14504502- | 14504703- | Exc | 0.987 | 0.870 | −0.117 | 1.47E−17 |
| | 14504501 | 14504702 | 14507230 | | | | | |
| TRIP10 | 6745006- | 6746040- | 6746208- | Exc | 0.353 | 0.207 | −0.146 | 9.68E−08 |
| | 6746039 | 6746207 | 6746462 | | | | | |
| TRIP12 | 230724291- | 230725122- | 230725248- | Inc | 0.564 | 0.760 | 0.195 | 1.78E−18 |
| | 230725121 | 230725247 | 230744697 | | | | | |
| TRMU | 46751486- | 46751887- | 46751970- | Unc | 0.826 | 0.836 | 0.010 | 9.49E−01 |
| | 46751886 | 46751969 | 46752738 | | | | | |
| TROVE2 | 193038765- | 193039684- | 193039750- | Inc | 0.039 | 0.308 | 0.269 | 3.64E−15 |
| | 193039683 | 193039749 | 193044949 | | | | | |
| TROVE2 | 193028907- | 193029004- | 193029353- | Unc | 0.156 | 0.148 | −0.008 | 9.76E−01 |
| | 193029003 | 193029352 | 193038163 | | | | | |
| TRPC1 | 142510001- | 142511666- | 142511810- | Exc | 0.886 | 0.735 | −0.151 | 1.77E−04 |
| | 142511665 | 142511809 | 142521010 | | | | | |
| TRPM4 | 49661517- | 49669298- | 49669473- | Exc | 1.000 | 0.794 | −0.206 | 4.10E−04 |
| | 49669297 | 49669472 | 49671516 | | | | | |
| TRPT1 | 63991440- | 63991572- | 63991683- | Exc | 0.955 | 0.795 | −0.160 | 2.78E−12 |
| | 63991571 | 63991682 | 63991756 | | | | | |
| TRPT1 | 63991440- | 63991572- | 63991635- | Exc | 0.927 | 0.704 | −0.223 | 2.99E−11 |
| | 63991571 | 63991634 | 63991756 | | | | | |
| TSEN15 | 184023998- | 184041291- | 184041433- | Exc | 0.766 | 0.618 | −0.149 | 5.24E−09 |
| | 184041290 | 184041432 | 184041960 | | | | | |
| TSEN15 | 184023998- | 184041291- | 184041329- | Exc | 0.636 | 0.451 | −0.186 | 4.32E−08 |
| | 184041290 | 184041328 | 184041960 | | | | | |
| TSEN15 | 184023998- | 184039750- | 184039867- | Inc | 0.032 | 0.171 | 0.139 | 1.01E−12 |
| | 184039749 | 184039866 | 184041290 | | | | | |
| TSPAN5 | 99403327- | 99407889- | 99408036- | Unc | 0.851 | 0.857 | 0.006 | 8.52E−01 |
| | 99407888 | 99408035 | 99428828 | | | | | |
| TSR1 | 2239025- | 2239331- | 2239435- | Exc | 0.967 | 0.825 | −0.142 | 2.43E−09 |
| | 2239330 | 2239434 | 2239624 | | | | | |
| TSTD3 | 99973986- | 99979239- | 99979413- | Exc | 0.692 | 0.476 | −0.216 | 3.86E−05 |
| | 99979238 | 99979412 | 99979507 | | | | | |
| TUBG2 | 40812726- | 40814991- | 40815071- | Exc | 0.929 | 0.811 | −0.119 | 1.94E−04 |
| | 40814990 | 40815070 | 40815394 | | | | | |
| TULP3 | 3043728- | 3046797- | 3046896- | Exc | 0.947 | 0.671 | −0.276 | 1.33E−24 |
| | 3046796 | 3046895 | 3047279 | | | | | |
| TULP3 | 3018747- | 3029929- | 3030089- | Unc | 0.766 | 0.765 | −0.001 | 9.89E−01 |
| | 3029928 | 3030088 | 3031427 | | | | | |
| TXNDC11 | 11792182- | 11794308- | 11794421- | Exc | 0.897 | 0.758 | −0.139 | 3.52E−06 |
| | 11794307 | 11794420 | 11815432 | | | | | |
| TXNDC11 | 11815527- | 11824501- | 11824631- | Exc | 0.896 | 0.728 | −0.168 | 8.33E−07 |
| | 11824500 | 11824630 | 11827837 | | | | | |
| TXNL4A | 77737702- | 77746602- | 77746751- | Inc | 0.109 | 0.212 | 0.102 | 2.71E−13 |
| | 77746601 | 77746750 | 77748239 | | | | | |
| U2AF1 | 44520630- | 44521476- | 44521543- | Unc | 0.143 | 0.143 | 0.001 | 9.72E−01 |
| | 44521475 | 44521542 | 44524424 | | | | | |
| U2AF1L4 | 36235323- | 36235527- | 36235640- | Exc | 0.519 | 0.093 | −0.426 | 1.77E−15 |
| | 36235526 | 36235639 | 36236025 | | | | | |
| U2AF1L4 | 36235323- | 36235527- | 36235594- | Exc | 0.538 | 0.066 | −0.471 | 1.56E−18 |
| | 36235526 | 36235593 | 36236025 | | | | | |
| UACA | 70968931- | 70969447- | 70969480- | Exc | 0.936 | 0.753 | −0.183 | 3.76E−17 |
| | 70969446 | 70969479 | 70970437 | | | | | |
| UBA7 | 49849977- | 49850081- | 49850172- | Exc | 0.971 | 0.826 | −0.145 | 6.17E−08 |
| | 49850080 | 49850171 | 49850494 | | | | | |
| UBE2A | 118709364- | 118715470- | 118715560- | Exc | 0.886 | 0.683 | −0.203 | 1.92E−45 |
| | 118715469 | 118715559 | 118716550 | | | | | |
| UBE2D1 | 60121162- | 60121255- | 60121287- | Exc | 0.992 | 0.865 | −0.126 | 1.19E−14 |
| | 60121254 | 60121286 | 60123368 | | | | | |
| UBN2 | 138921844- | 138936702- | 138936804- | Exc | 0.910 | 0.653 | −0.257 | 4.56E−04 |
| | 138936701 | 138936803 | 138943233 | | | | | |
| UBP1 | 33444397- | 33450182- | 33450290- | Inc | 0.356 | 0.568 | 0.212 | 2.16E−16 |
| | 33450181 | 33450289 | 33450727 | | | | | |
| UBQLN1 | 86280061- | 86281265- | 86281349- | Exc | 0.813 | 0.711 | −0.102 | 1.70E−10 |
| | 86281264 | 86281348 | 86284099 | | | | | |
| UBQLN1 | 86280061- | 86281265- | 86281310- | Exc | 0.687 | 0.557 | −0.130 | 2.08E−08 |
| | 86281264 | 86281309 | 86284099 | | | | | |
| UCHL5 | 192993077- | 192997005- | 192997086- | Unc | 0.119 | 0.122 | 0.003 | 9.32E−01 |
| | 192997004 | 192997085 | 192997214 | | | | | |
| UFD1L | 19459332- | 19462993- | 19463126- | Exc | 0.821 | 0.658 | −0.164 | 4.55E−06 |
| | 19462992 | 19463125 | 19466605 | | | | | |
| UNC5B | 73048490- | 73048715- | 73048748- | Exc | 0.964 | 0.839 | −0.125 | 2.93E−08 |
| | 73048714 | 73048747 | 73050671 | | | | | |
| UPF3A | 115048419- | 115051734- | 115051876- | Unc | 0.532 | 0.530 | −0.002 | 9.49E−01 |
| | 115051733 | 115051875 | 115051993 | | | | | |
| UPF3A | 115048419- | 115051777- | 115051876- | Unc | 0.630 | 0.622 | −0.008 | 9.23E−01 |
| | 115051776 | 115051875 | 115051993 | | | | | |
| UPP1 | 48134425- | 48141421- | 48141468- | Unc | 0.195 | 0.185 | −0.010 | 7.14E−01 |
| | 48141420 | 48141467 | 48146469 | | | | | |
| URGCP | 43927085- | 43927389- | 43927416- | Exc | 0.925 | 0.790 | −0.136 | 8.53E−05 |
| | 43927388 | 43927415 | 43946160 | | | | | |
| URGCP | 43927085- | 43927389- | 43927411- | Exc | 0.900 | 0.708 | −0.191 | 2.51E−05 |
| | 43927388 | 43927410 | 43946160 | | | | | |
| USMG5 | 105152224- | 105153956- | 105154152- | Exc | 0.530 | 0.311 | −0.220 | 1.87E−06 |
| | 105153955 | 105154151 | 105155502 | | | | | |
| USP24 | 55557800- | 55558494- | 55558590- | Exc | 0.990 | 0.881 | −0.108 | 2.57E−08 |
| | 55558493 | 55558589 | 55559609 | | | | | |
| USP25 | 17177578- | 17181128- | 17181205- | Exc | 0.983 | 0.835 | −0.148 | 1.91E−13 |
| | 17181127 | 17181204 | 17183455 | | | | | |
| USP3 | 63824907- | 63826002- | 63826118- | Unc | 0.101 | 0.094 | −0.006 | 8.98E−01 |
| | 63826001 | 63826117 | 63829223 | | | | | |
| USP33 | 78207434- | 78211106- | 78211285- | Inc | 0.414 | 0.558 | 0.144 | 9.65E−05 |
| | 78211105 | 78211284 | 78225327 | | | | | |
| USP53 | 120134029- | 120135225- | 120135378- | Exc | 0.631 | 0.332 | −0.300 | 1.17E−16 |
| | 120135224 | 120135377 | 120138704 | | | | | |
| USP8 | 50774263- | 50776472- | 50776559- | Inc | 0.466 | 0.594 | 0.127 | 1.87E−06 |
| | 50776471 | 50776558 | 50781997 | | | | | |
| VAMP7 | 155119276- | 155125286- | 155125344- | Exc | 0.957 | 0.830 | −0.127 | 4.67E−13 |
| | 155125285 | 155125343 | 155127775 | | | | | |
| VCAN | 82808216- | 82815168- | 82818129- | Exc | 0.685 | 0.357 | −0.328 | 7.07E−14 |
| | 82815167 | 82818128 | 82841355 | | | | | |
| VDR | 48272899- | 48276477- | 48276558- | Unc | 0.878 | 0.876 | −0.002 | 7.96E−01 |
| | 48276476 | 48276557 | 48298737 | | | | | |
| VEGFA | 43746656- | 43748469- | 43748592- | Unc | 0.115 | 0.123 | 0.009 | 8.73E−01 |
| | 43748468 | 43748591 | 43749692 | | | | | |
| VEGFA | 43746656- | 43748469- | 43748523- | Unc | 0.140 | 0.145 | 0.005 | 9.83E−01 |
| | 43748468 | 43748522 | 43749692 | | | | | |
| VEZT | 95645848- | 95650326- | 95650399- | Inc | 0.510 | 0.668 | 0.157 | 2.20E−07 |
| | 95650325 | 95650398 | 95650925 | | | | | |
| VIPAS39 | 77920446- | 77923438- | 77923757- | Unc | 0.130 | 0.125 | −0.005 | 9.28E−01 |
| | 77923437 | 77923756 | 77923837 | | | | | |
| VPS13D | 12395885- | 12398288- | 12398363- | Unc | 0.126 | 0.117 | −0.009 | 9.68E−01 |
| | 12398287 | 12398362 | 12401836 | | | | | |
| VPS29 | 110934009- | 110937340- | 110937352- | Inc | 0.356 | 0.468 | 0.113 | 3.40E−06 |
| | 110937339 | 110937351 | 110939853 | | | | | |
| WARS | 100828259- | 100841620- | 100841744- | Unc | 0.507 | 0.515 | 0.007 | 9.35E−01 |
| | 100841619 | 100841743 | 100842596 | | | | | |
| WARS | 100835596- | 100840473- | 100840582- | Unc | 0.249 | 0.240 | −0.009 | 5.31E−01 |
| | 100840472 | 100840581 | 100841619 | | | | | |
| WBP1 | 74685799- | 74686605- | 74686690- | Exc | 0.239 | 0.112 | −0.127 | 6.92E−05 |
| | 74686604 | 74686689 | 74686769 | | | | | |
| WDFY2 | 52234800- | 52249306- | 52249380- | Exc | 0.833 | 0.606 | −0.226 | 2.21E−06 |
| | 52249305 | 52249379 | 52277731 | | | | | |
| WDR27 | 170062521- | 170063659- | 170063746- | Unc | 0.353 | 0.358 | 0.005 | 9.09E−01 |
| | 170063658 | 170063745 | 170064261 | | | | | |
| WHSC1 | 1936990- | 1941381- | 1941506- | Unc | 0.739 | 0.741 | 0.001 | 9.07E−01 |
| | 1941380 | 1941505 | 1952798 | | | | | |
| WNK1 | 971437- | 980431- | 980515- | Unc | 0.768 | 0.770 | 0.002 | 9.77E−01 |
| | 980430 | 980514 | 987380 | | | | | |
| XIAP | 122993756- | 123019481- | 123020390- | Unc | 0.893 | 0.896 | 0.003 | 9.33E−01 |
| | 123019480 | 123020389 | 123022468 | | | | | |
| XPNPEP3 | 41265120- | 41266052- | 41266144- | Unc | 0.230 | 0.223 | −0.008 | 9.33E−01 |
| | 41266051 | 41266143 | 41277773 | | | | | |
| YEATS2 | 183476754- | 183479295- | 183479403- | Exc | 1.000 | 0.892 | −0.108 | 1.05E−06 |
| | 183479294 | 183479402 | 183479884 | | | | | |
| YIPF1 | 54354660- | 54354917- | 54355137- | Exc | 0.384 | 0.070 | −0.314 | 1.42E−15 |
| | 54354916 | 54355136 | 54355385 | | | | | |
| YME1L1 | 27425315- | 27431316- | 27431415- | Exc | 0.897 | 0.774 | −0.123 | 6.50E−26 |
| | 27431315 | 27431414 | 27434356 | | | | | |
| YTHDF2 | 29064851- | 29068915- | 29070499- | Unc | 0.891 | 0.896 | 0.006 | 7.03E−01 |
| | 29068914 | 29070498 | 29095440 | | | | | |
| YWHAB | 43514528- | 43516186- | 43516384- | Unc | 0.100 | 0.103 | 0.003 | 7.26E−01 |
| | 43516185 | 43516383 | 43530171 | | | | | |
| YWHAB | 43514528- | 43516289- | 43516384- | Unc | 0.151 | 0.150 | −0.001 | 8.69E−01 |
| | 43516288 | 43516383 | 43530171 | | | | | |
| YY1AP1 | 155649304- | 155650207- | 155650248- | Exc | 0.923 | 0.816 | −0.107 | 4.12E−04 |
| | 155650206 | 155650247 | 155657861 | | | | | |
| YY1AP1 | 155649304- | 155650173- | 155650248- | Exc | 0.845 | 0.673 | −0.172 | 1.08E−03 |
| | 155650172 | 155650247 | 155657861 | | | | | |
| ZBTB38 | 141122874- | 141157110- | 141157208- | Unc | 0.214 | 0.213 | 0.000 | 9.84E−01 |
| | 141157109 | 141157207 | 141161230 | | | | | |
| ZBTB38 | 141122874- | 141157085- | 141157208- | Unc | 0.211 | 0.202 | −0.009 | 7.68E−01 |
| | 141157084 | 141157207 | 141161230 | | | | | |
| ZBTB8OS | 33093146- | 33097428- | 33097481- | Exc | 0.713 | 0.581 | −0.132 | 9.29E−05 |
| | 33097427 | 33097480 | 33099245 | | | | | |
| ZC2HC1A | 79610749- | 79624225- | 79624342- | Inc | 0.062 | 0.219 | 0.156 | 3.70E−06 |
| | 79624224 | 79624341 | 79627455 | | | | | |
| ZC3H11A | 203764923- | 203765421- | 203765730- | Inc | 0.201 | 0.555 | 0.354 | 2.19E−13 |
| | 203765420 | 203765729 | 203770702 | | | | | |
| ZC3H11A | 203764923- | 203765421- | 203765625- | Inc | 0.351 | 0.673 | 0.322 | 5.26E−13 |
| | 203765420 | 203765624 | 203770702 | | | | | |
| ZCCHC6 | 88932192- | 88933873- | 88933974- | Exc | 0.924 | 0.819 | −0.104 | 2.10E−04 |
| | 88933872 | 88933973 | 88934498 | | | | | |
| ZCCHC6 | 88940430- | 88941286- | 88941407- | Unc | 0.159 | 0.154 | −0.005 | 9.59E−01 |
| | 88941285 | 88941406 | 88943254 | | | | | |
| ZCCHC8 | 122974011- | 122975009- | 122975115- | Exc | 0.977 | 0.868 | −0.110 | 1.26E−03 |
| | 122975008 | 122975114 | 122977261 | | | | | |
| ZDHHC16 | 99213421- | 99213556- | 99213604- | Exc | 0.810 | 0.587 | −0.223 | 1.09E−12 |
| | 99213555 | 99213603 | 99214470 | | | | | |
| ZDHHC20 | 21950795- | 21952800- | 21952878- | Inc | 0.085 | 0.281 | 0.197 | 1.13E−23 |
| | 21952799 | 21952877 | 21955572 | | | | | |
| ZDHHC7 | 85015601- | 85022369- | 85022480- | Exc | 0.536 | 0.415 | −0.121 | 3.36E−08 |
| | 85022368 | 85022479 | 85023909 | | | | | |
| ZEB1 | 31676196- | 31676649- | 31676728- | Unc | 0.233 | 0.235 | 0.002 | 9.27E−01 |
| | 31676648 | 31676727 | 31749965 | | | | | |
| ZFAND1 | 82627350- | 82629484- | 82629524- | Exc | 0.621 | 0.382 | −0.239 | 4.40E−12 |
| | 82629483 | 82629523 | 82630416 | | | | | |
| ZFAND5 | 74975704- | 74978386- | 74978523- | Exc | 0.559 | 0.450 | −0.109 | 4.90E−08 |
| | 74978385 | 74978522 | 74979611 | | | | | |
| ZFAS1 | 47897108- | 47897440- | 47897502- | Exc | 0.660 | 0.536 | −0.125 | 2.58E−49 |
| | 47897439 | 47897501 | 47905581 | | | | | |
| ZFAT | 135621123- | 135622713- | 135622899- | Unc | 0.792 | 0.792 | 0.000 | 9.29E−01 |
| | 135622712 | 135622898 | 135649703 | | | | | |
| ZFX | 24170948- | 24179832- | 24179960- | Unc | 0.264 | 0.261 | −0.003 | 9.35E−01 |
| | 24179831 | 24179959 | 24190831 | | | | | |
| ZMIZ1 | 81067329- | 81070681- | 81070942- | Exc | 0.635 | 0.512 | −0.124 | 1.40E−05 |
| | 81070680 | 81070941 | 81072398 | | | | | |
| ZMYM1 | 35545044- | 35558921- | 35559043- | Unc | 0.546 | 0.542 | −0.004 | 9.83E−01 |
| | 35558920 | 35559042 | 35559514 | | | | | |
| ZMYM4 | 35734687- | 35790961- | 35791007- | Exc | 0.939 | 0.631 | −0.309 | 4.74E−12 |
| | 35790960 | 35791006 | 35824525 | | | | | |
| ZMYM5 | 20426331- | 20436539- | 20436607- | Exc | 0.911 | 0.689 | −0.222 | 6.25E−05 |
| | 20436538 | 20436606 | 20437589 | | | | | |
| ZMYM5 | 20409830- | 20411796- | 20411962- | Exc | 0.914 | 0.557 | −0.358 | 1.76E−06 |
| | 20411795 | 20411961 | 20425494 | | | | | |
| ZNF140 | 133660148- | 133677573- | 133677640- | Unc | 0.502 | 0.500 | −0.002 | 9.78E−01 |
| | 133677572 | 133677639 | 133682095 | | | | | |
| ZNF160 | 53589575- | 53594666- | 53594783- | Exc | 0.605 | 0.362 | −0.244 | 3.05E−04 |
| | 53594665 | 53594782 | 53594889 | | | | | |
| ZNF207 | 30687987- | 30688487- | 30688535- | Exc | 0.935 | 0.477 | −0.459 | 1.66E−186 |
| | 30688486 | 30688534 | 30689932 | | | | | |
| ZNF207 | 30692507- | 30693684- | 30693777- | Inc | 0.629 | 0.872 | 0.243 | 9.12E−73 |
| | 30693683 | 30693776 | 30694790 | | | | | |
| ZNF217 | 52185804- | 52188260- | 52188393- | Unc | 0.836 | 0.835 | −0.001 | 9.72E−01 |
| | 52188259 | 52188392 | 52192265 | | | | | |
| ZNF248 | 38145266- | 38145363- | 38145461- | Exc | 0.985 | 0.762 | −0.223 | 1.31E−04 |
| | 38145362 | 38145460 | 38146112 | | | | | |
| ZNF260 | 37006602- | 37016057- | 37016276- | Unc | 0.879 | 0.886 | 0.008 | 8.79E−01 |
| | 37016056 | 37016275 | 37019120 | | | | | |
| ZNF266 | 9530895- | 9544741- | 9544912- | Unc | 0.564 | 0.568 | 0.004 | 8.76E−01 |
| | 9544740 | 9544911 | 9545473 | | | | | |
| ZNF280D | 56996466- | 56999280- | 56999349- | Exc | 0.954 | 0.846 | −0.108 | 6.55E−04 |
| | 56999279 | 56999348 | 56999460 | | | | | |
| ZNF280D | 56996466- | 56999280- | 56999342- | Exc | 0.942 | 0.822 | −0.120 | 9.46E−04 |
| | 56999279 | 56999341 | 56999460 | | | | | |
| ZNF3 | 99675057- | 99677159- | 99677280- | Unc | 0.818 | 0.824 | 0.007 | 9.83E−01 |
| | 99677158 | 99677279 | 99679257 | | | | | |
| ZNF346 | 176471535- | 176477752- | 176477938- | Unc | 0.903 | 0.899 | −0.004 | 9.51E−01 |
| | 176477751 | 176477937 | 176489058 | | | | | |
| ZNF37A | 38383419- | 38383859- | 38384153- | Unc | 0.530 | 0.521 | −0.009 | 9.87E−01 |
| | 38383858 | 38384152 | 38384465 | | | | | |
| ZNF384 | 6787627- | 6787829- | 6787877- | Exc | 0.781 | 0.593 | −0.188 | 2.07E−08 |
| | 6787828 | 6787876 | 6788111 | | | | | |
| ZNF419 | 58002966- | 58003481- | 58003580- | Unc | 0.617 | 0.624 | 0.007 | 9.78E−01 |
| | 58003480 | 58003579 | 58004223 | | | | | |
| ZNF426 | 9640313- | 9641661- | 9641744- | Unc | 0.898 | 0.901 | 0.003 | 8.37E−01 |
| | 9641660 | 9641743 | 9643520 | | | | | |
| ZNF512 | 27822921- | 27823558- | 27823683- | Exc | 0.970 | 0.865 | −0.106 | 1.55E−05 |
| | 27823557 | 27823682 | 27824222 | | | | | |
| ZNF516 | 74074513- | 74082483- | 74082551- | Inc | 0.831 | 1.000 | 0.169 | 7.19E−08 |
| | 74082482 | 74082550 | 74083421 | | | | | |
| ZNF529 | 37039225- | 37042779- | 37042880- | Unc | 0.104 | 0.111 | 0.008 | 9.72E−01 |
| | 37042778 | 37042879 | 37045571 | | | | | |
| ZNF532 | 56530983- | 56532536- | 56532618- | Inc | 0.796 | 0.928 | 0.132 | 9.81E−04 |
| | 56532535 | 56532617 | 56532701 | | | | | |
| ZNF585B | 37681053- | 37689841- | 37689957- | Unc | 0.897 | 0.896 | −0.001 | 9.20E−01 |
| | 37689840 | 37689956 | 37697941 | | | | | |
| ZNF638 | 71558996- | 71575557- | 71577402- | Unc | 0.860 | 0.858 | −0.001 | 9.30E−01 |
| | 71575556 | 71577401 | 71582848 | | | | | |
| ZNF655 | 99158319- | 99159511- | 99160118- | Unc | 0.765 | 0.762 | −0.003 | 9.61E−01 |
| | 99159510 | 99160117 | 99161485 | | | | | |
| ZNF720 | 31765222- | 31770678- | 31770788- | Exc | 0.925 | 0.725 | −0.200 | 9.99E−04 |
| | 31770677 | 31770787 | 31803950 | | | | | |
| ZNF75A | 3355644- | 3358313- | 3358837- | Unc | 0.782 | 0.778 | −0.004 | 9.35E−01 |
| | 3358312 | 3358836 | 3361752 | | | | | |
| ZNF767 | 149317129- | 149318137- | 149318264- | Unc | 0.844 | 0.835 | −0.009 | 9.22E−01 |
| | 149318136 | 149318263 | 149318350 | | | | | |
| ZSCAN32 | 3440518- | 3443649- | 3443815- | Unc | 0.854 | 0.852 | −0.001 | 9.08E−01 |
| | 3443648 | 3443814 | 3447191 | | | | | |
| ZZZ3 | 78105288- | 78107069- | 78107132- | Exc | 0.980 | 0.864 | −0.117 | 2.20E−08 |
| | 78107068 | 78107131 | 78107206 | | | | | |

Differential Splicing Analysis to Identify Drug-Responding Sequences from RNA Sequencing

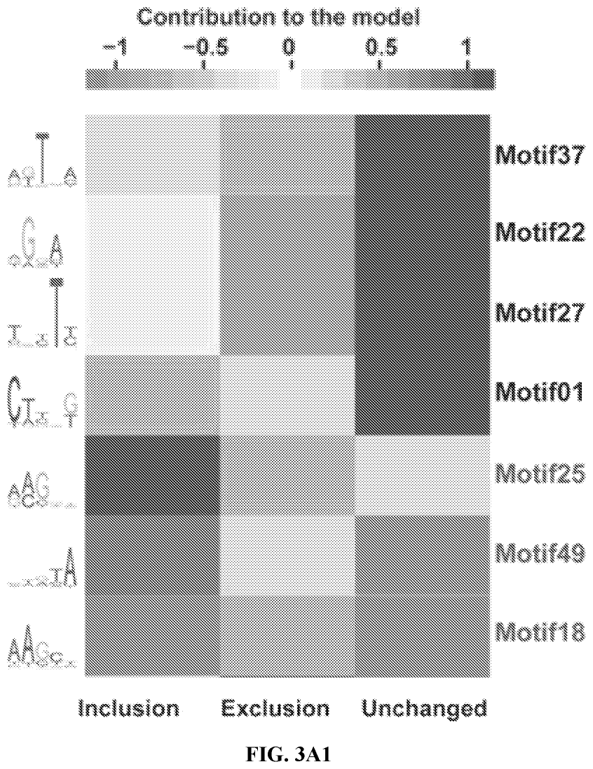

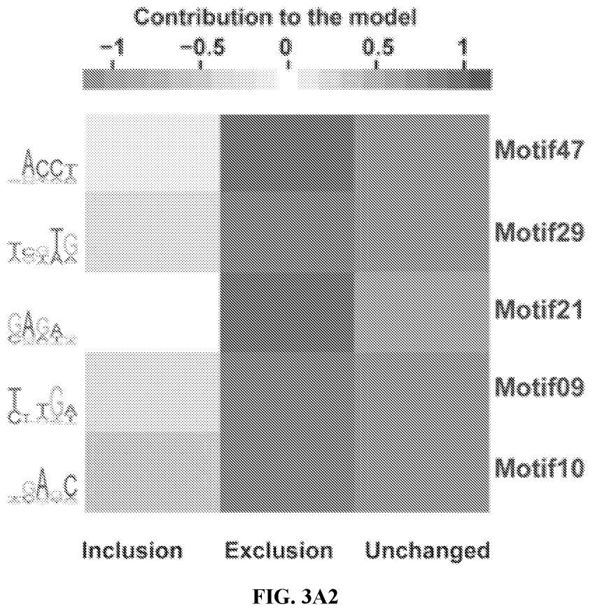

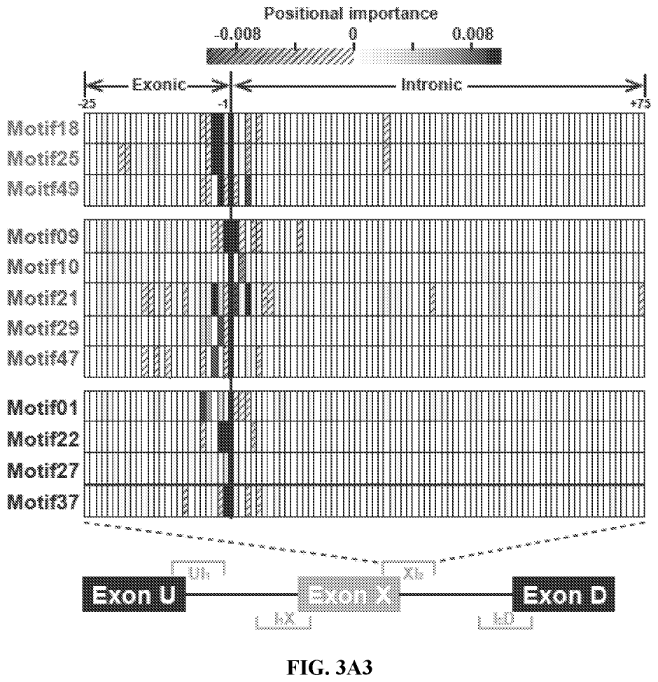

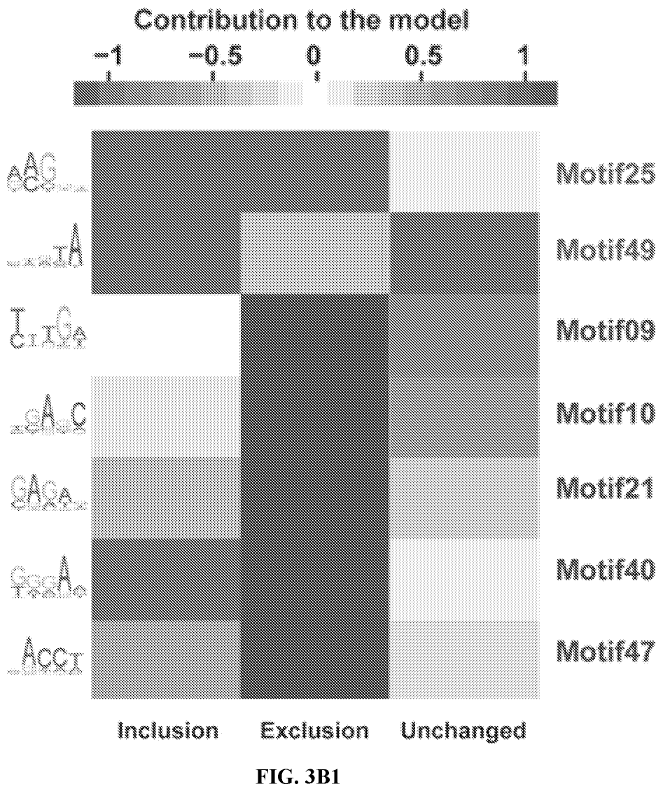

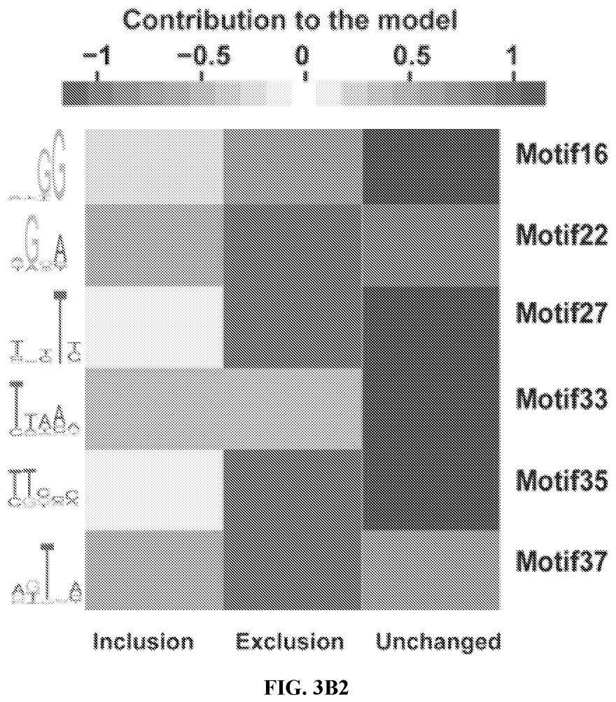

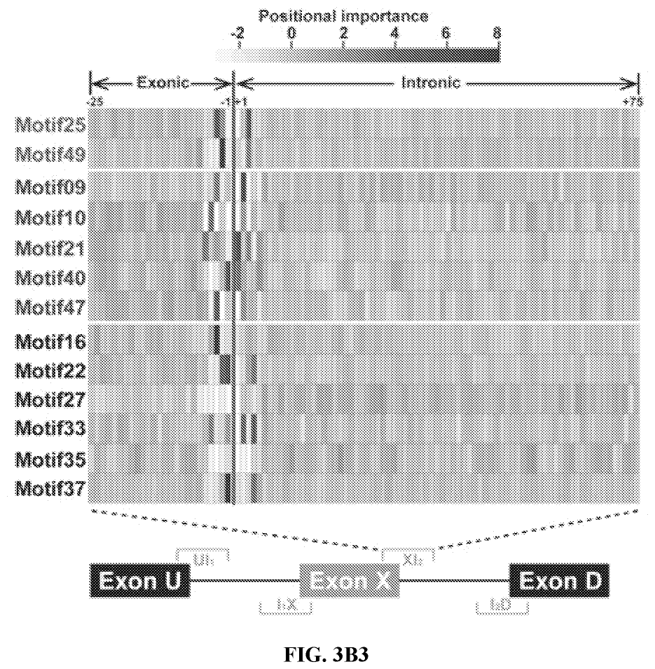

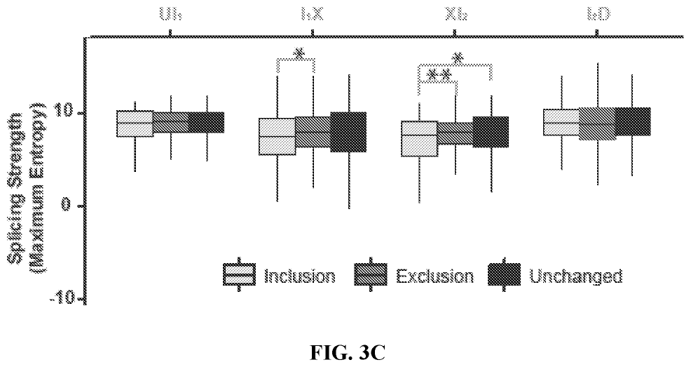

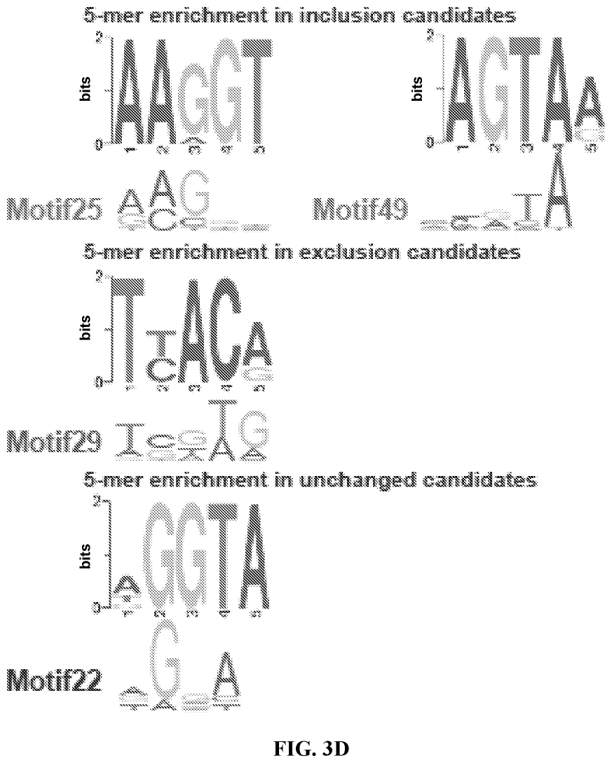

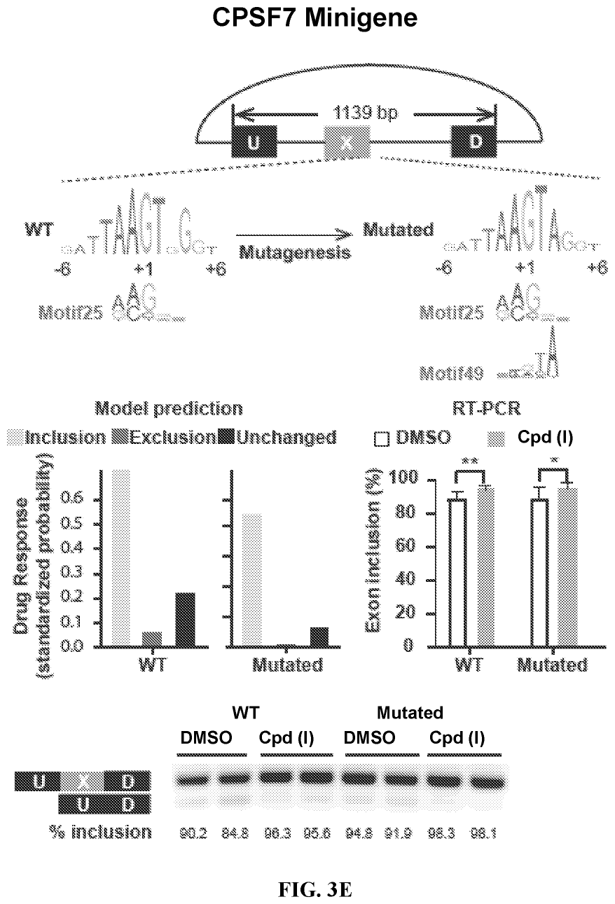

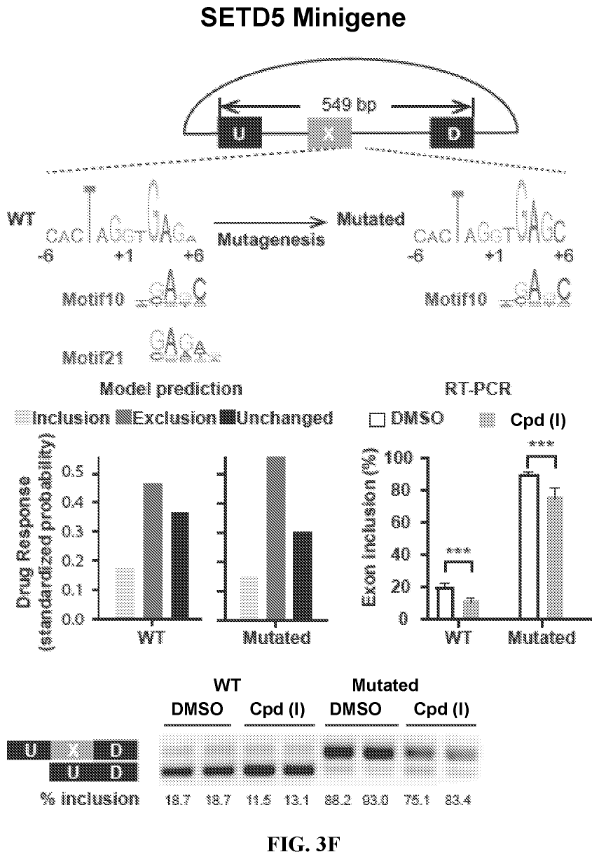

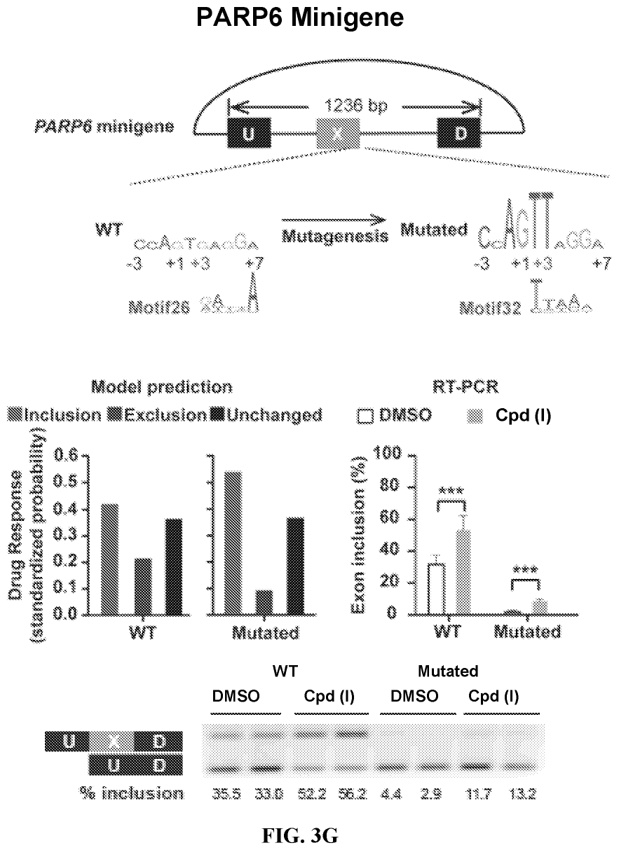

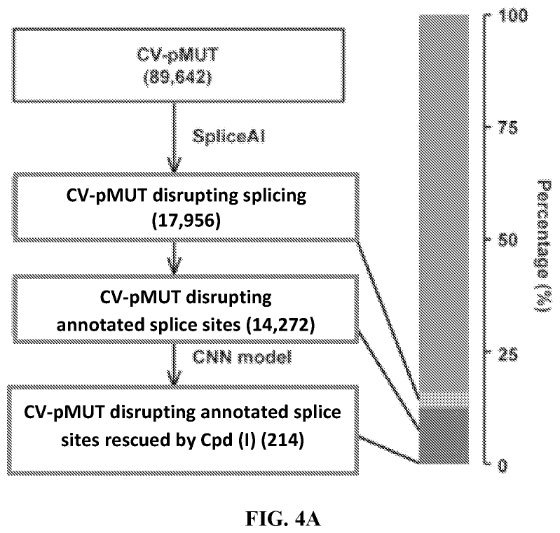

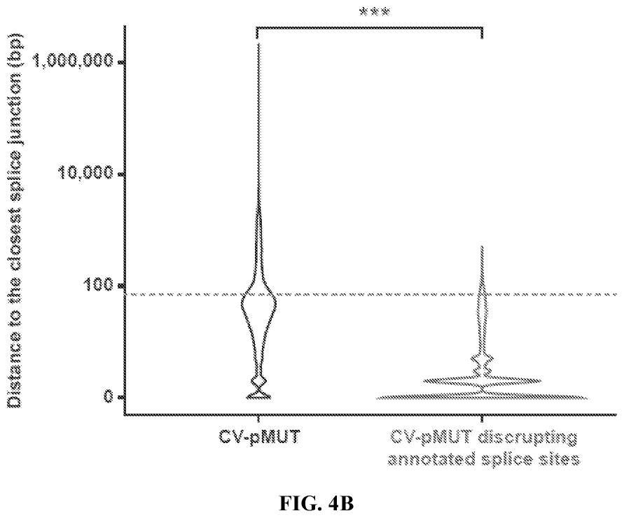

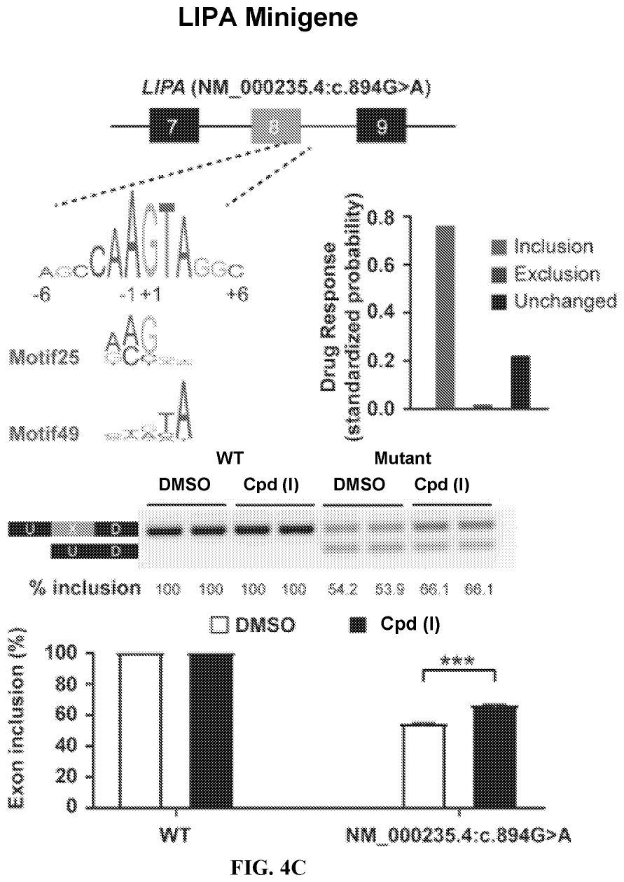

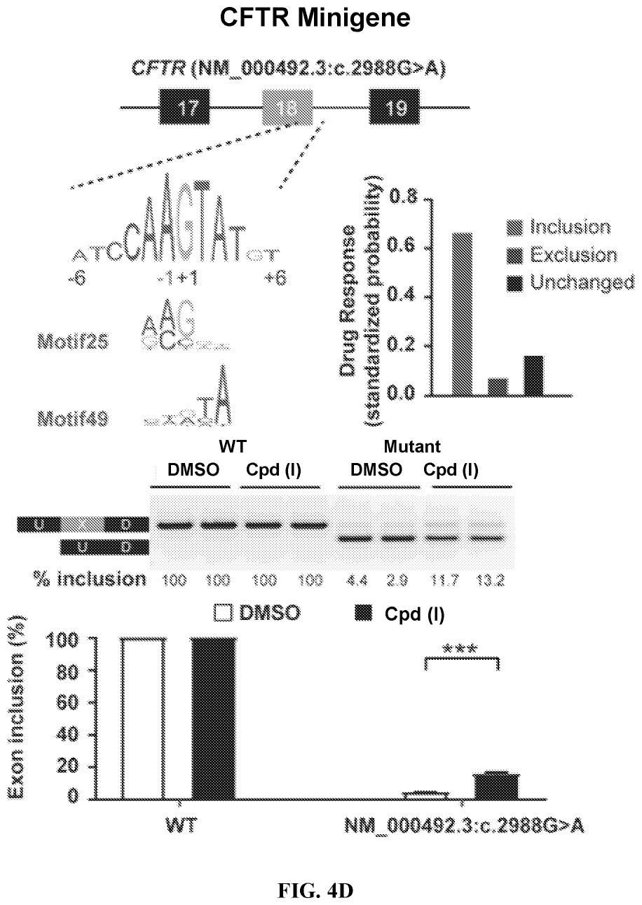

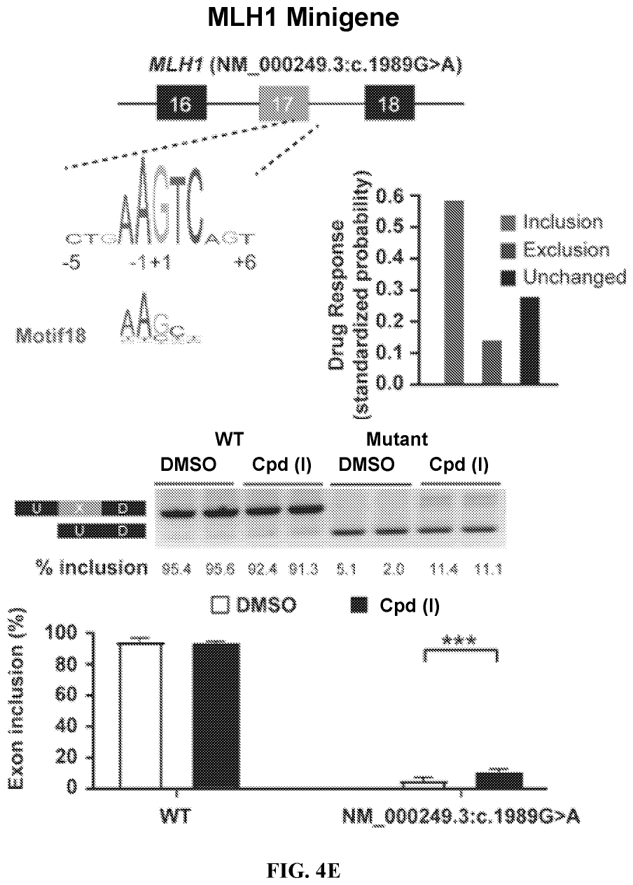

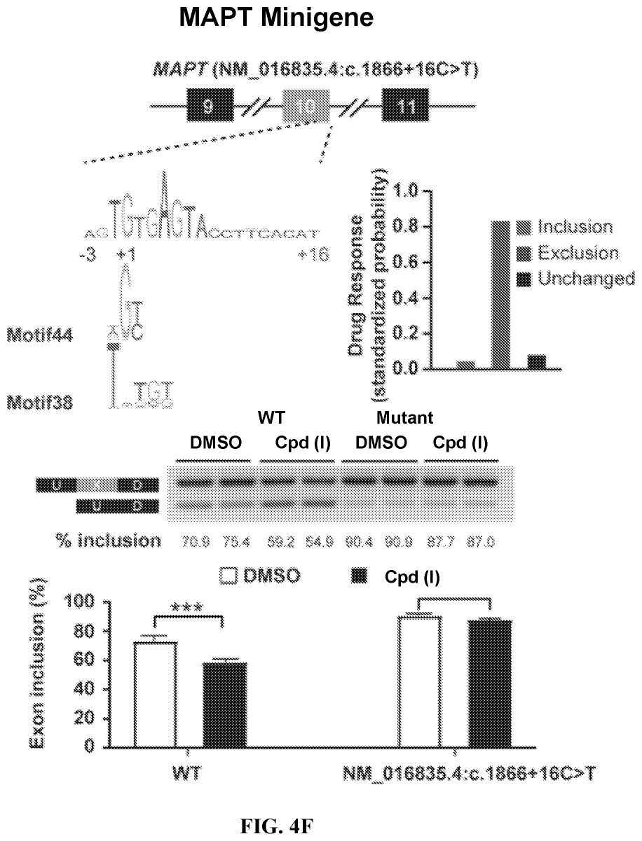

For differential splicing analysis as shown in FIG. 21B, a Cochran-Mantel-Haenszel test was applied followed by FDR (False-Discovery-Rate) correction. A FDR<0.1 and Δψ>=0.1 was considered as an inclusion-response after treatment (FIG. 21B). Any triplet with a FDR<0.1 and Δψ≤=−0.1 was considered as an exclusion-response after treatment (FIG. 21B). Any exon triplet, whose ψ before-treatment was in a range from 0.1 to 0.9 and the ψ change after treatment was less than 0.01, was considered to be an unchanged-response. For the Pearson correlation analysis shown in FIG. 2C, the grey zone indicated a 9500 confident interval. The p values were then adjusted by Bonferroni correction. For the splicing strength comparison illustrated in FIG. 3C, the maximum entropy was compared amongst the inclusion, exclusion and unchanged groups at each splice Junction, using an unpaired Welch's t test followed by Bonferroni correction. For the boxplots shown in FIG. 3C, the middle lines inside boxes indicate the medians. The lower and upper hinges correspond to the first and third quartiles. Each box extends to 1.5 times inter-quartile range (IQR) from the upper and lower hinges respectively. Outliers were not shown. For RT-PCR comparison, an unpaired Student's t test was applied. For the distance comparison shown in FIG. 4B), the Kolmogorov-Smirnov (K-S) test was applied, where all tests were two-tailed. In all plots, the error bars indicate the 95% confident interval and the significance levels were marked by * having p<0.05, ** p<0.01 and *** p<0.001, where the significance levels always refer to the adjusted p values if adjusted as described above. Training of CNN Model to Predict Potentially Responsive Genes

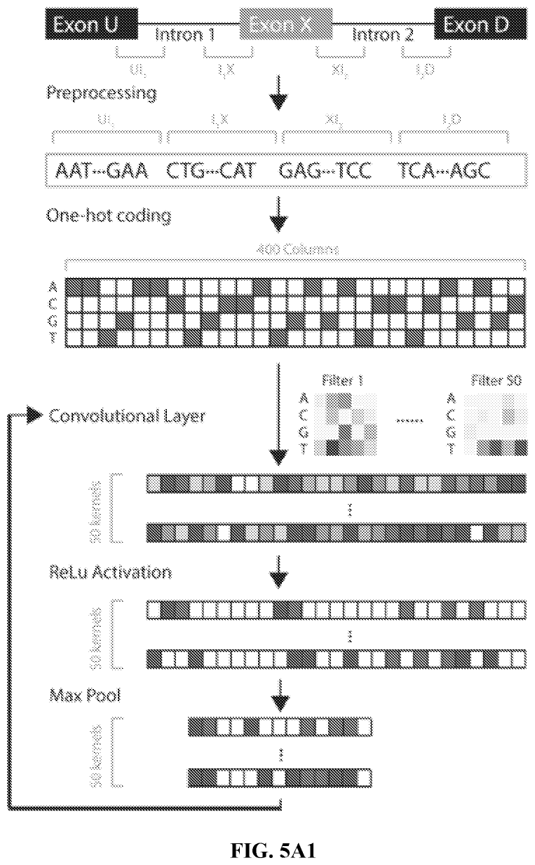

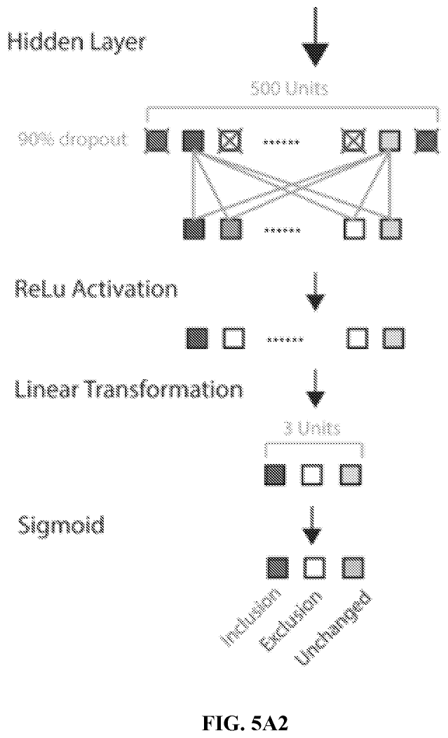

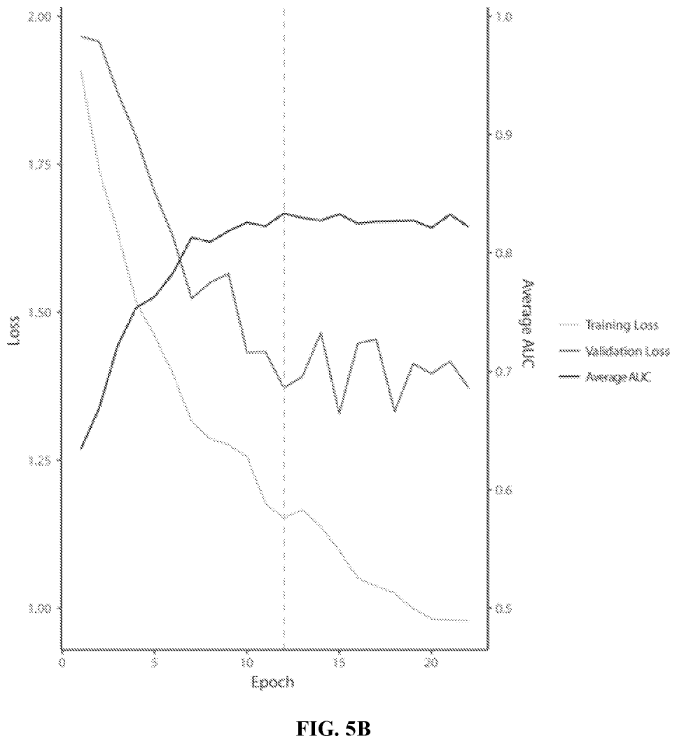

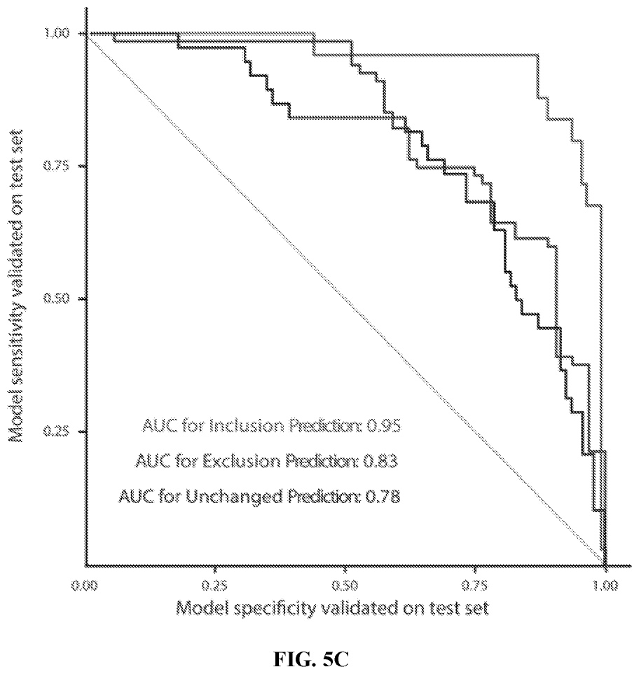

The CNN model (as shown in FIGS. 4A and 4B, and FIGS. 5A1, 5A2, 5B and 5C) consisted of two layers of convolutions with a total of 2.50 million trainable parameters optimized for predicting splicing changes after being trained using RNASeq values for effect on exon triplet splicing after Compound (I) treatment.

The two convolutional layers and one hidden layer (see FIGS. 5A1 and 5A2) were trained using the Basset framework⁴¹. The training set consisted of 178 inclusion-responded, 476 exclusion-responded and 268 unchanged exon triplets. The validation sets consisted of 51 inclusion-responded, 136 exclusion-responded and 76 unchanged exon triplets. The test set consisted of 25 inclusion-responded, 68 exclusion-responded and 38 unchanged exon triplets. The three sets were assigned randomly in Python using seed of 122. For each exon-triplet, the sequences consisting of an exonic 25 nucleotide sequence and an intronic 75 nucleotide sequence within the UI₁, I₁X, XI₂ and I₂D regions (see FIG. 2A) were concatenated and then one-hot coded into an input matrix with a size of 4×400. The first round of convolution was applied with fifty 4×5 weight matrices, converting the input matrix into a 50×396 convoluted matrix, in which each row represented the convolution of one weight matrix.

The convolution matrix was nonlinearly transformed using a rectified linear unit (ReLU) function, with the maximum pool stage taking the maximum of two adjacent positions of each row, shrinking the output matrix to a size of 50×198. The second round of matrix convolution then applied fifty 4×2 weight matrices, followed by the same ReLU transformation and maximum pool of the first round. The output was converted to a 1×500 matrix to initiate the hidden layer, where a fully connected network was built with 90% dropout rate. The output from the hidden layer was ReLU transformed again and was then linearly transformed into a vector of three values, representing the three different treatment responses. The final sigmoid nonlinearity mapped each element in the vector to a value between 0 and 1, considered as the probability of small molecule splicing modulator compound responsiveness. In each Epoch of training, an average of the area under the curve (AUC) was measured on the validation set across the prediction of three treatment responses. The training and validation loss in terms of binary cross-entropy were measured on the training set and validation set respectively. The training process stopped if there was no improvement in the AUC average over 10 consecutive Epochs. In this study, training was stopped at the 12^{th} Epoch to avoid overfitting (see FIG. 5B).

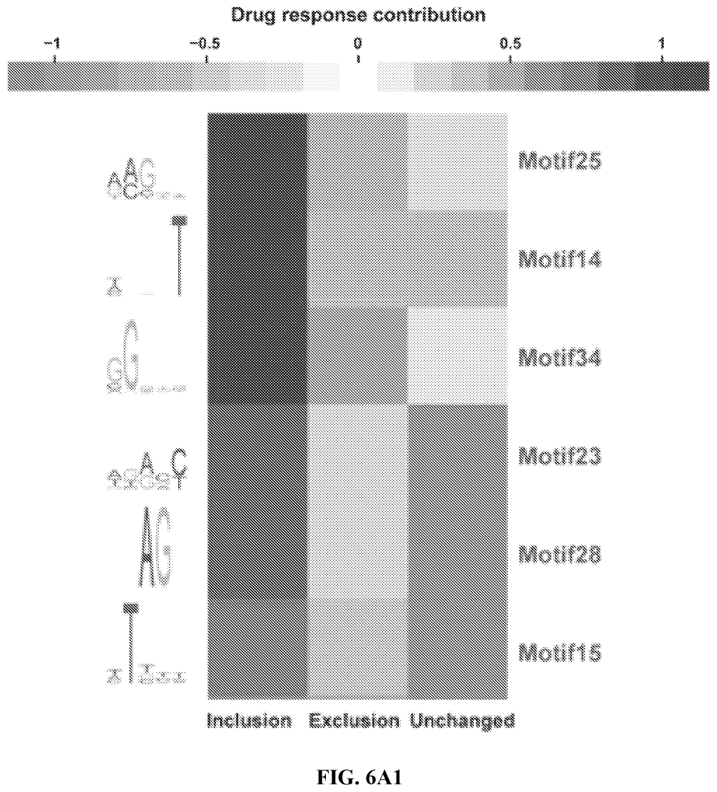

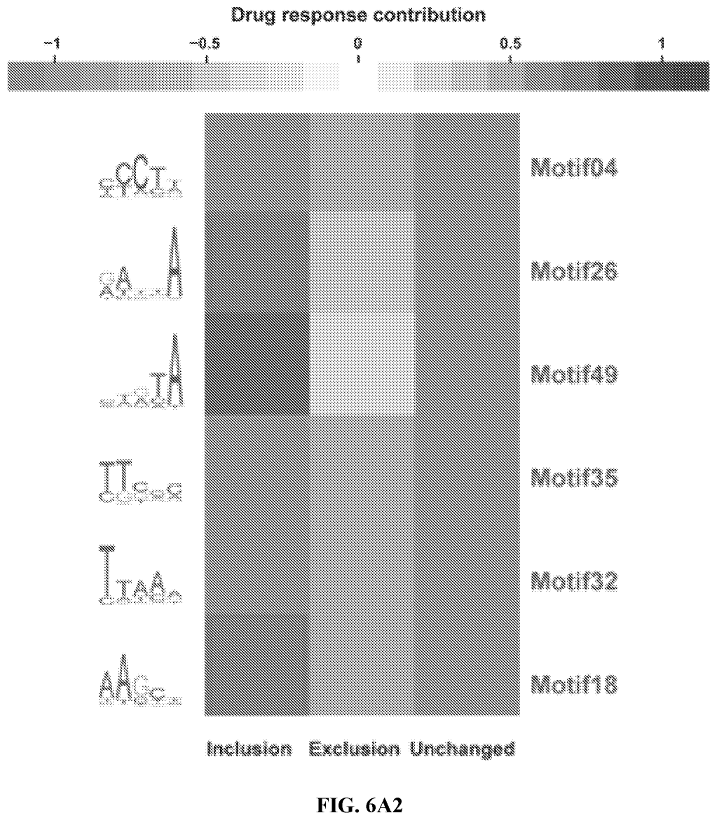

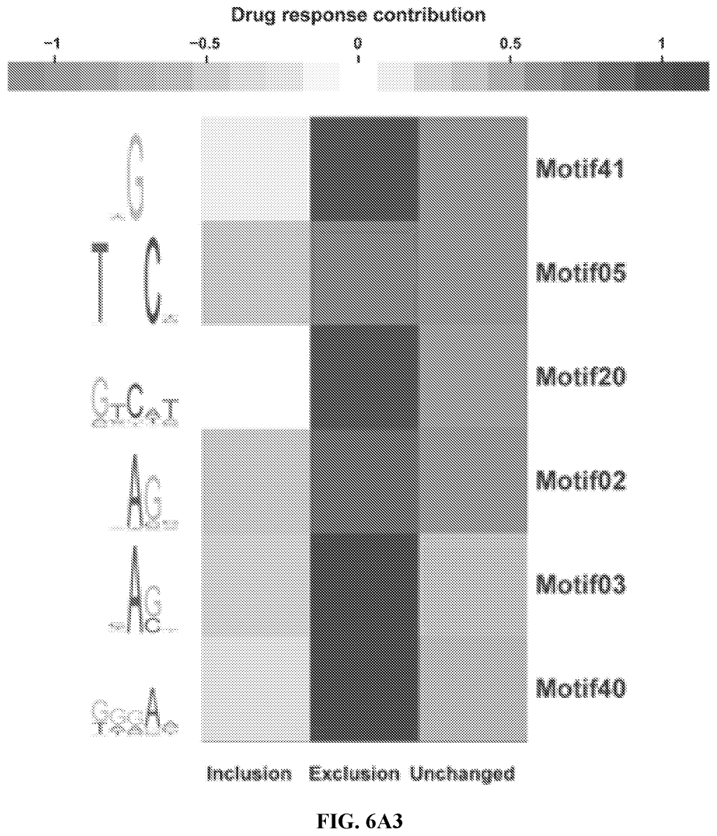

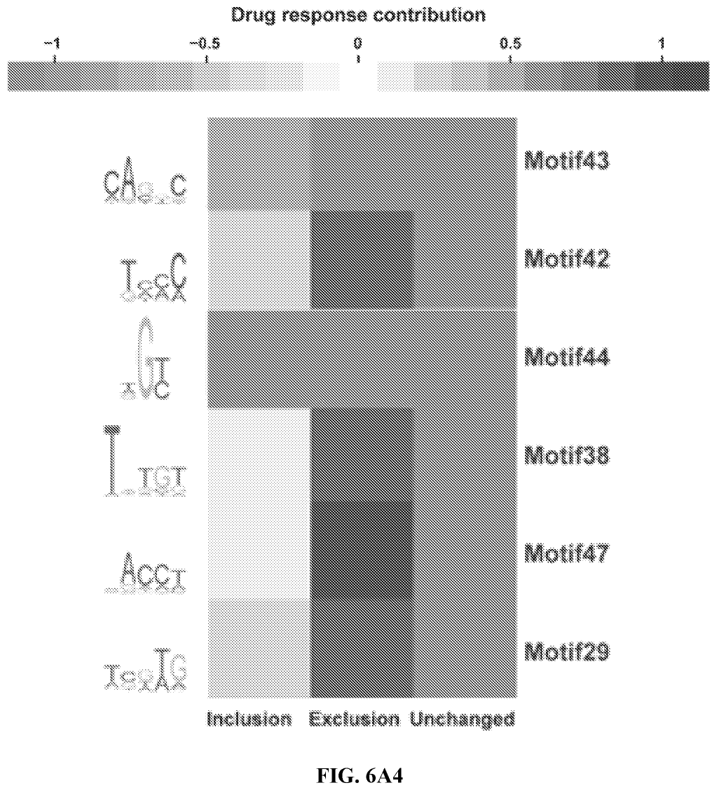

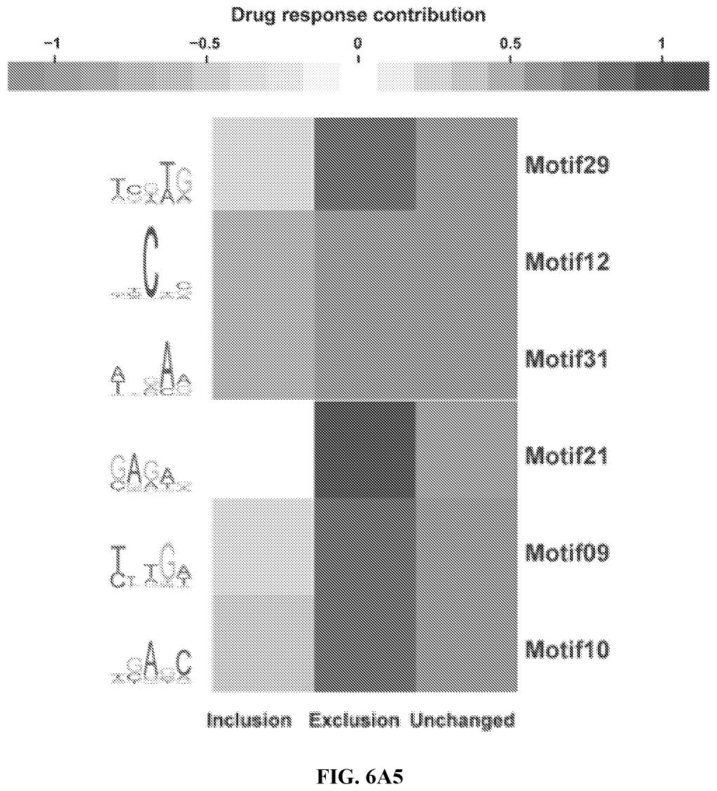

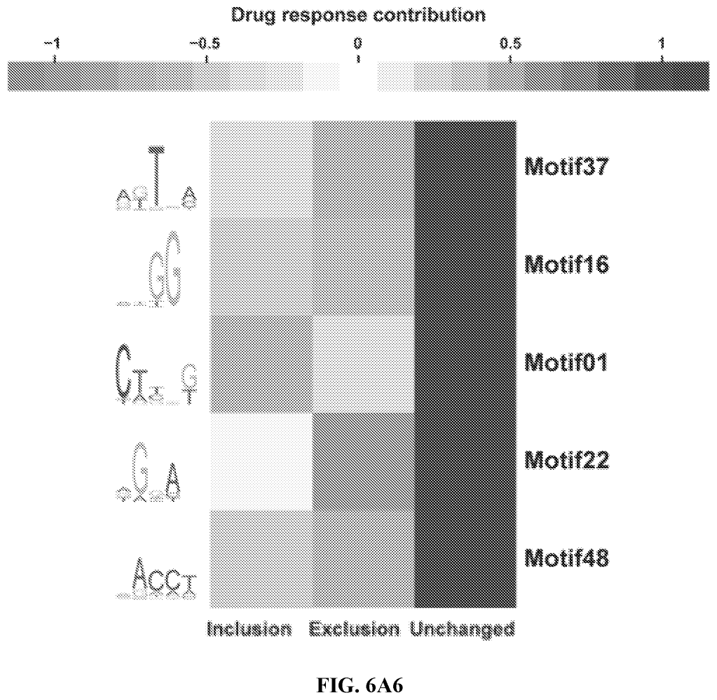

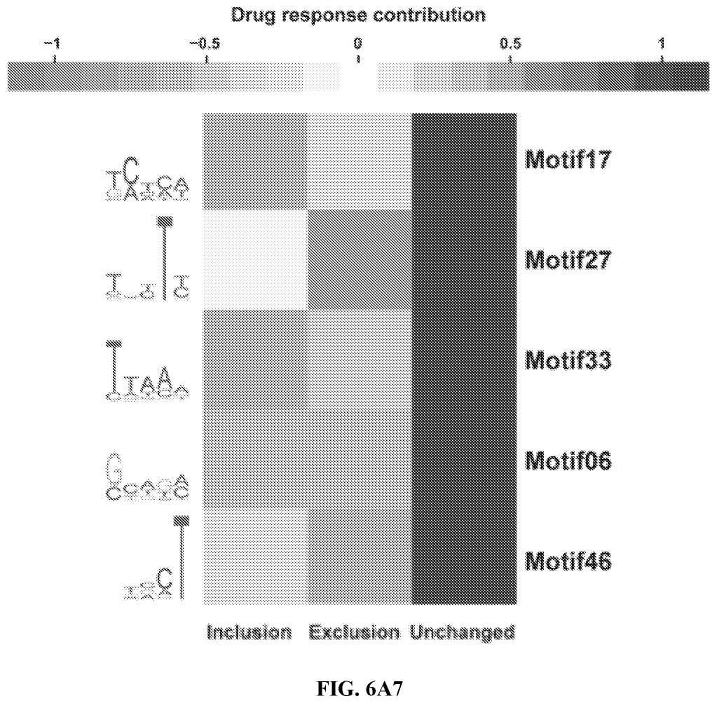

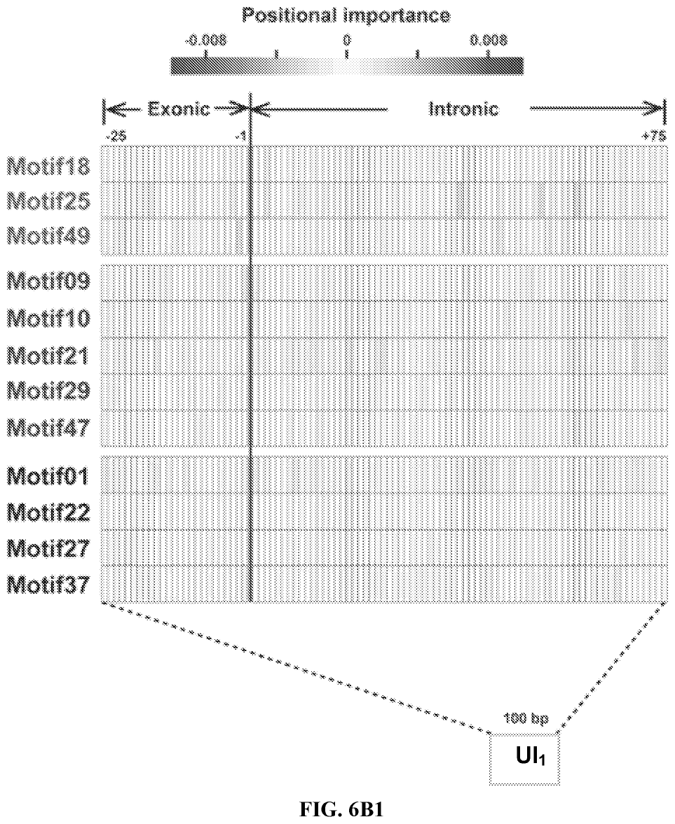

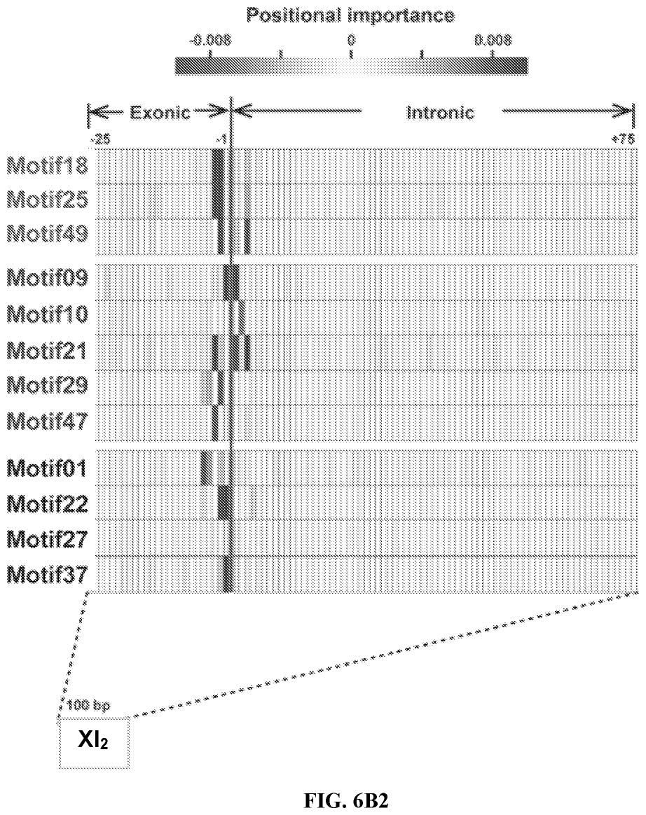

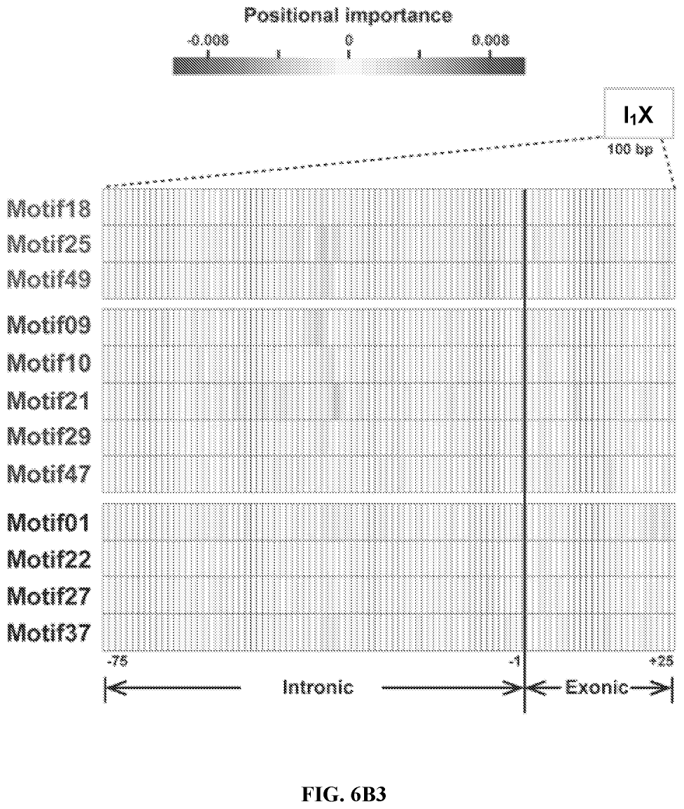

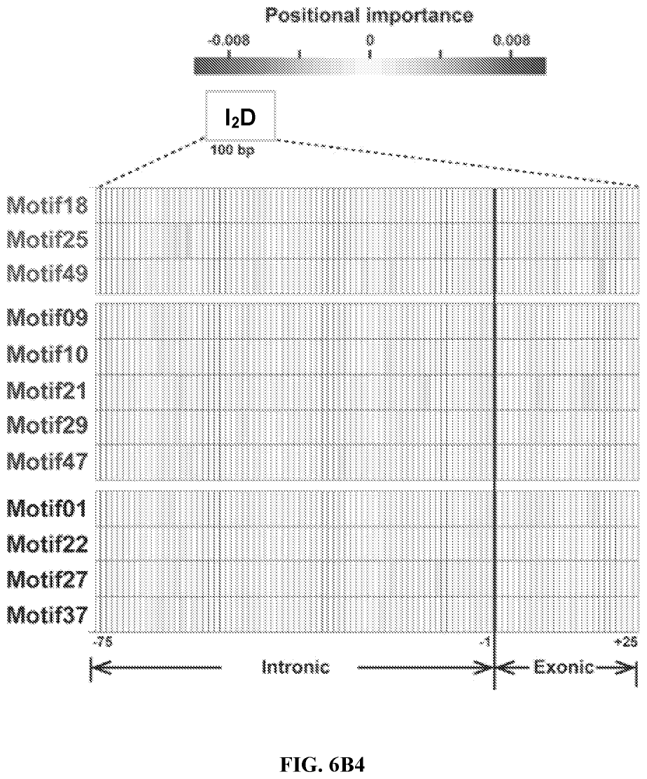

The CNN model obtained an average accuracy for an Area-Under-Curve (AUC) of 0.82 (as shown in FIG. 5C), identifying 39 5-mer CNN Motifs (as shown in FIG. 6A1-6A7) that suggest genes having similar motifs would also be affected by Compound (I) splicing modulation. The 39 CNN Motifs were identified based on their higher probability to cause a splicing reaction in the presence of Compound (I), yielding a prediction of whether the isoform produced will be the result of either an exclusion of an exon or inclusion of an exon or poison exon or whether there will be no change in the isoform produced. The 39 CNN Motifs present in wildtype or mutant genes described herein may cause changes or no change in isoform results in other genes not described herein depending on the structure of the small molecule splicing compound tested.

Without being limited by theory, the 39 CNN Motifs obtained using the CNN Model described herein may suggest other equivalents that may be obtained within the scope of the deep learning algorithm underlying the CNN Model, enabling others of ordinary skill in the art to predict or allow the prediction of the effect of other small molecule splicing compounds to modulate the production of one or more mature RNA isoforms from other wildtype or mutant gene transcripts than those described or predicted herein. In other words, the in vitro and in vivo minigene data provided herein demonstrates that the use of all 39 CNN Motifs in the CNN Model were sufficient and efficient to make correct and accurate predictions.

### CNN Motif Ranking

The 39 CNN Motifs may be ranked according to a predicted probability for modulating the production of one or more mature RNA isoforms from a wildtype or mutant gene transcript. Two analytic methods were used to rank the degree of Motif probability to predict an effect on splicing in the presence of a small molecule splicing compound. In the first instance, the analytic method used reset each Motifs activation probability level to the average level of all Motif activations. As shown in FIGS. 3A1-3A2, the 12 CNN Motifs having AUC changes greater than 0.1 were identified and ordered according to the AUC change before and after the reset. In the second instance, the analytic method used reset each Motifs activation level to zero. As shown in FIGS. 3B1-3B2, the 13 CNN Motifs having AUC changes greater than 0.05 were identified and ordered according to the AUC change before and after the reset.

The 39 CNN Motifs may be ranked according to positional importance for modulating the production of one or more mature RNA isoforms from a wildtype or mutant gene transcript. Two analytic methods were used to rank the positional importance of each Motif to predict an effect on splicing in the presence of a small molecule splicing compound. In the first instance, the AUC analytic method used reset each nucleotide's position activation level to the average activation level of that position. As shown in FIG. 3A3, the positional importance for the 12 CNN Motifs was ranked by the AUC change before and after the reset. In the second instance, the AUC analytic method used the highest activation of each nucleotide position to identify and order the Motifs. As shown in FIG. 3B3, the Motif positional importance for the 13 CNN Motifs was ranked according to the highest activation of each nucleotide position as shown in the heatmap results.

Moreover, without theoretical limitation, the ranking of the 39 Motifs used in the CNN Model according to the description herein and other embodiments may be ordered by those of ordinary skill in the art using a variety of analytic and mathematic modeling to predict or allow the prediction of the effect of other small molecule splicing compounds to modulate the production of one or more mature RNA isoforms from other wildtype or mutant gene transcripts than those described or predicted herein.

### Positional Importance in the CNN Model

To examine each motif contribution in classification, the validation set was used as model input. For each motif whose positional importance was to be measured, the position-wise output of the first convolutional layer from that motif was manually set as the mean of all the convolutional output. The model was then used without tuning other parameters and the new loss, measured by binary cross-entropy, was calculated. The importance of the motif at that tuned position was measured as the difference between the new loss of the model and the original loss of the model. All the positions of that motif were similarly investigated.

Determining the Standardized Probability from CNN Model Prediction

To determine the drug response class (inclusion, exclusion or unchanged) using the CNN Model prediction, the raw prediction score from the Model was standardized. For each class, a cutoff representing 95% specificity for that response was identified on the validation set. The intermediate score of each class was calculated as the raw prediction score divided by the cutoff of that response class. The standardized probability for each response was then calculated as the intermediate score divided by the sum of intermediate scores of the three classes.

### Identification of Enrichment Motif Using K-Mer Enrichment Analysis

The sequences of adjacent nucleotides from the −3 to +7 position of the 5′ splice sites of the middle exons for inclusion, exclusion and unchanged exon triplets were extracted. For each class response, 5-mer enrichment was estimated against the other two classes using Discriminative Regular Expression Motif Elicitation (DREME) from THE MEME Suite (PMID: 21543442) with the parameter “-p, -n, -dna -e 0.05 and -k 5”.

The 5-mer sequence enrichment analysis of adjacent nucleotides from the −3 to +7 position in the Exon X 5′ splice site also agreed with the 39 CNN Motifs identified by the CNN model (as shown in FIG. 6A1-6A7). These results suggest that 5′splice site sequences containing the CNN Motifs are predictively involved in non-canonical U1-5′splice site interaction.

### CNN Model Splice Site Strength Analysis

To determine the effect of the CNN Motifs on the splicing of responsive triplets, the strength of the four splice sites UI₁, I₁X, XI₂ and I₂D within the triplets was evaluated. Splice site strength for each motif was measured using a maximum entropy model⁷⁷. As described, the measurement used was either the short sequence of 9 nucleotides and 23 nucleotides for the flanking splice junctions, as determined from the 5′ or 3′ end, respectively. The resulting analysis found that exon triplets with inclusion responses had significantly lower strength in the Exon X 5′ splice site compared to the exon triplets with unchanged response (as shown in FIG. 3C). Consistent with previous findings that support the role of this class of splicing modulator compounds in promoting the recruitment of U1 snRNP in non-canonical ending exons^{46,56}, these data suggest that weak 5′ splice sites are more sensitive to treatment with Compound (I).

### CNN Model Prediction

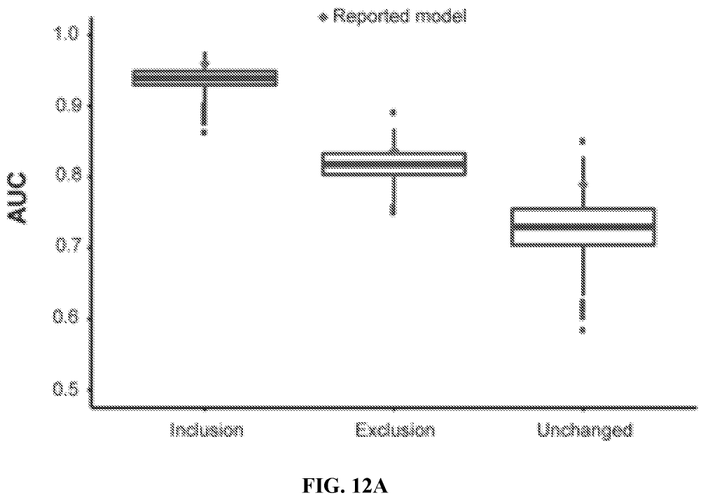

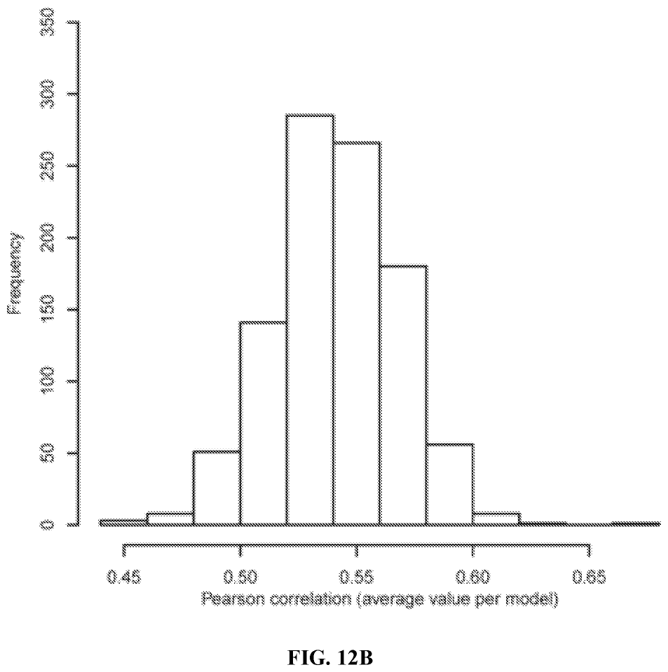

To evaluate the reproducibility of the model, random initialization of the training process was implemented 1,000 times. It was found that the performance of all models was tightly distributed and aligned with the original model (FIG. 12A). In addition, all of the top 10 first-layer filters contributing to the performance of the CNN model were highly correlated with those of the 1,000 random-initialized models (average Pearson correlation R2=0.55, FIG. 12B), suggesting that the deep learning framework was robust.

The 39 5-mer motifs (CNN Motifs) were identified from the first layer of the CNN model were identified (FIGS. 6A1-6A7). The treatment response was not determined by any of these motifs independently, all 39 CNN Motifs are used in the CNN Model to interrogate genes of interest. As such, the CNN model utilizes the synergistic effect of all 39 Motifs on a given sequence to make the Class decision. Thirteen motifs (see FIGS. 3B1-3B2) explained 92.62% of the AUC, each of which altered more than 0.105 of AUC for at least one class of prediction (FIG. 5C). Analysis of these motifs revealed that the sequence in proximity to, and in many cases encompassing, the 5′ splice site of the middle exon had the largest influence in modulating treatment response (FIGS. 3A, 3B and 3C). These results emphasized the importance of the 5′ splice site in determining treatment outcome. In silico saturation mutagenesis further supported these findings, revealing that base contribution to the treatment outcomes peaked around the 5′ splice sites of the middle exons, with distinct patterns amongst sequences with inclusion, exclusion and unchanged responses (FIG. 3C).

Table 17 lists the motifs (Motif) shown in FIGS. 6A1-6A7, with constant and most dominant nucleotides as shown in the LOGO plot. The 39 CNN Motifs were those most frequent 5-mer sequences (5-mer) predicted by the CNN Model to affect splicing in the presence of Compound (I) toward an inclusion, exclusion or unchanged Effect.

TABLE 17

| Motif | 5-Mer | SEQ ID NO. | Effect |
|---|---|---|---|
| 01 | CTTAG | 25 | Unchanged |
| 02 | NNAGC | 26 | Exclusion |
| 03 | NCAGA | 27 | Exclusion |
| 04 | CCCTT | 28 | Inclusion |
| 05 | TNNCT | 29 | Exclusion |
| 06 | GCAGA | 30 | Unchanged |
| 09 | TNTGA | 31 | Exclusion |
| 10 | TGAGC | 32 | Exclusion |
| 12 | CTCTC | 33 | Exclusion |
| 14 | TNNNT | 34 | Inclusion |
| 15 | TTTTT | 35 | Inclusion |
| 16 | ATGGN | 36 | Unchanged |
| 17 | TCTCA | 37 | Unchanged |
| 18 | AAGCT | 38 | Inclusion |
| 20 | GTCAT | 39 | Exclusion |
| 21 | GAGAG | 40 | Exclusion |
| 22 | AGGAN | 41 | Unchanged |
| 23 | AGACC | 42 | Inclusion |
| 25 | AAGGT | 43 | Inclusion |
| 26 | GATTA | 44 | Inclusion |
| 27 | TCTTT | 45 | Unchanged |
| 28 | NNAGN | 46 | Inclusion |
| 29 | TCGTG | 47 | Exclusion |
| 31 | ANGAA | 48 | Exclusion |
| 32 | TTAAA | 49 | Inclusion |
| 33 | AGACT | 50 | Unchanged |
| 34 | GGGTG | 51 | Inclusion |
| 35 | TTCCC | 52 | Inclusion |
| 37 | AGTNA | 53 | Unchanged |
| 38 | TATGT | 54 | Exclusion |
| 40 | GGGAA | 55 | Exclusion |
| 41 | NTGNN | 56 | Exclusion |
| 42 | NTCCC | 57 | Exclusion |
| 43 | CAGGC | 58 | Exclusion |
| 44 | NTGTN | 59 | Exclusion |
| 46 | NTCCT | 60 | Unchanged |
| 47 | AACCT | 61 | Exclusion |
| 48 | ACTCN | 62 | Unchanged |
| 49 | CTGTA | 63 | Inclusion |

### Identification of ClinVar Therapeutic Targets Harboring Disease-Causing Mutations Amenable to Compound (I) Splicing Modulation

As shown in FIG. 4A, from 89,642 potential CV-pMUTs within 5 kb upstream or downstream of the mutated site, SpliceAI scores identified 17,956 (20.03%) splice site mutations that had the potential to disrupt pre-mRNA splicing in the gene and result in a disease. Out of those 17,956 CV-pMUTs, there were 14,272 (79.48%) leading to splicing disruption of the Ensembl-annotated splice sites (GRCh37 version 75). These CV-pMUT disrupting annotated splice sites were then used to train the CNN model to predict splicing alterations in mutated splice sites that could be corrected by Compound (I) treatment.

As shown in FIG. 4B, the CV-pMUTs disrupting annotated splice sites demonstrated that the identified flanking regions UI₁, I₁X, XI₂ and I₂D were significantly closer to the splice junction than the CV-pMUTs that had no effect in disrupting annotated splice sites. Since about 98% of them were within 75 nucleotides of the splice site junction, the flanking regions were determined to include most of the targetable pathogenic splicing alterations for use in training the CNN model.

As illustrated in FIG. 4A, amongst the 14,272 CV-pMUTs predicted to disrupt annotated splice sites (corresponding to 11,616 exon triplets in 1,970 genes), the CNN model predicted that the altered splicing of 234 CV-pMUTs (1.64%) found in 295 exon triplets (2.54%) in 174 genes (8.83%) could be rescued by splicing modulation, toward either or both exon inclusion and exon exclusion, resulting from Compound (I) treatment.

### SpliceAI Prediction for ClinVar Pathogenic Mutations

The VCF file recording ClinVar (version 20190325) mutations was downloaded. The pathogenic/likely pathogenic mutations were extracted and fed to SpliceAI (https://github.com/illumina/SpliceAI). In the prediction from SpliceAI, any mutation with any SpliceAI score greater than 0.2 was considered to be the result of altering splicing. As shown in Table 19, those mutations, together with the influenced splice junction (ISJ) and SpliceAI score were recorded.

### Rescue Definition and Prediction

For an exon triplet, the coordinates of the two flanking domains of the middle exon for an exon triplet were compared to those ISJs discovered by SpliceAI. If either domain overlapped with an ISJ and the SpliceAI score indicated a splicing gain, the exon triplet was considered to likely promote exon inclusion as a result of the corresponding mutation. On the other hand, a 5′ splice site of the middle exon overlapping with an ISJ and having a SpliceAI score that indicated a splicing loss, the exon triplet was considered to likely promote exon skipping as a result of the corresponding mutation.

Three possible rescue outcomes were considered: 1) The mutated exon triplet was predicated (by SpliceAI) to cause exon skipping and the CNN model predicted an inclusion response after Compound (I) treatment; or, 2) The mutated exon triplet was predicted (by SpliceAI) to promote exon inclusion and the CNN model predicted an exclusion response after Compound (I) treatment; or, 3) the mutated exon triplet generated a pre-mature termination codon (PTC) inside the middle exon and the CNN model predicted an exclusion response after Compound (I) treatment without a reading frameshift after exclusion.

Allele Frequency From gnomAD

VCF files for both human exome and genome sequencing were downloaded from gnomAD (v2.1.1). The corresponding ClinVar mutations were located in the VCF files via their SNP IDs. If a short variant was found only in the exome or only in the genome sequencing VCF, the reported minor allele frequency was then used. If a short variant was found in both the exome and genome sequencing, the combined frequency was calculated as (AC1+AC2)/(AN1+AN2), where AC1 and AC2 were the allele counts for exome and genome sequencing, respectively, and AN1 and AN2 were the total sample sizes for exome and genome sequencing, respectively.

As described herein, wildtype genes were identified by RNA sequencing and ClinVar mutated genes were predicted by SpliceAI scores and the CNN model to harbor a mutation that will cause a splicing defect were analyzed, as described herein, to determine whether they could be responsive to Compound (I) splicing modulation. The CNN Model was used to predict whether the ClinVar SpliceAI identified splicing defect could be rescued by treatment with Compound (I) modulation of splicing toward either or both exon inclusion and exon exclusion. As described herein, certain genes were responsive to modulation of splicing as a result of treatment with Compound (I).

Table 18 lists for each mutated Gene (identified by ClinVar and Allele identification numbers) and associated Introns and Exon (where Intron 1 is upstream of the affected Exon and Intron 2 is downstream), predicted by SpliceAI scores based on frequency of occurrence (gnomAD Freq) and the CNN model, as described herein, that may be modulated by Compound (I) toward either or both exon inclusion and exon exclusion (Rescue).

TABLE 18

| | | Clin | | gnomAD | | | |
|---|---|---|---|---|---|---|---|
| Gene | Rescue | VAR | Allele | Freq | Intron 1 | Exon | Intron2 |
| ABCA4 | inclusion | 236104 | 237676 | −1 | 94502345- | 94502701- | 94502907- |
| | | | | | 94502700 | 94502906 | 94505598 |
| ABCC9 | inclusion | 31947 | 40612 | 3.99E−06 | 21991105- | 21995248- | 21995406- |
| | | | | | 21995247 | 21995405 | 21997416 |
| ABCC9 | inclusion | 31946 | 40611 | −1 | 21991105- | 21995248- | 21995406- |
| | | | | | 21995247 | 21995405 | 21997416 |
| ACADSB | inclusion | 9199 | 24238 | 2.95E−05 | 124810703- | 124812577- | 124812677- |
| | | | | | 124812576 | 124812676 | 124813210 |
| ADAM10 | inclusion | 88840 | 94409 | −1 | 58904191- | 58913670- | 58913821- |
| | | | | | 58913669 | 58913820 | 58919898 |
| AGK | inclusion | 209129 | 205751 | 1.42E−05 | 141313979- | 141315271- | 141315366- |
| | | | | | 141315270 | 141315365 | 141321531 |
| ALDH3A2 | inclusion | 189079 | 186958 | −1 | 19559888- | 19561034- | 19561176- |
| | | | | | 19561033 | 19561175 | 19564439 |
| ALDH3A2 | inclusion | 189079 | 186958 | −1 | 19559888- | 19561058- | 19561176- |
| | | | | | 19561057 | 19561175 | 19564439 |
| ALMS1 | inclusion | 191112 | 188916 | −1 | 73646447- | 73649985- | 73650103- |
| | | | | | 73649984 | 73650102 | 73651557 |
| ANTXR2 | inclusion | 2598 | 17637 | −1 | 80899329- | 80905032- | 80905125- |
| | | | | | 80905031 | 80905124 | 80905972 |
| APC | inclusion | 411344 | 394701 | −1 | 112116601- | 112136976- | 112137081- |
| | | | | | 112136975 | 112137080 | 112151191 |
| APC | inclusion | 411344 | 394701 | −1 | 112128227- | 112136976- | 112137081- |
| | | | | | 112136975 | 112137080 | 112151191 |
| APC | inclusion | 411344 | 394701 | −1 | 112128227- | 112136976- | 112137081- |
| | | | | | 112136975 | 112137080 | 112145822 |
| APC | inclusion | 433614 | 427251 | −1 | 112146009- | 112151192- | 112151291- |
| | | | | | 112151191 | 112151290 | 112154662 |
| APC | inclusion | 486792 | 473576 | −1 | 112151291- | 112154663- | 112155042- |
| | | | | | 112154662 | 112155041 | 112157592 |
| APC | inclusion | 411416 | 394347 | −1 | 112151291- | 112154663- | 112155042- |
| | | | | | 112154662 | 112155041 | 112157592 |
| APC | inclusion | 428128 | 419618 | −1 | 112157689- | 112170648- | 112170863- |
| | | | | | 112170647 | 112170862 | 112173249 |
| APC | inclusion | 428128 | 419618 | −1 | 112164670- | 112170648- | 112170863- |
| | | | | | 112170647 | 112170862 | 112173249 |
| ARMC9 | inclusion | 427935 | 419011 | 3.98E−06 | 232137792- | 232141349- | 232141489- |
| | | | | | 232141348 | 232141488 | 232143094 |
| ASAH1 | inclusion | 55908 | 70551 | −1 | 17916974- | 17917081- | 17917213- |
| | | | | | 17917080 | 17917212 | 17918885 |
| ASPM | inclusion | 402180 | 389111 | −1 | 197062392- | 197063214- | 197063311- |
| | | | | | 197063213 | 197063310 | 197065127 |
| ASPM | inclusion | 210353 | 206745 | −1 | 197091726- | 197093240- | 197093462- |
| | | | | | 197093239 | 197093461 | 197093999 |
| ASPM | inclusion | 21572 | 34424 | −1 | 197094086- | 197094176- | 197094322- |
| | | | | | 197094175 | 197094321 | 197097619 |
| ATM | inclusion | 3044 | 18083 | 4.39E−05 | 108124767- | 108126942- | 108127068- |
| | | | | | 108126941 | 108127067 | 108128207 |
| ATM | inclusion | 181974 | 180483 | 2.78E−05 | 108183226- | 108186550- | 108186639- |
| | | | | | 108186549 | 108186638 | 108186737 |
| ATM | inclusion | 3035 | 18074 | 1.59E−05 | 108150336- | 108151722- | 108151896- |
| | | | | | 108151721 | 108151895 | 108153436 |
| ATM | inclusion | 230851 | 233909 | 4.01E−06 | 108098616- | 108099905- | 108100051- |
| | | | | | 108099904 | 108100050 | 108106396 |
| ATM | inclusion | 230851 | 233909 | 4.01E−06 | 108098616- | 108099905- | 108100051- |
| | | | | | 108099904 | 108100050 | 108114679 |
| ATM | inclusion | 135778 | 139490 | 3.99E−06 | 108202285- | 108202606- | 108202765- |
| | | | | | 108202605 | 108202764 | 108203488 |
| ATRX | inclusion | 560933 | 552266 | −1 | 76907844- | 76909588- | 76909691- |
| | | | | | 76909587 | 76909690 | 76912049 |
| BBS4 | inclusion | 585184 | 576240 | −1 | 73002121- | 73004585- | 73004649- |
| | | | | | 73004584 | 73004648 | 73009118 |
| BBS4 | inclusion | 585184 | 576240 | −1 | 73002121- | 73004585- | 73004649- |
| | | | | | 73004584 | 73004648 | 73007631 |
| BFSP1 | inclusion | 425558 | 413913 | 0.00E+00 | 17475675- | 17477583- | 17477669- |
| | | | | | 17477582 | 17477668 | 17479464 |
| BMPR2 | inclusion | 425729 | 414116 | −1 | 203242274- | 203329532- | 203329703- |
| | | | | | 203329531 | 203329702 | 203332241 |
| BRCA1 | inclusion | 37604 | 46160 | 3.19E−05 | 41223256- | 41226348- | 41226539- |
| | | | | | 41226347 | 41226538 | 41228504 |
| BRCA1 | inclusion | 37604 | 46160 | 3.19E−05 | 41223256- | 41226348- | 41226539- |
| | | | | | 41226347 | 41226538 | 41277287 |
| BRCA1 | inclusion | 17660 | 32699 | 1.20E−05 | 41256974- | 41258473- | 41258551- |
| | | | | | 41258472 | 41258550 | 41276033 |
| BRCA1 | inclusion | 17660 | 32699 | 1.20E−05 | 41256974- | 41258473- | 41258551- |
| | | | | | 41258472 | 41258550 | 41262481 |
| BRCA1 | inclusion | 55131 | 69798 | −1 | 41234593- | 41242961- | 41243050- |
| | | | | | 41242960 | 41243049 | 41251791 |
| BRCA1 | inclusion | 55131 | 69798 | −1 | 41234593- | 41242961- | 41243050- |
| | | | | | 41242960 | 41243049 | 41246760 |
| BRCA1 | inclusion | 55131 | 69798 | −1 | 41234593- | 41242961- | 41243050- |
| | | | | | 41242960 | 41243049 | 41243451 |
| BRCA1 | inclusion | 55131 | 69798 | −1 | 41234593- | 41242961- | 41243050- |
| | | | | | 41242960 | 41243049 | 41247862 |
| BRCA1 | inclusion | 54471 | 69138 | −1 | 41256974- | 41258473- | 41258551- |
| | | | | | 41258472 | 41258550 | 41276033 |
| BRCA1 | inclusion | 54471 | 69138 | −1 | 41256974- | 41258473- | 41258551- |
| | | | | | 41258472 | 41258550 | 41262481 |
| BRCA1 | inclusion | 54471 | 69138 | −1 | 41256974- | 41258473- | 41258551- |
| | | | | | 41258472 | 41258550 | 41267742 |
| BRCA2 | inclusion | 38215 | 46771 | 4.03E−06 | 32953653- | 32953887- | 32954051- |
| | | | | | 32953886 | 32954050 | 32954143 |
| BRCA2 | inclusion | 51711 | 66379 | −1 | 32899322- | 32900238- | 32900288- |
| | | | | | 32900237 | 32900287 | 32900378 |
| BRCA2 | inclusion | 52058 | 66726 | −1 | 32900420- | 32900636- | 32900751- |
| | | | | | 32900635 | 32900750 | 32903579 |
| CA5A | inclusion | 127088 | 132598 | 1.66E−04 | 87935581- | 87936031- | 87936127- |
| | | | | | 87936030 | 87936126 | 87938391 |
| CAPN3 | inclusion | 523316 | 514049 | −1 | 42678484- | 42679951- | 42680085- |
| | | | | | 42679950 | 42680084 | 42681125 |
| CD3D | inclusion | 180674 | 178842 | 7.96E−06 | 118210210- | 118211090- | 118211309- |
| | | | | | 118211089 | 118211308 | 118211584 |
| CD3D | inclusion | 180674 | 178842 | 7.96E−06 | 118210622- | 118211090- | 118211309- |
| | | | | | 118211089 | 118211308 | 118213267 |
| CD3D | inclusion | 180674 | 178842 | 7.96E−06 | 118210210- | 118211090- | 118211309- |
| | | | | | 118211089 | 118211308 | 118213267 |
| CDH1 | inclusion | 185583 | 184420 | −1 | 68849663- | 68853183- | 68853329- |
| | | | | | 68853182 | 68853328 | 68855903 |
| CDH23 | inclusion | 45989 | 55154 | 8.05E−06 | 73544178- | 73544648- | 73544858- |
| | | | | | 73544647 | 73544857 | 73545387 |
| CERKL | inclusion | 438052 | 431648 | −1 | 182413332- | 182413407- | 182413585- |
| | | | | | 182413406 | 182413584 | 182414360 |
| CFTR | inclusion | 53750 | 68418 | 7.09E−06 | 117251863- | 117254667- | 117254768- |
| | | | | | 117254666 | 117254767 | 117267575 |
| CFTR | inclusion | 35857 | 44521 | 7.08E−06 | 117243837- | 117246728- | 117246808- |
| | | | | | 117246727 | 117246807 | 117250572 |
| CFTR | inclusion | 54012 | 68679 | −1 | 117171169- | 117174330- | 117174420- |
| | | | | | 117174329 | 117174419 | 117175301 |
| CHD7 | inclusion | 520773 | 511761 | −1 | 61754612- | 61757423- | 61757623- |
| | | | | | 61757422 | 61757622 | 61757808 |
| CLCN1 | inclusion | 447078 | 441048 | 1.41E−05 | 143021586- | 143027865- | 143027991- |
| | | | | | 143027864 | 143027990 | 143028324 |
| CLMP | inclusion | 50384 | 59506 | 3.98E−06 | 122944483- | 122945410- | 122945559- |
| | | | | | 122945409 | 122945558 | 122953792 |
| CLMP | inclusion | 50384 | 59506 | 3.98E−06 | 122944483- | 122945410- | 122945552- |
| | | | | | 122945409 | 122945551 | 122953792 |
| CLMP | inclusion | 50384 | 59506 | 3.98E−06 | 122944483- | 122945410- | 122945525- |
| | | | | | 122945409 | 122945524 | 122953792 |
| CLN3 | inclusion | 56265 | 70904 | 4.00E−06 | 28498863- | 28498983- | 28499063- |
| | | | | | 28498982 | 28499062 | 28500610 |
| CLN3 | inclusion | 56265 | 70904 | 4.00E−06 | 28498863- | 28498983- | 28499063- |
| | | | | | 28498982 | 28499062 | 28499911 |
| CLN3 | inclusion | 56265 | 70904 | 4.00E−06 | 28498863- | 28498983- | 28499063- |
| | | | | | 28498982 | 28499062 | 28502802 |
| CLN3 | inclusion | 56265 | 70904 | 4.00E−06 | 28498863- | 28498983- | 28499063- |
| | | | | | 28498982 | 28499062 | 28500610 |
| CNGB3 | inclusion | 427674 | 417119 | −1 | 87656915- | 87660029- | 87660116- |
| | | | | | 87660028 | 87660115 | 87666239 |
| COG6 | inclusion | 493009 | 485965 | 7.10E−06 | 40254183- | 40256308- | 40256402- |
| | | | | | 40256307 | 40256401 | 40261639 |
| COL11A1 | inclusion | 547210 | 537669 | 0.00E+00 | 103364330- | 103364497- | 103364551- |
| | | | | | 103364496 | 103364550 | 103377714 |
| COL3A1 | inclusion | 101376 | 107122 | −1 | 189863046- | 189863400- | 189863445- |
| | | | | | 189863399 | 189863444 | 189864010 |
| COL4A3 | inclusion | 551759 | 541906 | −1 | 228125834- | 228128496- | 228128661- |
| | | | | | 228128495 | 228128660 | 228131132 |
| COL4A5 | inclusion | 38768 | 47373 | 5.55E−06 | 107863657- | 107865033- | 107865123- |
| | | | | | 107865032 | 107865122 | 107865905 |
| COL4A5 | inclusion | 587202 | 578111 | −1 | 107827756- | 107829845- | 107829978- |
| | | | | | 107829844 | 107829977 | 107834287 |
| COL4A5 | inclusion | 587163 | 578120 | −1 | 107829978- | 107834288- | 107834462- |
| | | | | | 107834287 | 107834461 | 107834790 |
| COL4A5 | inclusion | 24409 | 35750 | −1 | 107834462- | 107834791- | 107834875- |
| | | | | | 107834790 | 107834874 | 107838738 |
| COL4A5 | inclusion | 24530 | 35871 | −1 | 107850123- | 107858141- | 107858255- |
| | | | | | 107858140 | 107858254 | 107863488 |
| COL4A5 | inclusion | 24550 | 35891 | −1 | 107863657- | 107865033- | 107865123- |
| | | | | | 107865032 | 107865122 | 107865905 |
| COL4A5 | inclusion | 24696 | 36037 | −1 | 107920864- | 107923909- | 107923982- |
| | | | | | 107923908 | 107923981 | 107924114 |
| COL4A5 | inclusion | 24773 | 36114 | −1 | 107936156- | 107938037- | 107938152- |
| | | | | | 107938036 | 107938151 | 107939526 |
| COL4A5 | inclusion | 24782 | 36123 | −1 | 107938152- | 107938497- | 107938670- |
| | | | | | 107938496 | 107938669 | 107939526 |
| COL5A2 | inclusion | 213101 | 209548 | −1 | 189926342- | 189927591- | 189927645- |
| | | | | | 189927590 | 189927644 | 189927735 |
| CSTB | inclusion | 55958 | 70597 | −1 | 45194212- | 45194539- | 45194641- |
| | | | | | 45194538 | 45194640 | 45196084 |
| CTNS | inclusion | 556587 | 548124 | −1 | 3543562- | 3558515- | 3558647- |
| | | | | | 3558514 | 3558646 | 3559780 |
| CTU2 | inclusion | 585016 | 576071 | 9.29E−06 | 88779314- | 88779720- | 88779856- |
| | | | | | 88779719 | 88779855 | 88780054 |
| CUBN | inclusion | 56321 | 70960 | −1 | 17142239- | 17145124- | 17145237- |
| | | | | | 17145123 | 17145236 | 17146417 |
| CWC27 | inclusion | 426071 | 414475 | 3.80E−06 | 64079807- | 64081308- | 64081407- |
| | | | | | 64081307 | 64081406 | 64082350 |
| CYBB | inclusion | 10933 | 25972 | −1 | 37639376- | 37642743- | 37642854- |
| | | | | | 37642742 | 37642853 | 37651227 |
| DCX | inclusion | 158456 | 169903 | −1 | 110644559- | 110653263- | 110653649- |
| | | | | | 110653262 | 110653648 | 110655365 |
| DCX | inclusion | 158456 | 169903 | −1 | 110644559- | 110653263- | 110653649- |
| | | | | | 110653262 | 110653648 | 110653981 |
| DCX | inclusion | 158456 | 169903 | −1 | 110644559- | 110653263- | 110653649- |
| | | | | | 110653262 | 110653648 | 110655197 |
| DGKE | inclusion | 548648 | 539159 | −1 | 54923171- | 54925283- | 54925427- |
| | | | | | 54925282 | 54925426 | 54926056 |
| DGUOK | inclusion | 214286 | 210897 | 4.95E−05 | 74174034- | 74177712- | 74177860- |
| | | | | | 74177711 | 74177859 | 74184251 |
| DGUOK | inclusion | 214286 | 210897 | 4.95E−05 | 74174034- | 74177754- | 74177860- |
| | | | | | 74177753 | 74177859 | 74185272 |
| DGUOK | inclusion | 214286 | 210897 | 4.95E−05 | 74174034- | 74177728- | 74177860- |
| | | | | | 74177727 | 74177859 | 74185272 |
| DMD | inclusion | 501566 | 492990 | −1 | 32717411- | 32827610- | 32827729- |
| | | | | | 32827609 | 32827728 | 32834584 |
| DNAH5 | inclusion | 228251 | 229258 | 1.99E−05 | 13829814- | 13830135- | 13830323- |
| | | | | | 13830134 | 13830322 | 13830705 |
| DNAH5 | inclusion | 572293 | 564698 | 1.99E−05 | 13701546- | 13708232- | 13708445- |
| | | | | | 13708231 | 13708444 | 13714513 |
| DNAH9 | inclusion | 617523 | 608891 | 1.45E−04 | 11543702- | 11547948- | 11548017- |
| | | | | | 11547947 | 11548016 | 11550388 |
| DYNC2H1 | inclusion | 437419 | 430981 | 4.15E−06 | 102996025- | 102999639- | 102999735- |
| | | | | | 102999638 | 102999734 | 103004283 |
| DYNC2LI1 | inclusion | 518440 | 509032 | 1.32E−05 | 44023935- | 44027980- | 44028057- |
| | | | | | 44027979 | 44028056 | 44028777 |
| EBF3 | inclusion | 488452 | 481314 | −1 | 131676114- | 131755522- | 131755591- |
| | | | | | 131755521 | 131755590 | 131757197 |
| EP300 | inclusion | 378053 | 380144 | −1 | 41560135- | 41562603- | 41562671- |
| | | | | | 41562602 | 41562670 | 41564452 |
| F10 | inclusion | 12059 | 27098 | −1 | 113798410- | 113801693- | 113801801- |
| | | | | | 113801692 | 113801800 | 113803229 |
| F13A1 | inclusion | 16524 | 31563 | 3.98E−06 | 6146006- | 6152046- | 6152183- |
| | | | | | 6152045 | 6152182 | 6167690 |
| FBN1 | inclusion | 437420 | 430986 | −1 | 48725186- | 48726791- | 48726911- |
| | | | | | 48726790 | 48726910 | 48729157 |
| FUT8 | inclusion | 545467 | 535727 | −1 | 66188740- | 66190865- | 66191042- |
| | | | | | 66190864 | 66191041 | 66199948 |
| G6PC | inclusion | 214465 | 211810 | 1.77E−05 | 41059646- | 41061320- | 41061436- |
| | | | | | 41061319 | 41061435 | 41062931 |
| GAA | inclusion | 188044 | 185936 | 4.03E−06 | 78082628- | 78083744- | 78083855- |
| | | | | | 78083743 | 78083854 | 78084525 |
| GABRG2 | inclusion | 433102 | 426665 | −1 | 161529680- | 161530895- | 161531033- |
| | | | | | 161530894 | 161531032 | 161569169 |
| GABRG2 | inclusion | 433102 | 426665 | −1 | 161528324- | 161530895- | 161531033- |
| | | | | | 161530894 | 161531032 | 161569169 |
| GCK | inclusion | 36224 | 44888 | −1 | 44189664- | 44190555- | 44190675- |
| | | | | | 44190554 | 44190674 | 44191869 |
| GLA | inclusion | 180842 | 179870 | −1 | 100653556- | 100653773- | 100653935- |
| | | | | | 100653772 | 100653934 | 100654731 |
| GLA | inclusion | 180842 | 179870 | −1 | 100653556- | 100653773- | 100653935- |
| | | | | | 100653772 | 100653934 | 100655653 |
| GNB5 | inclusion | 268098 | 263624 | −1 | 52442123- | 52446137- | 52446274- |
| | | | | | 52446136 | 52446273 | 52471964 |
| GNPTAB | inclusion | 397556 | 384468 | 3.99E−06 | 102174400- | 102179790- | 102179996- |
| | | | | | 102179789 | 102179995 | 102182325 |
| GNPTAB | inclusion | 2761 | 17800 | −1 | 102164936- | 102173930- | 102174065- |
| | | | | | 102173929 | 102174064 | 102174334 |
| GPR143 | inclusion | 10517 | 25556 | −1 | 9716707- | 9727372- | 9727467- |
| | | | | | 9727371 | 9727466 | 9728756 |
| GPX4 | inclusion | 140615 | 150320 | 3.59E−05 | 1105510- | 1105657- | 1105809- |
| | | | | | 1105656 | 1105808 | 1106240 |
| GRHPR | inclusion | 548673 | 539143 | 3.98E−06 | 37425992- | 37426535- | 37426652- |
| | | | | | 37426534 | 37426651 | 37428480 |
| GRN | inclusion | 599616 | 590962 | −1 | 42427709- | 42428918- | 42429164- |
| | | | | | 42428917 | 42429163 | 42429382 |
| GRN | inclusion | 599616 | 590962 | −1 | 42428829- | 42428918- | 42429164- |
| | | | | | 42428917 | 42429163 | 42429382 |
| GUCY2C | inclusion | 31604 | 40288 | 3.99E−06 | 14822768- | 14825807- | 14825893- |
| | | | | | 14825806 | 14825892 | 14827558 |
| GYPA | inclusion | 17714 | 32753 | 3.60E−05 | 145039906- | 145040839- | 145040935- |
| | | | | | 145040838 | 145040934 | 145041642 |
| HBB | inclusion | 36337 | 45001 | 1.03E−04 | 5248030- | 5248160- | 5248270- |
| | | | | | 5248159 | 5248269 | 5248741 |
| IDH1 | inclusion | 375891 | 362770 | 0.00E+00 | 209110149- | 209113093- | 209113385- |
| | | | | | 209113092 | 209113384 | 209116153 |
| IDH1 | inclusion | 375893 | 362772 | 0.00E+00 | 209110149- | 209113093- | 209113385- |
| | | | | | 209113092 | 209113384 | 209116153 |
| IFT57 | inclusion | 506288 | 497913 | −1 | 107886712- | 107910368- | 107910491- |
| | | | | | 107910367 | 107910490 | 107925474 |
| IL36RN | inclusion | 40005 | 48518 | 9.55E−04 | 113817045- | 113818429- | 113818515- |
| | | | | | 113818428 | 113818514 | 113819700 |
| KDSR | inclusion | 427791 | 418321 | 2.83E−05 | 60999135- | 61002490- | 61002592- |
| | | | | | 61002489 | 61002591 | 61006032 |
| KIAA0586 | inclusion | 208813 | 205374 | 1.38E−05 | 58924698- | 58925191- | 58925264- |
| | | | | | 58925190 | 58925263 | 58926560 |
| KIAA0586 | inclusion | 208813 | 205374 | 1.38E−05 | 58924698- | 58925191- | 58925264- |
| | | | | | 58925190 | 58925263 | 58927703 |
| KIAA1109 | inclusion | 487550 | 480599 | −1 | 123145833- | 123147862- | 123147991- |
| | | | | | 123147861 | 123147990 | 123150275 |
| KIF14 | inclusion | 503567 | 495007 | −1 | 200530009- | 200534197- | 200534382- |
| | | | | | 200534196 | 200534381 | 200534572 |
| KIT | inclusion | 13843 | 28882 | −1 | 55594094- | 55594177- | 55594288- |
| | | | | | 55594176 | 55594287 | 55595500 |
| KMT2D | inclusion | 547418 | 537993 | −1 | 49441853- | 49442442- | 49442553- |
| | | | | | 49442441 | 49442552 | 49442887 |
| KRIT1 | inclusion | 590717 | 581356 | −1 | 91843294- | 91843925- | 91844092- |
| | | | | | 91843924 | 91844091 | 91851215 |
| KRIT1 | inclusion | 590718 | 581357 | −1 | 91843294- | 91843925- | 91844092- |
| | | | | | 91843924 | 91844091 | 91851215 |
| LAMB3 | inclusion | 14543 | 29582 | −1 | 209806122- | 209806415- | 209806479- |
| | | | | | 209806414 | 209806478 | 209807791 |
| LDLR | inclusion | 252233 | 246528 | 7.99E−06 | 11230910- | 11231046- | 11231199- |
| | | | | | 11231045 | 11231198 | 11233849 |
| LIPA | inclusion | 203361 | 199794 | 1.03E−04 | 90975767- | 90982268- | 90982340- |
| | | | | | 90982267 | 90982339 | 90983440 |
| LMNA | inclusion | 519180 | 509123 | −1 | 156106228- | 156106712- | 156107024- |
| | | | | | 156106711 | 156107023 | 156107444 |
| LMNA | inclusion | 519180 | 509123 | −1 | 156106820- | 156106904- | 156107024- |
| | | | | | 156106903 | 156107023 | 156107444 |
| LMNA | inclusion | 519180 | 509123 | −1 | 156106820- | 156106904- | 156107024- |
| | | | | | 156106903 | 156107023 | 156108278 |
| LMNA | inclusion | 66854 | 77751 | −1 | 156106228- | 156106712- | 156107024- |
| | | | | | 156106711 | 156107023 | 156107444 |
| LMNA | inclusion | 66854 | 77751 | −1 | 156106820- | 156106904- | 156107024- |
| | | | | | 156106903 | 156107023 | 156107444 |
| LMNA | inclusion | 66854 | 77751 | −1 | 156106820- | 156106904- | 156107024- |
| | | | | | 156106903 | 156107023 | 156108278 |
| MCFD2 | inclusion | 2865 | 17904 | 1.19E−05 | 47135109- | 47136162- | 47136317- |
| | | | | | 47136161 | 47136316 | 47139681 |
| MCFD2 | inclusion | 2865 | 17904 | 1.19E−05 | 47135109- | 47136162- | 47136317- |
| | | | | | 47136161 | 47136316 | 47143119 |
| MCFD2 | inclusion | 2865 | 17904 | 1.19E−05 | 47135109- | 47136162- | 47136317- |
| | | | | | 47136161 | 47136316 | 47142861 |
| MCFD2 | inclusion | 2865 | 17904 | 1.19E−05 | 47135109- | 47136162- | 47136317- |
| | | | | | 47136161 | 47136316 | 47168710 |
| MLH1 | inclusion | 90285 | 95759 | −1 | 37048555- | 37053311- | 37053354- |
| | | | | | 37053310 | 37053353 | 37053501 |
| MLH1 | inclusion | 90285 | 95759 | −1 | 37050397- | 37053311- | 37053354- |
| | | | | | 37053310 | 37053353 | 37053501 |
| MLH1 | inclusion | 90415 | 95889 | −1 | 37056036- | 37058997- | 37059091- |
| | | | | | 37058996 | 37059090 | 37061800 |
| MLH1 | inclusion | 90415 | 95889 | −1 | 37056036- | 37058997- | 37059091- |
| | | | | | 37058996 | 37059090 | 37067127 |
| MLH1 | inclusion | 89616 | 95090 | −1 | 37056036- | 37061801- | 37061955- |
| | | | | | 37061800 | 37061954 | 37067127 |
| MLH1 | inclusion | 89616 | 95090 | −1 | 37059091- | 37061801- | 37061955- |
| | | | | | 37061800 | 37061954 | 37067127 |
| MLH1 | inclusion | 89979 | 95453 | −1 | 37089175- | 37090008- | 37090101- |
| | | | | | 37090007 | 37090100 | 37090394 |
| MMAB | inclusion | 218326 | 215033 | −1 | 110002982- | 110006575- | 110006669- |
| | | | | | 110006574 | 110006668 | 110011151 |
| MMAB | inclusion | 218326 | 215033 | −1 | 110002982- | 110006575- | 110006669- |
| | | | | | 110006574 | 110006668 | 110009453 |
| MMAB | inclusion | 218326 | 215033 | −1 | 110002982- | 110006575- | 110006669- |
| | | | | | 110006574 | 110006668 | 110007776 |
| MPC1 | inclusion | 35561 | 44234 | −1 | 166778942- | 166779462- | 166779595- |
| | | | | | 166779461 | 166779594 | 166780282 |
| MSH2 | inclusion | 483664 | 472825 | −1 | 47690294- | 47693797- | 47693948- |
| | | | | | 47693796 | 47693947 | 47698103 |
| MSH2 | inclusion | 483724 | 472839 | −1 | 47693948- | 47698104- | 47698202- |
| | | | | | 47698103 | 47698201 | 47702163 |
| MTM1 | inclusion | 158960 | 169974 | −1 | 149765035- | 149783062- | 149783173- |
| | | | | | 149783061 | 149783172 | 149787510 |
| MTM1 | inclusion | 158960 | 169974 | −1 | 149767151- | 149783062- | 149783173- |
| | | | | | 149783061 | 149783172 | 149787510 |
| MTM1 | inclusion | 158966 | 169981 | −1 | 149765035- | 149783062- | 149783173- |
| | | | | | 149783061 | 149783172 | 149787510 |
| MTM1 | inclusion | 158966 | 169981 | −1 | 149767151- | 149783062- | 149783173- |
| | | | | | 149783061 | 149783172 | 149787510 |
| MYBPC3 | inclusion | 454301 | 461685 | −1 | 47365176- | 47367758- | 47367922- |
| | | | | | 47367757 | 47367921 | 47368177 |
| MYO7A | inclusion | 228280 | 231385 | 7.19E−06 | 76891528- | 76892426- | 76892636- |
| | | | | | 76892425 | 76892635 | 76892996 |
| NF1 | inclusion | 480206 | 478059 | −1 | 29486112- | 29490204- | 29490395- |
| | | | | | 29490203 | 29490394 | 29508439 |
| NF1 | inclusion | 480206 | 478059 | −1 | 29486112- | 29490204- | 29490395- |
| | | | | | 29490203 | 29490394 | 29496908 |
| NF1 | inclusion | 428998 | 420767 | −1 | 29509684- | 29527440- | 29527614- |
| | | | | | 29527439 | 29527613 | 29528054 |
| NF1 | inclusion | 431976 | 426213 | −1 | 29546137- | 29548868- | 29548948- |
| | | | | | 29548867 | 29548947 | 29550461 |
| NF1 | inclusion | 220498 | 222614 | −1 | 29626625- | 29652838- | 29653271- |
| | | | | | 29652837 | 29653270 | 29654516 |
| NF1 | inclusion | 220498 | 222614 | −1 | 29592358- | 29652838- | 29653271- |
| | | | | | 29652837 | 29653270 | 29654516 |
| NF1 | inclusion | 220498 | 222614 | −1 | 29645583- | 29652838- | 29653271- |
| | | | | | 29652837 | 29653270 | 29654516 |
| NF1 | inclusion | 185354 | 184694 | −1 | 29653271- | 29654517- | 29654858- |
| | | | | | 29654516 | 29654857 | 29657313 |
| NIPBL | inclusion | 446431 | 439877 | −1 | 36971139- | 36972047- | 36972144- |
| | | | | | 36972046 | 36972143 | 36975877 |
| NPHP1 | inclusion | 3510 | 18549 | 1.06E−05 | 110919275- | 110920625- | 110920713- |
| | | | | | 110920624 | 110920712 | 110922264 |
| NPHP1 | inclusion | 3510 | 18549 | 1.06E−05 | 110919275- | 110920625- | 110920710- |
| | | | | | 110920624 | 110920709 | 110922096 |
| NPHP1 | inclusion | 3510 | 18549 | 1.06E−05 | 110919275- | 110920625- | 110920710- |
| | | | | | 110920624 | 110920709 | 110922264 |
| NPHP1 | inclusion | 3510 | 18549 | 1.06E−05 | 110919275- | 110920625- | 110922308- |
| | | | | | 110920624 | 110922307 | 110922628 |
| NPHP1 | inclusion | 3510 | 18549 | 1.06E−05 | 110919275- | 110920625- | 110920713- |
| | | | | | 110920624 | 110920712 | 110922096 |
| NR3C2 | inclusion | 8564 | 23603 | −1 | 149116014- | 149181130- | 149181270- |
| | | | | | 149181129 | 149181269 | 149356255 |
| NR3C2 | inclusion | 8564 | 23603 | −1 | 149116014- | 149181130- | 149181270- |
| | | | | | 149181129 | 149181269 | 149289416 |
| NSD1 | inclusion | 573155 | 560277 | 5.96E−05 | 176710930- | 176715820- | 176715927- |
| | | | | | 176715819 | 176715926 | 176718954 |
| OCA2 | inclusion | 965 | 16004 | 1.19E−05 | 28231790- | 28234747- | 28234813- |
| | | | | | 28234746 | 28234812 | 28259921 |
| OCA2 | inclusion | 965 | 16004 | 1.19E−05 | 28231790- | 28234747- | 28234813- |
| | | | | | 28234746 | 28234812 | 28235721 |
| OFD1 | inclusion | 41060 | 49482 | −1 | 13753203- | 13753367- | 13753466- |
| | | | | | 13753366 | 13753465 | 13754226 |
| OFD1 | inclusion | 41060 | 49482 | −1 | 13753203- | 13753367- | 13753466- |
| | | | | | 13753366 | 13753465 | 13754596 |
| OFD1 | inclusion | 41061 | 49483 | −1 | 13753203- | 13753367- | 13753466- |
| | | | | | 13753366 | 13753465 | 13754226 |
| OFD1 | inclusion | 41061 | 49483 | −1 | 13753203- | 13753367- | 13753466- |
| | | | | | 13753366 | 13753465 | 13754596 |
| OFD1 | inclusion | 41116 | 49538 | −1 | 13757034- | 13757121- | 13757152- |
| | | | | | 13757120 | 13757151 | 13758207 |
| OFD1 | inclusion | 41120 | 49542 | −1 | 13757034- | 13757121- | 13757152- |
| | | | | | 13757120 | 13757151 | 13762533 |
| OFD1 | inclusion | 41120 | 49542 | −1 | 13757034- | 13757121- | 13757152- |
| | | | | | 13757120 | 13757151 | 13758207 |
| OGT | inclusion | 428572 | 420006 | −1 | 70757923- | 70764417- | 70764486- |
| | | | | | 70764416 | 70764485 | 70766060 |
| OGT | inclusion | 428572 | 420006 | −1 | 70757923- | 70764417- | 70764486- |
| | | | | | 70764416 | 70764485 | 70767756 |
| ORC6 | inclusion | 253273 | 247699 | 1.63E−04 | 46726458- | 46727005- | 46727095- |
| | | | | | 46727004 | 46727094 | 46729473 |
| ORC6 | inclusion | 253273 | 247699 | 1.63E−04 | 46726458- | 46727005- | 46727095- |
| | | | | | 46727004 | 46727094 | 46728850 |
| ORC6 | inclusion | 253273 | 247699 | 1.63E−04 | 46726458- | 46727005- | 46727095- |
| | | | | | 46727004 | 46727094 | 46729928 |
| OTC | inclusion | 97286 | 103178 | −1 | 38260682- | 38262871- | 38262994- |
| | | | | | 38262870 | 38262993 | 38267994 |
| OTC | inclusion | 97301 | 103193 | −1 | 38262994- | 38267995- | 38268049- |
| | | | | | 38267994 | 38268048 | 38268128 |
| OTC | inclusion | 97091 | 102983 | −1 | 38268279- | 38271115- | 38271253- |
| | | | | | 38271114 | 38271252 | 38280275 |
| OTOGL | inclusion | 39780 | 48379 | 5.82E−06 | 80733045- | 80735728- | 80735943- |
| | | | | | 80735727 | 80735942 | 80746074 |
| PAFAH1B1 | inclusion | 159550 | 169377 | −1 | 2576052- | 2577354- | 2577583- |
| | | | | | 2577353 | 2577582 | 2579798 |
| PAFAH1B1 | inclusion | 159550 | 169377 | −1 | 2576052- | 2577354- | 2577583- |
| | | | | | 2577353 | 2577582 | 2583457 |
| PAH | inclusion | 102892 | 108628 | 3.98E−06 | 103240730- | 103245465- | 103245535- |
| | | | | | 103245464 | 103245534 | 103246592 |
| PARN | inclusion | 190291 | 178830 | 4.38E−05 | 14700384- | 14702138- | 14702177- |
| | | | | | 14702137 | 14702176 | 14702914 |
| PARN | inclusion | 190291 | 178830 | 4.38E−05 | 14698084- | 14702138- | 14702177- |
| | | | | | 14702137 | 14702176 | 14702914 |
| PDCD10 | inclusion | 468332 | 452270 | −1 | 167402178- | 167405022- | 167405105- |
| | | | | | 167405021 | 167405104 | 167405402 |
| PIGN | inclusion | 101048 | 106813 | 8.03E−05 | 59807713- | 59810539- | 59810580- |
| | | | | | 59810538 | 59810579 | 59813141 |
| PIGN | inclusion | 264636 | 259229 | 1.77E−05 | 59777207- | 59780367- | 59780550- |
| | | | | | 59780366 | 59780549 | 59781793 |
| PMS2 | inclusion | 237932 | 240133 | 3.98E−06 | 6031689- | 6035165- | 6035265- |
| | | | | | 6035164 | 6035264 | 6036956 |
| PMS2 | inclusion | 192316 | 190109 | −1 | 6022623- | 6026390- | 6027252- |
| | | | | | 6026389 | 6027251 | 6029430 |
| POLG | inclusion | 587863 | 580246 | 3.98E−05 | 89862331- | 89862459- | 89862582- |
| | | | | | 89862458 | 89862581 | 89863996 |
| POMGNT1 | inclusion | 3988 | 19027 | 3.98E−06 | 46655662- | 46656145- | 46656190- |
| | | | | | 46656144 | 46656189 | 46656391 |
| PPT1 | inclusion | 56191 | 70830 | −1 | 40555185- | 40557001- | 40557072- |
| | | | | | 40557000 | 40557071 | 40557716 |
| PPT1 | inclusion | 56191 | 70830 | −1 | 40555255- | 40557001- | 40557072- |
| | | | | | 40557000 | 40557071 | 40557716 |
| PRMT7 | inclusion | 266022 | 260845 | 1.44E−05 | 68345003- | 68349800- | 68349978- |
| | | | | | 68349799 | 68349977 | 68355328 |
| PRMT7 | inclusion | 266022 | 260845 | 1.44E−05 | 68345150- | 68349800- | 68349978- |
| | | | | | 68349799 | 68349977 | 68355328 |
| PRMT7 | inclusion | 266022 | 260845 | 1.44E−05 | 68349553- | 68349828- | 68349978- |
| | | | | | 68349827 | 68349977 | 68355328 |
| PRMT7 | inclusion | 266022 | 260845 | 1.44E−05 | 68345003- | 68349800- | 68349978- |
| | | | | | 68349799 | 68349977 | 68358585 |
| PTEN | inclusion | 280031 | 264490 | 4.02E−06 | 89693009- | 89711875- | 89712017- |
| | | | | | 89711874 | 89712016 | 89717609 |
| PTEN | inclusion | 427622 | 416983 | −1 | 89693009- | 89711875- | 89712017- |
| | | | | | 89711874 | 89712016 | 89717609 |
| RAD51B | inclusion | 221910 | 223600 | 4.03E−06 | 68289048- | 68290259- | 68290345- |
| | | | | | 68290258 | 68290344 | 68292180 |
| RAD51B | inclusion | 221910 | 223600 | 4.03E−06 | 68286571- | 68290259- | 68290345- |
| | | | | | 68290258 | 68290344 | 68292180 |
| RAD51C | inclusion | 128201 | 133658 | 1.19E−05 | 56801462- | 56809845- | 56809906- |
| | | | | | 56809844 | 56809905 | 56811478 |
| RAD51C | inclusion | 128201 | 133658 | 1.19E−05 | 56798174- | 56809845- | 56809906- |
| | | | | | 56809844 | 56809905 | 56811478 |
| RAD51C | inclusion | 128201 | 133658 | 1.19E−05 | 56801462- | 56809842- | 56809906- |
| | | | | | 56809841 | 56809905 | 56811478 |
| RB1 | inclusion | 428730 | 420512 | −1 | 48881543- | 48916735- | 48916851- |
| | | | | | 48916734 | 48916850 | 48919215 |
| RB1 | inclusion | 527935 | 528310 | −1 | 49050659- | 49050837- | 49050980- |
| | | | | | 49050836 | 49050979 | 49051490 |
| RB1 | inclusion | 527935 | 528310 | −1 | 49047527- | 49050837- | 49050980- |
| | | | | | 49050836 | 49050979 | 49051490 |
| RHAG | inclusion | 13066 | 28105 | 3.99E−06 | 49573544- | 49574561- | 49574635- |
| | | | | | 49574560 | 49574634 | 49574862 |
| RNF216 | inclusion | 183356 | 181427 | −1 | 5692142- | 5751392- | 5751471- |
| | | | | | 5751391 | 5751470 | 5752345 |
| RTTN | inclusion | 219187 | 217210 | 7.18E−06 | 67692085- | 67695960- | 67696038- |
| | | | | | 67695959 | 67696037 | 67697247 |
| SCN1A | inclusion | 190009 | 187855 | −1 | 166905460- | 166908229- | 166908499- |
| | | | | | 166908228 | 166908498 | 166909361 |
| SLC10A7 | inclusion | 623252 | 612215 | 8.47E−06 | 147204418- | 147214081- | 147214133- |
| | | | | | 147214080 | 147214132 | 147215081 |
| SLC12A1 | inclusion | 8752 | 23791 | 1.42E−05 | 48541874- | 48543812- | 48543968- |
| | | | | | 48543811 | 48543967 | 48548007 |
| SLC4A11 | inclusion | 1311 | 16350 | 1.20E−05 | 3208503- | 3208905- | 3209075- |
| | | | | | 3208904 | 3209074 | 3209157 |
| SMS | inclusion | 11623 | 26662 | −1 | 21990109- | 21990625- | 21990690- |
| | | | | | 21990624 | 21990689 | 21995178 |
| SPAST | inclusion | 468575 | 451183 | 0.00E+00 | 32323961- | 32339707- | 32339895- |
| | | | | | 32339706 | 32339894 | 32340770 |
| SPAST | inclusion | 468575 | 451183 | 0.00E+00 | 32314675- | 32339707- | 32339895- |
| | | | | | 32339706 | 32339894 | 32340770 |
| SPG11 | inclusion | 41362 | 49786 | 7.98E−06 | 44855500- | 44856745- | 44856897- |
| | | | | | 44856744 | 44856896 | 44858051 |
| SPTA1 | inclusion | 544819 | 535240 | 3.19E−05 | 158585194- | 158587327- | 158587379- |
| | | | | | 158587326 | 158587378 | 158587828 |
| SPTA1 | inclusion | 12865 | 27904 | −1 | 158622444- | 158623064- | 158623216- |
| | | | | | 158623063 | 158623215 | 158624400 |
| SPTA1 | inclusion | 544822 | 535243 | −1 | 158624539- | 158626354- | 158626447- |
| | | | | | 158626353 | 158626446 | 158627266 |
| SPTB | inclusion | 544811 | 535260 | −1 | 65268119- | 65268472- | 65268553- |
| | | | | | 65268471 | 65268552 | 65268943 |
| SRD5A2 | inclusion | 3341 | 18380 | 4.88E−05 | 31754528- | 31756441- | 31756543- |
| | | | | | 31756440 | 31756542 | 31758672 |
| STXBP1 | inclusion | 520996 | 511787 | −1 | 130434396- | 130435460- | 130435541- |
| | | | | | 130435459 | 130435540 | 130438082 |
| SYNGAP1 | inclusion | 375528 | 362330 | −1 | 33400030- | 33400462- | 33400584- |
| | | | | | 33400461 | 33400583 | 33402928 |
| TGFBR2 | inclusion | 12504 | 27543 | −1 | 30715739- | 30729876- | 30730004- |
| | | | | | 30729875 | 30730003 | 30732911 |
| TJP2 | inclusion | 397505 | 384407 | 7.96E−06 | 71840324- | 71840938- | 71841092- |
| | | | | | 71840937 | 71841091 | 71842680 |
| TMEM138 | inclusion | 31190 | 40147 | −1 | 61133689- | 61135395- | 61135471- |
| | | | | | 61135394 | 61135470 | 61135723 |
| TMEM138 | inclusion | 31190 | 40147 | −1 | 61133689- | 61135395- | 61135471- |
| | | | | | 61135394 | 61135470 | 61136068 |
| TMPRSS6 | inclusion | 30802 | 39759 | 1.59E−05 | 37467073- | 37469572- | 37469686- |
| | | | | | 37469571 | 37469685 | 37470649 |
| TP53 | inclusion | 80709 | 91600 | 3.98E−06 | 7577609- | 7578177- | 7578290- |
| | | | | | 7578176 | 7578289 | 7578370 |
| TP53 | inclusion | 428868 | 420639 | −1 | 7574034- | 7576853- | 7576927- |
| | | | | | 7576852 | 7576926 | 7577018 |
| TP53 | inclusion | 428868 | 420639 | −1 | 7576658- | 7576853- | 7576927- |
| | | | | | 7576852 | 7576926 | 7577018 |
| TP53 | inclusion | 428868 | 420639 | −1 | 7576585- | 7576853- | 7576927- |
| | | | | | 7576852 | 7576926 | 7577018 |
| TP53 | inclusion | 428868 | 420639 | −1 | 7569563- | 7576853- | 7576927- |
| | | | | | 7576852 | 7576926 | 7577018 |
| TPO | inclusion | 4045 | 19084 | 3.18E−05 | 1488627- | 1491593- | 1491764- |
| | | | | | 1491592 | 1491763 | 1497573 |
| TRAPPC2 | inclusion | 11512 | 26551 | 0.00E+00 | 13734798- | 13737990- | 13738102- |
| | | | | | 13737989 | 13738101 | 13752162 |
| TRAPPC2 | inclusion | 11512 | 26551 | 0.00E+00 | 13734798- | 13737990- | 13738102- |
| | | | | | 13737989 | 13738101 | 13752643 |
| TRAPPC2 | inclusion | 11512 | 26551 | 0.00E+00 | 13734798- | 13737990- | 13738102- |
| | | | | | 13737989 | 13738101 | 13738546 |
| TRAPPC2 | inclusion | 11512 | 26551 | 0.00E+00 | 13734798- | 13737990- | 13738102- |
| | | | | | 13737989 | 13738101 | 13752168 |
| TRIM37 | inclusion | 5246 | 20285 | 8.62E−06 | 57140010- | 57141716- | 57141767- |
| | | | | | 57141715 | 57141766 | 57148183 |
| TYR | inclusion | 99582 | 105471 | 3.99E−06 | 88911941- | 88924370- | 88924587- |
| | | | | | 88924369 | 88924586 | 88960990 |
| TYR | inclusion | 99582 | 105471 | 3.99E−06 | 88911941- | 88924370- | 88924587- |
| | | | | | 88924369 | 88924586 | 88933430 |
| UROD | inclusion | 72 | 15111 | 3.98E−06 | 45480509- | 45480612- | 45480679- |
| | | | | | 45480611 | 45480678 | 45481008 |
| VMA21 | inclusion | 208798 | 205359 | −1 | 150565530- | 150572103- | 150572213- |
| | | | | | 150572102 | 150572212 | 150573387 |
| VMA21 | inclusion | 208799 | 205360 | −1 | 150565530- | 150572103- | 150572213- |
| | | | | | 150572102 | 150572212 | 150573387 |
| VMA21 | inclusion | 208806 | 205367 | −1 | 150565120- | 150572103- | 150572213- |
| | | | | | 150572102 | 150572212 | 150573387 |
| VMA21 | inclusion | 208806 | 205367 | −1 | 150565530- | 150572103- | 150572213- |
| | | | | | 150572102 | 150572212 | 150573387 |
| VMA21 | inclusion | 208720 | 205332 | −1 | 150565120- | 150572103- | 150572213- |
| | | | | | 150572102 | 150572212 | 150573387 |
| VMA21 | inclusion | 208720 | 205332 | −1 | 150565530- | 150572103- | 150572213- |
| | | | | | 150572102 | 150572212 | 150573387 |
| VPS13B | inclusion | 68084 | 78975 | −1 | 100147366- | 100147824- | 100147962- |
| | | | | | 100147823 | 100147961 | 100148892 |
| WDR35 | inclusion | 617905 | 609306 | −1 | 20151246- | 20153595- | 20153740- |
| | | | | | 20153594 | 20153739 | 20166484 |
| WDR35 | inclusion | 617905 | 609306 | −1 | 20151246- | 20153595- | 20153740- |
| | | | | | 20153594 | 20153739 | 20160314 |
| WDR73 | inclusion | 208470 | 204991 | 4.01E−06 | 85191186- | 85191768- | 85191857- |
| | | | | | 85191767 | 85191856 | 85195944 |
| XPC | inclusion | 260 | 15299 | −1 | 14208754- | 14209757- | 14209881- |
| | | | | | 14209756 | 14209880 | 14211937 |
| BRCA1 | exclusion | 55387 | 70054 | 3.98E−06 | 41215391- | 41215891- | 41215969- |
| | | | | | 41215890 | 41215968 | 41234420 |
| BRCA1 | exclusion | 55387 | 70054 | 3.98E−06 | 41215391- | 41215891- | 41215969- |
| | | | | | 41215890 | 41215968 | 41277198 |
| BRCA1 | exclusion | 55419 | 70086 | −1 | 41215391- | 41215891- | 41215969- |
| | | | | | 41215890 | 41215968 | 41234420 |
| BRCA1 | exclusion | 55419 | 70086 | −1 | 41215391- | 41215891- | 41215969- |
| | | | | | 41215890 | 41215968 | 41277198 |
| BRIP1 | exclusion | 578521 | 571401 | 3.98E−06 | 59876661- | 59878614- | 59878836- |
| | | | | | 59878613 | 59878835 | 59885827 |
| BRIP1 | exclusion | 491400 | 485084 | −1 | 59876661- | 59878614- | 59878836- |
| | | | | | 59878613 | 59878835 | 59885827 |
| CDAN1 | exclusion | 3182 | 18221 | −1 | 43026545- | 43027298- | 43027377- |
| | | | | | 43027297 | 43027376 | 43027458 |
| CLN3 | exclusion | 56290 | 70929 | 2.47E−05 | 28488957- | 28493798- | 28493867- |
| | | | | | 28493797 | 28493866 | 28493946 |
| COL6A1 | exclusion | 17169 | 32208 | 3.99E−06 | 47418086- | 47418312- | 47418348- |
| | | | | | 47418311 | 47418347 | 47418810 |
| COL7A1 | exclusion | 17437 | 32476 | −1 | 48619947- | 48620042- | 48620087- |
| | | | | | 48620041 | 48620086 | 48620445 |
| CTSK | exclusion | 8424 | 23463 | −1 | 150778493- | 150778578- | 150778701- |
| | | | | | 150778577 | 150778700 | 150779161 |
| EBP | exclusion | 158530 | 170113 | −1 | 48380296- | 48382311- | 48382461- |
| | | | | | 48382310 | 48382460 | 48385376 |
| ERCC6 | exclusion | 1700 | 16739 | −1 | 50701299- | 50708584- | 50708743- |
| | | | | | 50708583 | 50708742 | 50713929 |
| F8 | exclusion | 10303 | 25342 | −1 | 154129718- | 154130326- | 154130443- |
| | | | | | 154130325 | 154130442 | 154132180 |
| FBN1 | exclusion | 547347 | 538033 | 3.98E−06 | 48707965- | 48712884- | 48713004- |
| | | | | | 48712883 | 48713003 | 48713754 |
| FBN1 | exclusion | 36119 | 44783 | −1 | 48707965- | 48712884- | 48713004- |
| | | | | | 48712883 | 48713003 | 48713754 |
| FIG4 | exclusion | 447336 | 440905 | 2.48E−05 | 110113868- | 110117968- | 110118055- |
| | | | | | 110117967 | 110118054 | 110146290 |
| FLT3 | exclusion | 16271 | 31310 | 4.01E−06 | 28589839- | 28592604- | 28592727- |
| | | | | | 28592603 | 28592726 | 28597486 |
| GLA | exclusion | 10768 | 25807 | 4.53E−05 | 100653935- | 100654732- | 100654789- |
| | | | | | 100654731 | 100654788 | 100655653 |
| HFE | exclusion | 407073 | 395090 | −1 | 26091333- | 26092955- | 26093189- |
| | | | | | 26092954 | 26093188 | 26093346 |
| HFE | exclusion | 407073 | 395090 | −1 | 26091333- | 26092913- | 26093189- |
| | | | | | 26092912 | 26093188 | 26093346 |
| LHCGR | exclusion | 492757 | 485710 | 0.00E+00 | 48950836- | 48952815- | 48952890- |
| | | | | | 48952814 | 48952889 | 48958365 |
| LHCGR | exclusion | 492757 | 485710 | 0.00E+00 | 48950836- | 48952815- | 48952890- |
| | | | | | 48952814 | 48952889 | 48956291 |
| MAPT | exclusion | 14253 | 29292 | 2.62E−05 | 44074031- | 44087676- | 44087769- |
| | | | | | 44087675 | 44087768 | 44091608 |
| MAPT | exclusion | 98218 | 104110 | 5.64E−06 | 44074031- | 44087676- | 44087769- |
| | | | | | 44087675 | 44087768 | 44091608 |
| MAPT | exclusion | 14254 | 29293 | −1 | 44074031- | 44087676- | 44087769- |
| | | | | | 44087675 | 44087768 | 44091608 |
| OTC | exclusion | 97139 | 103031 | −1 | 38226683- | 38229049- | 38229094- |
| | | | | | 38229048 | 38229093 | 38240612 |
| SCN5A | exclusion | 448976 | 442549 | −1 | 38662463- | 38663891- | 38663981- |
| | | | | | 38663890 | 38663980 | 3867180 1 |
| SDCCAG8 | exclusion | 212141 | 206783 | −1 | 243449700- | 243456393- | 243456522- |
| | | | | | 243456392 | 243456521 | 243468014 |
| SDCCAG8 | exclusion | 212141 | 206783 | −1 | 243449700- | 243456393- | 243456522- |
| | | | | | 243456392 | 243456521 | 243456700 |
| SDHD | exclusion | 6903 | 21942 | −1 | 111957684- | 111958581- | 111958698- |
| | | | | | 111958580 | 111958697 | 111959590 |
| SDHD | exclusion | 6903 | 21942 | −1 | 111957684- | 111958581- | 111958698- |
| | | | | | 111958580 | 111958697 | 111959590 |
| SDHD | exclusion | 6903 | 21942 | −1 | 111957684- | 111958581- | 111958698- |
| | | | | | 111958580 | 111958697 | 111965528 |
| SNX10 | exclusion | 139565 | 143196 | 2.39E−05 | 26386087- | 26400595- | 26400682- |
| | | | | | 26400594 | 26400681 | 26404154 |
| SNX10 | exclusion | 139565 | 143196 | 2.39E−05 | 26396748- | 26400595- | 26400682- |
| | | | | | 26400594 | 26400681 | 26404154 |
| SNX10 | exclusion | 139565 | 143196 | 2.39E−05 | 26393805- | 26400595- | 26400682- |
| | | | | | 26400594 | 26400681 | 2640415 4 |
| STK11 | exclusion | 216070 | 213437 | −1 | 1219413- | 1220372- | 1220717- |
| | | | | | 1220371 | 1220716 | 1221211 |
| TCIRG1 | exclusion | 5462 | 20501 | 4.03E−05 | 67806587- | 67808735- | 67808814- |
| | | | | | 67808734 | 67808813 | 67809219 |
| TECTA | exclusion | 228404 | 230005 | 1.99E−05 | 121038927- | 121039386- | 121039635- |
| | | | | | 121039385 | 121039634 | 121058540 |
| TFR2 | exclusion | 21379 | 34231 | −1 | 100229569- | 100229705- | 100229822- |
| | | | | | 100229704 | 100229821 | 100230623 |
| TP53 | exclusion | 428868 | 420639 | −1 | 7574034- | 7576525- | 7576585- |
| | | | | | 7576524 | 7576584 | 7576852 |

Table 19 lists those ClinVar pathogenic mutations predicted to be rescued by Compound (I) treatment selected based on top populational allele frequencies in gnomAD v2.1.1, as described herein, that may be modulated by Compound (I) toward either or both exon inclusion and exon exclusion.

TABLE 19
ATM, BRCA1, CA5A, DGUOK, DNAH9, GLA,
GPX4, GYPA, HBB, IL36RN, KDSR, LIPA, MYO7A,
NPHP1, NSD1, ORC6, PARN, PIGN, POLG,
SLC12A1, SPTA1, SRD5A2, TCIRG1, and TPO.

One aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 19.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 19.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 19.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 19.

Another aspect described herein is Compound (I) for use in the method, wherein Compound (I) modulates the production of one or more mature RNA isoforms from a predicted wildtype or mutant gene transcript in a cell, wherein the method comprises, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 19.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

Another aspect described herein is Compound (I) for use in the method, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

Another aspect described herein is Compound (I) for use in the method, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

One aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 19.

Another aspect described herein is Compound (I) for use in the method, wherein Compound (I) modulates the production of one or more mature RNA isoforms from a wildtype gene transcript in a cell, wherein the method comprises, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 19.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 19.

Another aspect described herein is Compound (I) for use in the method, wherein Compound (I) modulates the production of one or more mature RNA isoforms from a mutant gene transcript in a cell, wherein the method comprises, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 19.

One aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 19.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 19.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 19.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 19.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), wherein N represents any DNA nucleotide, and wherein the predicted wildtype or mutant gene transcript is transcribed from a gene selected from Table 19.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), wherein N represents any DNA nucleotide, and wherein the predicted wildtype or mutant gene transcript is transcribed from a gene selected from Table 8.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), wherein N represents any RNA nucleotide, and wherein the predicted wildtype or mutant gene transcript is transcribed from a gene selected from Table 8.

Another aspect described herein is use of Compound (I) to modulate the production of one or more mature RNA isoforms from a predicted wildtype or mutant gene transcript in a cell comprising, contacting the cell with Compound (I), wherein the predicted wildtype or mutant gene transcript includes a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), wherein N represents any RNA nucleotide, and wherein the predicted wildtype or mutant gene transcript is transcribed from a gene selected from Table 8.

One aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4), and wherein the wildtype gene transcript is transcribed from a gene selected from Table 19.

Another aspect described herein is use of Compound (I) to modulate the production of one or more mature RNA isoforms from a wildtype gene transcript in a cell comprising, contacting the cell with Compound (I), wherein the wildtype gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8), and wherein the wildtype gene transcript is transcribed from a gene selected from Table 19.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16), and wherein the mutant gene transcript is transcribed from a gene selected from Table 19.

Another aspect described herein is use of Compound (I) to modulate the production of one or more mature RNA isoforms from a mutant gene transcript in a cell comprising, contacting the cell with Compound (I), wherein the mutant gene transcript comprises, three exons and two introns operably linked in the following order: Exon 1, Intron 1, Exon 2, Intron 2, and Exon 3, wherein a 5′ splice site of Exon 2 comprises, a sequence other than a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24), and wherein the mutant gene transcript is transcribed from a gene selected from Table 19.

### Identification of Potential Therapeutic Targets of Compound (I)

About 20% of all CV-pMUTs were predicted to alter splicing within 50 nucleotides of the mutation, and that about 80% of these disrupt Ensembl-annotated (GRCh37 version 75) splice sites (FIG. 4A). The responsive genes (Gene) containing the top 20 most frequent mutations (gnomAD v2.1.1) (Mutation) are shown in Table 20. The human diseases associated with the Mutations are shown in Table 14.

Table 20 lists ClinVar pathogenic mutations (ClinVar) predicted by Splice AI (Prediction) to be rescued by either Gain or Loss after Compound (I) treatment, with corresponding isoform change (Response) were selected based on top populational allele frequencies in gnomAD (v2.1.1) (Frequency).

TABLE 20
ClinVar Pathogenic Mutations

| | | | | SpliceAI | |
|---|---|---|---|---|---|
| Gene | Mutation | Frequency | ClinVar | Prediction | Response |
| ATM | c.2250G > A | 4.39E−05 | Synonymous | Loss | Inclusion |
| CA5A | c.555G > A | 1.67E−04 | Synonymous | Loss | Inclusion |
| DGUOK | c.591G > A | 1.27E−04 | Intronic/ | Loss | Inclusion |
| | | | Synonymous | | |
| DNAH9 | c.1970 + | 3.18E−04 | Intronic | Loss | Inclusion |
| | 4A > G | | | | |
| GLA | c.639 + | 4.53E−05 | Intronic | Gain | Exclusion |
| | 919G > A | | | | |
| GPX4 | c.476 + | 3.65E−05 | Intronic | Loss | Inclusion |
| | 5G > A | | | | |
| GYPA | c.232G > A | 3.60E−05 | Missense | Loss | Inclusion |
| HBB | c.92G > A | 1.03E−04 | Missense | Loss | Inclusion |
| IL36RN | c.115 + | 1.00E−03 | Intronic | Loss | Inclusion |
| | 6T > C | | | | |
| KDSR | c.879G > A | 6.37E−05 | Synonymous | Loss | Inclusion |
| LIPA | c.894G > A | 8.28E−04 | Synonymous | Loss | Inclusion |
| MYO7A | c.2904G > A | 3.20E−05 | Synonymous | Loss | Inclusion |
| NPHP1 | c.1027G > A | 6.37E−05 | Missense | Loss | Inclusion |
| NSD1 | c.6152 − | 5.96E−05 | Intronic | Loss | Inclusion |
| | 5T > G | | | | |
| ORC6 | c.449 + | 1.63E−04 | Intronic | Loss | Inclusion |
| | 5G > A | | | | |
| PARN | c.659 + | 4.63E−05 | Intronic | Loss | Inclusion |
| | 4_659 + | | | | |
| | 7delAGTA | | | | |
| PIGN | c.963G > A | 8.31E−05 | Synonymous | Loss | Inclusion |
| POLG | c.3104 + | 3.98E−05 | Intronic | Loss | Inclusion |
| | 3A > T | | | | |
| SLC12A1 | c.1942G > A | 6.37E−05 | Missense | Loss | Inclusion |
| SRD5A2 | c.547G > A | 1.59E−04 | Missense | Loss | Inclusion |

### Minigene Validation of the CNN Model

To validate whether the CNN model correctly predicted mutated exon triplet response to Compound (I) using the minigene system and RT-PCR, the following rules were used to select suitable exon triplets: 1) Exon triplet total length, including introns, was less than 1.5 kb and suitable for cloning, 2) Exon triplet splicing changes were detectable in fibroblast RNASeq against Compound (I), where the response classes could be used as quality checks of minigene expression; and 3) Wild-type exon triplet splicing of the minigene recapitulated the response observed in the fibroblast RNASeq before and after treatment with Compound (I), thus confirming the splicing process in the minigene was intact.

Minigene splicing assays for certain genes having exon triplets harboring mutations were used to confirm the predictive accuracy of the CNN model results. The genes were chosen based on their genomic triplet length which enabled them to be cloned into a splice vector. Minigenes for CPSF7, SETD5 and PARP6 were developed (as shown in FIGS. 3E-3G, respectively) with alternatively spliced triplets that the CNN model had predicted would respond in the presence of Compound (I). One nucleotide in the 5′ splice site (in adjacent nucleotides from the +2 to +6 position) was randomly mutated for each minigene, resulting in mutations in the +3 position (CPSF7 and PARP6) and the +6 position (SETD5), then the response to treatment in both wild-type and mutant constructs was predicted by the CNN model and subsequently confirmed by RT-PCR. For example, FIG. 3E illustrates that the mutated CPSF7 minigene was predicted to be affected by the presence of Compound (I) toward inclusion and subsequently confirmed by RT-PCR. FIG. 3F indicates that the mutated SETD5 minigene was predicted to be affected by the presence of Compound (I) toward exclusion, which was subsequently confirmed by RT-PCR. FIG. 3G shows that the mutated PARP6 gene isolated from patient cells was predicted to be affected by the presence of Compound (I) toward inclusion, which was also subsequently confirmed by RT-PCR.

### Results of Minigene Validation of CNN Model

In one example from Table 18, the IL36RN gene harbors the mutation c.115+6T>C, which is 6 nucleotides downstream of exon 3 and was annotated as an intronic mutation in a patient with psoriasis (ClinVar ID: 40005). SpliceAI predicted that this mutation would cause skipping of Exon 3 and the CNN model predicted that the mutated sequence would be responsive to Compound (I) treatment, promoting exon 3 inclusion.

Similarly, in another example from Table 18, the c.894G>A mutation located at the last nucleotide of exon 8 in LIPA was annotated as a synonymous mutation (ClinVar ID: 203361), leading to deficient activity of lysosomal acid lipase (LAL)^{56,57,59,60} and characterized by the accumulation of harmful amounts of lipids in cells and tissues throughout the body. Mutations in LIPA are expressed in two major phenotypes: the severe infantile-onset Wolman disease and the milder late-onset cholesterol ester storage disease (CESD)⁵⁸⁻⁶⁰. The severity of the condition depends on the residual LAL activity⁵⁷. The c.894G>A LIPA gene mutation is the most common mutation in the LIPA gene, found in about 50% of individuals with LAL deficiency⁵⁸. This mutation is responsible for the milder disease CESD and leads to skipping of exon 8. SpliceAI predicted the mutation would cause exon 8 skipping while the CNN model predicted the mutated sequence would be responsive to Compound (I) treatment, promoting exon 8 inclusion (see FIG. 4C). To confirm the prediction for LIPA, a cell line (Coriell Cell Repository) carrying the c.894G>A mutation in the LIPA gene was treated with Compound (I) (60 μM) for 24 hours. As predicted and shown in FIG. 4C, the WT sequence was unaffected, but Compound (I) promoted the inclusion of exon 8 for the mutated gene, with cells showing a significant 10% increase in normal transcript levels.

In another example from Table 18, the ClinVar and CNN model predictions related to the CFTR gene carrying a c.2988G>A mutation (see FIG. 4D) were confirmed by stably expressing a minigene containing the full length CFTR coding sequence carrying the c.2988G>A mutation and flanking introns (EMG-MUT)⁶³ in HEK293 cells. The c.2988G>A mutation was reported to cause skipping of exon 18 in the CFTR gene and is associated with abnormal CFTR function and a mild form of Cystic Fibrosis⁶⁴. The CNN model predicted that CFTR exon skipping would be rescued by Compound (I) treatment (as shown in FIG. 4D)⁵⁹. Using RT-PCR, the minigene was confirmed to harbor the mutation (EMG-MUT), showing only 5% exon 18 inclusion. As shown in FIG. 4D, treatment with Compound (I) (60 μM) for 24 hours significantly increased exon inclusion in the EMG-MUT.

In another example from Table 18, a minigene encompassing MLH1 exons 16 to 18 and flanking exon 17 intronic sequences, with the c.1989 G>A mutation was prepared. This mutation was predicted by SpliceA1 to cause skipping of exon 17 in the MLH1 gene. The CNN model predicted that the skipping of this exon would be rescued by Compound (I) treatment. The c.1989 G>A mutation in MLH1 is associated with hereditary nonpolyposis colorectal cancer (HNPCC) or Lynch syndrome (MIM #120435). Individuals with MLH1 mutations also have an increased risk of cancers of the endometrium, ovaries, stomach, small intestine, liver, gallbladder duct, upper urinary tract, and brain^{14-16,65}. In order to validate the treatment effect on the splicing of exon 17, transfected cell lines were treated with Compound (I) (60 μM) for 24 hours. As shown in FIG. 4E, the treatment significantly promoted exon 17 inclusion.

In another example from Table 18, several MAPT minigenes encompassing exons 9 to 11 and flanking exon 10 intronic sequences, with the three mutations listed in Table 17: c.1866+3 G>A, c.1865 G>A or c.1788 T>G were prepared. These mutations were predicted by SpliceA1 to promote inclusion of MAPT exon 10. The CNN model predicted that Compound (I) would promote exon 10 exclusion. An increase in MAPT exon 10 inclusion is responsible for familial frontotemporal dementia and Parkinsonism linked to chromosome 17 (FTDP-17, MIM #600274). Alternative splicing of MAPT exon 10 is tightly regulated and generates two tau isoforms with three (3R tau, exon 10 skipped) or four (4R tau, exon 10 included) microtubule-binding repeats, the latter having an increased affinity for microtubules. Exon 10 is expressed only in adult human brain and 3R and 4R tau isoforms are expressed in approximately equal amounts. Mutations affecting exon 10 splicing result in an up to 6-fold excess of 4R tau and in an elevated 4R/3R ratio⁶⁸⁻⁷². Elevated 4R/3R ratio leads to saturation of microtubule binding sites and increase of unbound 4R tau which assembles into filaments. The most common mutation in MAPT gene is the C to T substitution in intron 10, c.1866+16C>T, commonly referred to as IVS10+16. This mutation increases inclusion of exon 10. The MAPT c.1866+16C>T mutation was not initially identified by SpliceAI to affect splicing, but the CNN model for this mutation predicted the treatment effect of Compound (I) on exon 10 splicing to promote exon 10 exclusion. The MAPT minigene carrying the c.1866+16C>T mutation was used to validate the CNN Model prediction. The use of Compound (I) treatment to affect splicing of MAPT exon 10 was validated in transfected cell lines treated with Compound (I) (60 μM) for 24 hours. As shown in FIG. 4F, the treatment significantly promoted exon 10 exclusion for all three mutations tested.

### ELP1 Gene Modulation Using Kinetin

Splicing alterations identified at three 5′ splice site junctions in triplets of consecutive exons in the Elongator complex Protein 1 gene (ELP1), having a 5′ splice site mutation resulting in Exon 20 exclusion, after treatment with kinetin, a small molecule 6-furfurylaminopurine splicing modulator compound, have demonstrated corrective splicing^{44,46-50,93} A Percent Spliced In (PSI) change of 0.57 was assessed by RNA sequencing for ELP1 exon 20 splicing after kinetin treatment^{44,46-50,93}. 42 additional exon-usage differences in response to kinetin were observed: 11 of the usage differences promoted inclusion of the middle exon and 31 usage differences induced skipping of the middle exon^{44,46-50,93}.

Kinetin is a naturally occurring small molecule splicing modulator compound with a safe absorption, distribution, metabolism, and excretion (ADME) profile; however, extremely high doses are necessary to achieve inclusion of Exon 20 in vivo^{44,46-50,93}. As described herein, in vitro and in vivo splicing assays in various cell lines, including patient cells, have identified Compound (I) as a potent and efficacious small molecule splicing modulator compound.

### ELP1 Gene Modulation Using Compound (I)

Compound (I) was evaluated in vivo to determine whether splicing correction can lead to a concomitant increase in ELP1 protein. In brief, TgFD9 transgenic mice, which carry the human ELP1 gene with a Exon 20 splice mutation, were treated by oral gavage once daily for 7 days with Compound (I) as a suspension in 0.5% HPMC, 0.1% Tween 80 at a dose of 10, 30, 60 or 100 mg/kg. The mice used for this study were housed in an animal facility, provided with access to food and water ad libitum, and maintained on a 12-hour light/dark cycle. For routine genotyping, genomic DNA was prepared from tail biopsies and PCR was carried out to detect the TgFD9 transgene using the following primers—forward 5′-GCCATTGTACTGTTTGCGACT-3′; reverse, 5′-TGAGTGTCACGATTCTTTCTGC-3′. Mice were sacrificed 1 hour after the last dose.

Brain, liver, lung, kidney, heart and skin tissues were removed and snap frozen in liquid nitrogen. Tissues were homogenized in ice-cold QIAzol Lysis Reagent (Qiagen), using Qiagen TissueLyser II (Qiagen). Total RNA was extracted using the QIAzol reagent procedure. The yield, purity and quality of the total RNA for each sample were determined using a Nanodrop ND-1000 spectrophotometer. Full-length and mutant ELP1 mRNA expression was quantified by quantitative real-time PCR (qRT-PCR) analysis using CFX384 Touch Real-Time PCR Detection System (BioRad). Reverse transcription and qPCR were carried out using One Step RT-qPCR (BioRad). The mRNA levels of full-length ELP1, mutant Δ20 ELP1 and GAPDH were quantified using Taqman-based RT-qPCR with a cDNA equivalent of 25 ng of starting RNA in a 20-μl reaction. To amplify the full-length ELP1 isoform, FL ELP1 forward primer 5′-GAGCCCTGGTTTTAGCTCAG-3′; reverse primer 5′-CATGCATTCAAATGCCTCTTT-3′, and FL ELP1 probe 5′-TCGGAAGTGGTTGGACAAACTTATGTTT-3′ were used. To amplify the mutant (Δ20) ELP1 spliced isoforms, Δ20 ELP1 forward primer, 5′-CACAAAGCTTGTATTACAGACT-3′; reverse primer 5′-GAAGGTTTCCACATTTCCAAG -3′ and Δ20 ELP1 probe 5′-CTCAATCTGATTTATGATCATAACCCTAAGGTG-3′ were used. The ELP1 forward and reverse primers were each used at a final concentration of 0.4 μM. The ELP1 probes were used at a final concentration of 0.15 μM. Mouse GAPDH mRNA was amplified using 20× gene expression PCR assay (Life Technologies, Inc.). RT-qPCR was carried out at the following temperatures for indicated times: Step 1: 48° C. (15 min); Step 2: 95° C. (15 min); Step 3: 95° C. (15 sec); Step 4: 60° C. (1 min); Steps 3 and 4 were repeated for 39 cycles. The Ct values for each mRNA were converted to mRNA abundance using actual PCR efficiencies. ELP1 FL and Δ20 mRNAs were normalized to GAPDH and vehicle controls and plotted as fold change compared to vehicle treatment. Data were analyzed using SDS software.

### Compound (I) In Vivo ELP1 Protein Quantification

Tissue samples were collected, snap frozen in liquid nitrogen, weighed, and homogenized on the TissueLyzer II (Qiagen) in RIPA buffer (Tris-HCl 50 mM, pH 7.4; NaCl 150 mM; NP-40 1%; sodium deoxycholate 0.5%; SDS 0.1%) containing a cocktail of protease inhibitors (Roche) at a tissue weight to RIPA buffer volume of 50 mg/mL. The samples were then centrifuged for 20 min at 14,000×g in a microcentrifuge. The homogenates were transferred to a 96-well plate and diluted in RIPA buffer to ˜1 mg/mL for ELP1-HTRF and ˜0.5 mg/mL for total protein measurement using the BCA protein assay (Pierce). Samples were run in duplicate and averaged. For the ELP1-HTRF assay, 35 μL of tissue homogenate were transferred to a 384-well plate containing 5 μL of the antibody solution (1:50 dilution of anti-ELP1 D2 and anti-ELP1 cryptate from Cisbio). The plate was incubated overnight at room temperature. Fluorescence was measured at 665 nm and 620 nm on an EnVision multilabel plate reader (Perkin Elmer). Total protein content was quantified in each tissue homogenate using the BCA assay. The total protein normalized change in ELP1 protein signal for Compound (I) and vehicle treated tissue sample was calculated as ratio of the signal in the presence of the test compound (e.g., Compound (I)) over the signal in the absence of the Compound (I) (vehicle control).

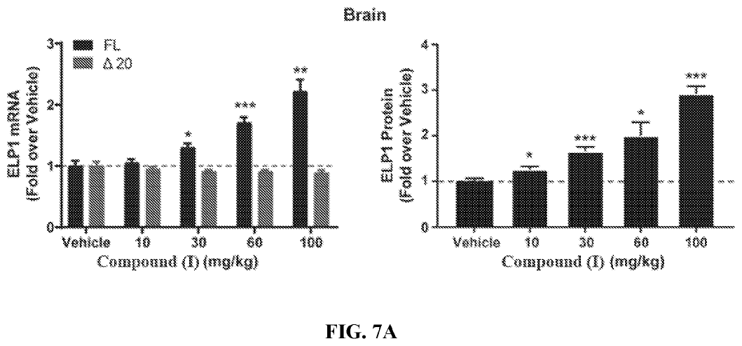

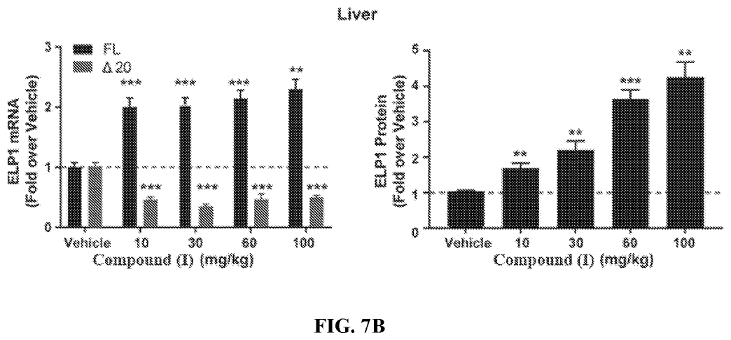

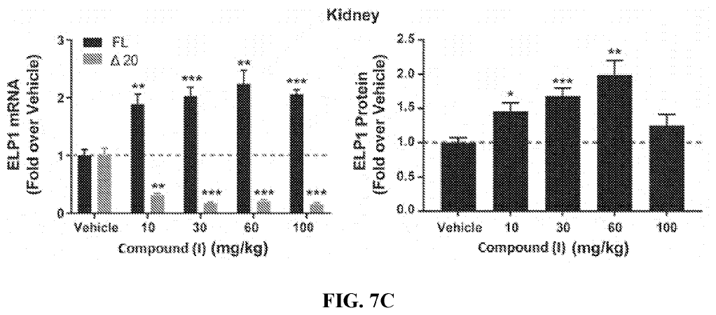

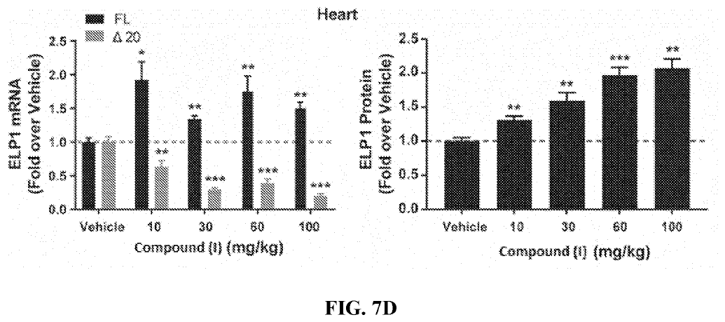

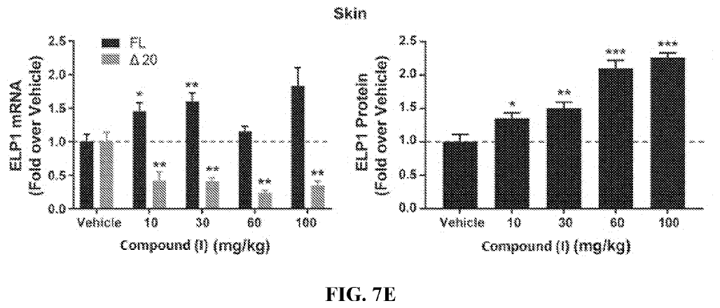

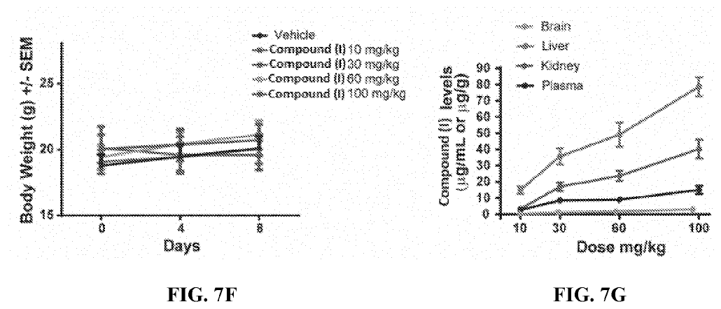

Treatment increased full-length ELP1 transcript in a dose-dependent manner and led to at least a two-fold increase in functional ELP1 protein in brain, liver, kidney, heart and skin (FIGS. 7A-7E). The treatment was found to be well tolerated with no weight loss or adverse effects observed in the treated groups. The level of splicing correction correlated with Compound (I) tissue distribution (FIGS. 7F-7G). These results demonstrated that treatment with Compound (I), which corrects splicing of the ELP1 transcript, significantly increased the level of functional protein in vivo in all tissues, including the brain (FIGS. 7A-7B).

### Materials and Methods

### HEL-293 Cell Culture

HEK-293T (ATCC) cells were cultured in Dulbecco's modified Eagle's medium (11995-065, D-MEM, Gibco) supplemented with 10% fetal bovine serum (FBS, 12306C, SIGMA) and 1% penicillin/streptomycin (30-009-CI, Corning).

### Minigene Generation and Site Direct Mutagenesis

Wild-type and mutant double-stranded DNA (dsDNA) fragments, selected based on low nucleotide length and exon-skipping probability, were ordered through GENEWIZ (FragmentGENE). Adenosine was enzymatically attached to DNA fragment 3′ ends with Taq Polymerase in the presence of 200 nM dATP and 2 mM MgCl₂ at 70° C. for 30 min. Fragments were ligated into linearized pcDNA™3.1/V5-His TOPO® TA plasmid (K480001 ThermoFisher Scientific) according to manufacturer's instructions. After colony selection and sequence confirmation, each plasmid was finally purified using MIDIprep kit (740410, NucleoBond® Xtra Midi, Takara, Mountain View, Calif.). Concentrations were determined using a nanodrop spectrometer.

### Wild-Type and Mutant CFTR Minigene

HEK-293 cells stably transfected with the expression minigenes (EMGs) for the full-length coding sequence and flanking intron sequence of the Cystic Fibrosis Transmembrane Conductance Regulator (CFTR) (courtesy of Dr. Garry R. Cutting, Johns Hopkins University school of Medicine, Baltimore, Md.) were cultured in D-MEM supplemented with 10% FBS (FBS), 1% penicillin/streptomycin and 0.1 mg/mL Hygromycin (SIGMA).

### Wild-Type and Mutant ELP1 and LIPA Culture in Patient Cells

Patient human fibroblast cells GM04663 (Coriell Cell Repository) carrying the c.2204+6T>C mutation in ELP1 and patient human fibroblast cells GM03111 (Coriell Cell Repository) carrying the c.894G>A mutation in LIPA and human wild type fibroblasts (Coriell Cell Repository) were used for the RNA sequence and were cultured in D-MEM supplemented with 10% FBS and 1% penicillin/streptomycin.

### HEK293 Transfection

HEK293T cells were seeded in 6-well culture plates at 1.20×10⁶ cells/well in D-MEM, 10% FBS, without antibiotics and incubated overnight to reach approximately 90% confluence. Transfection was performed with FuGENE® HD Transfection Reagent (E2311, Promega) using the FuGENE-DNA ratio at 3.5:1 and following manufacturer protocol. After 4 hours of incubation at 37° C., cells were plated at a density of 3×10⁴ cells/well in a poly-L-lysine coated 96-well plate for the dual luciferase minigene assay or at the density of 8.5×10⁵ cells/well into 6-well plates for minigene transfection. After 16 hours incubation at 37° C., Compound (I) or DMSO was added at the desired concentrations as described in the next paragraph and kept in culture for other 24 hours.

### Fibroblast Cell Lines

Six different human fibroblast cell lines from healthy individuals were obtained from Coriell Institute and cultured in D-MEM (Gibco) supplemented with 10% FBS and 1% penicillin/streptomycin. Cells were counted and plated in order to achieve semi-confluence after eight days. Twenty-four hours after plating, the medium was changed and cells were treated with Compound (I) or DMSO to a final concentration of 30 μM and 0.5%, respectively. DMSO was used as vehicle. The concentration of Compound (I) was chosen to induce splicing changes and ELP1 protein increase. After seven days of treatment, cells were collected, and RNA was extracted using the QIAzol Reagent following the manufacturer's instructions. RNASeq libraries were prepared using the strand-specific dUTP method^{24,90,91}.

The six different wildtype (WT) human fibroblast cell lines shown in Table 22 included three wildtype human fibroblast cell lines from three caucasian males (10, 12, and 32 years of age) and three caucasian females (3 month, 11, and 11 years of age).

TABLE 22
Wildtype (WT) Human Fibroblast Cell Lines

| Coriell # | Genotype | Sex | Age | Race |
|---|---|---|---|---|
| AG16409 | WT | Male | 12 years | Caucasian |
| GM03348 | WT | Male | 10 years | Caucasian |
| GM08402 | WT | Male | 32 years | Caucasian |
| GM01652 | WT | Female | 11 years | Caucasian |
| GM02036 | WT | Female | 11 years | Caucasian |
| GM00041 | WT | Female | 3 months | Caucasian |

### RNA Isolation and RT-PCR Analysis

After treatment, the cells were collected and RNA was extracted with QIAzol Lysis Reagent (79306, Qiagen) following the manufacturer's instructions. The yields of the total RNA for each sample were determined using a Nanodrop ND-1000 spectrophotometer.

Reverse transcription was performed using 0.5-1 μg of total RNA, Random Primers (C1181, Promega), Oligo(dT)15 Primer (SEQ ID NO: 103) (C1101, Promega), and Superscript III reverse transcriptase (18080093, ThermoFisher Scientific) according to the manufacturer's protocol. cDNA was used to perform PCR reaction in a 20-25 μL volume, using GoTaq® green master mix (MT123, Promega). The primers and melting temperature (Tm) used were according to the manufacturer's protocol. To measure minigene splicing, forward and reverse primers were designed to include the TOPO/V5 plasmid vector and flanking exon sequence in order to avoid endogenous gene detection. PCR reaction was performed as follows: 32 cycles of (95° C. for 30 s, Tₘ for 30 s, 72° C. for 30 s), products were resolved on a 1.5-3% agarose gel, depending on the dimension of the bands to be separated, and visualized using ethidium bromide staining.

Ratios between isoforms having the middle exon included or excluded were obtained using the integrated density value (IDV) for each correspondent band, and then assessed using Alpha 2000™ Image Analyzer and quantified by ImageJ software. The level of exon inclusion was calculated as previously described⁴, where the relative density value of the band represents inclusion and is expressed as a percentage. Validation of the semi-quantitative RT-PCR method for the measurement of exon inclusion was performed using RT-qPCR⁴.

### RNA Sequencing

Briefly, RNA sample quality (based on RNA Integrity Number, or RIN) and quantity was determined using the Agilent 2200 TapeStation, with between 100-1000 ng of total RNA used for library preparation. Each RNA sample was spiked with 1 μL of diluted (1:100) External RNA Controls Consortium (ERCC) RNA Spike-In Mix (4456740, ThermoFisher Scientific), alternating between mix 1 and mix 2 for each well in the batch. Samples were then enriched for mRNA using polyA capture, followed by stranded reverse transcription and chemical shearing to make appropriate stranded cDNA inserts. Libraries were finished by adding Y-adapters, with sample specific barcodes, followed by between 10-15 rounds of PCR amplification. Libraries were evaluated for final concentration and size distribution by Agilent 2200 TapeStation and/or qPCR, using Library Quantification Kit (KK4854, Kapa Biosystems), and multiplexed by pooling equimolar amounts of each library prior to sequencing. Pooled libraries were 50 base pair paired-end sequenced on Illumina HiSeq 2500 across multiple lanes. Real time image analysis and base calling were performed on the HiSeq 2500 instrument using the HiSeq Sequencing Control Software (HCS) and FASTQ files demultipled using CASAVA software version 1.8. RNASeq reads were mapped to the human genome Ensembl GRCh37 by STAR v2.5.2a allowing 5% mismatch^{24,90,91}. The exon triplet index was built according to transcriptome Ensembl GRCh37 version 75. Reads spliced at each exon triplet splice junction were calculated by STAR on the fly.

### Differential Splicing Analysis

For each exon triplet in a certain biological replicate, the ψ (Percent Splice In) value was calculated as (0.5*(R1+R2)/(0.5*(R1+R2)+R3). The average ψ was calculated for treated and untreated conditions, followed by calculation of ψ change. For a certain exon triplet in a certain biological replicate, a 2×2 table was created, where the four cells of the table represent number of reads supporting middle exon inclusion and skipping before and after treatment. Thus, for each exon triplet, a total of six 2×2 tables were created for each of the six biological replicates. A Cochran-Mantel-Haenszel test was applied to test whether an association between treatment and splicing across all replicates (namely whether the cross-replicate odds ratio is 1 or not) could be determined. For each exon triplet, a p value for the Cochran-Mantel-Haenszel test was reported. Benjamini-Hochberg false-discovery-rate (BH FDR) correction was finally applied top values of all triplets.

The primers and melting temperature (Tm) that were used for RNA Isolation and RT-PCR Analysis are shown in Table 20A AND 20B.

TABLE 20A
Forward Primers

| | | SEQ | |
|---|---|---|---|
| | | ID | |
| Gene | Forward 5′-3′ | NO | Tm |
| ELP1 | CCTGAGCAGCAATCATGTG | 111 | 58 C° |
| KLC1 | CGCAGTGGTTCCTTTAGC | 112 | 60 C° |
| LPIN1 | GCTGTGATTTACCCTCAGTCAGC | 113 | 64 C° |
| HSD17B4 | GCAGAAAGAGGAGCGTTA | 114 | 56 C° |
| SLC4A7 | GCTACAGAGGACTGGACG | 115 | 60 C° |
| LRRC28 | GATATAGTGCTGCAGCGTGC | 116 | 60 C° |
| AFMID | GCCTTTCTTCCTGTTCTTTCACG | 117 | 60 C° |
| CD99P1 | CGACCCAGCACCTCTTAATTC | 118 | 62 C° |
| CRYZ | GCACTGCTGGTACTGAGGAAG | 119 | 64 C° |
| IP6K2 | AACAAGCCAAGGAGCCAAGA | 120 | 62 C° |
| EVC | TGCCCTGAAGCTGATGAAGG | 121 | 62 C° |
| EPB41L2 | GGAGAAGTACCTGATGCCGAC | 122 | 60 C° |
| CYLD | GATGGTTCTACACAGCCACC | 123 | 62 C° |
| MEGF6 | CTGGTTTGGAGAGGCCTGTG | 124 | 60 C° |
| MYEF2 | GTACCGTGGTGCGATGACTA | 125 | 60 C° |
| ASXL1 | GCCTCGAGTTGTCCTGACTC | 126 | 60 C° |
| SPTAN1 | CGATCGTCAGGGTTTTGTGC | 127 | 60 C° |
| COPS8 | CTGAGGGACAGTCTGGGGTT | 128 | 60 C° |
| PDZD11 | GTCAGTGAGCGGAGTCTGAG | 129 | 60 C° |
| KTN1 | AGGCAGAGATGGAACGATCT | 130 | 60 C° |
| SPPL2A | TCATGGTTGAACTCGCAGCT | 131 | 60 C° |
| LIPA | CCCAGAGTGCGTTTTTGAA | 132 | 60 C° |
| CFTR | GCAGTGATTATCACCAGC | 133 | 58 C° |
| PTEN | TTGCACAATATCCTTTTGAAGACCA | 134 | 60 C° |
| CPSF7 | ATTGCCCTTGACCCAGAGTT | 135 | 60 C° |
| SETD5 | TGTGGTGGAATTGCCCTTAC | 136 | 60 C° |
| MLH1 | GTGGAATTGCCCTTGAGC | 137 | 60 C° |
| PARP6 | GGAATTGCCCTTGATCATCT | 138 | 60 C° |
| MAPT | ACCCAAGTCGCCGTCTTCCGCC | 139 | 60 C° |

TABLE 20B
Reverse Primers

| | | SEQ | |
|---|---|---|---|
| | | ID | |
| Gene | Reverse 5′-3′ | NO | Tm |
| ELP1 | TACATGGTCTTCGTGACATC | 140 | 58 C° |
| KLC1 | CACTGCTGCTGCTGTCG | 141 | 60 C° |
| LPIN1 | CTTAGCAGCCTGCGGCAGC | 142 | 64 C° |
| HSD17B4 | GTTGGCCACTGCTTTTCC | 143 | 56 C° |
| SLC4A7 | CTAGAACTGGACCTGTGCTCC | 144 | 60 C° |
| LRRC28 | CAACTATGTTATTTGAGTGCAGG | 145 | 60 C° |
| AFMID | GGTGAGCACGTTGTCCTTCT | 146 | 60 C° |
| CD99P1 | CGGTGGAATCAGGCTGCTTG | 147 | 62 C° |
| CRYZ | CTTTGCCATGGTGTCTCGTGG | 148 | 64 C° |
| IP6K2 | ATTCAGGCCACACTTCCCTG | 149 | 62 C° |
| EVC | GGTGCCAGCGTCTGCTTC | 150 | 62 C° |
| EPB41L2 | CTCACTCTCACTGCTGCTG | 151 | 60 C° |
| CYLD | CTTCCCAGTAGGGTGAAGTGAC | 152 | 62 C° |
| MEGF6 | GGGACGGACTGCAACCTCA | 153 | 60 C° |
| MYEF2 | TCTGACAAATATCTGGTTGCCT | 154 | 60 C° |
| ASXL1 | TTCAGGCAGGAGGAAGAGGA | 155 | 60 C° |
| SPTAN1 | TTCTGGAGCACCTCAACCTG | 156 | 60 C° |
| COPS8 | ATGGAGCAAATATAAAGCTAGAAGC | 157 | 60 C° |
| PDZD11 | TGAGGAGGAATCCATGCTGG | 158 | 60 C° |
| KTN1 | TGCAAATCACCAGCTACCTTCT | 159 | 60 C° |
| SPPL2A | TGAGGCACACACTCATTACTGA | 160 | 60 C° |
| LIPA | CCCAGTCAAAGGCTTGAAAC | 161 | 60 C° |
| CFTR | GGAGGAAATATGCTCTCAAC | 162 | 58 C° |
| PTEN | TTAGCATCTTGTTCTGTTTGTGGA | 163 | 60 C° |
| CPSF7 | GAGCGAATAACCTGGATCAGC | 164 | 60 C° |
| SETD5 | GCAGAATTGCCCTTCTGATA | 165 | 60 C° |
| MLH1 | CACTGTGCTGGATATCTGCTG | 166 | 60 C° |
| PARP6 | AATTGCCCTTCTGCAGTTTG | 167 | 60 C° |
| MAPT | CACCTTGCTCAGGTCAACTGGT | 168 | 60 C° |

### In Vitro and In Vivo Compound (1) Treatment

Compound (I) (>9900 pure) was mixed in 100% DMSO to yield 40 mM stock solutions. Working solutions (10×) were prepared by dilution to 500 DMSO in phosphate-buffered saline (PBS, GIBCO). The final DMSO concentration in the treated or untreated cells was 0.5%. Kinetin was purchased from Sigma (K3253).

Cells to be treated with Compound (I) were seeded at the appropriate density in specific vessels to reach semiconfluency at the time of treatment. BEK293T cells transfected with minigenes were seeded in 6 wells and patient fibroblasts were seeded in 10 cm dishes using the described media. The following day, the media was changed with regular growth media supplemented with Compound (I) or DMSO working solutions to obtain final concentrations of 60 μM Compound (I) and 0.05% DMSO. The concentration of 60 μM for dosing Compound (I) was chosen to maximize the possible effect on splicing. Cells were collected for RNA extraction 24 hours after compound or DMSO addition.

### Compound (I) ELP1 Dual-Luciferase Splicing Assay

The Rluc-FD-Fluc plasmid used for the dual-luciferase splicing assay was derived using the ELP1 FD minigene^{44,45,93} containing the ELP1 genomic sequence spanning exon 19-21 inserted into spcDNA3.1/V5-His Topo (Invitrogen). The firefly luciferase (FLuc) coding sequence was inserted immediately after exon 21 and Renilla luciferase (RLuc) upstream of exon 19. Characterization of the assay demonstrated that RLuc was expressed each time a transcript was generated from the reporter plasmid, while FLuc was only expressed when exon 20 was included in the transcript, thereby keeping FLuc in-frame. Evaluation of FLuc/RLuc expression provided the percent exon inclusion in the splicing assays. To perform the dual-luciferase assay, HEK-293T cells were transfected with the minigene plasmid and treated with Compound (I) for 24 hours as described above. After treatment, cells were washed once in PBS and lysed for 25 minutes at room temperature using 50 μL/well of passive lysis buffer (E1941, Promega). Luciferase activity was measured in each sample (20 μL) using the Dual-Luciferase® Reporter Assay reagents (Promega) and the GloMax® 96 Microplate Luminometer (Promega), following manufacturer's instruction, in a 96 well format⁴⁹. The integration time on the luminometer was set at 10-seconds. Compound (I) and kinetin were serially diluted in DMSO and PBS to generate concentration-response curves over eight concentrations, with each point run in quadruplicate. Cells cultured in the presence of 0.5% DMSO were used as controls and run in each plate in quadruplicate.

### Minigene Protein Isolation and Western Blot Analysis

Protein extracts were obtained by homogenizing cells in RIPA buffer (Tris-HCl 50 mM, pH 7.4; NaCl 150 mM; NP-40 1%; sodiumdeoxycholate 0.5%; SDS 0.1%, 1 mM DTT) containing protease and phosphatase inhibitor cocktail (Roche). Insoluble debris were discarded after centrifugation and protein concentration was determined using Pierce® BCA Protein Assay Kit (Thermo Scientific). For LIPA WB, 30 μg of protein lysate was separated on NuPage 10% Bis-Tris gel (Invitrogen) and transferred into nitrocellulose membrane (Thermo Scientific). Membrane was blocked in Odyssey blocking buffer (Licor biosciences) for 1 h at room temperature and incubated overnight at 4° C. with a mouse monoclonal antibody against LIPA (Abnova clone 9G7F12, 1:200) and with a rabbit polyclonal antibody against actin (Sigma, 1:5000). Membranes were washed 3 times in PBS with 0.1% tween 20 and incubated with IRDye secondary antibodies (Licor biosciences) for 1 h at room temperature. Protein bands were visualized by Odyssey CLx imager (Licor biosciences).

### Compound (I) Treatment in a LIPA Cell Model

Wolman disease and CESD are both caused by mutations in LIPA. Wolman is lethal in infancy, whereas CESD patients have some residual enzyme activity and therefore have a milder clinical course. Patients with only 3% of the normal level of LIPA transcript have the much milder disease CESD.

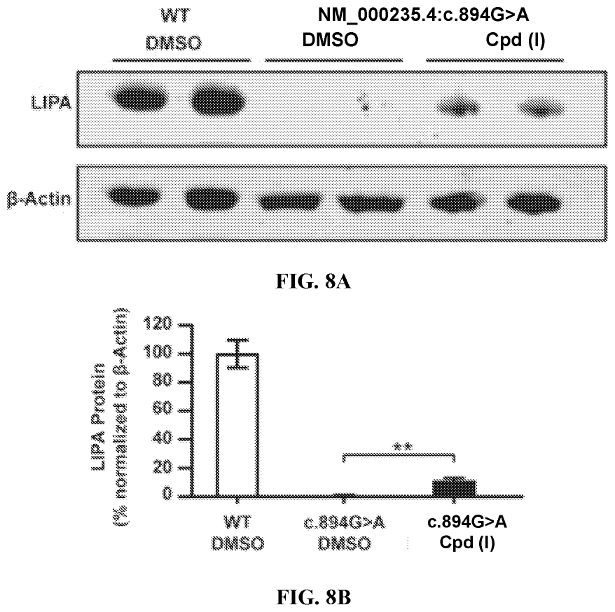

Patient fibroblasts carrying the major LIPA splicing mutation, c.894G>A were treated with 60 μM of Compound (I) for seven days (FIG. 8A). The treatment led to a 10% increase in functional LAL enzyme in mutated cells (FIG. 8B). Given that a 3% increase in residual LAL enzyme activity is enough to distinguish Wolman disease, which is lethal in infancy, from the much milder CESD, a 10% increase in functional LAL can be predicted to have clinical benefit, and Compound (I) increased exon 8 inclusion and lead to a 10% increase in functional protein in a patient cell line, suggesting high potential therapeutic efficacy.

### CFTR Minigene Preparation

For CFTR WB, 40 μg of protein lysate was separated on 7.5% Criterion TGX protein gel (BioRad) and transferred to a PVDF membrane using a Trans-Blot Turbo Transfer System (BioRad). After blocking in 5% non-fat dry milk (BioRad), the membrane was probed with mouse monoclonal anti-CFTR antibody (1:5000 in PBST) that recognizes amino acids 1204-1211 (596, CFFT, North Carolina Chapel Hill). Rabbit monoclonal anti-Na+K+ ATPase (Abcam) (1:50,000 in PBST) was used as a loading control. Secondary antibodies were anti-mouse (1:150,000 GE Healthcare) and anti-rabbit (1:100,000 GE Healthcare), respectively. Blots were exposed on film using ECL Primer Western Blotting Detection Reagent (GE Healthcare)

### Creation of 293-Flp-In and CFBE-Flp-In Stable Cell Lines

WT CFTR-EMG-i14-i18, CFTR-EMG-i14-i18 harboring c.2988G>A, Flp-In-CFBE, and Flp-In-293 cells stably expressing WT-EMG and c.2988G>A EMG were generated. Both Flpin-293 and Flp-In-CFBE cells lack endogenous CFTR expression (CFBE41o-) and contain a single Flp recombinase target site. Co-transfection of EMG plasmid with pOG44 (a plasmid encoding Flp recombinase) allowed for genomic integration at the FRT site. Hygromycin was used for stable cell selection.

### CFTR Functional Assessment

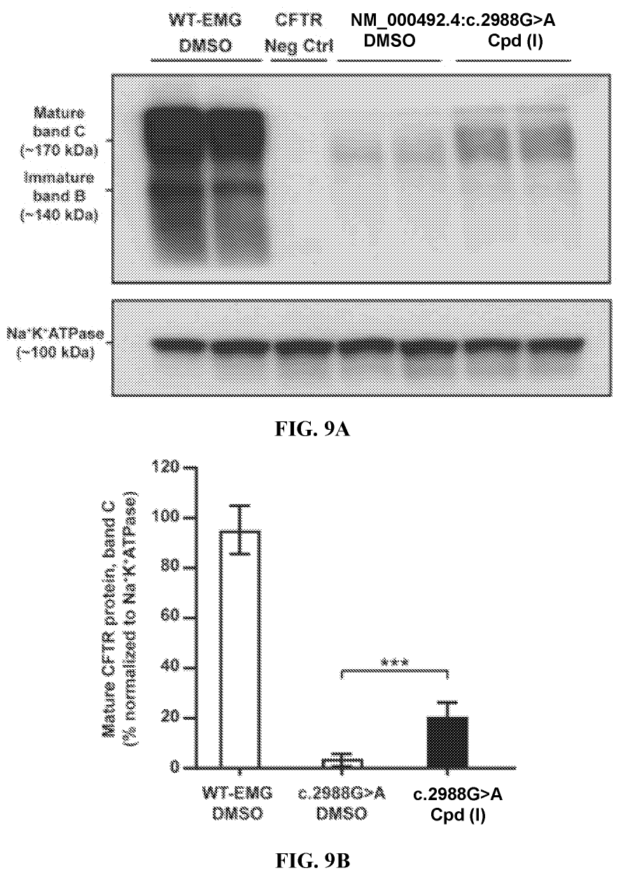

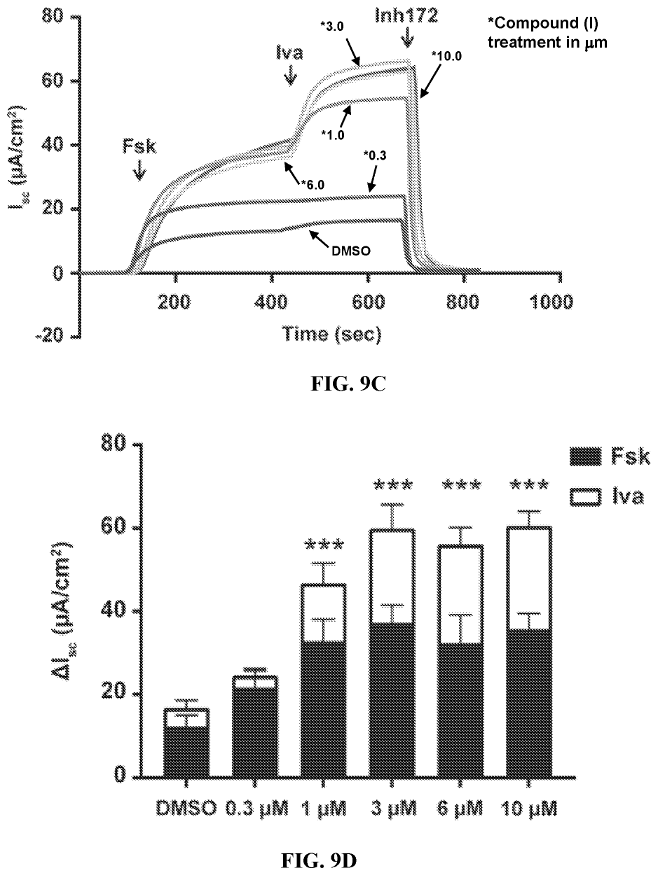

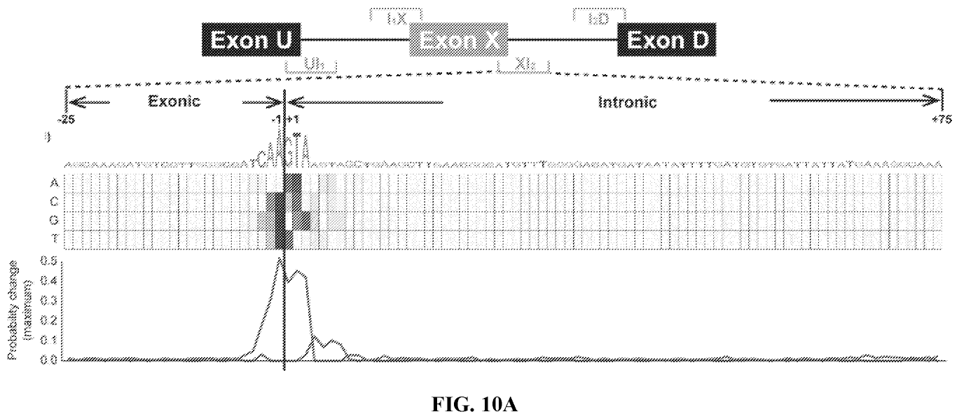

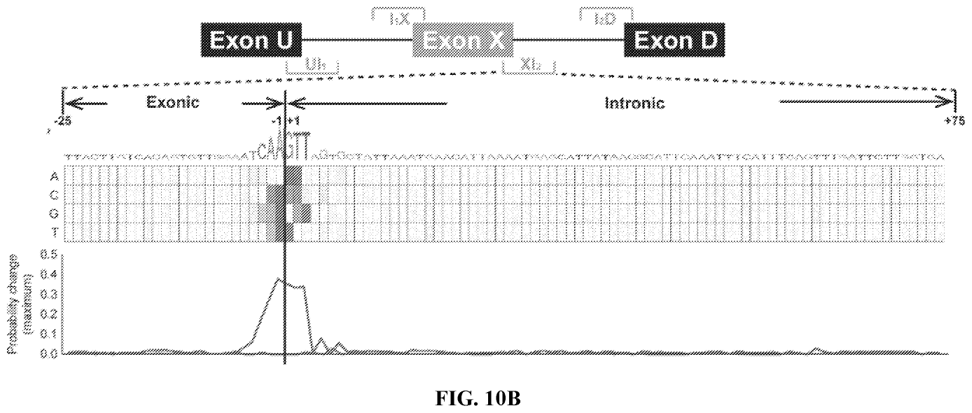

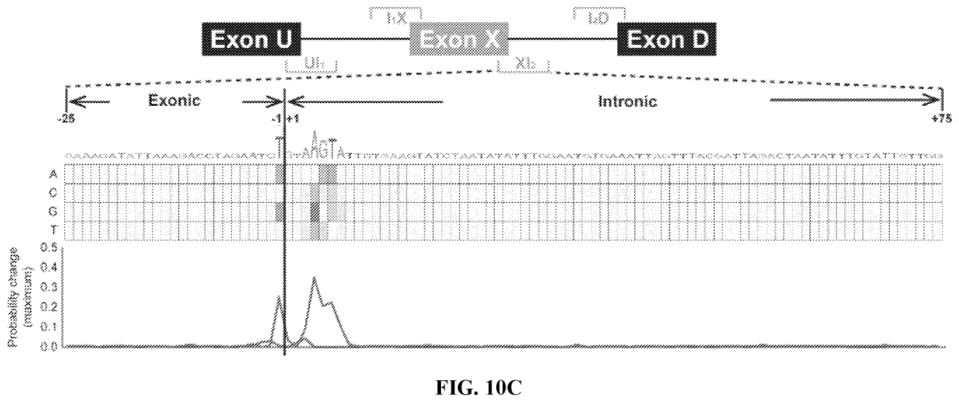

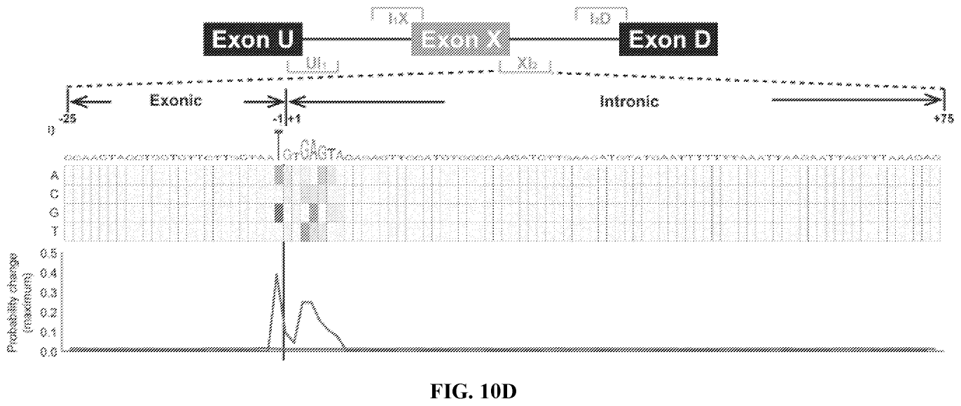

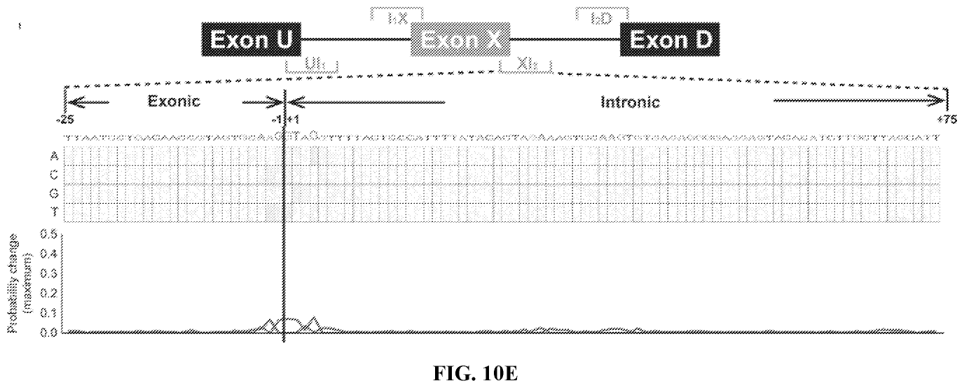

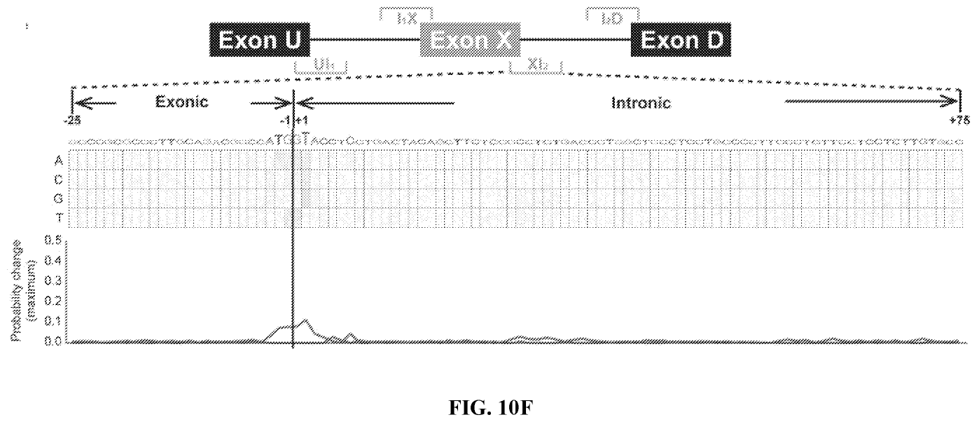

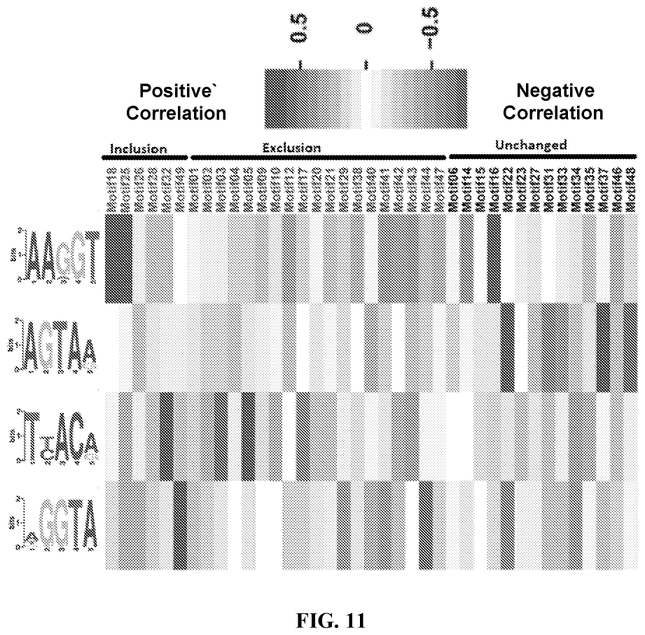

Assessment of CFTR channel function and response to Compound (I) was performed in CFBEs stably expressing c.2988G>A. Briefly, CFBE stable cell lines were plated on snapwell filters. When transepithelial resistance reached ˜200Ω (˜ 5-7 days) as measured using Voltammeter (Physiologic Instruments), cells were treated with Compound (I) at varying doses (0.3 μM to 10 μM) for next 3 days. Filters were mounted in Ussing chambers (Physiological Instruments). A high chloride solution was added to the basolateral chamber and a low chloride solution was added to the apical chamber. After equilibration of currents, 10 μM forskolin (Selleckchem) was added to the basolateral side to activate CFTR channels via cAMP signaling. Currents were allowed to plateau, followed by acute addition of 10 μM ivacaftor at apical side for CFTR potentiation (Selleckchem). Inhibition of CFTR was performed using 10 μM Inh-172 (Selleckchem) added to the apical chamber. FIG. 9C shows a drop in short-current (ΔIsc), defined as the current inhibited by Inh-172 after sustained Isc responses were achieved upon stimulation with forskolin alone or sequentially with ivacaftor, was a quantifiable measurement assigned to CFTR channel function.

### Validation of CFTR as a Therapeutic Target for Compound (I)

To evaluate the efficacy of Compound (I) to correct CFTR aberrant splicing caused by the c.2988G>A mutation (FIG. 9) in a Flp-In-293 stable cell line expressing c.2988G>A CFTR-EMG-i14-i18 were generated to contain full-length introns 14 and 16, and abridged introns 15, 17 and 18. The c.2988G>A variant is located in the last nucleotide position of exon 18 and results in a synonymous change (Gln996Gln) at the protein level. RT-PCR using CFTR specific primers revealed about 3% normal spliced transcript in the Flp-In-293 stable cells expressing 2988G>A (FIG. 9B). Treatment with Compound (I) at 60 μM for 5 days increased exon 18 inclusion by 10% (FIG. 9B) confirming the CNN model prediction. Further, the results suggested that an optimized form of the SMC can be beneficial for other forms of FTD caused by gain of function mutations in exon 10, such as P301L, P301S or the S305N, since treatment could reduce the level of mutated transcript.

### Compound (I) Treatment in a CFTR Cell Model

CFTR protein levels was assessed in Flp-In-293 cells stably expressing the c.2988G>A splicing variant after treatment with Compound (I) for 5 days. FIG. 9A shows that the WT CFTR EMG_il4-i18 control cell line produced predominantly the higher molecular weight, complex-glycosylated mature protein (band C˜170 kDa) as well as some lower molecular weight, core-glycosylated immature protein (band B). Flp-In-293 c.2988G>A stable cells treated with DMSO produce ˜3% of WT complex-glycosylated mature CFTR protein while treatment with 60 μM of Compound (I) for 5 days increased the amount of mature CFTR protein to ˜20% of WT (FIG. 9B).

The ability of Compound (I) to rescue chloride channel function was also assessed. CF bronchial epithelial (CFBE) cell lines was generated to stably express the splicing mutation c.2988G>A. Cells were grown in monolayers on filters and treated with increasing doses of Compound (I) (0.3 μM-10 μM) or DMSO for 3 days. Chloride channel function was assessed by measuring short circuit current (I_{sc}) on treated CFBE cells. Forskolin was added to initiate CFTR channel activity via cAMP-mediated signaling, with further channel activation by Ivacaftor and inhibition with Inh-172, a CFTR-specific inhibitor (FIG. 9C, representative Isc tracing). CFTR-specific change in current (ΔI_{sc}±SD) allows for measurement of chloride channel function (FIG. 9C). Residual CFTR channel activity was observed in DMSO treated CFBE stable cells expressing c.2988G>A (ΔI_{sc}=12.3±2.7 μA/cm², FIG. 9C). Significant recovery of CFTR function (˜3 fold) was observed following treatment with Compound (I) at 1, 3, 6, and 10 μM for 3 days, with a maximal increase in CFTR function achieved using 3 μM of Compound (I) (ΔI_{sc}=37.168±4.32 μA/cm², FIG. 9C). Importantly, the acute addition of Ivacaftor resulted in ˜2 fold improvement in CFTR function in Compound (I) treated cells. These results showed that Compound (I) treatment alone increased chloride channel function to ˜20% of WT, and to ˜30% of WT in combination with Ivacaftor. Given that slight residual CFTR function can lead to a mild form of CF, the increase in chloride channel function to 20% of WT by treatment with Compound (I) can be a prediction of clinical significance and indicates that the splicing-targeted approach of the present disclosure is potentially efficacious.

### Compound (I) Treatment in an ELP1 Minigene

Compound (I) corrected splicing of ELP1 in a minigene system. In vivo splicing correction of ELP1 in a humanized transgenic mouse model led to an increase of ELP1 protein in all tissues, including the brain (FIGS. 7A-7B).

### Results

The results demonstrated herein indicate that the identification of splicing potential for a SMC can provide druggable targets, where even a small increase in functional protein may provide a dramatic therapeutic effect for a neurologic disease phenotype. To determine the potential of Compound (I) to correct splicing of other genes, the machine learning approach using sequence signatures to predict targetable splicing defects was developed as described. The resulting CNN Model identified 39 CNN Motifs important for drug response, with 2 sets of 12 and 13 CNN Motifs accounting for most of the Compound (I) sensitivity when the CNN Motif is located close to the 5′ splice site. Evaluation of splice site strength in drug responsive triplets where middle exon inclusion is increased showed that such exons generally have weaker 5′ splice sites.

The CNN Model predictions of the present disclosure (combined with the fact that kinetin and analog Compound (I) are shown herein to promote the recognition of ELP1 exon 20 through recruitment of U1 snRNP at the 5′ splice site) strongly suggesting that small molecule compounds can act by promoting, either directly or indirectly, the recognition of weakly defined exons.

Application of the CNN Model to all ClinVar pathogenic mutations that disrupt splicing, identified 214 human disease-causing mutations in 155 unique genes as potential therapeutic targets of Compound (I), proving that a deep learning model such as the CNN described herein provide a powerful approach to explore novel therapeutic targets for drugs that modify RNA splicing. As such, the treatment effect on splicing was validated for several disease-causing mutations using patient cell lines and minigenes, and demonstrated the potential therapeutic feasibility of targeting splicing in patients with cystic fibrosis (CFTR), cholesterol ester storage disease (LIPA), Lynch syndrome (MLH1) and familial frontotemporal dementia (MAPT), amongst others. These findings could have significant impact for patients carrying these mutations.

It will be appreciated that, although specific aspects have been described herein for purposes of illustration, the present description is not to be limited in scope by the specific illustrations and examples herein disclosed. These illustrations and examples are intended to merely represent several aspects of the invention. Any equivalent aspects of the illustrations and examples are also intended to be within the scope of the present description. Indeed, various modifications of the aspects of the illustrations and examples, in addition to those shown and described herein, will become apparent to those skilled in the art from the foregoing description, modification of which is intended to be within the scope.

All references cited herein are incorporated herein by reference in their entirety and for all purposes to the same extent as if each individual publication, patent or patent application was specifically and individually indicated to be incorporated by reference in its entirety for all purposes.

### REFERENCES

1. Cooper, T. A., Wan, L. & Dreyfuss, G. RNA and disease. Cell 136, 777-793 (2009).
2. Ritchie, D. B., Schellenberg, M. J. & MacMillan, A. M. Spliceosome structure: piece by piece. Biochim. Biophys. Acta 1789, 624-633 (2009).
3. Monani, U. R., et al. A single nucleotide difference that alters splicing patterns distinguishes the SMA gene SMN1 from the copy gene SMN2. Hum. Mol. Genet. 8, 1177-1183 (1999).
4. Cuajungco, M. P., et al. Tissue-specific reduction in splicing efficiency of IKBKAP due to the major mutation associated with familial dysautonomia. Am. J. Hum. Genet. 72, 749-758 (2003).
5. Flanigan, K. M., et al. Mutational spectrum of DMD mutations in dystrophinopathy patients: application of modern diagnostic techniques to a large cohort. Hum. Mutat. 30, 1657-1666 (2009).
6. Juan-Mateu, J., et al. Interplay between DMD point mutations and splicing signals in Dystrophinopathy phenotypes. PLoS One 8, e59916 (2013).
7. Thornton, C. A. Myotonic dystrophy. Neurol. Clin. 32, 705-719, viii (2014).
8. Pros, E., et al. NF1 mutation rather than individual genetic variability is the main determinant of the NF1-transcriptional profile of mutations affecting splicing. Hum. Mutat. 27, 1104-1114 (2006).
9. Bottillo, I., et al. Functional analysis of splicing mutations in exon 7 of NF1 gene. BMC Med. Genet. 8, 4 (2007).
10. Tzetis, M., Efthymiadou, A., Doudounakis, S. & Kanavakis, E. Qualitative and quantitative analysis of mRNA associated with four putative splicing mutations (621+3A->G, 2751+2T->A, 296+1G->C, 1717-9T->C-D565G) and one nonsense mutation (E822X) in the CFTR gene. Hum. Genet. 109, 592-601 (2001).
11. Cabello, G. M., Cabello, E. J., Jr., Fernande, O. & Harris, A. The 3120+1G->A splicing mutation in CFTR is common in Brazilian cystic fibrosis patients. Hum. Biol. 73, 403-409 (2001).
12. Giorgi, G., et al. Validation of CFTR intronic variants identified during cystic fibrosis population screening by a minigene splicing assay. Clin. Chem. Lab. Med. 53, 1719-1723 (2015).
13. Goina, E., Fernandez-Alanis, E. & Pagani, F. Approaches to study CFTR pre-mRNA splicing defects. Methods Mol. Biol. 741, 155-169 (2011).
14. Rhine, C. L., et al. Hereditary cancer genes are highly susceptible to splicing mutations. PLoS Genet 14, e1007231 (2018).
15. Srebrow, A. & Kornblihtt, A. R. The connection between splicing and cancer. J. Cell Sci. 119, 2635-2641 (2006).
16. Skotheim, R. I. & Nees, M. Alternative splicing in cancer: noise, functional, or systematic? Int. J. Biochem. Cell Biol. 39, 1432-1449 (2007).
17. Dlamini, Z., Mokoena, F. & Hull, R. Abnormalities in alternative splicing in diabetes: therapeutic targets. J. Mol. Endocrinol. 59, R93-R107 (2017).
18. Juan-Mateu, J., Villate, O. & Eizirik, D. L. MECHANISMS IN ENDOCRINOLOGY: Alternative splicing: the new frontier in diabetes research. Eur. J. Endocrinol. 174, R225-238 (2016).
19. Bamshad, M. J., et al. The Centers for Mendelian Genomics: a new large-scale initiative to identify the genes underlying rare Mendelian conditions. Am. J. Med. Genet. A 158A, 1523-1525 (2012).
20. Sanders, S. J., et al. Insights into Autism Spectrum Disorder Genomic Architecture and Biology from 71 Risk Loci. Neuron 87, 1215-1233 (2015).
21. Fresard, L., et al. Identification of rare-disease genes using blood transcriptome sequencing and large control cohorts. Nat. Med. 25, 911-919 (2019).
22. Landrum, M. J., et al. ClinVar: public archive of relationships among sequence variation and human phenotype. Nucleic Acids Res. 42, D980-985 (2014).
23. Jaganathan, K., et al. Predicting Splicing from Primary Sequence with Deep Learning. Cell 176, 535-548 e524 (2019).
24. Johnson, N. T., Dhroso, A., Hughes, K. J. & Korkin, D. Biological classification with RNA-seq data: Can alternatively spliced transcript expression enhance machine learning classifiers? RNA 24, 1119-1132 (2018).
25. Paggi, J. M. & Bejerano, G. A sequence-based, deep learning model accurately predicts RNA splicing branchpoints. RNA 24, 1647-1658 (2018).
26. Wang, J. & Wang, L. Deep Learning of the Back-splicing Code for Circular RNA Formation. Bioinformatics (2019).
27. Palacino, J., et al. SMN2 splice modulators enhance U1-pre-mRNA association and rescue SMA mice. Nat. Chem. Biol. 11, 511-517 (2015).
28. Hua, Y., et al. Antisense correction of SMN2 splicing in the CNS rescues necrosis in a type III SMA mouse model. Genes Dev. 24, 1634-1644 (2010).
29. Passini, M. A., et al. Antisense oligonucleotides delivered to the mouse CNS ameliorate symptoms of severe spinal muscular atrophy. Sci. Transl. Med. 3, 72ra18 (2011).
30. Aartsma-Rus, A. & van Ommen, G. J. Antisense-mediated exon skipping: a versatile tool with therapeutic and research applications. RNA 13, 1609-1624 (2007).
31. Dal Mas, A., Rogalska, M. E., Bussani, E. & Pagani, F. Improvement of SMN2 pre-mRNA processing mediated by exon-specific U1 small nuclear RNA. Am. J. Hum. Genet. 96, 93-103 (2015).
32. Havens, M. A., Duelli, D. M. & Hastings, M. L. Targeting RNA splicing for disease therapy. Wiley interdisciplinary reviews. RNA 4, 247-266 (2013).
33. Sinha, R., et al. Antisense oligonucleotides correct the familial dysautonomia splicing defect in IKBKAP transgenic mice. Nucleic Acids Res. 46, 4833-4844 (2018).
34. Vigevani, L. & Valcarcel, J. Molecular biology. A splicing magic bullet. Science 345, 624-625.
35. Naryshkin, N. A., et al. Motor neuron disease. SMN2 splicing modifiers improve motor function and longevity in mice with spinal muscular atrophy. Science 345, 688-693.
36. Woll, M. G., et al. Discovery and Optimization of Small Molecule Splicing Modifiers of Survival Motor Neuron 2 as a Treatment for Spinal Muscular Atrophy. J. Med. Chem. 59, 6070-6085 (2016).
37. Ratni, H., et al. Discovery of Risdiplam, a Selective Survival of Motor Neuron-2 (SMN2) Gene Splicing Modifier for the Treatment of Spinal Muscular Atrophy (SMA). J. Med. Chem. 61, 6501-6517 (2018).
38. Esteva, A., et al. A guide to deep learning in healthcare. Nat. Med. 25, 24-29 (2019).
39. Ozaki, K., et al. Functional SNPs in the lymphotoxin-alpha gene that are associated with susceptibility to myocardial infarction. Nature genetics 32, 650-654 (2002).
40. Golub, T. R., et al. Molecular classification of cancer: class discovery and class prediction by gene expression monitoring. Science 286, 531-537 (1999).
41. Kelley, D. R., Snoek, J. & Rinn, J. L. Basset: learning the regulatory code of the accessible genome with deep convolutional neural networks. Genome Res. 26, 990-999 (2016).
42. Chiriboga, C. A., et al. Results from a phase 1 study of nusinersen (ISIS-SMN(Rx)) in children with spinal muscular atrophy. Neurology 86, 890-897 (2016).
43. Cheung, A. K., et al. Discovery of Small Molecule Splicing Modulators of Survival Motor Neuron-2 (SMN2) for the Treatment of Spinal Muscular Atrophy (SMA). J. Med. Chem. 61, 11021-11036 (2018).
44. Slaugenhaupt, S. A., et al. Rescue of a human mRNA splicing defect by the plant cytokinin kinetin. Hum. Mol. Genet. 13, 429-436 (2004).
45. Yoshida, M., et al. Rectifier of aberrant mRNA splicing recovers tRNA modification in familial dysautonomia. Proc. Natl. Acad. Sci. U.S.A. 112, 2764-2769 (2015).
46. Hims, M. M., et al. Therapeutic potential and mechanism of kinetin as a treatment for the human splicing disease familial dysautonomia. J. Mol. Med. (Berl.) 85, 149-161 (2007).
47. Axelrod, F. B., et al. Kinetin improves IKBKAP mRNA splicing in patients with familial dysautonomia. Pediatr. Res. 70, 480-483 (2011).
48. Gold-von Simson, G., et al. Kinetin in familial dysautonomia carriers: implications for a new therapeutic strategy targeting mRNA splicing. Pediatr. Res. 65, 341-346 (2009).
49. Salani, M., et al. Development of a Screening Platform to Identify Small Molecules That Modify ELP1 Pre-mRNA Splicing in Familial Dysautonomia. SLAS Discov, 2472555218792264 (2018).
50. Morini, E., et al. ELP1 Splicing Correction Reverses Proprioceptive Sensory Loss in Familial Dysautonomia. Am. J. Hum. Genet. (2019).
51. Hims, M. M., et al. A humanized IKBKAP transgenic mouse models a tissue-specific human splicing defect. Genomics 90, 389-396 (2007).
52. Wang, E. T., et al. Alternative isoform regulation in human tissue transcriptomes. Nature 456, 470-476 (2008).
53. Sakuma, M., Iida, K. & Hagiwara, M. Deciphering targeting rules of splicing modulator compounds: case of TG003. BMC Mol. Biol. 16, 16 (2015).
54. Chiara, M. D., et al. Identification of proteins that interact with exon sequences, splice sites, and the branchpoint sequence during each stage of spliceosome assembly. Mol. Cell. Biol. 16, 3317-3326 (1996).
55. Yeo, G. & Burge, C. B. Maximum entropy modeling of short sequence motifs with applications to RNA splicing signals. J. Comput. Biol. 11, 377-394 (2004).
56. Bowden, K. L., et al. Lysosomal acid lipase deficiency impairs regulation of ABCA1 gene and formation of high density lipoproteins in cholesteryl ester storage disease. J. Biol. Chem. 286, 30624-30635 (2011).
57. Reiner, Z., et al. Lysosomal acid lipase deficiency—an under-recognized cause of dyslipidaemia and liver dysfunction. Atherosclerosis 235, 21-30 (2014).
58. Saito, S., Ohno, K., Suzuki, T. & Sakuraba, H. Structural bases of Wolman disease and cholesteryl ester storage disease. Mol. Genet. Metab. 105, 244-248 (2012).
59. Zhang, B. & Porto, A. F. Cholesteryl ester storage disease: protean presentations of lysosomal acid lipase deficiency. J. Pediatr. Gastroenterol. Nutr. 56, 682-685 (2013).
60. Aslanidis, C., et al. Genetic and biochemical evidence that CESD and Wolman disease are distinguished by residual lysosomal acid lipase activity. Genomics 33, 85-93 (1996).
61. Scott, S. A., et al. Frequency of the cholesteryl ester storage disease common LIPA E8SJM mutation (c.894G>A) in various racial and ethnic groups. Hepatology 58, 958-965 (2013).
62. Sosnay, P. R., et al. Defining the disease liability of variants in the cystic fibrosis transmembrane conductance regulator gene. Nat. Genet. 45, 1160-1167 (2013).
63. Masvidal, L., et al. Assessing the residual CFTR gene expression in human nasal epithelium cells bearing CFTR splicing mutations causing cystic fibrosis. Eur. J. Hum. Genet. 22, 784-791 (2014).
64. Sharma, N., et al. Experimental assessment of splicing variants using expression minigenes and comparison with in silico predictions. Hum. Mutat. 35, 1249-1259 (2014).
65. Lee, M., et al. Systematic Computational Identification of Variants That Activate Exonic and Intronic Cryptic Splice Sites. Am. J. Hum. Genet. 100, 751-765 (2017).
66. Sharma, N., et al. Capitalizing on the heterogeneous effects of CFTR nonsense and frameshift variants to inform therapeutic strategy for cystic fibrosis. PLoS Genet 14, e1007723 (2018).
67. Pande, M., et al. Cancer spectrum in DNA mismatch repair gene mutation carriers: results from a hospital based Lynch syndrome registry. Fam. Cancer 11, 441-447 (2012).
68. Hutton, M., et al. Association of missense and 5′-splice-site mutations in tau with the inherited dementia FTDP-17. Nature 393, 702-705 (1998).
69. Spillantini, M. G., et al. Mutation in the tau gene in familial multiple system tauopathy with presenile dementia. Proc. Natl. Acad. Sci. U.S.A. 95, 7737-7741 (1998).
70. Hong, M., et al. Mutation-specific functional impairments in distinct tau isoforms of hereditary FTDP-17. Science 282, 1914-1917 (1998).
71. Connell, J. W., et al. Quantitative analysis of tau isoform transcripts in sporadic tauopathies. Brain Res. Mol. Brain Res. 137, 104-109 (2005).
72. Neumann, M., et al. A new family with frontotemporal dementia with intronic 10+3 splice site mutation in the tau gene: neuropathology and molecular effects. Neuropathol. Appl. Neurobiol. 31, 362-373 (2005).
73. McCague, A. F., et al. Correlating Cystic Fibrosis Transmembrane Conductance Regulator Function with Clinical Features to Inform Precision Treatment of Cystic Fibrosis. Am. J. Respir. Crit. Care Med. 199, 1116-1126 (2019).
74. Ramsey, B. W., et al. A CFTR potentiator in patients with cystic fibrosis and the G551D mutation. N. Engl. J. Med. 365, 1663-1672 (2011).
75. Keating, D., et al. VX-445-Tezacaftor-Ivacaftor in Patients with Cystic Fibrosis and One or Two Phe508del Alleles. N. Engl. J. Med. 379, 1612-1620 (2018).
76. Wainwright, C. E., et al. Lumacaftor-Ivacaftor in Patients with Cystic Fibrosis Homozygous for Phe508del CFTR. N. Engl. J. Med. 373, 220-231 (2015).
77. Taylor-Cousar, J. L., et al. Tezacaftor-Ivacaftor in Patients with Cystic Fibrosis Homozygous for Phe508del. N. Engl. J. Med. 377, 2013-2023 (2017).
78. Davies, J. C., et al. VX-659-Tezacaftor-Ivacaftor in Patients with Cystic Fibrosis and One or Two Phe508del Alleles. N. Engl. J. Med. 379, 1599-1611 (2018).
79. Heaney, D. L., Flume, P., Hamilton, L., Lyon, E. & Wolff, D. J. Detection of an apparent homozygous 3120G>A cystic fibrosis mutation on a routine carrier screen. J. Mol. Diagn. 8, 137-140 (2006).
80. Ibrahim, E. C., et al. Weak definition of IKBKAP exon 20 leads to aberrant splicing in familial dysautonomia. Hum. Mutat. 28, 41-53 (2007).
81. Mitchell, R. J., Farrington, S. M., Dunlop, M. G. & Campbell, H. Mismatch repair genes hMEH and hMSH2 and colorectal cancer: a HuGE review. Am. J. Epidemiol. 156, 885-902 (2002).
82. Gottschalk, L. B., et al. Creation and characterization of an airway epithelial cell line for stable expression of CFTR variants. J Cyst Fibros 15, 285-294 (2016).
83. Raraigh, K. S., et al. Functional Assays Are Essential for Interpretation of Missense Variants Associated with Variable Expressivity. Am J Hum Genet (2018).
84. Bailey, T. L. DREME: motif discovery in transcription factor ChIP-seq data. Bioinformatics 27, 1653-1659 (2011).
85. Collins, R. L., et al. A structural variation reference for medical and population genetics. Nature 581, 444-451 (2020).
86. Karczewski, K. J., et al. The mutational constraint spectrum quantified from variation in 141,456 humans. Nature 581, 434-443 (2020).
87. Dowty, J. G., et al. Cancer risks for MLH1 and MSH2 mutation carriers. Hum. Mutat. 34, 490-497 (2013).
88. Dachsel, J. C., et al. Lrrk2 G2019S substitution in frontotemporal lobar degeneration with ubiquitin-immunoreactive neuronal inclusions. Acta Neuropathol. 113, 601-606 (2007).
89. Whitwell, J. L., et al. Atrophy patterns in IVS10+16, IVS10+3, N279K, S305N, P301L, and V337M MAPT mutations. Neurology 73, 1058-1065 (2009).
90. Jiang, L., et al. Synthetic spike-in standards for RNA-seq experiments. Genome Res. 21, 1543-1551 (2011).
91. Dobin, A., et al. STAR: ultrafast universal RNA-seq aligner. Bioinformatics 29, 15-21 (2013).
92. Yeo, G. & Burge, C. B. Maximum entropy modeling of short sequence motifs with applications to RNA splicing signals. J. Comput. Biol. 11, 377-394 (2004).
93. Morini, E., et al. ELP1 Splicing Correction Reverses Proprioceptive Sensory Loss in Familial Dysautonomia. Am J. Human Genetics. 104(4), 638-650 (April 2019).
94. CNN Model Source Code on GitHub: https://github.com/talkowski-lab/SMC_CNN_Model

What is claimed is:

1. A compound for use in a method to modulate the production of one or more mature RNA isoforms from a gene transcript in a cell, wherein the method comprises, contacting the cell with the compound, and wherein the compound is 2-chloro-N-(pyridin-4-ylmethyl)-7H-pyrrolo[2,3-d]pyrimidin-4-amine, having the formula of Compound (I):

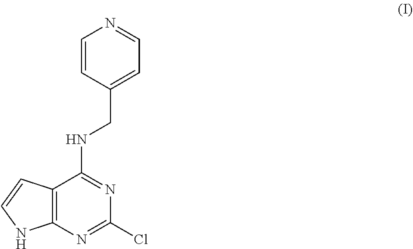 [SMILES: Clc1nc(NCc2ccncc2)c2cc[nH]c2n1]

2. The compound for use in the method of claim 1, wherein the gene transcript is a wildtype gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagt (SEQ ID NO: 1), GATTAAgtgggt (SEQ ID NO: 2), CACTAGgtgaga (SEQ ID NO: 3), and CCAgtgagga (SEQ ID NO: 4).

3. The compound for use in the method of claim 1, wherein the 5′ splice site of Exon 2 comprises, a sequence selected from the group consisting of: CAAguaagu (SEQ ID NO: 5), GAUUAAgugggu (SEQ ID NO: 6), CACUAGgugaga (SEQ ID NO: 7), and CCAgugagga (SEQ ID NO: 8).

4. The compound for use in the method of claim 1, wherein the gene transcript is a mutant gene transcript comprising, three exons and two introns operably linked in the following order: Exon 1, Intron 1, Exon 2, Intron 2, and Exon 3, and wherein a 5′ splice site of Exon 2 comprises, a sequence selected from the group consisting of: CAAgtaagc (SEQ ID NO: 9), GATTAAgtaggt (SEQ ID NO: 10), CACTAGgtgagc (SEQ ID NO: 11), CCAgttagga (SEQ ID NO: 12), AGCCAAgtatgt (SEQ ID NO: 13), ATCCAAgtatgt (SEQ ID NO: 14), CTGAAgtcagt (SEQ ID NO: 15), and AGTgtaagta (SEQ ID NO: 16).

5. The compound for use in the method of claim 1, wherein the 5′ splice site of Exon 2 comprises, a sequence selected from the group consisting of: CAAguaagc (SEQ ID NO: 17), GAUUAAguaggu (SEQ ID NO: 18), CACUAGgugagc (SEQ ID NO: 19), CCAguuagga (SEQ ID NO: 20), AGCCAAguaugu (SEQ ID NO: 21), AUCCAAguaugu (SEQ ID NO: 22), CUGAAgucagu (SEQ ID NO: 23), and AGUguaagua (SEQ ID NO: 24).

6. The compound for use in the method of claim 1, wherein the gene transcript is a predicted wildtype or mutant gene transcript in a cell comprising, a nucleotide sequence selected from the group consisting of: CTTAG (SEQ ID NO: 25), NNAGC (SEQ ID NO: 26), NCAGA (SEQ ID NO: 27), CCCTT (SEQ ID NO: 28), TNNCT (SEQ ID NO: 29), GCAGA (SEQ ID NO: 30), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), ATGGN (SEQ ID NO: 36), TCTCA (SEQ ID NO: 37), AAGCT (SEQ ID NO: 38), GTCAT (SEQ ID NO: 39), GAGAG (SEQ ID NO: 40), AGGAN (SEQ ID NO: 41), AGACC (SEQ ID NO: 42), AAGGT (SEQ ID NO: 43), GATTA (SEQ ID NO: 44), TCTTT (SEQ ID NO: 45), NNAGN (SEQ ID NO: 46), TCGTG (SEQ ID NO: 47), ANGAA (SEQ ID NO: 48), TTAAA (SEQ ID NO: 49), AGACT (SEQ ID NO: 50), GGGTG (SEQ ID NO: 51), TTCCC (SEQ ID NO: 52), AGTNA (SEQ ID NO: 53), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), NTCCT (SEQ ID NO: 60), AACCT (SEQ ID NO: 61), ACTCN (SEQ ID NO: 62), and CTGTA (SEQ ID NO: 63), and wherein N represents any DNA nucleotide.

7. The compound for use in the method of claim 1, wherein the gene transcript is a predicted wildtype or mutant gene transcript in a cell comprising, a nucleotide sequence selected from the group consisting of: NNAGC (SEQ ID NO: 26), NCAGA (SEQ ID NO: 27), CCCTT (SEQ ID NO: 28), TNNCT (SEQ ID NO: 29), TNTGA (SEQ ID NO: 31), TGAGC (SEQ ID NO: 32), CTCTC (SEQ ID NO: 33), TNNNT (SEQ ID NO: 34), TTTTT (SEQ ID NO: 35), AAGCT (SEQ ID NO: 38), GTCAT (SEQ ID NO: 39), GAGAG (SEQ ID NO: 40), AGACC (SEQ ID NO: 42), AAGGT (SEQ ID NO: 43), GATTA (SEQ ID NO: 44), NNAGN (SEQ ID NO: 46), TCGTG (SEQ ID NO: 47), ANGAA (SEQ ID NO: 48), TTAAA (SEQ ID NO: 49), GGGTG (SEQ ID NO: 51), TTCCC (SEQ ID NO: 52), TATGT (SEQ ID NO: 54), GGGAA (SEQ ID NO: 55), NTGNN (SEQ ID NO: 56), NTCCC (SEQ ID NO: 57), CAGGC (SEQ ID NO: 58), NTGTN (SEQ ID NO: 59), AACCT (SEQ ID NO: 61), and CTGTA (SEQ ID NO: 63), and wherein N represents any DNA nucleotide.

8. The compound for use in the method of claim 1, wherein the gene transcript is a predicted wildtype or mutant gene transcript in a cell comprising, a nucleotide sequence selected from the group consisting of: CUUAG (SEQ ID NO: 64), NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AUGGN (SEQ ID NO: 75), UCUCA (SEQ ID NO: 76), AAGCU (SEQ ID NO: 77), GUCAU (SEQ ID NO: 78), AGGAN (SEQ ID NO: 80), AAGGU (SEQ ID NO: 82), GAUUA (SEQ ID NO: 83), UCUUU (SEQ ID NO: 84), NNAGN (SEQ ID NO: 85), UCGUG (SEQ ID NO: 86), ANGAA (SEQ ID NO: 87), UUAAA (SEQ ID NO: 88), AGACU (SEQ ID NO: 89), GGGUG (SEQ ID NO: 90), UUCCC (SEQ ID NO: 91), AGUNA (SEQ ID NO: 92), UAUGU (SEQ ID NO: 93), NUGNN (SEQ ID NO: 95), NUCCC (SEQ ID NO: 96), NUGUN (SEQ ID NO: 98), NUCCU (SEQ ID NO: 99), AACCU (SEQ ID NO: 100), ACUCN (SEQ ID NO: 101), and CUGUA (SEQ ID NO: 102), and wherein N represents any RNA nucleotide.

9. The compound for use in the method of claim 1, wherein the gene transcript is a predicted wildtype or mutant gene transcript in a cell comprising, a nucleotide sequence selected from the group consisting of: NNAGC (SEQ ID NO: 65), NCAGA (SEQ ID NO: 66), CCCUU (SEQ ID NO: 67), UNNCU (SEQ ID NO: 68), UNUGA (SEQ ID NO: 70), UGAGC (SEQ ID NO: 71), CUCUC (SEQ ID NO: 72), UNNNU (SEQ ID NO: 73), UUUUU (SEQ ID NO: 74), AAGCU (SEQ ID NO: 77), GUCAU (SEQ ID NO: 78), AAGGU (SEQ ID NO: 82), GAUUA (SEQ ID NO: 83), NNAGN (SEQ ID NO: 85), UCGUG (SEQ ID NO: 86), ANGAA (SEQ ID NO: 87), UUAAA (SEQ ID NO: 88), GGGUG (SEQ ID NO: 90), UUCCC (SEQ ID NO: 91), UAUGU (SEQ ID NO: 93), NUGNN (SEQ ID NO: 95), NUCCC (SEQ ID NO: 96), NUGUN (SEQ ID NO: 98), AACCU (SEQ ID NO: 100), and CUGUA (SEQ ID NO: 102), and wherein N represents any RNA nucleotide.

10. The compound for use in the method of claim 1, wherein Compound (I) modulates exon inclusion or exon exclusion to produce one or more mature RNA isoforms from a gene transcript in a cell, and wherein the method comprises contacting the cell with Compound (I).

11. The compound for use in the method of claim 1, wherein Compound (I) increases exon inclusion to produce one or more mature RNA isoforms from a gene transcript in a cell, and wherein the method comprises contacting the cell with Compound (I).

12. The compound for use in the method of claim 1, wherein Compound (I) increases exon exclusion to produce one or more mature RNA isoforms from a gene transcript in a cell, and wherein the method comprises contacting the cell with Compound (I).

13. The compound for use in the method of claim 1, wherein Compound (I) modulates exon inclusion or exon exclusion to produce one or more mature RNA isoforms from a gene transcript in a cell, and wherein the method comprises contacting the cell in vivo or in vitro with Compound (I).

14. The compound for use in the method of claim 1, wherein the method treats a disease associated with or mediated by a mutation in a gene transcript in a subject in need thereof comprising, administering Compound (I) to the subject, wherein exon inclusion or exon exclusion is modulated in one or more mature RNA isoforms produced from the gene transcript, and wherein the subject is human.

15. The compound for use in the method of claim 1, wherein the method treats a disease associated with or mediated by a mutation in a wildtype gene transcript in a subject in need thereof comprising, administering Compound (I) to the subject, wherein exon inclusion or exon exclusion is modulated in one or more mature RNA isoforms produced from the gene transcript, and wherein one or more of the mature RNA isoforms produce a functional protein.

16. A pharmaceutical composition comprising a compound and a pharmaceutically acceptable carrier, excipient, or diluent for use in a method of treating a disease associated with or mediated by a mutation in a gene transcript in a subject in need thereof, wherein the compound is 2-chloro-N-(pyridin-4-ylmethyl)-7H-pyrrolo[2,3-d]pyrimidin-4-amine having the formula of Compound (I): 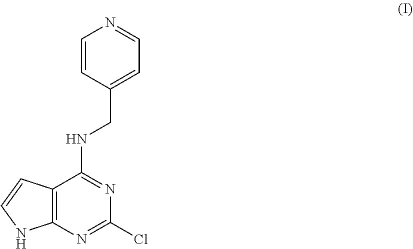 [SMILES: Clc1nc(NCc2ccncc2)c2cc[nH]c2n1]
wherein exon inclusion or exon exclusion is modulated in one or more mature RNA isoforms produced from a gene transcript in a cell.
